(19) United States
(12) Patent Application Publication
(10) Pub. No.: US 2022/0356169 A1
(43) Pub. Date: Nov. 10, 2022

(54) TETRAZINES FOR HIGH CLICK RELEASE SPEED AND YIELD

(71) Applicant: Tagworks Pharmaceuticals B.V., Nijmegen (NL)

(72) Inventors: Marc Stefan ROBILLARD, Eindhoven (NL); Freek Johannes Maria HOEBEN, Eindhoven (NL); Raffaella ROSSIN, Eindhoven (NL); Ronny Mathieu VERSTEEGEN, Eindhoven (NL); Henricus Marie JANSSEN, Nijmegen (NL)

(73) Assignee: Tagworks Pharmaceuticals B.V., Nijmegen (NL)

(21) Appl. No.: 17/619,794

(22) PCT Filed: Jun. 17, 2020
(86) PCT No.: PCT/NL2020/050386

§ 371 Date: Dec. 16, 2021

(30) Foreign Application Priority Data
Jun. 17, 2019 (EP) 19180678.5

(51) Int. Cl. C07D 401/14; A61K 47/68; C07D 403/14

(52) U.S. Cl. CPC C07D 401/14; A61K 47/6889; A61K 47/6817; C07D 403/14

(57) ABSTRACT

Disclosed herein are tetrazines substituted with groups that result in a high click conjugation yield and high click release yields. In some of several other aspects, the invention relates to combinations and kits having the tetrazines and a dienophile, preferably a trans-cyclooctene. In another aspect, the compounds, combinations, and kits are for use as a medicament.

### FIELD OF THE INVENTION

The invention disclosed herein relates to tetrazines for high click release speed and yield.

### BACKGROUND OF THE INVENTION

Selective chemical reactions that are orthogonal to the diverse functionalities of biological systems are called bio-orthogonal reactions and occur between two abiotic groups with exclusive mutual reactivity. These can be used to selectively modify biochemical structures, such as proteins or nucleic acids, which typically proceed in water and at near-ambient temperature, and may be applied in complex chemical environments, such as those found in living organisms.

Bio-orthogonal reactions are broadly useful tools with applications that span chemical synthesis, materials science, chemical biology, diagnostics, and medicine. Especially prominent application areas for bioorthogonal reactions include drug delivery agents and prodrugs for pharmaceutical applications, as well as various reversible bioconjugates and sophisticated spectroscopic probes for applications in the field of biological analysis.

One prominent bioorthogonal reaction is the inverse-electron-demand Diels Alder (IEDDA) reaction between a trans-cyclooctene (TCO) and a tetrazine (TZ). In previous studies the IEDDA reaction was used for pretargeted radioimmunoimaging, treating tumor-bearing mice with trans-cyclooctene (TCO)-tagged antibody or antibody fragments, followed one or more days later by administration and selective conjugation of a radiolabeled tetrazine probe to the TCO tag of the tumor-bound antibody [R. Rossin, M. S. Robillard, Curr. Opin. Chem. Biol. 2014, 21, 161-169].

Based on the IEDDA conjugation a release reaction has been developed, which was termed the IEDDA pyridazine elimination, a “click-to-release” approach that affords instantaneous and selective release of a Construct upon conjugation [R. M. Versteegen, R. Rossin, W. ten Hoeve, H. M. Janssen, M. S. Robillard, Angew. Chem. Int. Ed. 2013, 52, 14112-14116]. IEDDA reactions between tetrazines (i.e. diene) and alkenes (i.e. dienophile) afford 4,5-dihydropyridazines, which usually tautomerize to 1,4- and 2,5-dihydropyridazines. It was demonstrated that the 1,4-dihydropyridazine product derived from a TCO containing a carbamate-linked doxorubicin (Dox) at the allylic position and tetrazine is prone to eliminate CO₂ and Dox via an electron cascade mechanism eventually affording aromatic pyridazine. The triggered Construct release has been demonstrated in PBS (phosphate buffered saline), serum, cell culture and in mice and holds promise for a range of applications in medicine, molecular diagnostics, chemical biology, material sciences, and synthetic chemistry, including triggered drug release, biomolecule uncaging and capture&release strategies.

In general the IEDDA pyridazine elimination enables the controlled manipulation of a wide range of substrates in relatively complex environments, in the presence of a range of other chemical functional groups. This control can be temporal and, optionally, also spatial. The manipulation can be versatile, e.g. for a variety of purposes including but not limited to activating, deactivating, releasing, trapping, or otherwise altering a Construct attached to a chemically cleavable group.

The IEDDA pyridazine elimination has been applied in triggered drug (i.e. Construct) release from antibody-drug conjugates (ADCs) capable of participating in an IEDDA reaction (FIG. 1). ADCs are a promising class of biopharmaceuticals that combine the target-specificity of monoclonal antibodies (mAbs) or mAb fragments with the potency of small molecule toxins. Classical ADCs are designed to bind to an internalizing cancer cell receptor leading to uptake of the ADC and subsequent intracellular release of the drug by enzymes, thiols, or lysosomal pH. Routing the toxin to the tumor, while minimizing the peripheral damage to healthy tissue, allows the use of highly potent drugs resulting in improved therapeutic outcomes. The use of the IEDDA pyridazine elimination for ADC activation allows the targeting of non-internalizing receptors, as the drug is cleaved chemically instead of biologically.

In general, prodrugs, which may comprise ADCs, are an interesting application for the IEDDA pyridazine elimination reaction, in which a drug is deactivated, bound or masked by a moiety, and is reactivated, released or unmasked after an IEDDA reaction has taken place.

Background art for the aforementioned IEDDA pyridazine elimination technology further includes WO2012/156919, WO2012156918A1, WO 2014/081303, and US20150297741. Herein a dienophile, the aforementioned TCO, is used as a chemically cleavable group in e.g. a protecting group in synthetic chemistry, a cleavable linker or mask in chemical biology, and in vitro diagnostics. The group is attached to a Construct (e.g. molecule, protein, peptide, polymer, dye, surface) in such a way that the release of the dienophile from the Construct can be provoked by allowing the dienophile to react with a diene, the aforementioned TZ. The dienophile is an eight-membered non-aromatic cyclic alkene or alkenyl group, particularly a TCO group.

In some applications, the TCO is part of a prodrug which is first injected in the blood stream of a subject and may be targeted to a certain part of the body, e.g. a tumor. Then, a certain percentage of the prodrug is immobilized at the targeted spot, while another percentage is cleared by the body. After several hours or days, an activator comprising a tetrazine is added to release a drug from the prodrug, preferably only at the targeted spot. The tetrazine itself is also subject to clearance by the body at a certain clearance rate.

In general, the tetrazine reacts in an initial step with a dienophile-bound Construct (e.g. a dienophile-containing prodrug) to form a conjugate. This is referred to as the click conjugation step. Next, via one or multiple mechanisms, the Construct is preferably released from the Construct-dienophile (e.g. prodrug). It will be understood that a high yield in the click conjugation step, i.e. a high click conjugation yield, does not necessarily result in a high yield of released Construct, i.e. a high drug release yield.

From the viewpoint of bio-orthogonality the chemistry works well.

However, it is desired that better IEDDA reactions are developed.

In general, achieving high Construct release yields in IEDDA reactions remains a challenge both in vivo and in vitro. In particular, the reaction between a Construct-bearing dienophile and a tetrazine preferably results in a high Construct release yield in vitro and/or in vivo.

Typically, the tetrazine motives that typically give high release are less reactive than the tetrazines that have successfully been used for click conjugations in vivo. These more reactive tetrazines give a good click conjugation yield, but result in a poor click release yield. In the abovementioned study [R. M. Versteegen, R. Rossin, W. ten Hoeve, H. M. Janssen, M. S. Robillard, Angew. Chem. Int. Ed. 2013, 52, 14112-14116], it was shown that for example 3,6-bis-(2-pyridyl)-1,2,4,5-tetrazine gives high click conjugation rate and yield, but a very poor click release yield of only 7% in PBS/MeCN (3:1) or only 12% in serum.

In addition, for tetrazines that give a high release yield, for example 3,6-bis-methyl-1,2,4,5-tetrazine, this release typically takes several hours and typically exhibits a maximum 80-90% release yield R. M. Versteegen, R. Rossin, W. ten Hoeve, H. M. Janssen, M. S. Robillard, Angew. Chem. Int. Ed. 2013, 52, 14112-14116].

For at least that reason, it is a desire to improve the click release rate and yield of tetrazine motifs with relatively high reactivity towards dienophiles (i.e. high click conjugation yield) in vitro and/or in vivo.

Also, some tetrazines giving a good click release yield afford release within hours, not minutes or seconds. For at least that reason, it is a desire to improve the click release rate and possibly the click release yield of tetrazines with relatively good click release yields in vitro and/or in vivo.

Another desire is to provide a tetrazine that achieves a combination of a high click conjugation yield with a Construct-bearing dienophile and a high Construct release yield both in vitro and in vivo.

Yet another desire is to provide a tetrazine that achieves an increased conjugation reaction rate with a dienophile as compared to known tetrazine-dienophile reaction pairs.

Yet another desire is to provide a tetrazine that achieves an increased Construct release rate after a reaction with a Construct-bearing dienophile as compared to known tetrazine-dienophile reaction pairs.

Another desire is to provide a tetrazine that achieves a combination of a high click conjugation reaction rate with a dienophile, a high click conjugation yield between a Construct-bearing dienophile and the tetrazine and a high Construct release yield is preferred both in vitro and in vivo.

Another desire is to provide a tetrazine that is readily soluble in an aqueous solution.

Another desire is to provide a tetrazine that has a favorable clearing rate in vivo.

Yet another desire is to provide a combination of a tetrazine and a dienophile that achieves one or more of the abovementioned desires.

It is desired that compounds are developed that address one or more of the abovementioned problems and/or desires.

### SUMMARY OF THE INVENTION

In one aspect, the invention pertains to a compound according to Formula (1):

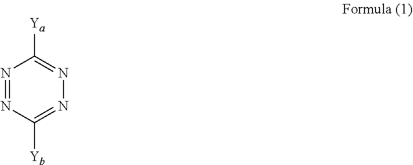

including pharmaceutically acceptable salts thereof,
wherein, Yₐ is selected from the group consisting of Y₁, Y₂, Y₃, Y₄, Y₅ and Y₆:

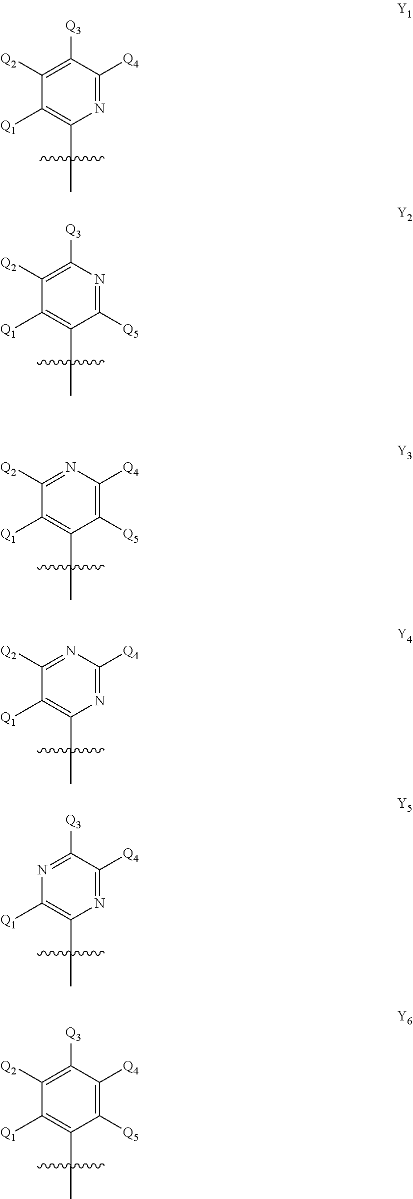

wherein, Y_{b} is selected from the group consisting of Y₁, Y₂, Y₃, Y₄, Y₅, Y₆, hydrogen, R₃, and a —(S^{P})_{D}—R₈₇; wherein S^{P} is a spacer and D is 0 or 1;
wherein when Yₐ is Y₆, then Y_{b} is hydrogen,
wherein each Q₁ and Q₅, are individually selected from the group consisting of R₁, hydrogen, R₃ and —(S^{P})_{D}—R₈₇;
wherein each Q₂ and Q₄, are individually selected from the group consisting of R₂, hydrogen, R₃, and —(S^{P})_{D}—R₈₇;
wherein each Q₃ is individually selected from the group consisting of hydrogen, R₃, and —(S^{P})_{D}—R₈₇;
wherein, the compound of Formula (1) comprises at least one R₁, and at least one R₈₇;
wherein each R₈₇ is individually selected from the group consisting of biomolecule, polymer, peptide, peptoid, dendrimer, protein, carbohydrate, oligonucleotide, oligosaccharide, lipid, micelle, liposomes, polymersome, nanoparticle, microparticle, bead, gel, resin, metal complex, organometallic moiety, organic compound, albumin-binding moiety, dye moiety, fluorescent moiety, radionuclide-containing moiety and imaging probe;
wherein each R₁ individually is selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂;
wherein each R₂ individually is selected from the group consisting of N(X₅₀)₂, C(X₅₁)₂N(X₅₀)₂, NX₅₀C(O)X₅₁, NX₅₀C(S)X₅₁—OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂;
wherein each X₅₀ and X₅₁ individually is selected from the group consisting of hydrogen, R₆, and —(S^{P})_{D}—R₈₇;
wherein each R₆ is preferably independently selected from the group consisting of hydrogen, C₁-C₄ alkyl groups, C₂-C₄ alkenyl groups, and C₄₋₆ (hetero)aryl groups;
wherein for R₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂, and —NO₂; and optionally contain at most two heteroatoms selected from the group consisting of —O—, —S—, —NH—, —P—, and —Si—, wherein the N, S, and P atoms are optionally oxidized, wherein each R₃ is individually selected from the group consisting of —F, —Cl, —Br, —I, —OR₇, —N(R₇)₂, —SO₃, —PO₃⁻, —NO₂, —CF₃, —SR₇, —S(═O)₂N(R₇)₂, OC(═O)R₇, SC(═O)R₇, OC(═S)R₇, SC(═S)R₇, NR₇C(═O)—R₇, NR₇C(═S)—R₇, NR₇C(═O)O—R₇, NR₇C(═S)O—R₇, NR₇C(═O)S—R₇, NR₇C(═S)S—R₇, OC(═O)N(R₇)₂, SC(═O)N(R₇)₂, OC(═S)N(R₇)₂, SC(═S)N(R₇)₂, NR₇C(═O)N(R₇)₂, NR₇C(═S)N(R₇)₂, C(═O)R₇, C(═S)R₇, C(═O)N(R₇)₂, C(═S)N(R₇)₂, C(═O)O—R₇, C(═O)S—R₇, C(═S)O—R₇, C(═S)S—R₇, —S(O)R₇, —S(O)₂R₇, NR₇S(O)₂R₇, —ON(R₇)₂, —NR₇OR₇, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₇, —N(R₇)₂, —SO₃R₇, —PO₃(R₇)₂, —PO₄(R₇)₂, —NO₂, —CF₃, ═O, ═NR₇, and —SR₇, and optionally contain one or more heteroatoms selected from the group consisting of O, S, NR₇, P, and Si, wherein the N, S, and P atoms are optionally oxidized, wherein the N atoms are optionally quaternized;
wherein preferably R₇ is individually selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

In yet another aspect, the invention pertains to a combination comprising the compound according to the invention, and a dienophile.

In yet another aspect, the invention relates to a kit comprising the combination according to the invention.

In yet another aspect, the invention relates to a combination as defined herein, or the kit as defined herein for use in the treatment of a subject, wherein said subject is preferably a human.

In yet another aspect, the invention relates to the use of a compound according to the invention, or a combination according to the invention, or the kit according to the invention in a bioorthogonal reaction.

In yet another aspect, the invention relates to an in vitro method for releasing a moiety from a dienophile, said in vitro method comprising the step of contacting a compound according to the invention, with a dienophile as defined herein.

### BRIEF DESCRIPTION OF THE FIGURES

FIG. 1 depicts a preferred embodiment of this invention. In both panels an ADC is administered to a cancer patient, and is allowed to circulate and bind to a target on the cancer cell. After the freely circulating ADC has sufficiently cleared from circulation, for example after 2 days post injection, the Activator, is administered and distributes systemically, allowing the reaction with the Trigger of cancer-bound Prodrug or ADC, releasing the Drug, after which the Drug can penetrate and kill neighbouring cancer cells. Panel A depicts the cleavage of a carbamate-linked Drug and Panel B depicts the cleavage of an ether-linked Drug. For sake of clarity R₈₇ is omitted from the tetrazine structure.

FIG. 2 depicts a preferred embodiment of this invention. An antibody construct comprising a bi-specific (anti-tumor and anti-CD3) antibody and a masking moiety (blocking protein) is administered to a cancer patient, and is allowed to circulate and bind to a target on the cancer cell. After the freely circulating construct has sufficiently cleared from circulation, for example after 2 days post injection, the Activator, is administered and distributes systemically, allowing the reaction with the Trigger of cancer-bound Prodrug, releasing the mask, after which T-cells bind the bi-specific antibody resulting in tumor killing. For sake of clarity R₈₇ is omitted from the tetrazine structure.

FIG. 3 depicts the in vivo assembly of a functional cell penetration peptide (CPP) at the target site, leading to triggered CPP-induced drug internalization.

FIG. 4 depicts the in vivo unmasking of a functional cell penetration peptide (CPP) at the target site, leading to triggered CPP-induced drug internalization. For sake of clarity R₈₇ is omitted from the tetrazine structure.

FIG. 5 depicts the use of the compounds of this invention for the site specific antibody conjugation with e.g. a drug, for ADC production. For sake of clarity R₈₇ is omitted from the tetrazine structure.

FIG. 6 depicts the results of (A) an in vivo reactivity study between diabody antibody-drug conjugate (ADC) and various Activators in mice bearing colon cancer xenograft (tumor activation study) and (B) the concentrations of released drug (MMAE) achieved in tumors upon reaction between Trigger and Activator in vivo.

FIG. 7 depicts the results of a therapy study (A: mean tumor sizes with SEM; B: survival curves; C relative body weights) in mice bearing colon cancer xenografts treated with diabody ADC followed by one Activator of this invention (4 cycles in two weeks) and controls.

### DETAILED DESCRIPTION OF THE INVENTION

The invention, in a broad sense, is based on the judicious insight that a compound according to Formula (1) as described herein meets one or more of the abovementioned desires and/or resolves one or more of the abovementioned problems.

In another aspect, it is found that a combination of a compound according to Formula (1) as described herein and a dienophile as defined herein meets one or more of the abovementioned desires and/or resolves one or more of the abovementioned problems.

In yet another aspect, it is found that a kit comprising a combination according to the invention meets one or more of the abovementioned desires and/or resolves one or more of the abovementioned problems.

In a further aspect still, it is found that a combination according to the invention, and a kit according to the invention is useful in the treatment of patients.

In yet a further aspect, it is found that a compound according to the invention, a combination according to the invention and a kit according to the invention are useful in bioorthogonal reactions in vitro and/or in vivo.

Without wishing to be bound by theory, the inventors believe that the presence of the R₁ or R₂ groups as defined herein in the structures according to Formula (1) results in a higher click release yield and/or click release rate when contacted with a dienophile carrying a releasable Construct, as compared to known tetrazines, in particular as compared to the same tetrazines lacking the said R₁ or R₂ group. Still without wishing to be bound by theory, the inventors currently believe that this is the result of a destabilizing effect on the dihydropyridazine tautomer intermediates, in particular the 4,5- and/or the 1,4- and the 2,5-dihydropyridazine tautomer intermediate that is formed upon conjugation of the tetrazine to a dienophile, in particular an eight-membered non-aromatic cyclic alkene.

With reference to an example in Scheme 3A and without wishing to be bound to theory, the inventors believe that upon formation of the 4,5-dihydropyridazine tautomer 3a, the hydroxyl moiety R₁ induces the protonation of the neighbouring pyridazine nitrogen, and thereby catalyzes the tuatomerization to specifically the 1,4-dihydropyridazine moiety 4a, which then eliminates C^{A}. The inventors also believe that the R₁ moiety can accelerate the elimination from 4a.

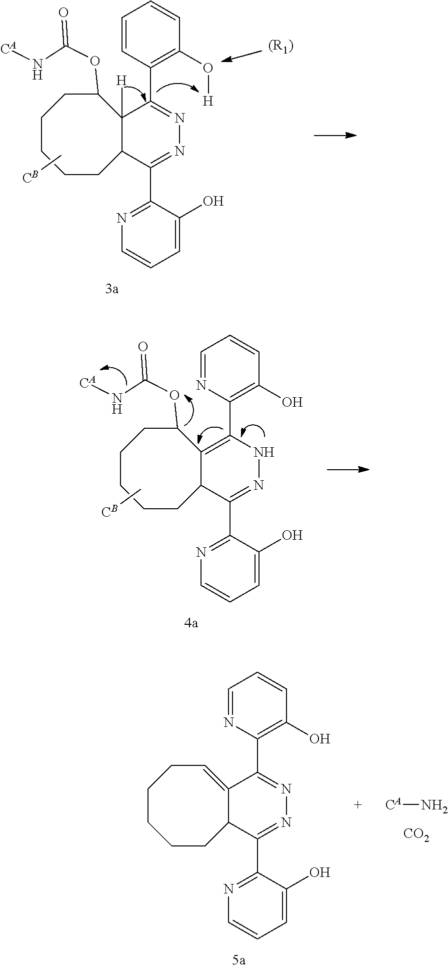

Scheme 3A. Proposed IEDDA pyridazine elimination mechanism of this invention. For reasons of clarity, the moiety R⁸⁷ is not shown.

In another aspect, it was found that compounds according to Formula (1) as described herein give high click conjugation yields when being contacted with a dienophile, in particular an eight-membered non-aromatic cyclic alkene, more particularly a trans-cyclooctene.

### Definitions

The present invention will further be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where an indefinite or definite article is used when referring to a singular noun e.g. “a” or “an”, “the”, this includes a plural of that noun unless something else is specifically stated.

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

Thus, the scope of the expression “a device comprising means A and B” should not be limited to devices consisting only of components A and B. It means that with respect to the present invention, the only relevant components of the device are A and B.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer, unless stated otherwise. When the structure of a compound is depicted as a specific diastereomer, it is to be understood that the invention of the present application is not limited to that specific diastereomer, unless stated otherwise.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer, unless stated otherwise.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer, unless stated otherwise.

Unless stated otherwise, the compounds of the invention and/or groups thereof may be protonated or deprotonated. It will be understood that it is possible that a compound may bear multiple charges which may be of opposite sign. For example, in a compound containing an amine and a carboxylic acid, the amine may be protonated while simultaneously the carboxylic acid is deprotonated.

In several formulae, groups or substituents are indicated with reference to letters such as “A”, “B”, “X”, “Y”, and various (numbered) “R” groups. In addition, the number of repeating units may be referred to with a letter, e.g. n in —(CH₂)ₙ—. The definitions of these letters are to be read with reference to each formula, i.e. in different formulae these letters, each independently, can have different meanings unless indicated otherwise.

In several chemical formulae and texts below reference is made to “alkyl”, “heteroalkyl”, “aryl”, “heteroaryl”, “alkenyl”, “alkynyl”, “alkylene”, “alkenylene”, “alkynylene”, “arylene”, “cycloalkyl”, “cycloalkenyl”, “cycloakynyl”, arenetriyl, and the like. The number of carbon atoms that these groups have, excluding the carbon atoms comprised in any optional substituents as defined below, can be indicated by a designation preceding such terms (e.g. “C₁-C₈, alkyl” means that said alkyl may have from 1 to 8 carbon atoms). For the avoidance of doubt, a butyl group substituted with a —OCH₃ group is designated as a C₄ alkyl, because the carbon atom in the substituent is not included in the carbon count.

Unsubstituted alkyl groups have the general formula CₙH₂ₙ₊₁ and may be linear or branched. Optionally, the alkyl groups are substituted by one or more substituents further specified in this document. Examples of alkyl groups include methyl, ethyl, propyl, 2-propyl, t-butyl, 1-hexyl, 1-dodecyl, etc. Unless stated otherwise, an alkyl group optionally contains one or more heteroatoms independently selected from the group consisting of O, NR₅, S, P, and Si, wherein the N, S, and P atoms are optionally oxidized and the N atoms are optionally quaternized. In preferred embodiments, up to two heteroatoms may be consecutive, such as in for example —CH₂—NH—OCH₃ and —CH₂—O—Si(CH₃)₃. In some preferred embodiments the heteroatoms are not directly bound to one another. Examples of heteroalkyls include —CH₂CH₂—O—CH₃, —CH₂CH₂—NH—CH₃, —CH₂CH₂—S(O)—CH₃, —CH═CH—O—CH₃, —Si(CH₃)₃. In preferred embodiments, a C₁-C₄ alkyl contains at most 2 heteroatoms.

A cycloalkyl group is a cyclic alkyl group. Unsubstituted cycloalkyl groups comprise at least three carbon atoms and have the general formula CₙH₂ₙ₋₁. Optionally, the cycloalkyl groups are substituted by one or more substituents further specified in this document. Examples of cycloalkyl groups include cyclopropyl, cyclobutyl, cyclopentyl and cyclohexyl. Unless stated otherwise, a cycloalkyl group optionally contains one or more heteroatoms independently selected from the group consisting of O, NR₅, S, P, and Si, wherein the N, S, and P atoms are optionally oxidized and the N atoms are optionally quaternized.

An alkenyl group comprises one or more carbon-carbon double bonds, and may be linear or branched. Unsubstituted alkenyl groups comprising one C—C double bond have the general formula CₙH₂ₙ₋₁. Unsubstituted alkenyl groups comprising two C—C double bonds have the general formula CₙH₂ₙ₋₃. An alkenyl group may comprise a terminal carbon-carbon double bond and/or an internal carbon-carbon double bond. A terminal alkenyl group is an alkenyl group wherein a carbon-carbon double bond is located at a terminal position of a carbon chain. An alkenyl group may also comprise two or more carbon-carbon double bonds. Examples of an alkenyl group include ethenyl, propenyl, isopropenyl, t-butenyl, 1,3-butadienyl, 1,3-pentadienyl, etc. Unless stated otherwise, an alkenyl group may optionally be substituted with one or more, independently selected, substituents as defined below. Unless stated otherwise, an alkenyl group optionally contains one or more heteroatoms independently selected from the group consisting of O, NR₅, S, P, and Si, wherein the N, S, and P atoms are optionally oxidized and the N atoms are optionally quaternized.

An alkynyl group comprises one or more carbon-carbon triple bonds, and may be linear or branched. Unsubstituted alkynyl groups comprising one C—C triple bond have the general formula CₙH₂ₙ₋₃. An alkynyl group may comprise a terminal carbon-carbon triple bond and/or an internal carbon-carbon triple bond. A terminal alkynyl group is an alkynyl group wherein a carbon-carbon triple bond is located at a terminal position of a carbon chain. An alkynyl group may also comprise two or more carbon-carbon triple bonds. Unless stated otherwise, an alkynyl group may optionally be substituted with one or more, independently selected, substituents as defined below. Examples of an alkynyl group include ethynyl, propynyl, isopropynyl, t-butynyl, etc. Unless stated otherwise, an alkynyl group optionally contains one or more heteroatoms independently selected from the group consisting of O, NR₅, S, P, and Si, wherein the N, S, and P atoms are optionally oxidized and the N atoms are optionally quaternized.

An aryl group refers to an aromatic hydrocarbon ring system that comprises six to twenty-four carbon atoms, more preferably six to twelve carbon atoms, and may include monocyclic and polycyclic structures. When the aryl group is a polycyclic structure, it is preferably a bicyclic structure. Optionally, the aryl group may be substituted by one or more substituents further specified in this document. Examples of aryl groups are phenyl and naphthyl.

Arylalkyl groups and alkylaryl groups comprise at least seven carbon atoms and may include monocyclic and bicyclic structures. Optionally, the arylalkyl groups and alkylaryl may be substituted by one or more substituents further specified in this document. An arylalkyl group is for example benzyl. An alkylaryl group is for example 4-tert-butylphenyl.

Preferably, heteroaryl groups comprise five to sixteen carbon atoms and contain between one to five heteroatoms. Heteroaryl groups comprise at least two carbon atoms (i.e. at least C₂) and one or more heteroatoms N, O, P or S. A heteroaryl group may have a monocyclic or a bicyclic structure. Optionally, the heteroaryl group may be substituted by one or more substituents further specified in this document. Examples of suitable heteroaryl groups include pyridinyl, quinolinyl, pyrimidinyl, pyrazinyl, pyrazolyl, imidazolyl, thiazolyl, pyrrolyl, furanyl, triazolyl, benzofuranyl, indolyl, purinyl, benzoxazolyl, thienyl, phospholyl and oxazolyl.

Heteroarylalkyl groups and alkylheteroaryl groups comprise at least three carbon atoms (i.e. at least C₃) and may include monocyclic and bicyclic structures. Optionally, the heteroaryl groups may be substituted by one or more substituents further specified in this document.

Where an aryl group is denoted as a (hetero)aryl group, the notation is meant to include an aryl group and a heteroaryl group. Similarly, an alkyl(hetero)aryl group is meant to include an alkylaryl group and an alkylheteroaryl group, and (hetero)arylalkyl is meant to include an arylalkyl group and a heteroarylalkyl group. A C₂-C₂₄ (hetero)aryl group is thus to be interpreted as including a C₂-C₂₄ heteroaryl group and a C₆-C₂₄ aryl group. Similarly, a C₃-C₂₄ alkyl(hetero)aryl group is meant to include a C₇-C₂₄ alkylaryl group and a C₃-C₂₄ alkylheteroaryl group, and a C₃-C₂₄ (hetero)arylalkyl is meant to include a C₇-C₂₄ arylalkyl group and a C₃-C₂₄ heteroarylalkyl group.

A cycloalkenyl group is a cyclic alkenyl group. An unsubstituted cycloalkenyl group comprising one double bond has the general formula CₙH₂ₙ₋₃. Optionally, a cycloalkenyl group is substituted by one or more substituents further specified in this document. An example of a cycloalkenyl group is cyclopentenyl. Unless stated otherwise, a cycloalkenyl group optionally contains one or more heteroatoms independently selected from the group consisting of O, NR₅, S, P, and Si, wherein the N, S, and P atoms are optionally oxidized and the N atoms are optionally quaternized.

A cycloalkynyl group is a cyclic alkynyl group. An unsubstituted cycloalkynyl group comprising one triple bond has the general formula CₙH₂ₙ₋₅. Optionally, a cycloalkynyl group is substituted by one or more substituents further specified in this document. An example of a cycloalkynyl group is cyclooctynyl. Unless stated otherwise, a cycloalkynyl group optionally contains one or more heteroatoms independently selected from the group consisting of 0, NR₅, S, P, and Si, wherein the N, S, and P atoms are optionally oxidized and the N atoms are optionally quaternized.

When referring to a (hetero)aryl group the notation is meant to include an aryl group and a heteroaryl group. An alkyl(hetero)aryl group refers to an alkylaryl group and an alkylheteroaryl group. A (hetero)arylalkyl group refers to an arylalkyl group and a heteroarylalkyl group. In general, when (hetero) is placed before a group, it refers to both the variant of the group without the prefix hetero- as well as the group with the prefix hetero-.

Herein, the prefix hetero- denotes that the group contains one or more heteroatoms selected from the group consisting of O, N, S, P, and Si. It will be understood that groups with the prefix hetero- by definition contain heteroatoms. Hence, it will be understood that if a group with the prefix hetero- is part of a list of groups that is defined as optionally containing heteroatoms, that for the groups with the prefix hetero- it is not optional to contain heteroatoms, but is the case by definition.

Herein, it will be understood that when the prefix hetero- is used for combinations of groups, the prefix hetero- only refers to the one group before it is directly placed. For example, heteroarylalkyl denotes the combination of a heteroaryl group and an alkyl group, not the combination of a heteroaryl and a heteroalkyl group. As such, it will be understood that when the prefix hetero- is used for a combination of groups that is part of a list of groups that are indicated to optionally contain heteroatoms, it is only optional for the group within the combination without the prefix hetero- to contain a heteroatom, as it is not optional for the group within the combination with the prefix hetero- by definition (see above). For example, if heteroarylalkyl is part of a list of groups indicated to optionally contain heteroatoms, the heteroaryl part is considered to contain heteroatoms by definition, while for the alkyl part it is optional to contain heteroatoms.

Herein, the prefix cyclo- denotes that groups are cyclic. It will be understood that when the prefix cyclo- is used for combinations of groups, the prefix cyclo- only refers to the one group before it is directly placed. For example, cycloalkylalkenylene denotes the combination of a cycloalkylene group (see the definition of the suffix -ene below) and an alkenylene group, not the combination of a cycloalkylene and a cycloalkenylene group.

In general, when (cyclo) is placed before a group, it refers to both the variant of the group without the prefix cyclo- as well as the group with the prefix cyclo-.

Herein, the suffix -ene denotes divalent groups, i.e. that the group is linked to at least two other moieties. An example of an alkylene is propylene (—CH₂—CH₂—CH₂—), which is linked to another moiety at both termini. It is understood that if a group with the suffix -ene is substituted at one position with —H, then this group is identical to a group without the suffix. For example, an alkylene substituted with —H is identical to an alkyl group. I.e. propylene, —CH₂—CH₂—CH₂—, substituted with —H at one terminus, —CH₂—CH₂—CH₂—H, is logically identical to propyl, —CH₂—CH₂—CH₃.

Herein, when combinations of groups are listed with the suffix -ene, it refers to a divalent group, i.e. that the group is linked to at least two other moieties, wherein each group of the combination contains one linkage to one of these two moieties. As such, for example alkylarylene is understood as a combination of an arylene group and an alkylene group. An example of an alkylarylene group is -phenyl-CH₂—, and an example of an arylalkylene group is —CH₂-phenyl-.

Herein, the suffix -triyl denotes trivalent groups, i.e. that the group is linked to at least three other moieties. An example of an arenetriyl is depicted below:

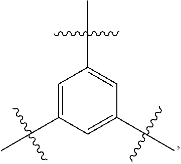 [SMILES: *c1cc(*)cc(*)c1]

wherein the wiggly lines denote bonds to different groups of the main compound.

It is understood that if a group with the suffix -triyl is substituted at one position with —H, then this group is identical to a divalent group with the suffix -ene. For example, an arenetriyl substituted with —H is identical to an arylene group. Similarly, it is understood that if a group with the suffix -triyl is substituted at two positions with —H, then this group is identical to a monovalent group. For example, an arenetriyl substituted with two —H is identical to an aryl group.

It is understood that if a group, for example an alkyl group, contains a heteroatom, then this group is identical to a hetero-variant of this group. For example, if an alkyl group contains a heteroatom, this group is identical to a heteroalkyl group. Similarly, if an aryl group contains a heteroatom, this group is identical to a heteroaryl group. It is understood that “contain” and its conjugations mean herein that when a group contains a heteroatom, this heteroatom is part of the backbone of the group. For example, a C₂ alkylene containing an N refers to —NH—CH₂—CH₂—, —CH₂—NH—CH₂—, and —CH₂—CH₂—NH—.

Unless indicated otherwise, a group may contain a heteroatom at non-terminal positions or at one or more terminal positions. In this case, “terminal” refers to the terminal position within the group, and not necessarily to the terminal position of the entire compound. For example, if an ethylene group contains a nitrogen atom, this may refer to —NH—CH₂—CH₂—, —CH₂—NH—CH₂—, and —CH₂—CH₂—NH—. For example, if an ethyl group contains a nitrogen atom, this may refer to —NH—CH₂—CH₃, —CH₂—NH—CH₃, and —CH₂—CH₂—NH₂.

Herein, it is understood that cyclic compounds (i.e. aryl, cycloalkyl, cycloalkenyl, etc.) are understood to be monocyclic, polycyclic or branched. It is understood that the number of carbon atoms for cyclic compounds not only refers to the number of carbon atoms in one ring, but that the carbon atoms may be comprised in multiple rings. These rings may be fused to the main ring or substituted onto the main ring. For example, C₁₀ aryl optionally containing heteroatoms may refer to inter alia a naphthyl group (fused rings) or to e.g. a bipyridyl group (substituted rings, both containing an N atom).

Unless stated otherwise, (hetero)alkyl groups, (hetero)alkenyl groups, (hetero)alkynyl groups, (hetero)cycloalkyl groups, (hetero)cycloalkenyl groups, (hetero)cycloalkynyl groups, (hetero) alkylcycloalkyl groups, (hetero)alkylcycloalkenyl groups, (hetero) alkylcycloalkynyl groups, (hetero)cycloalkylalkyl groups, (hetero)cycloalkenylalkyl groups, (hetero)cycloalkynylalkyl groups, (hetero) alkenylcycloalkyl groups, (hetero)alkenylcycloalkenyl groups, (hetero) alkenylcycloalkynyl groups, (hetero)cycloalkylalkenyl groups, (hetero)cycloalkenylalkenyl groups, (hetero)cycloalkynylalkenyl groups, (hetero) alkynylcycloalkyl groups, (hetero)alkynylcycloalkenyl groups, (hetero) alkynylcycloalkynyl groups, (hetero)cycloalkylalkynyl groups, (hetero)cycloalkenylalkynyl groups, (hetero)cycloalkynylalkynyl groups, (hetero)aryl groups, (hetero)arylalkyl groups, (hetero)arylalkenyl groups, (hetero)arylalkynyl groups, alkyl(hetero)aryl groups, alkenyl(hetero)aryl groups, alkynyl(hetero)aryl groups, cycloalkyl(hetero)aryl groups, cycloalkenyl(hetero)aryl groups, cycloalkynyl(hetero)aryl groups, (hetero)arylcycloalkyl groups, (hetero)arylcycloalkenyl groups, (hetero)arylcycloalkynyl groups, (hetero)alkylene groups, (hetero)alkenylene groups, (hetero)alkynylene groups, (hetero)cycloalkylene groups, (hetero)cycloalkenylene groups, (hetero)cycloalkynylene groups, (hetero)arylene groups, alkyl(hetero)arylene groups, (hetero)arylalkylene groups, (hetero)arylalkenylene groups, (hetero)arylalkynylene groups, alkenyl(hetero)arylene, alkynyl(hetero)arylene, (hetero)arenetriyl groups, (hetero)cycloalkanetriyl groups, (hetero)cycloalkenetriyl and (hetero)cycloalkynetriyl groups are optionally substituted with one or more substituents independently selected from the group consisting of —Cl, —F, —Br, —I, —OH, —NH₂, —SO₃H, —PO₃H, —PO₄H₂, —NO₂,
—CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero) arylcycloalkynyl groups. Unless stated otherwise, the substituents disclosed herein optionally contain one or more heteroatoms selected from the group consisting of O, S, NR₅, P, and Si, wherein the N, S, and P atoms are optionally oxidized, wherein the N atoms are optionally quaternized. Preferably, these substituents optionally contain one or more heteroatoms selected from the group consisting of O, S, and NR₅.

In preferred embodiments, the substituents are selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₄-C₁₂ (hetero)arylalkenyl groups, C₄-C₁₂ (hetero)arylalkynyl groups, C₄-C₁₂ alkenyl(hetero)aryl groups, C₄-C₁₂ alkynyl(hetero)aryl groups, C₄-C₁₂ alkylcycloalkyl groups, C₆-C₁₂ alkylcycloalkenyl groups, C₁₃-C₁₆ alkylcycloalkynyl groups, C₄-C₁₂ cycloalkylalkyl groups, C₆-C₁₂ cycloalkenylalkyl groups, C₁₃-C₁₆ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₅-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₁₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero) arylcycloalkynyl groups.

In preferred embodiments, the substituents are selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₇ alkyl groups, C₂-C₇ alkenyl groups, C₂-C₇ alkynyl groups, C₆-C₇ aryl groups, C₂-C₇ heteroaryl groups, C₃-C₇ cycloalkyl groups, C₅-C₇ cycloalkenyl groups, C₁₂ cycloalkynyl groups, C₃-C₇ alkyl(hetero)aryl groups, C₃-C₇ (hetero)arylalkyl groups, C₄-C₇ (hetero)arylalkenyl groups, C₄-C₇ (hetero)arylalkynyl groups, C₄-C₇ alkenyl(hetero)aryl groups, C₄-C₇ alkynyl(hetero)aryl groups, C₄-C₇ alkylcycloalkyl groups, C₆-C₇ alkylcycloalkenyl groups, C₁₃-C₁₆ alkylcycloalkynyl groups, C₄-C₇ cycloalkylalkyl groups, C₆-C₇ cycloalkenylalkyl groups, C₁₃-C₁₆ cycloalkynylalkyl groups, C₅-C₇ alkenylcycloalkyl groups, C₇-C₇ alkenylcycloalkenyl groups, C₁₄-C₁₆ alkenylcycloalkynyl groups, C₅-C₇ cycloalkylalkenyl groups, C₇-C₈ cycloalkenylalkenyl groups, C₁₄-C₁₆ cycloalkynylalkenyl groups, C₅-C₇ alkynylcycloalkyl groups, C₇-C₈ alkynylcycloalkenyl groups, C₁₄-C₁₆ alkynylcycloalkynyl groups, C₅-C₇ cycloalkylalkynyl groups, C₇-C₈, cycloalkenylalkynyl groups, C₁₄-C₁₆ cycloalkynylalkynyl groups, C₅-C₇ cycloalkyl(hetero)aryl groups, C₇-C₈ cycloalkenyl(hetero)aryl groups, C₁₄-C₁₆ cycloalkynyl(hetero)aryl groups, C₅-C₇ (hetero)arylcycloalkyl groups, C₇-C₈, (hetero)arylcycloalkenyl groups, and C₁₄-C₁₆ (hetero) arylcycloalkynyl groups, C₄-C₈ (hetero)arylalkenyl groups, C₄-C₈ (hetero)arylalkynyl groups, C₄-C₈ alkenyl(hetero)aryl groups, C₄-C₈ alkynyl(hetero)aryl groups, C₅-C₉ cycloalkyl(hetero)aryl groups, C₇-C₁₁ cycloalkenyl(hetero)aryl groups, C₁₄-C₁₈ cycloalkynyl(hetero)aryl groups, C₅-C₉ (hetero)arylcycloalkyl groups, C₇-C₁₁ (hetero)arylcycloalkenyl groups, and C₁₄-C₁₈ (hetero) arylcycloalkynyl groups.

Unless stated otherwise, any group disclosed herein that is not cyclic is understood to be linear or branched. In particular, hetero)alkyl groups, (hetero)alkenyl groups, (hetero)alkynyl groups, (hetero)alkylene groups, (hetero)alkenylene groups, (hetero)alkynylene groups, and the like are linear or branched, unless stated otherwise.

The general term “sugar” is herein used to indicate a monosaccharide, for example glucose (Glc), galactose (Gal), mannose (Man) and fucose (Fuc). The term “sugar derivative” is herein used to indicate a derivative of a monosaccharide sugar, i.e. a monosaccharide sugar comprising substituents and/or functional groups. Examples of a sugar derivative include amino sugars and sugar acids, e.g. glucosamine (GlcNH₂), galactosamine (GalNH₂)N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (ldoA).

A sugar may be without further substitution, and then it is understood to be a monosaccharide. A sugar may be further substituted with at one or more of its hydroxyl groups, and then it is understood to be a disaccharide or an oligosaccharide. A disaccharide contains two monosaccharide moieties linked together. An oligosaccharide chain may be linear or branched, and may contain from 3 to 10 monosaccharide moieties.

The term “protein” is herein used in its normal scientific meaning. Herein, polypeptides comprising about 10 or more amino acids are considered proteins. A protein may comprise natural, but also unnatural amino acids. The term “protein” herein is understood to comprise antibodies and antibody fragments.

The term “peptide” is herein used in its normal scientific meaning. Herein, peptides are considered to comprise a number of amino acids in a range of from 2 to 9.

The term “peptoids” is herein used in its normal scientific meaning.

An antibody is a protein generated by the immune system that is capable of recognizing and binding to a specific antigen. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as Drug or Targeting Agent T^{T}, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. For the avoidance of doubt, in the context of this invention the term “antibody” is meant to encompass all of the antibody variations, fragments, derivatives, fusions, analogs and mimetics outlined in this paragraph, unless specified otherwise.

A linker is herein defined as a moiety that connects two or more elements of a compound. For example in a bioconjugate, a biomolecule and a targeting moiety are covalently connected to each other via a linker.

A biomolecule is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

As used herein, an organic molecule is defined as a molecule comprising a C—H bond. Organic compound and organic molecule are used synonymously. It will be understood that “organic molecule” as used herein includes biomolecules, such as nucleic acids (oligonucleotides, polynucleotides, DNA, RNA), peptides, proteins (in particular antibodies), carbohydrates (monosaccharides, oligosaccharides, and polysaccharides), aptamers, hormones, toxins, steroids, cytokines, and lipids; small organic molecules as defined herein; polymers (in particular polyethylene glycol); LNA and PNA; amino acids; peptoids; molecules comprising a radionuclide; fluorescent dyes; drugs; resins (in particular polystyrene and agarose); beads; particles (in particular polymersomes, liposomes, and beads); gels; surfaces; organometallic compounds; cells; and combinations thereof.

As used herein, an inorganic molecule is defined as any molecule not being an organic molecule, i.e. not comprising a C—H bond. It will be understood that “inorganic molecule” as used herein includes surfaces (in particular chips, wafers, gold, metal, silica-based surfaces such as glass); particles such as beads (in particular magnetic beads, gold beads), silica-based particles, polymer-based materials, iron oxide particles; caron nanotubes; allotropes of carbon (in particular fullerenes such as Buckminsterfullerene; graphite, graphene, diamond, Lonsdaleite, Q-carbon, linearn acetylenic carbon, amorphous carbon, and carbon nanotubes); drugs (in particular cisplatin); and combinations thereof.

As used herein, “particle” is preferably defined as a microparticle or a nanoparticle.

The term “salt thereof” means a compound formed when an acidic proton, typically a proton of an acid, is replaced by a cation, such as a metal cation or an organic cation and the like. The term “salt thereof” also means a compound formed when an amine is protonated. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter-ions having acceptable mammalian safety for a given dosage regime). Such salts may be derived from pharmaceutically acceptable inorganic or organic bases and from pharmaceutically acceptable inorganic or organic acids.

“Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The logarithm of the partition-coefficient, i.e. Log P, is herein used as a measure of the hydrophobicity of a compound. Typically, the Log P is defined as

$$log ( \frac{{[ Solute ]}_{octanol}^{un - ionized}}{{[ Solute ]}_{water}^{un - ionized}} )$$

The skilled person is aware of methods to determine the partition-coefficient of compounds without undue experimentation. Alternatively, the skilled person knows that software is available to reliably estimate the Log P value, for example as a function within ChemDraw® software or online available tools.

The unified atomic mass unit or Dalton is herein abbreviated to Da. The skilled person is aware that Dalton is a regular unit for molecular weight and that 1 Da is equivalent to 1 g/mol (grams per mole).

It will be understood that herein, the terms “moiety” and “group” are used interchangeably when referring to a part of a molecule.

It will be understood that when a heteroatom is denoted as —X(R′)₂—, wherein X is the heteroatom and R′ is a certain moiety, then this denotes that two moieties R′ are attached to the heteroatom.

It will be understood that when a group is denoted as, for example, —((R₅₁)₂—R₅₂)₂— or a similar notation, in which R₅₁ and R₅₂ are certain moieties, then this denotes that first, it should be written as —R₅₁—R₅₁—R₅₂—R₅₁—R₅₁—R₅₂-before the individual R₅₁ and R₅₂ moieties are selected, rather than first selecting moieties R₅₁ and R₅₂ and then writing out the formula.

It will be understood that the disclosure is divided into several Sections. It will be understood that the embodiments in each Section can be combined with embodiments from other sections, or in general with any embodiment disclosed herein. In the event that the symbols describing variables (e.g. R-groups, Formula numbers, single letters describing an integer, and the like) in a Section are identical to those of a different Section or another part of the disclosure, it will be understood that said symbols are as defined within the same Section. For example, if Section 1 and Section 3 both describe an R₁-group with different definitions, R₁ in Section 1 should be interpreted as defined in Section 1. Regardless of these possible identical symbols, it will be understood that the different embodiments between sections may be combined, and the symbols may be redefined (e.g. renumbered) if necessary.

### Section 1—Compounds in Relation to the Invention

As defined herein, a compound according to the invention is one according to Formula 1:

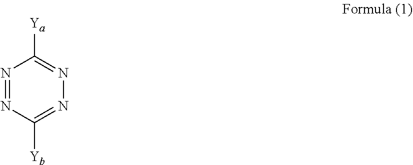

and preferably including pharmaceutically acceptable salts thereof,
wherein, Yₐ is selected from the group consisting of Y₁, Y₂, Y₃, Y₄, Y₅ and Y₆:

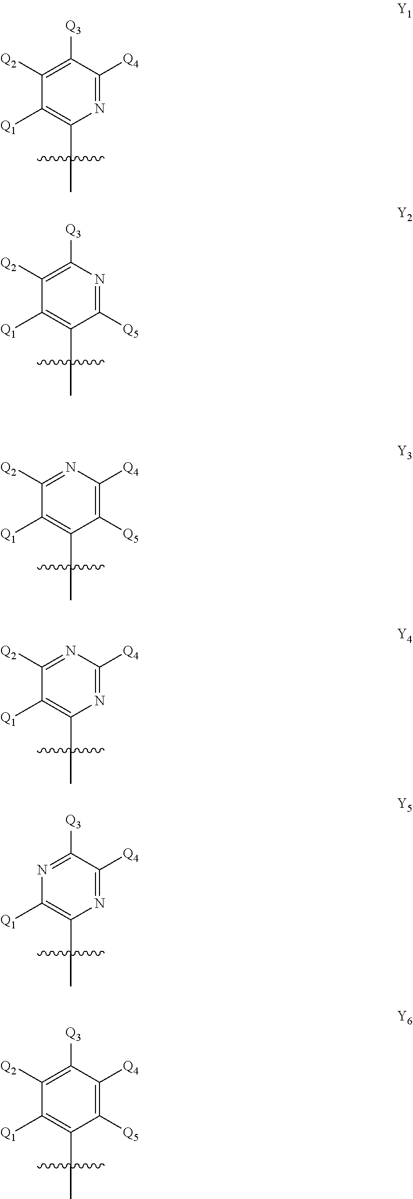

wherein, Y_{b} is selected from the group consisting of Y₁, Y₂, Y₃, Y₄, Y₅, Y₆, hydrogen, R₃, and R₈₇;
wherein when Yₐ is Y₆, then Y_{b} is hydrogen,
wherein each Q₁ and Q₅, are individually selected from the group consisting of R₁, hydrogen, R₃ and R₈₇;
wherein each Q₂ and Q₄, are individually selected from the group consisting of R₂, hydrogen, R₃, and R₈₇;
wherein each Q₃ is individually selected from the group consisting of hydrogen, R₃, and R₈₇;
wherein the compound of Formula (1) comprises at least one R₁ or R₂ group,
wherein each R₁ individually is selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂;
wherein each R₂ individually is selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂;
wherein each X₅₀ and X₅₁ individually is selected from the group consisting of hydrogen, R₆, and R₈₇;
wherein each R₆ is preferably independently selected from the group consisting of hydrogen, C₁-C₄ alkyl groups, C₂-C₄ alkenyl groups, and C₄₋₆ (hetero)aryl groups;
wherein for R₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂, and —NO₂; and optionally contain at most two heteroatoms selected from the group consisting of —O—, —S—, —NH—, —P—, and —Si—, wherein the N, S, and P atoms are optionally oxidized, wherein each R₃ is individually selected from the group consisting of —F, —Cl, —Br, —I, —OR₇, —N(R₇)₂, —SO₃, —PO₃⁻, —NO₂, —CF₃, —SR₇, —S(═O)₂N(R₇)₂, OC(═O)R₇, SC(═O)R₇, OC(═S)R₇, SC(═S)R₇, NR₇C(═O)—R₇, NR₇C(═S)—R₇, NR₇C(═O)O—R₇, NR₇C(═S)O—R₇, NR₇C(═O)S—R₇, NR₇C(═S)S—R₇, OC(═O)N(R₇)₂, SC(═O)N(R₇)₂, OC(═S)N(R₇)₂, SC(═S)N(R₇)₂, NR₇C(═O)N(R₇)₂, NR₇C(═S)N(R₇)₂, C(═O)R₇, C(═S)R₇, C(═O)N(R₇)₂, C(═S)N(R₇)₂, C(═O)O—R₇, C(═O)S—R₇, C(═S)O—R₇, C(═S)S—R₇, —S(O)R₇, —S(O)₂R₇, NR₇S(O)₂R₇, —ON(R₇)₂, —NR₇OR₇, C₁-C₈ alkyl groups, C₂-C₈, alkenyl groups, C₂-C₈ alkynyl groups, C₆-C₁₂ aryl, C₂-C₁₂ heteroaryl, C₃-C₈ cycloalkyl groups, C₅-C₈, cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₇, —N(R₇)₂, —SO₃R₇, —PO₃(R₇)₂, —PO₄(R₇)₂, —NO₂, —CF₃, ═O, ═NR₇, and —SR₇, and optionally contain one or more heteroatoms selected from the group consisting of O, S, NR₇, P, and Si, wherein the N, S, and P atoms are optionally oxidized, wherein the N atoms are optionally quaternized;
wherein R₇ is individually selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized;
wherein for each individual Yₐ and Y_{b} preferably at most two, more preferably at most one of Q₁, Q₂, Q₃, Q₄, and Q₅ are said R₈₇;
wherein the compound according to Formula (1) preferably comprises at most four R₈₇ groups, more preferably at most two R₈₇ groups, most preferably at most one R₈₇;
wherein the compound according to Formula (1) preferably comprises at least one R₈₇;
preferably with the proviso that the compound of Formula (1) is not

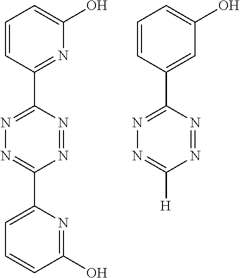 [SMILES: Oc1cccc(-c2nnc(-c3cccc(O)n3)nn2)n1 ; Oc1cccc(-c2nncnn2)c1]

wherein preferably for each individual Yₐ and Y_{b} at most three, more preferably at most two of Q₁, Q₂, Q₃, Q₄, and Q₅ are not hydrogen;
wherein preferably for each individual Yₐ and Y_{b} at most two of Q₁, Q₂, Q₄, and Q₅ are R₁ or R₂,
wherein preferably for each individual Yₐ and Y_{b} one of Q₁, Q₂, Q₄, and Q₅ is R₁ or R₂,
wherein preferably both Yₐ and Y_{b} comprise at least one R₁ or R₂,
wherein preferably both Yₐ and Y_{b} comprise one R₁ or R₂,
wherein preferably both Yₐ and Y_{b} comprise one R₁ or R₂, whereby the R₁ comprised in Yₐ is the same as the R₁ comprised in Y_{b}, and/or the R₂ comprised in Yₐ is the same as the R₂ comprised in Y_{b},
wherein preferably Yₐ and Y_{b} are both independently selected Y₁, or both independently selected Y₂, or both independently selected Y₃, or both independently selected Y₄, or both independently selected Y₅.
In preferred embodiments Yₐ equals Y_{b}.
In preferred embodiments Yₐ is selected from Y₁, Y₂, Y₃, Y₄ or Y₅ and Y_{b} is hydrogen, R₃ or R₈₇.
In preferred embodiments Yₐ is selected from Y₁, Y₂, Y₃, Y₄ or Y₅ and Y_{b} is hydrogen.
In preferred embodiments the compound according to Formula (1) does not comprise R₈₇.
In preferred embodiments, X₅₀ is hydrogen.
In preferred embodiments when R₁ or R₂ is N(X₅₀)₂, then one X₅₀ is hydrogen and one X₅₀ is R₆ or R₈₇.
In a preferred embodiment, when Q₁ is a R₃ or R₈₇, then for Q₁ the R₃ and R₈₇ are not a group in accordance with the definition of R₁.
In a preferred embodiment, when Q₅ is a R₃ or R₈₇, then for Q₅ the R₃ and R₈₇ are not a group in accordance with the definition of R₁.
In a preferred embodiment, when Q₂ is a R₃ or R₈₇, then for Q₂ the R₃ and R₈₇ are not a group in accordance with the definition of R₂.
In a preferred embodiment, when Q₄ is a R₃ or R₈₇, then for Q₄ the R₃ and R₈₇ are not a group in accordance with the definition of R₂.
In preferred embodiments Formula (1) does not comprise R₂.

In preferred embodiments, a compound according to the invention is one according to any one of Formulae (2)-(7):

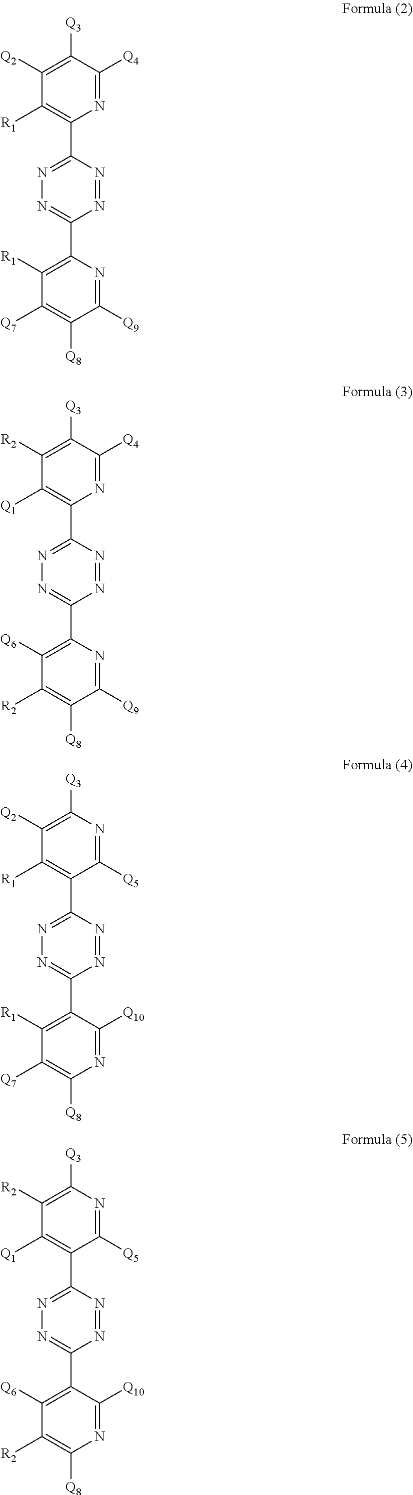

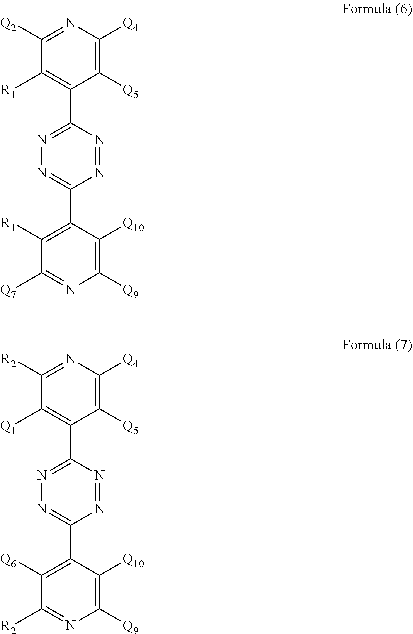

wherein Q₆ is as defined for Q₁, Q₇ is as defined for Q₂, Q₈, is as defined for Q₃, Q₉ is as defined for Q₄, and Q₁₀ is as defined for Q₅,
wherein preferably at most two, more preferably at most one of Q₁, Q₂, Q₃, Q₄, and Q₅ are said R₈₇;
wherein preferably at most two, more preferably at most one of Q₆, Q₇, Q₈, Q₉, and Q₁₀ are said R₈₇;
wherein preferably the compound according to any one of Formulae (2) to (7) comprises at most four R₈₇ groups, more preferably at most two R₈₇ groups;
wherein the compound according to Formulae (2)-(7) preferably comprises at least one R₈₇;
wherein preferably at most six, more preferably at most four of Q₁, Q₂, Q₃, Q₄, Q₅, Q₆, Q₇, Q₈, Q₉, Q₁₀ are not hydrogen, wherein preferably the R₁ or R₂ groups are identical.
In a preferred embodiment, when Q₆ is a R₃ or R₈₇, then for Q₆ the R₃ and R₈₇ are not a group in accordance with the definition of R₁.
In a preferred embodiment, when Q₁₀ is a R₃ or R₈₇, then for Q₁₀ the R₃ and R₈₇ are not a group in accordance with the definition of R₁.
In a preferred embodiment, when Q₇ is a R₃ or R₈₇, then for Q₇ the R₃ and R₈₇ are not a group in accordance with the definition of R₂.
In a preferred embodiment, when Q₉ is a R₃ or R₈₇, then for Q₉ the R₃ and R₈₇ are not a group in accordance with the definition of R₂.
In a preferred embodiment:

(a) Q₁ and Q₆ are the same R₈₇, and the other moieties Q are H; or

(b) Q₂ and Q₇ are the same R₈₇, and the other moieties are H;

(c) Q₃ and Q₈ are the same R₈₇, and the other moieties Q are H;

(d) Q₄ and Q₉ are the same R₈₇, and the other moieties Q are H; or

(e) Q₅ and Q₁₀ are the same R₈₇, and the other moieties Q are H.

In a preferred embodiment, when at least one of R₁ and R₂ is C(X₅₁)₂N(X₅₀)₂, then within this group at most two, preferably at most one, of X₅₀ and X₅₁ are not H.
In a preferred embodiment, each individual R₁ and R₂ comprises at most one R₈₇.

It will be understood that a compound according to the invention may herein also be referred to as an Activator.

R₁ In a preferred embodiment, each R₁ individually is selected from the group
consisting of N(X₅₀)₂, NX₅₀C(O)X₅₁, NX₅₀C(S)X₅₁, OH, SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁,
In a preferred embodiment, each R₁ individually is selected from the group consisting of N(X₅₀)₂, NX₅₀C(O)X₅₁, NX₅₀C(S)X₅₁, OH and SH.
In a preferred embodiment, each R₁ individually is selected from the group consisting of NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁,
In a preferred embodiment, R₁ is selected from the group consisting of NHX₅₀, C(X₅₁)₂NH₂, CHX₅₁NH₂, CH₂N(X₅₀)₂, CH₂NHX₅₀, NHC(O)X₅₁, NHC(S)X₅₁, NX₅₀SO₂X₅₁, OH, and SH.
In a preferred embodiment, R₁ is NHX₅₀.
In a preferred embodiment, R₁ is C(X₅₁)₂NH₂.
In a preferred embodiment, R₁ is CHX₅₁NH₂.
In a preferred embodiment, R₁ is CH₂N(X₅₀)₂
In a preferred embodiment, R₁ is CH₂NHX₅₀.
In a preferred embodiment, R₁ is NH₂.
In a preferred embodiment, R₁ is CH₂NH₂.
In a preferred embodiment, R₁ is NHC(O)X₅₁.
In a preferred embodiment, R₁ is NHC(S)X₅₁.
In a preferred embodiment, R₁ is NX₅₀SO₂X₅₁.
In a preferred embodiment, R₁ is OH.
In a preferred embodiment, R₁ is SH.

### R₂

In a preferred embodiment, R₂ is individually selected from the group consisting of N(X₅₀)₂, NX₅₀C(O)X₅₁, NX₅₀C(O)OX₅₁, and NX₅₀C(O)N(X₅₁)₂. In a preferred embodiment, R₂ is selected from the group consisting of N(X₅₀)₂, and NX₅₀C(O)X₅₁. In a preferred embodiment, R₂ is selected from the group consisting of NHX₅₀ and NHC(O)X₅₁.
In a preferred embodiment, R₂ is NHX₅₀.
In a preferred embodiment, R₂ is NH₂.
In a preferred embodiment, R₂ is NHC(O)X₅₁.

### R₃

In a preferred embodiment, each R₃ is individually selected from the group consisting of F, —OH, —NH₂, —SO₃⁻, —NO₂, —CF₃, —SH, C₁-C₆ alkyl groups, C₆ aryl groups, C₄-C₅ heteroaryl groups, C₅-C₈, alkyl(hetero)aryl groups, C₅-C₈ (hetero)arylalkyl groups, C₄-C₈, alkylcycloalkyl groups, and C₄-C₈ cycloalkylalkyl groups.
In a more preferred embodiment, each R₃ is individually selected from the group consisting of F, —SO₃⁻, —NO₂, —CF₃, C₁-C₆ alkyl groups, C₆ aryl groups, C₄-C₅ heteroaryl groups, C₅-C₈, alkyl(hetero)aryl groups, C₅-C₈, (hetero)arylalkyl groups, C₄-C₈, alkylcycloalkyl groups, and C₄-C₈ cycloalkylalkyl groups.

### X₅₀

In a preferred embodiment, each X₅₀ is individually selected from the group consisting of hydrogen, R₆, and R₈₇.
In a preferred embodiment, X₅₀ is R₆.
In a preferred embodiment, X₅₀ is R₈₇.
In a preferred embodiment, X₅₀ is H.

### X₅₁

In a preferred embodiment, each X₅₁ is individually selected from the group consisting of hydrogen, R₆, and R₈₇.
In a preferred embodiment, X₅₁ is R₆.
In a preferred embodiment, X₅₁ is R₈₇.
In a preferred embodiment, X₅₁ is H.

### R₆

In a preferred embodiment, each R₆ is independently selected from the group consisting of hydrogen, C₁-C₄ alkyl groups, C₂-C₄ alkenyl groups, and C₄₋₆ (hetero)aryl groups. For R₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂; and optionally contain at most two heteroatoms selected from the group consisting of —O—, —S—, —NH—, —P—, and —Si—, wherein the N, S, and P atoms are optionally oxidized.
In a preferred embodiment, R₆ is C₁-C₄ alkyl.
In a preferred embodiment, R₆ does not contain heteroatoms and is not substituted with a moiety selected from the group consisting of —Cl, —F, —Br, —I, —OH, —NH₂, ═O, —SH, —SO₃H, —PO₃H, —PO₄H₂ and —NO₂.

### R₇

In preferred embodiments, R₇ is selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

In preferred embodiments, R₇ is selected from the group consisting of hydrogen, C₁-C₄ alkyl groups, C₂-C₄ alkenyl groups, C₂-C₄ alkynyl groups, C₆-C₈ aryl, C₂-C₈ heteroaryl, C₃-C₆ cycloalkyl groups, C₅-C₆ cycloalkenyl groups, C₃-C₁₀ alkyl(hetero)aryl groups, C₃-C₁₀ (hetero)arylalkyl groups, C₄-C₈ alkylcycloalkyl groups, C₄-C₈ cycloalkylalkyl groups, C₅-C₁₀ cycloalkyl(hetero)aryl groups and C₅-C₁₀ (hetero)arylcycloalkyl groups, wherein the R₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

In preferred embodiments, the R₇ groups not being hydrogen are not substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and do not contain heteroatoms.

### Q₁

In a preferred embodiment, Q₁ is selected from the group consisting of hydrogen, R₃, and R₈₇. In a preferred embodiment, Q₁ is hydrogen. In a preferred embodiment, Q₁ is R₃. In a preferred embodiment, Q₁ is R₈₇, and preferably Q₂, Q₃, Q₄, Q₅, Q₆, Q₇, Q₈, Q₉, and Q₁₀ are R₁, R₂, or hydrogen.

### Q₂

In a preferred embodiment, Q₂ is selected from the group consisting of hydrogen R₃, and R₈₇. In a preferred embodiment, Q₂ is hydrogen. In a preferred embodiment, Q₂ is R₃. In a preferred embodiment, Q₂ is R₈₇, and preferably Q₁, Q₃/Q₄/Q₅, Q₆, Q₇, Q₈, Q₉, and Q₁₀ are R₁, R₂, or hydrogen.

### Q₃

In a preferred embodiment, Q₃ is selected from the group consisting of hydrogen R₃, and R₈₇. In a preferred embodiment, Q₃ is hydrogen. In a preferred embodiment, Q₃ is R₃. In a preferred embodiment, Q₃ is R₈₇, and preferably Q₁, Q₂, Q₄, Q₅, Q₆, Q₇, Q₈, Q₉, and Q₁₀ are R₁, R₂, or hydrogen.

### Q₄

In a preferred embodiment, Q₄ is selected from the group consisting of hydrogen R₃, and R₈₇. In a preferred embodiment, Q₄ is hydrogen. In a preferred embodiment, Q₄ is R₃. In a preferred embodiment, Q₄ is R₈₇, and preferably Q₁, Q₂, Q₃, Q₅, Q₆, Q₇, Q₈, Q₉ and Q₁₀ are R₁, R₂, or hydrogen.

### Q₅

In a preferred embodiment, Q₅ is selected from the group consisting of hydrogen R₃, and R₈₇. In a preferred embodiment, Q₅ is hydrogen. In a preferred embodiment, Q₅ is R₃. In a preferred embodiment, Q₅ is R₈₇, and preferably Q₁, Q₂, Q₃, Q₄, Q₆, Q₇, Q₈, Q₉ and Q₁₀ are R₁, R₂, or hydrogen.

### Q₆

In a preferred embodiment, Q₆ is selected from the group consisting of hydrogen R₃, and R₈₇. In a preferred embodiment, Q₆ is hydrogen. In a preferred embodiment, Q₆ is R₃. In a preferred embodiment, Q₆ is R₈₇, and preferably Q₁, Q₂, Q₃, Q₄, Q₅, Q₇, Q₈, Q₉ and Q₁₀ are R₁, R₂, or hydrogen.

### Q₇

In a preferred embodiment, Q₇ is selected from the group consisting of hydrogen R₃, and R₈₇. In a preferred embodiment, Q₇ is hydrogen. In a preferred embodiment, Q₇ is R₃. In a preferred embodiment, Q₇ is R₈₇, and preferably Q₁, Q₂, Q₃, Q₄, Q₅, Q₆, Q₈, Q₉ and Q₁₀ are R₁, R₂, or hydrogen.

### Q₈

In a preferred embodiment, Q₈ is selected from the group consisting of hydrogen R₃, and R₈₇. In a preferred embodiment, Q₈ is hydrogen. In a preferred embodiment, Q₈ is R₃. In a preferred embodiment, Q₈ is R₈₇, and preferably Q₁, Q₂, Q₃, Q₄, Q₅, Q₆, Q₇, Q₉ and Q₁₀ are R₁, R₂, or hydrogen.

### Q₉

In a preferred embodiment, Q₉ is selected from the group consisting of hydrogen R₃, and R₈₇. In a preferred embodiment, Q₉ is hydrogen. In a preferred embodiment, Q₉ is R₃. In a preferred embodiment, Q₉ is R₈₇, and preferably Q₁, Q₂, Q₃, Q₄, Q₅, Q₆, Q₇, Q₈ and Q₁₀ are R₁, R₂, or hydrogen.

### Q₁₀

In a preferred embodiment, Q₁₀ is selected from the group consisting of hydrogen R₃, and R₈₇. In a preferred embodiment, Q₁₀ is hydrogen. In a preferred embodiment, Q₁₀ is R₃. In a preferred embodiment, Q₁₀ is R₈₇, and preferably Q₁, Q₂, Q₃, Q₄, Q₅, Q₆, Q₇, Q₈ and Q₉ are R₁, R₂, or hydrogen.

### Formula (2)

In a preferred embodiment, the compound according to the invention is a compound according to Formula (2), wherein preferably, each individual R₁ and Q₂-Q₁, Q₇-Q₉ are as described herein.

In a preferred embodiment, the compound according to the invention is a compound according to Formula (2), wherein both R₁ are the same and are selected from the group consisting of NH₂, NHC(O)X₅₁, NX₅₀C(O)OX₅₁, NX₅₀C(O)N(X₅₁)₂, NX₅₀C(S)N(X₅₁)₂, OH, and SH; and Q₂-Q₁, Q₇-Q₉ are hydrogen.

In a preferred embodiment, the compound according to the invention is a compound according to Formula (2), wherein both R₁ are the same and are selected from the group consisting of NH₂, NHC(O)X₅₁, and OH; and Q₂-Q₁, Q₇-Q₉ are hydrogen.

### Formula (3)

In a preferred embodiment, the compound according to the invention is a compound according to Formula (3), wherein preferably, each individual R₂ and Q₁, Q₃-Q₄, Q₆, Q₈-Q₉ are as described herein.

In a preferred embodiment, the compound according to the invention is a compound according to Formula (3), wherein both R₂ are the same and are NH₂ or NHC(O)X₅₁, and Q₁, Q₃-Q₄, Q₆, Q₈-Q₉ are hydrogen.

### Formula (4)

In a preferred embodiment, the compound according to the invention is a compound according to Formula (4), wherein preferably, each individual R₁ and Q₂-Q₃, Q₅, Q₇, Q₈, Q₁₀ are as described herein.

In a preferred embodiment, the compound according to the invention is a compound according to Formula (4), wherein both R₁ are the same and are selected from the group consisting of NH₂, NHC(O)X₅₁, NX₅₀C(O)OX₅₁, NX₅₀C(O)N(X₅₁)₂, NX₅₀C(S)N(X₅₁)₂, OH, and SH; and Q₂-Q₃, Q₅, Q₇, Q₈, Q₁₀ are hydrogen.

In a preferred embodiment, the compound according to the invention is a compound according to Formula (4), wherein both R₁ are the same and are NH₂, NHC(O)X₅₁, and OH, and Q₂-Q₃, Q₅, Q₇, Q₈, Q₁₀ are hydrogen.

### Formula (5)

In a preferred embodiment, the compound according to the invention is a compound according to Formula (5), wherein preferably each individual R₂ and Q₁, Q₃, Q₅, Q₆, Q₈, Q₁₀ are as described herein.

In a preferred embodiment, the compound according to the invention is a compound according to Formula (5), wherein both R₂ are the same and are NH₂ or NHC(O)X₅₁, and Q₁, Q₃, Q₅, Q₆, Q₈, Q₁₀ are hydrogen.

### Formula (6)

In a preferred embodiment, the compound according to the invention is a compound according to Formula (6), wherein preferably each individual R₁ and Q₂, Q₄, Q₅, Q₇, Q₉, Q₁₀ are as described herein.

In a preferred embodiment, the compound according to the invention is a compound according to Formula (6), wherein both R₁ are the same and are selected from the group consisting of NH₂, NHC(O)X₅₁, NX₅₀C(O)OX₅₁, NX₅₀C(O)N(X₅₁)₂, NX₅₀C(S)N(X₅₁)₂, OH, and SH; and Q₂, Q₄, Q₅, Q₇, Q₉, Q₁₀ are hydrogen.

In a preferred embodiment, the compound according to the invention is a compound according to Formula (6), wherein both R₁ are the same and are NH₂, NHC(O)X₅₁, and OH, and Q₂, Q₄, Q₅, Q₇, Q₉, Q₁₀ are hydrogen.

### Formula (7)

In a preferred embodiment, the compound according to the invention is a compound according to Formula (7), wherein preferably each individual R₂ and Q₁, Q₄, Q₅, Q₆, Q₉, Q₁₀ are as described herein.

In a preferred embodiment, the compound according to the invention is a compound according to Formula (7), wherein both R₂ are the same and are NH₂ or NHC(O)X₅₁, and Q₁, Q₄, Q₅, Q₆, Q₉, Q₁₀ are hydrogen.

### R₈₇

Preferably, R₈₇ has a molecular weight of at least 100 Da, more preferably of at least 200 Da, more preferably at least 300 Da, more preferably at least 400 Da, more preferably at least 500 Da, and most preferably at least 1 kDa.

Preferably, R₈₇ has a molecular weight of at most 100 kDa, more preferably of at most 75 kDa, more preferably at most 50 kDa, more preferably at most 25 kDa, more preferably at most 10 kDa, and most preferably at most 3 kDa. Preferably, R₈₇ has a molecular weight a molecular weight in a range of from 100 Da to 3000 Da.

In a preferred embodiment, R₈₇ is a polymer, more preferably polyethylene glycol. In another preferred embodiment, R₈₇ is a carbohydrate. In another preferred embodiment, R₈₇ is a peptide or a protein, more preferably an antibody.
In a preferred embodiment, R₈₇ is a pharmacokinetics-modulating moiety (a P^{K} moiety). It will be understood that if R₈₇ in relation to the invention is a P^{K} moiety, then it is a moiety that modulates the pharmacokinetics of a compound according to any one of Formulae (1)-(7). The functions of R₈₇ include, but are not limited to, one or more of delaying clearance of said compound, affecting the volume of distribution of said compound (e.g. reducing or increasing the volume of distribution), achieving spatial control over its reaction with the Trigger, affecting (more particularly avoiding) the metabolism of said compound, and/or affecting (more particularly avoiding) the (undesired) sticking or (undesired) uptake of said compound to tissues. The skilled person is well aware of such groups, and how to synthesize these.

In preferred embodiments, each R⁸⁷ is independently selected from the group consisting of organic molecules, inorganic molecules, organometallic molecules, resins, beads, glass, microparticles, nanoparticles, gels, surfaces, and cells. Preferably, R⁸⁷ is independently selected from the group consisting of organic molecules, and inorganic molecules.

In preferred embodiments, each R⁸⁷ is independently selected from the group consisting of small molecules, proteins, carbohydrates, peptides, peptoids, oligosaccharides, molecules comprising a radionuclide, fluorescent dyes, inorganic molecules, organometallic molecules, polymers, lipids, oligonucleotides, DNA, RNA, PNA, LNA, drugs, resins, beads, glass, microparticles, nanoparticles, gels, surfaces, and cells.

Preferably, a small molecule is a small organic molecule. Preferably, a small molecule has a molecular weight of at most 2 kDa, more preferably at most 1 kDa, more preferably at most 750 Da, more preferably at most 500 Da, and most preferably at most 300 Da. Preferably, a small molecule has a molecular weight of at least 15 Da, more preferably at least 50 Da, more preferably at least 75 Da, and most preferably at least 100 Da.

In a preferred embodiment R₈₇ serves to increase the blood circulation time, increasing reaction time with the Trigger.

In a preferred embodiment R₈₇ serves to modulate the pharmacokinetics of a reaction product between a dienophile of this invention and a compound according to any one of Formulae (1)-(7).

The skilled person is well aware of such groups, and how to synthesize these.

Without wishing to be bound by theory, it is believed that the function and performance of the tetrazine moiety of the compounds according to any one of Formulae (1)-(7) in a bioorthogonal reaction is not significantly affected by the nature of R₈₇.

In a preferred embodiment, each R₈₇ is individually selected from the group consisting of biomolecule, polymer, peptide, peptoid, dendrimer, protein, carbohydrate, oligonucleotide, oligosaccharide, lipid, albumin, albumin-binding moiety, dye moiety, fluorescent moiety, imaging probe, and a Targeting Agent (T^{T}); and wherein R₈₇ is optionally bound to the tetrazine via a Spacer (S^{P}). Typically, a suitable polymer as R₈₇ is polyethyleneglycol (PEG). Such suitable PEG includes PEG with a number of repeating units in a range of from 2 to 4000, and PEG with a molecular weight in a range of from 200 Da to 100,000 Da.

In a preferred embodiment, R₈₇ is a moiety according to Formula (9).

In a preferred embodiment, R₈₇ is a moiety according to Formula (9), and is directly linked to the remainder of a compound according to any one of Formulae (1)-(7), for example without a spacer S^{P} between R₈₇ and the remainder of the moiety Yₐ or Y_{b} of Formula (1) or the pyridyl moiety of the compound according to any one of Formulae (2)-(7), and preferably if attached to an amine functionality of R₁ or R₂, z in Formula (9) is not 0.

In a preferred embodiment, R₈₇ is linked to the remainder of a compound according to any one of Formulae (1)-(7) via a spacer S^{P} as defined herein, i.e. D equals 1. In other preferred embodiments, D equals 0, and there is no spacer between R₈₇ and the remainder of a compound according to any one of Formulae (1)-(7).

In a preferred embodiment, R₈₇ is linked to the remainder of a compound according to any one of Formulae (1)-(7) optionally via a spacer S^{P} as defined herein and each R₈₇ is individually selected from the group consisting of biomolecule, polymer, peptide, peptoid, dendrimer, protein, carbohydrate, oligonucleotide, oligosaccharide, lipid, micelle, liposomes, polymersome, particle, nanoparticle, microparticle, bead, gel, resin, metal complex, organometallic moiety, albumin, albumin-binding moiety, dye moiety, fluorescent moiety, imaging probe, and a Targeting Agent (T^{T}).

In a preferred embodiment, one or multiple copies of the compound of the invention, i.e. the tetrazine, may be conjugated to R₈₇ groups that are gels, resins, polymers.
In a preferred embodiment, one or multiple copies of the compound of the invention may be conjugated to R₈₇ that is a Targeting Agent to selectively activate a Prodrug and selected locations in the body.
In a preferred embodiment, one or multiple copies of the compound of the invention may be conjugated to R₈₇ that is a membrane translocation moiety (e.g. adamantine, poly-lysine/arginine, TAT, human lactoferrin) to reach an intracellular Prodrug. Exemplary references regarding such moieties include: Trends in Biochemical Sciences, 2015. 40, 12, 749; J. Am. Chem. Soc. 2015, 137, 12153-12160; Pharmaceutical Research, 2007, 24, 11, 1977.
With respect to application in a cellular environment, such as in vivo, depending on the position of the Trigger-Construct (e.g. inside the cell or outside the cell) the Activator is designed to be able to effectively reach this Trigger-Construct. Therefore, the Activator can for example be tailored by varying its log P value, its reactivity or its charge, and this can optionally be achieved by R₈₇.
According to one embodiment, the Activator can be a multimeric compound, comprising a plurality of tetrazines. These multimeric compounds can be peptide, peptoid, protein, oligonucleotide, oligosaccharide, polymersome, biomolecules, polymers, dendrimers, liposomes, micelles, particles, nanoparticles, microparticles, polymer particles, or other polymeric constructs.

In a preferred embodiment, R₈₇ comprises a chelating moiety, preferably a chelating moiety as described herein.

In a preferred embodiment, R₈₇ includes but is not limited to amino acids, nucleosides, nucleotides, carbohydrates, and biopolymer fragments, such as oligo- or polypeptides, oligo- or polypeptoids, or oligo- or polylactides, or oligo- or poly-carbohydrates, oligonucleotides, varying from 2 to 200, particularly 2 to 113, preferably 2 to 50, more preferably 2 to 24 and more preferably 2 to 12 repeating units.

In a preferred embodiment, R₈₇ is a polymer. This includes linear or branched polyalkylene glycols such as polyethylene glycol (PEG) or polypropylene glycol (PPG) chains varying from 2 to 200, particularly 2 to 113, preferably 2 to 50, more preferably 2 to 24 and more preferably 2 to 12 repeating units. It is preferred that when polyalkylene glycols such as PEG and PPG polymers are only bound via one end of the polymer chain, that the other end is terminated with —OCH₃, —OCH₂CH₃, OCH₂CH₂CO₂H.

Other polymeric R₈₇ groups are polymers and copolymers such as poly-(2-oxazoline, poly(N-(2-hydroxypropyl)methacrylamide) (HPMA), polylactic acid (PLA), polylactic-glycolic acid (PLGA), polyglutamic acid (PG), dextran, polyvinylpyrrolidone (PVP), poly(1-hydroxymethylethylene hydroxymethyl-formal (PHF). Other exemplary polymers are polysaccharides, glycopolysaccharides, glycolipids, polyglycoside, polyacetals, polyketals, polyamides, polyethers, polyesters. Examples of naturally occurring polysaccharides that can be used are cellulose, amylose, dextran, dextrin, levan, fucoidan, carraginan, inulin, pectin, amylopectin, glycogen, lixenan, agarose, hyaluronan, chondroitinsulfate, dermatansulfate, keratansulfate, alginic acid and heparin. In yet other exemplary embodiments, the polymer is a copolymer of a polyacetal/polyketal and a hydrophilic polymer selected from the group consisting of polyacrylates, polyvinyl polymers, polyesters, polyorthoesters, polyamides, oligopeptides, polypeptides and derivatives thereof. Exemplary preferred polymeric R₈₇ groups are PEG, HPMA, PLA, PLGA, PVP, PHF, dextran, oligopeptides, and polypeptides.

In some aspects of the invention polymeric R₈₇ groups have a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa.
Other exemplary R₈₇ groups are dendrimers, such as poly(propylene imine) (PPI) dendrimers, PAMAM dendrimers, and glycol based dendrimers.
In a preferred embodiment, the tetrazine compounds of the invention comprise a Drug D^{D} instead of R₈₇. In this preferred embodiment the Drug is bound to the remainder of the compounds of the invention in the same way as R₈₇. In this embodiment R₈₇ equals a Drug. In a preferred embodiment, the compounds of the invention can comprise one or more Drugs and one or more R₈₇ groups. In a preferred embodiment, the is Drug is a prodrug that becomes a Drug upon reaction of the tetrazine with the Trigger. In a preferred embodiment the Drug is a moiety comprising a therapeutic radionuclide, preferably a radiometal-chelate complex. In other preferred embodiments the moiety comprising a therapeutic radionuclide, is an organic molecule comprising ¹³¹I.
In a preferred embodiment, the tetrazine compounds of the invention comprise an imaging moiety instead of R₈₇. In other embodiments, R₈₇ is or comprises an imaging moiety. In the context of Prodrug activation in vivo, a tetrazine activator comprising an imaging moiety can be used to activate the Prodrug and at the same to measure the extent of Prodrug activation. In this preferred embodiment the imaging moiety is bound to the remainder of the compounds of the invention in the same way as R₈₇. In this embodiment R₈₇ equals an imaging moiety. In a preferred embodiment, the compounds of the invention can comprise one or more imaging moieties and one or more R₈₇ groups.
Preferred imaging moieties are radionuclide-chelates complexes, radiolabeled molecules (e.g. with 18F, 1241), and fluorescent dyes.

In a preferred embodiment, R₈₇ is or comprises an imaging probe that comprises at least one ¹⁸F isotope.

### Formula (9)

Formula (9) in relation to the invention is

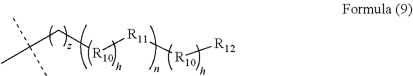

wherein the dashed line indicates a bond to the remaining part of the molecules satisfying any of the Formulae (1), (2), (3), (4), (5), (6) or (7),

In preferred embodiments, z is an integer in a range of from 0 to 12, preferably from 0 to 10, more preferably from 0 to 8, even more preferably from 1 to 6, most preferably from 2 to 4. In preferred embodiments, z is 0. In case the compound according to the invention comprises more than one moiety satisfying Formula (9), each z is independently selected.

In preferred embodiments, h is 0 or 1. In case the compound according to the invention comprises more than one moiety satisfying Formula (9), each h, z, and n is independently selected.

In preferred embodiments, each n belonging to a moiety according to Formula (9) is an integer independently selected from a range of from 0 to 24, preferably from 1 to 12, more preferably from 1 to 6, even more preferably from 1 to 3. In a preferred embodiment, n is 1. In other preferred embodiments n is an integer in the range from 12 to 24.

In a preferred embodiment, z is 0, and n is 1.

In a preferred embodiment, z is 1, and n is 1.

In a preferred embodiment, the moiety according to Formula (9) has a molecular weight in a range of from 100 Da to 3000 Da, preferably, in a range of from 100 Da to 2000 Da, more preferably, in a range of from 100 Da to 1500 Da, even more preferably in a range of from 150 Da to 1500 Da. Even more preferably still, the moiety according to Formula (9) has a molecular weight in a range of from 150 Da to 1000 Da, most preferably in a range of from 200 Da to 1000 Da.

In preferred embodiments, the group —((R₁₀)ₕ—R₁₁)ₙ—(R₁₀)ₕ—R₁₂ satisfies molecules from Group R^{M} shown below:

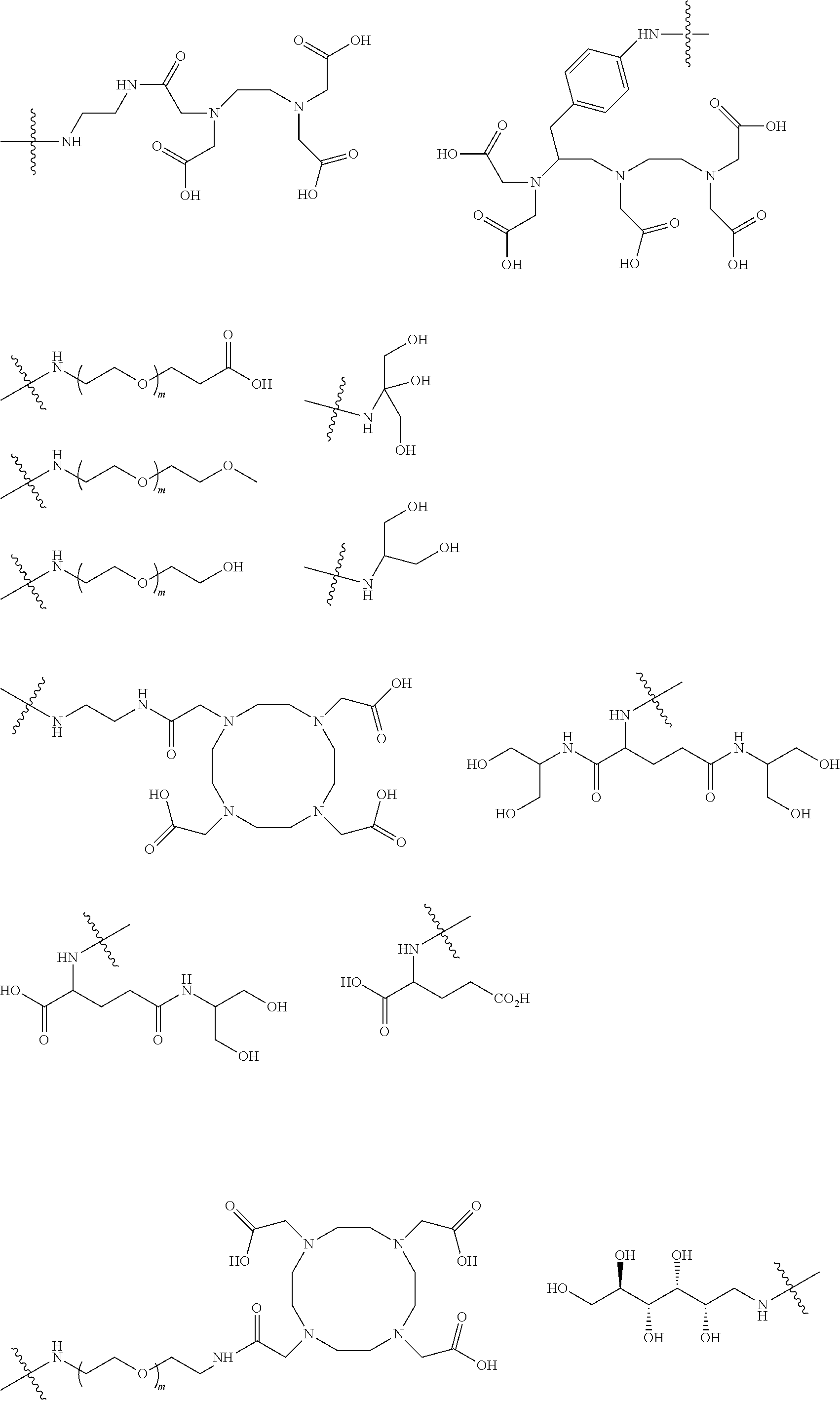

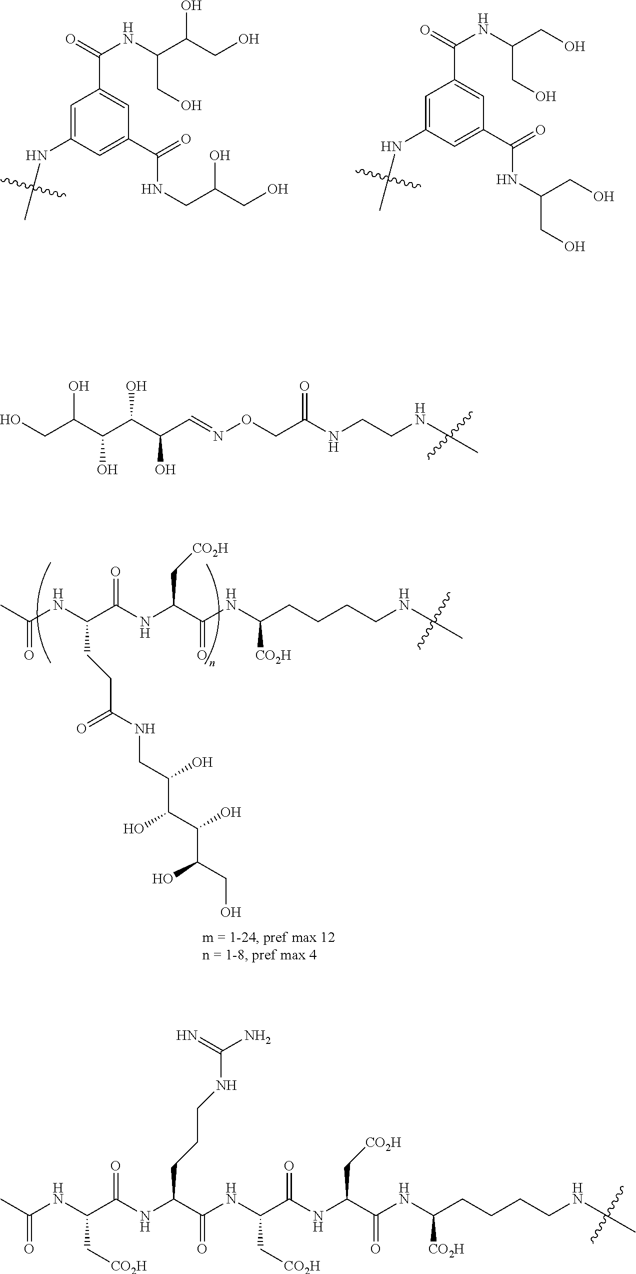

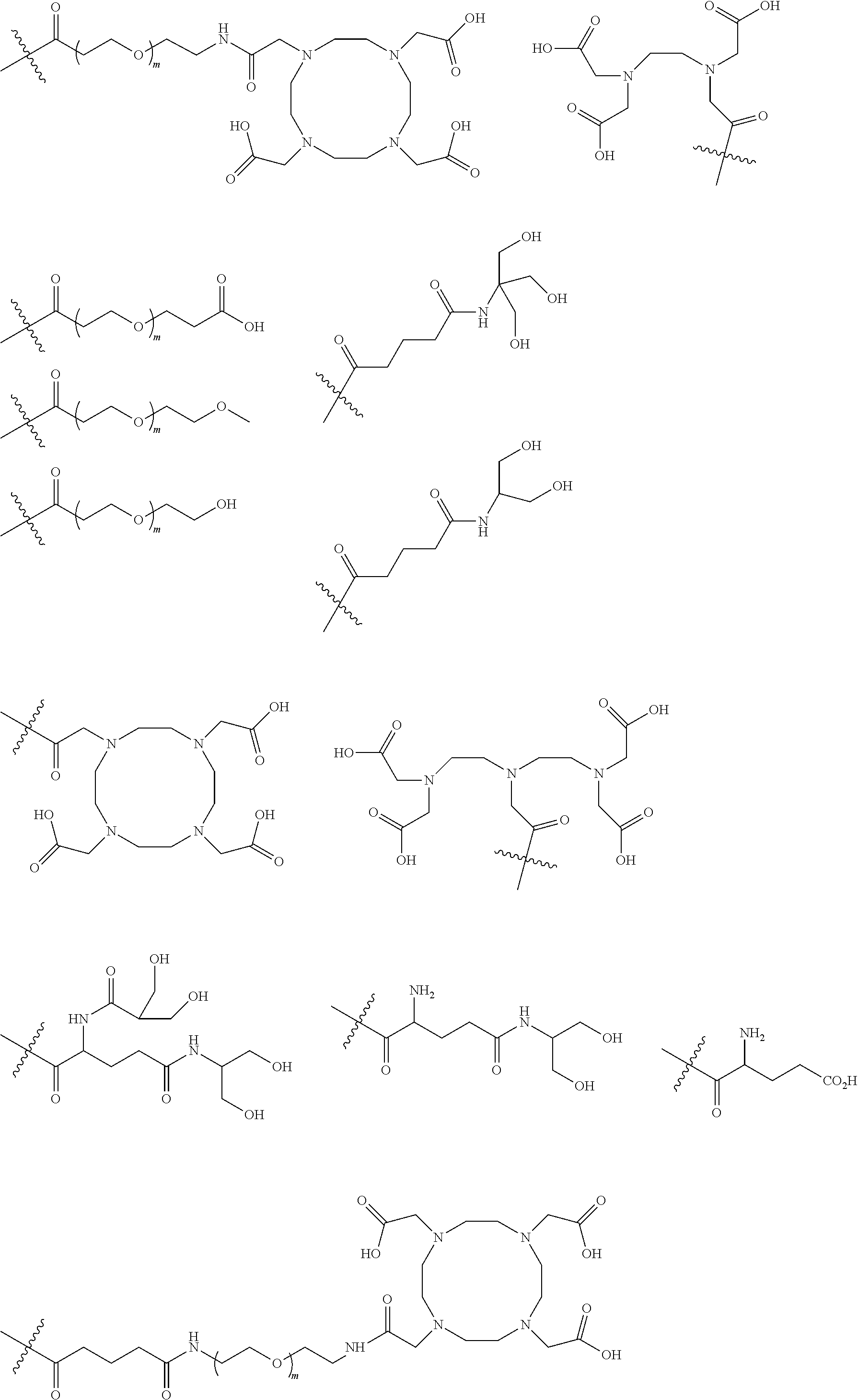

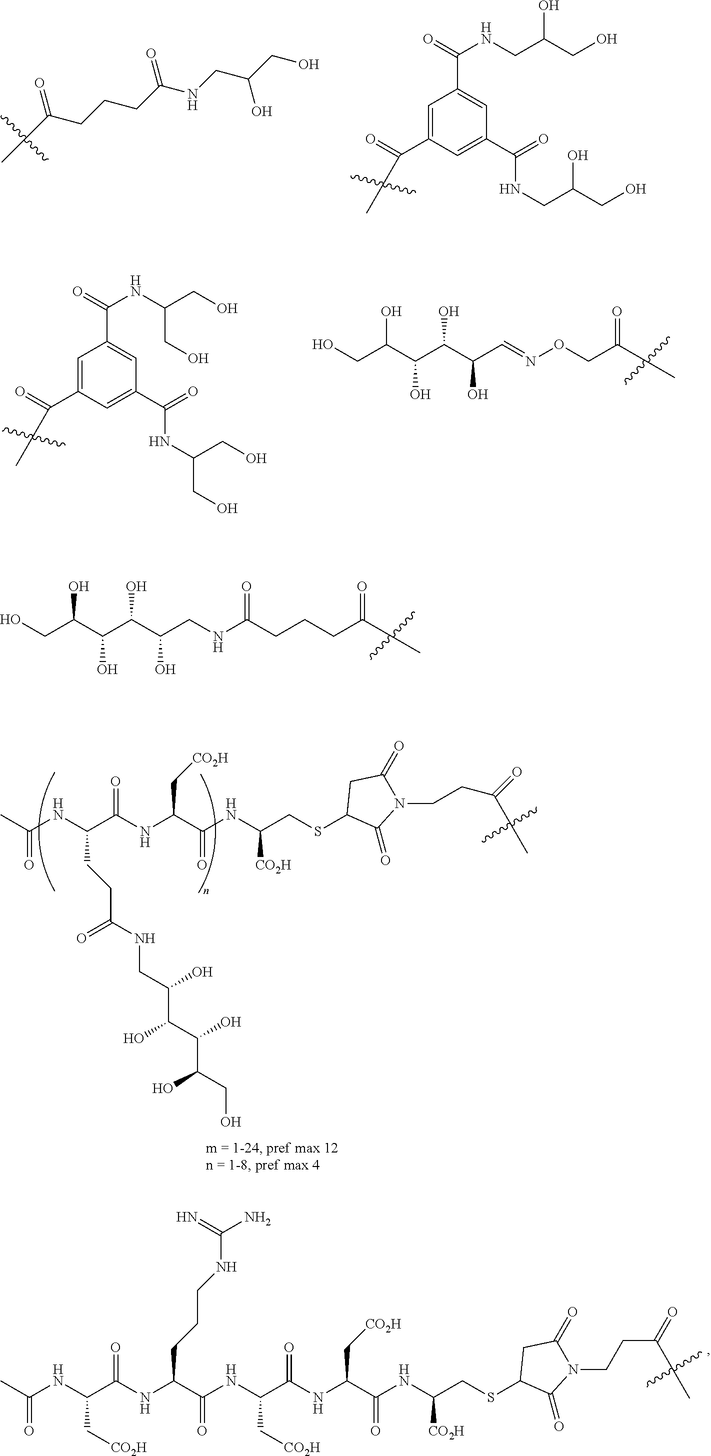

wherein the wiggly line denotes a bond to the remainder of the molecule.

In preferred embodiments, the group —((R₁₀)ₕ—R₁₁)ₙ—(R₁₀)ₕ—R₁₂ satisfies molecules from Group R^{M}, wherein it is understood that when n is more than 1, —((R₁₀)ₕ—R₁₁)ₙ—(R₁₀)ₕ—R₁₂ may be preceded by a group —(R₁₀)ₕ—R₁₁— so as to form a group —(R₁₀)ₕ—R₁₁—((R₁₀)ₕ—R₁₁)ₙ—(R₁₀)ₕ—R₁₂. It is understood that this follows from the definition of how to write out the repeating units, i.e. —((R₁₀)ₕ—R₁₁)₂— would first be written as —(R₁₀)ₕ—R₁₁—(R₁₀)ₕ—R₁₁— before R₁₀, h, and R₁₁ are independently selected.

### R₁₀

In preferred embodiments, each R₁₀ is independently selected from the group consisting of —O—, —S—, —SS—, —NR₄—, —N═N—, —C(O)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, —C(S)O—, —OC(S)O—, —OC(S)NR₄—, —NR₄C(S)—, —NR₄C(S)O—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —OP(O)(R₄)₂—, —SP(O)(R₄)₂—, —NR₄P(O)(R₄)₂—, and combinations thereof, wherein R₄ is defined as described herein.

In preferred embodiments, each R₁₀ is independently selected from the group consisting of —O—, —S—, —SS—, —NR₄—, —N═N—, —C(O)—, —C(O)NR₄—, —OC(O)—, —C(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —C(O)NR₄S(O)₂NR₄—, —OC(O)NR₄S(O)₂NR₄—, —OC(S)—, —C(S)O—, —OC(S)O—, —OC(S)NR₄—, —NR₄C(S)—, —NR₄C(S)O—, and —SS(O)₂—.

### R₁₁

In preferred embodiments, each R₁₁ is independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups, wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In preferred embodiments, each R₁₁ is independently selected from the group consisting of C₁-C₁₂ alkylene groups, C₂-C₁₂ alkenylene groups, C₂-C₁₂ alkynylene groups, C₆-C₁₂ arylene, C₂-C₁₂ heteroarylene, C₃-C₁₂ cycloalkylene groups, C₅-C₁₂ cycloalkenylene groups, and C₁₂ cycloalkynylene groups; and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In preferred embodiments, each R₁₁ is independently selected from the group consisting of C₁-C₆ alkylene groups, C₂-C₆ alkenylene groups, C₂-C₆ alkynylene groups, C₆ arylene, C₂-C₆ heteroarylene, C₃-C₆ cycloalkylene groups, and C₅-C₆ cycloalkenylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₁₁ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero) arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized;

In preferred embodiments, the R₁₁ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₄-C₁₂ (hetero)arylalkenyl groups, C₄-C₁₂ (hetero)arylalkynyl groups, C₄-C₁₂ alkenyl(hetero)aryl groups, C₄-C₁₂ alkynyl(hetero)aryl groups, C₄-C₁₂ alkylcycloalkyl groups, C₆-C₁₂ alkylcycloalkenyl groups, C₁₃-C₁₈ alkylcycloalkynyl groups, C₄-C₁₂ cycloalkylalkyl groups, C₆-C₁₂ cycloalkenylalkyl groups, C₁₃-C₁₈ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₅-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₁₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero) arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₁₁ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₆ alkyl(hetero)aryl groups, C₃-C₆ (hetero)arylalkyl groups, C₄-C₆ (hetero)arylalkenyl groups, C₄-C₆ (hetero)arylalkynyl groups, C₄-C₆ alkenyl(hetero)aryl groups, C₄-C₆ alkynyl(hetero)aryl groups, C₄-C₆ alkylcycloalkyl groups, C₆ alkylcycloalkenyl groups, C₄-C₆ cycloalkylalkyl groups, C₆ cycloalkenylalkyl groups, C₅-C₆ alkenylcycloalkyl groups, C₇ alkenylcycloalkenyl groups, C₅-C₆ cycloalkylalkenyl groups, C₇ cycloalkenylalkenyl groups, C₅-C₆ alkynylcycloalkyl groups, C₇ alkynylcycloalkenyl groups, C₅-C₆ cycloalkylalkynyl groups, C₅-C₆ cycloalkyl(hetero)aryl groups, and C₅-C₆ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

It is preferred that when in Formula (9) n>2, that R₁₁ is independently selected from the group consisting of C₁-C₆ alkylene groups, C₂-C₆ alkenylene groups, C₂-C₆ alkynylene groups, C₆-C₆ arylene, C₂-C₆ heteroarylene, C₃-C₆ cycloalkylene groups, and C₅-C₆ cycloalkenylene groups; and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₃₆, P, and Si, wherein the N, S, and P atoms are optionally oxidized, wherein the N atoms are optionally quaternized.

In a preferred embodiment, the R₁₁ substituents do not contain heteroatoms.
In a preferred embodiment, the R₁₁ groups are not substituted.
In another preferred embodiment, the R₁₁ groups do not contain heteroatoms.

### R₁₂

R₁₂ is selected from the group consisting of —H, —OH, —NH₂, —N₃, —Cl, —Br, —F, —I, and a chelating moiety.

Non-limiting examples of chelating moieties for use in R₁₂ are DTPA (diethylenetriaminepentaacetic acid), DOTA (1,4,7,10-tetraazacyclododecane-N,N′,N″,N″-tetraacetic acid), NOTA (1,4,7-triazacyclononane-N,N′,N″-triacetic acid), TETA (1,4,8,11-tetraazacyclotetradecane-N,N′,N″,N′-tetraacetic acid), OTTA (N1-(p-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide), and EDTA (ethylenediaminetetraacetic acid).

Preferably, R₁₂ is a chelator moiety selected from the group consisting of

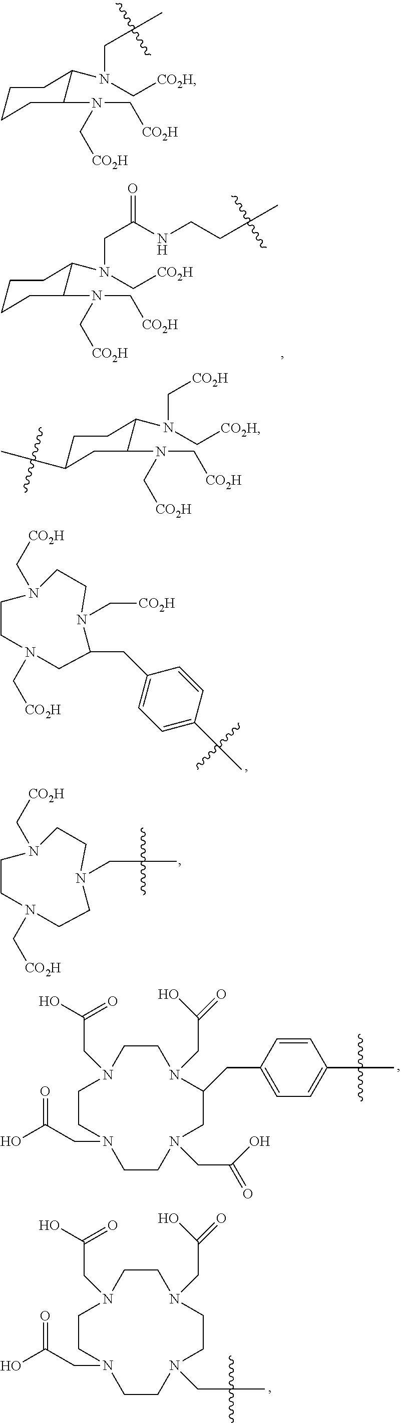

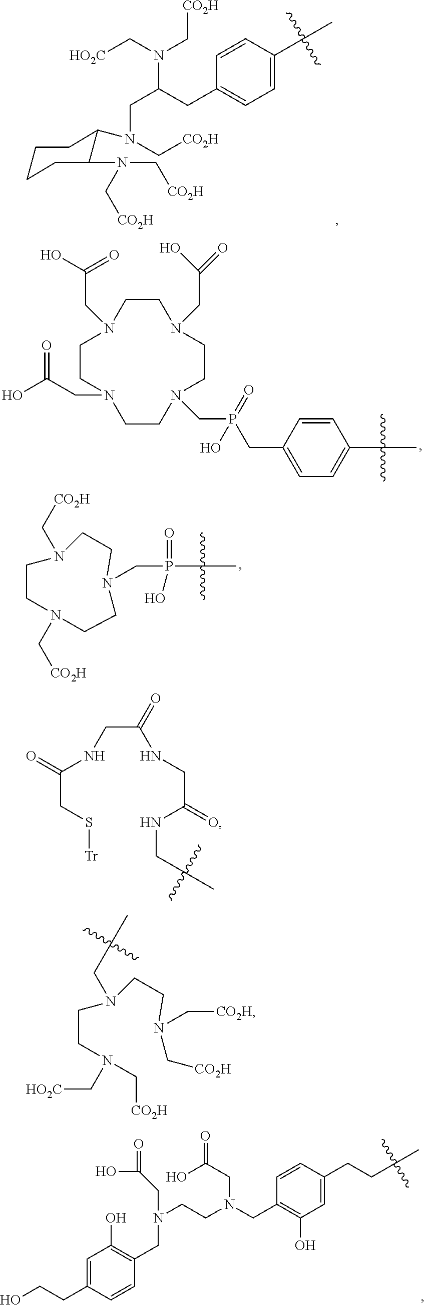

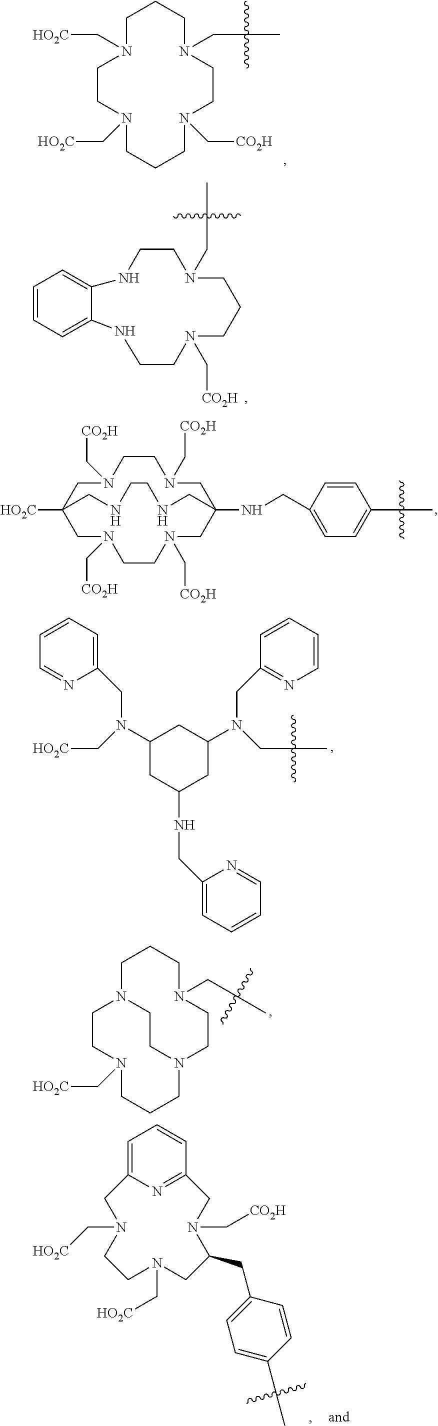

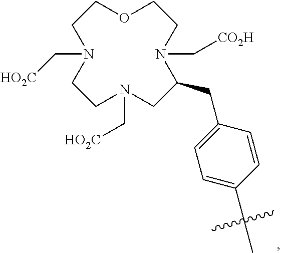 [SMILES: *c1ccc(C[C@H]2CN(CC(=O)O)CCN(CC(=O)O)CCOCCN2CC(=O)O)cc1]

wherein the wiggly line denotes a bond to the remaining part of the molecule, optionally bound via —C(O)NH—,
wherein the chelator moieties according to said group optionally chelate a metal, wherein the metal is preferably selected from the group consisting of ⁴⁴Sc, ⁶²Cu, ⁶⁴Cu, ⁶⁶Ga, ⁶⁷Ga, ⁶⁷Cu, ⁶⁸Ga, ⁸⁶Y, ⁸⁹Zr, ⁹⁰Y, ^{99m}Tc, ¹¹¹In, ¹⁶⁶Ho, ¹⁷⁷Lu, ¹⁸⁶Re, ¹⁸⁸Re, ²¹¹Bi, ²¹²Bi, ²¹²Pb, ²¹³Bi, ²¹⁴Bi, and ²²⁵Ac.

### R₄

In preferred embodiments, each R₄ is independently selected from the group consisting of hydrogen, C₁-C₂₄ alkyl groups, C₂-C₂₄ alkenyl groups, C₂-C₂₄ alkynyl groups, C₆-C₂₄ aryl, C₂-C₂₄ heteroaryl, C₃-C₂₄ cycloalkyl groups, C₅-C₂₄ cycloalkenyl groups, and C₁₂-C₂₄ cycloalkynyl groups; wherein the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized.

In preferred embodiments, each R₄ is independently selected from the group consisting of hydrogen, C₁-C₁₂ alkyl groups, C₂-C₁₂ alkenyl groups, C₂-C₁₂ alkynyl groups, C₆-C₁₂ aryl, C₂-C₁₂ heteroaryl, C₃-C₁₂ cycloalkyl groups, C₅-C₁₂ cycloalkenyl groups, and C₁₂ cycloalkynyl groups; wherein the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized.

In preferred embodiments, each R₄ is independently selected from the group consisting of hydrogen, C₁-C₆ alkyl groups, C₂-C₆ alkenyl groups, C₂-C₆ alkynyl groups, C₆ aryl, C₂-C₆ heteroaryl, C₃-C₆ cycloalkyl groups, and C₅-C₆ cycloalkenyl groups; wherein the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₄ groups not being hydrogen, are optionally further substituted with one or more substituents selected from the group consisting of —Cl, —F, —Br, —I, —OH, —NH₂, —SO₃H, —PO₃H, —PO₄H₂, —NO₂, —CF₃, ═O, —NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero) arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₄ groups not being hydrogen are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₄-C₁₂ (hetero)arylalkenyl groups, C₄-C₁₂ (hetero)arylalkynyl groups, C₄-C₁₂ alkenyl(hetero)aryl groups, C₄-C₁₂ alkynyl(hetero)aryl groups, C₄-C₁₂ alkylcycloalkyl groups, C₆-C₁₂ alkylcycloalkenyl groups, C₁₃-C₁₈ alkylcycloalkynyl groups, C₄-C₁₂ cycloalkylalkyl groups, C₆-C₁₂ cycloalkenylalkyl groups, C₁₃-C₁₈ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₅-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₁₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero) arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₄ groups not being hydrogen are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₆ alkyl(hetero)aryl groups, C₃-C₆ (hetero)arylalkyl groups, C₄-C₆ (hetero)arylalkenyl groups, C₄-C₆ (hetero)arylalkynyl groups, C₄-C₆ alkenyl(hetero)aryl groups, C₄-C₆ alkynyl(hetero)aryl groups, C₄-C₆ alkylcycloalkyl groups, C₆ alkylcycloalkenyl groups, C₄-C₆ cycloalkylalkyl groups, C₆ cycloalkenylalkyl groups, C₅-C₆ alkenylcycloalkyl groups, C₇ alkenylcycloalkenyl groups, C₅-C₆ cycloalkylalkenyl groups, C₇ cycloalkenylalkenyl groups, C₅-C₆ alkynylcycloalkyl groups, C₇ alkynylcycloalkenyl groups, C₅-C₆ cycloalkylalkynyl groups, C₅-C₆ cycloalkyl(hetero)aryl groups, and C₅-C₆ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In a preferred embodiment, the R₄ substituents do not contain heteroatoms.
In a preferred embodiment, the R₄ groups are not substituted.
In another preferred embodiment, the R₄ groups do not contain heteroatoms.
In preferred embodiments, R₄ is hydrogen.

### R₅

In preferred embodiments, each R₅ is independently selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₅ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.
In a preferred embodiment, the R₅ substituents do not contain heteroatoms.
In a preferred embodiment, the R₅ groups are not substituted.
In another preferred embodiment, the R₅ groups do not contain heteroatoms.
In preferred embodiments, R₅ is hydrogen.

### Section 2—Combinations in Relation to the Invention

The invention pertains to a combination comprising the compound according to the invention, and a dienophile, preferably as defined herein, more preferably as defined in Sections 7 and 8.

It will be understood that the compound according to the invention is a compound according to any one of Formulae (1)-(7) as described in Section 1.

### Section 3—Kits in Relation to the Invention

The invention also relates to kits comprising a combination of the invention as defined in Section 2.

### Section 4—Compounds, Combinations and Kits in Relation to the Invention for Use in the Treatment of Subjects

The invention further pertains to the combination according to the invention or the kit according to the invention for use in the treatment of a subject, wherein said subject is preferably a human.

Preferably, the method of administering is as defined herein. Preferably, the combination according to the invention or the kit according to the invention is for use in the treatment of a disease or disorder in a subject, wherein the disease or disorder is as defined herein.

### Section 5—Method for Imaging

The invention also pertains to a method for imaging in a subject, wherein said method comprises first administering to said subject a dienophile as defined herein, wherein said dienophile comprises a targeting agent, and wherein said dienophile preferably is an eight-membered non-aromatic cyclic alkene that comprises a releasable group, preferably comprising a Construct A, on the allylic position, wherein said method further comprises sequentially administering to said subject a compound according to the invention, wherein said subject is preferably a human.
It will be understood that the compound according to the invention is a compound according to any one of Formulae (1)-(7) as described in Section 1.
Preferably, the method of administering is as defined herein.

### Section 6—Use of Compounds, Combinations, and Kits in Relation to the Invention

The invention further pertains to the use of a compound according to the invention, or a combination according to the invention, or the kit according to the invention in a bioorthogonal reaction.

In a preferred embodiment, the use is in vitro. In particular, the in vitro use is for applications in chemical synthesis, radiochemistry, surface modification, chemical biology, capture and release resins, in vitro diagnostics kits, and the like. Further examples on in vitro applications are found in Section 22.

In a preferred embodiment, the use is in a chemical environment.
In a preferred embodiment, the use is in a biological environment.

In a preferred embodiment, the use is in a physiological environment.

In a preferred embodiment, the use is in vitro.

In a preferred embodiment, the use is in vivo.

### Section 7—Dienophiles

Suitable dienophiles for combinations and kits disclosed herein are known to the skilled person.

It will be understood that a dienophile useful in a combination according to some embodiments of the invention may herein be referred to as a Prodrug.

In a preferred embodiment, the dienophile is an eight-membered non-aromatic cyclic alkene, preferably a cyclooctene, and more preferably a trans-cyclooctene.

In a preferred embodiment, the dienophile is an eight-membered non-aromatic cyclic alkene, wherein preferably the eight-membered non-aromatic cyclic alkene carries a releasable group on the allylic position. Preferably, this releasable group is connected to the allylic carbon via an ether, thioether, ester, thioester, thionoester, carbonate, thiocarbonate, carbamate, thiocarbamate, or carbodithio (i.e. —S(S)—). Preferably the eight-membered non-aromatic cyclic alkene is a cyclooctene, and more preferably a trans-cyclooctene, that carries a releasable group (herein termed Construct-A) on the allylic position. Such dienophiles are known to the skilled person.

In a preferred embodiment, the dienophile is as defined in WO 2012/156918 A1, in particular as in any one of Claims 5-7 thereof.

In a preferred embodiment, the dienophile is as defined in WO 2012/156919 A1, in particular as in any one of Claims 1-4 thereof.

In a preferred embodiment, the dienophile is as defined in WO 2012/156920 A1, in particular as in any one of Claims 1-6 thereof.

In a preferred embodiment, the dienophile is as defined in WO 2014/081303 A1, in particular as in any one of Claims 1-9 thereof.

Herein, the entire contents of WO 2012/156918 A1, WO 2012/156919 A1, WO 2012/156920 A1, and WO 2014/081303 A1 are incorporated by reference.

### General Formulae of Suitable Dienophiles

The following structures are non limiting examples of suitable dienophiles:

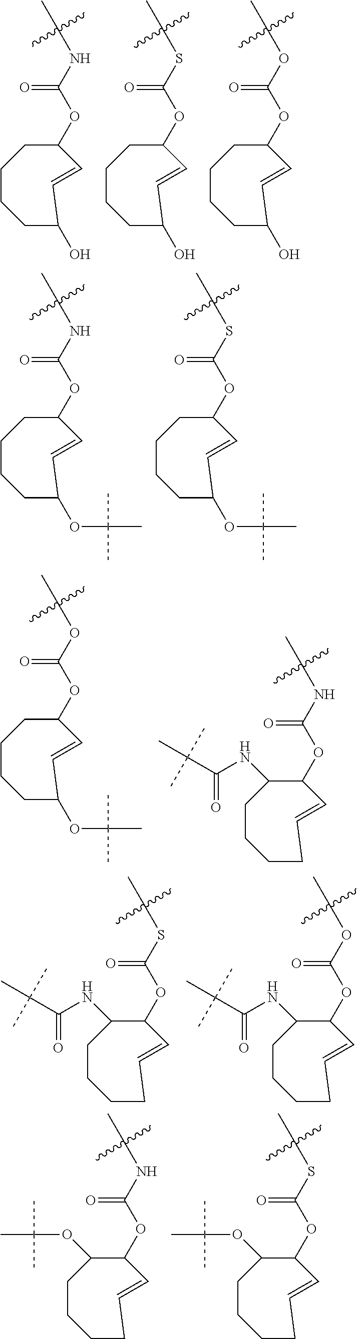

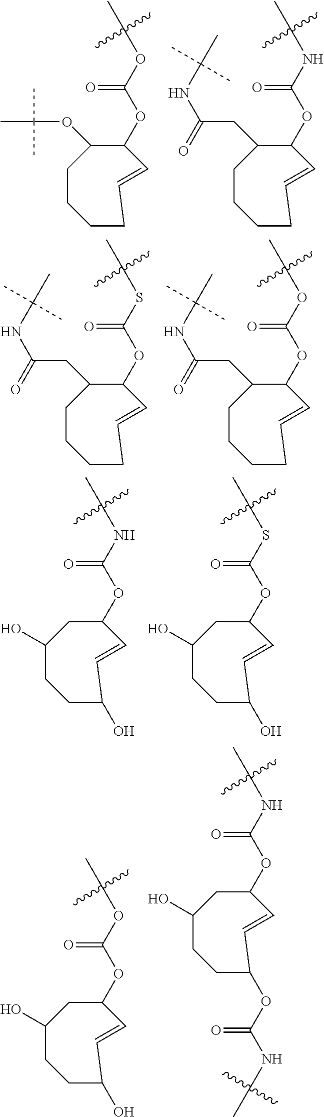

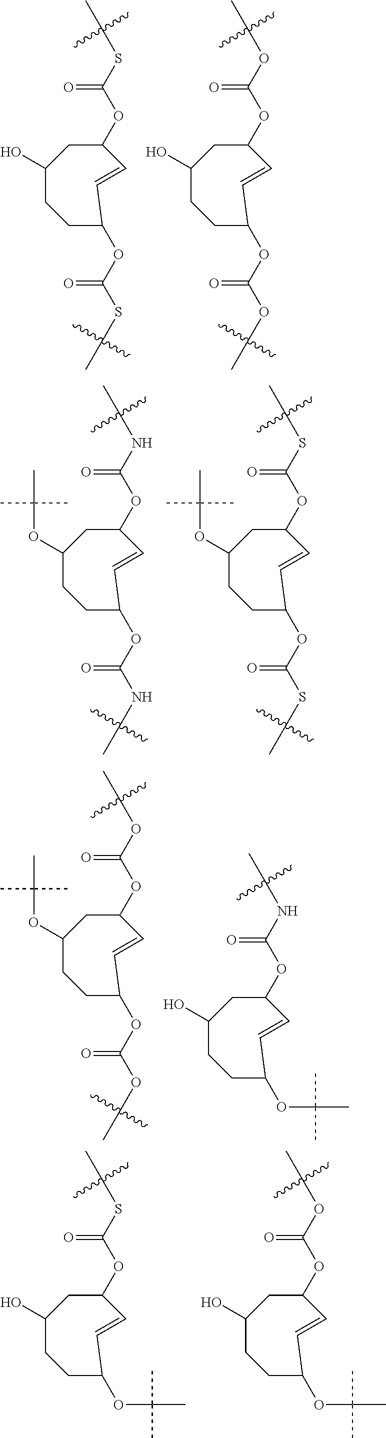

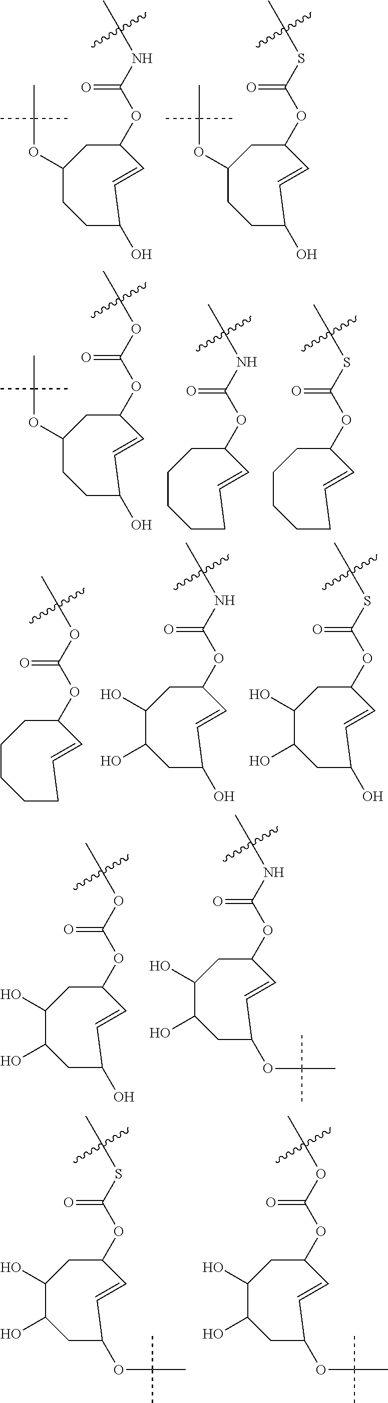

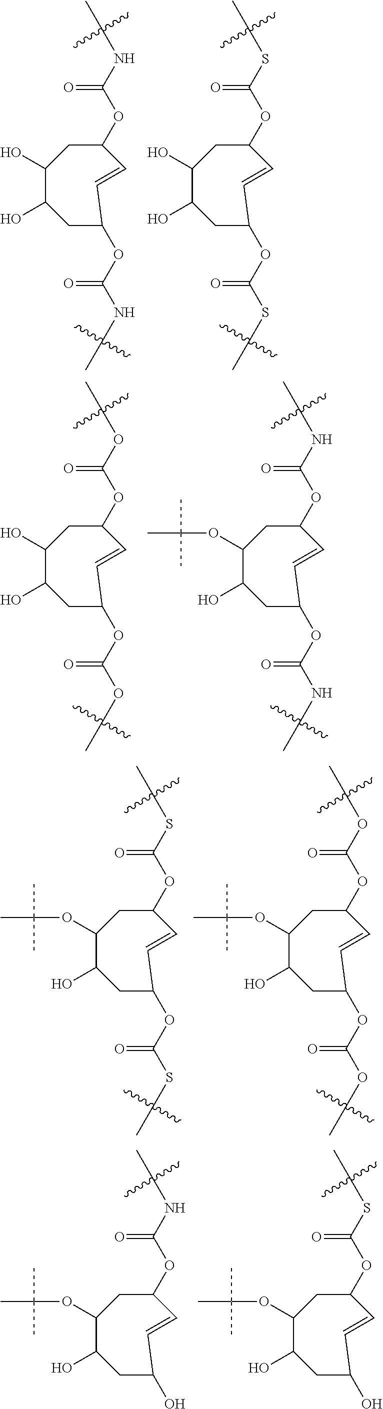

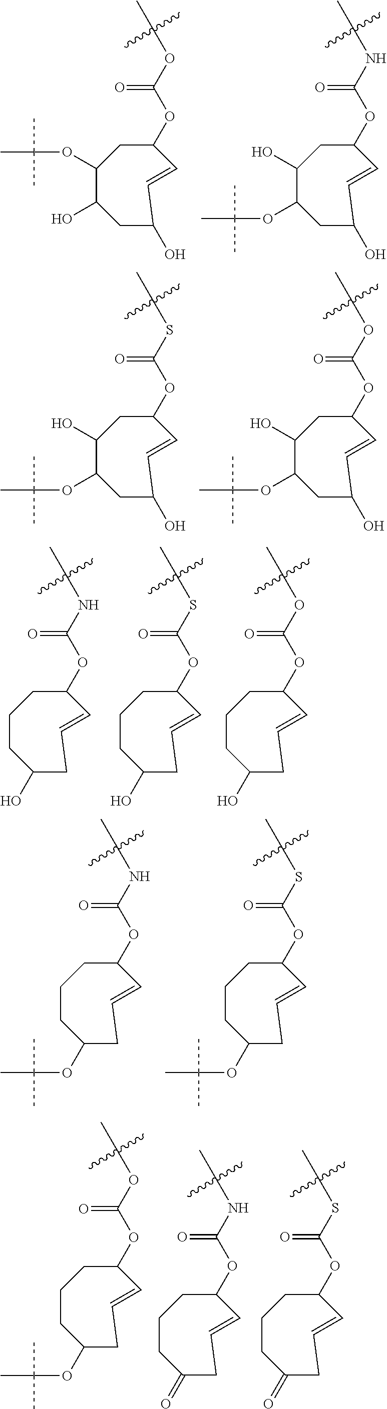

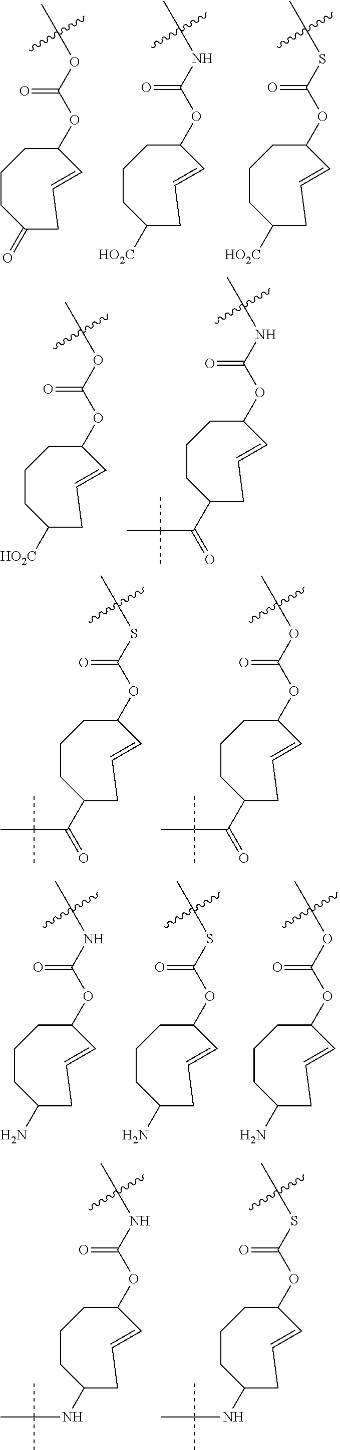

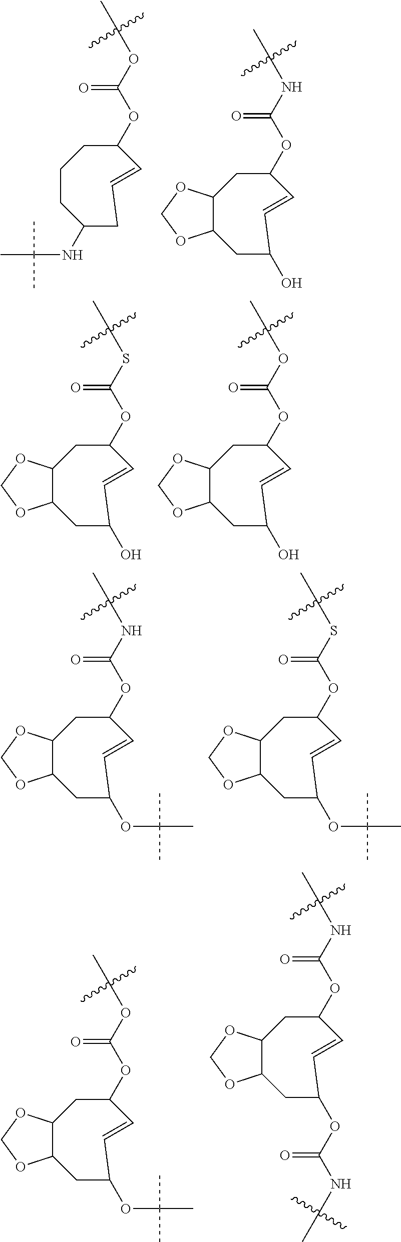

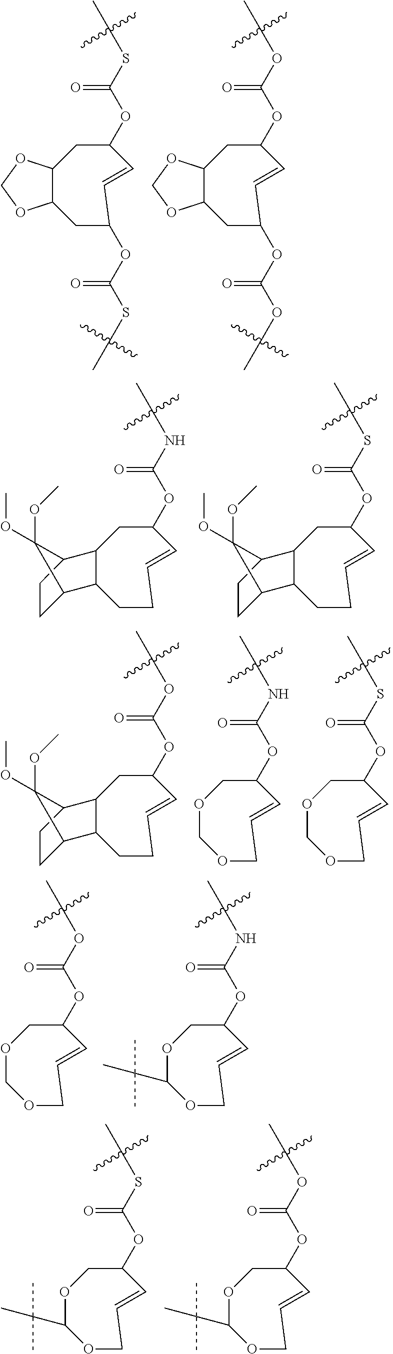

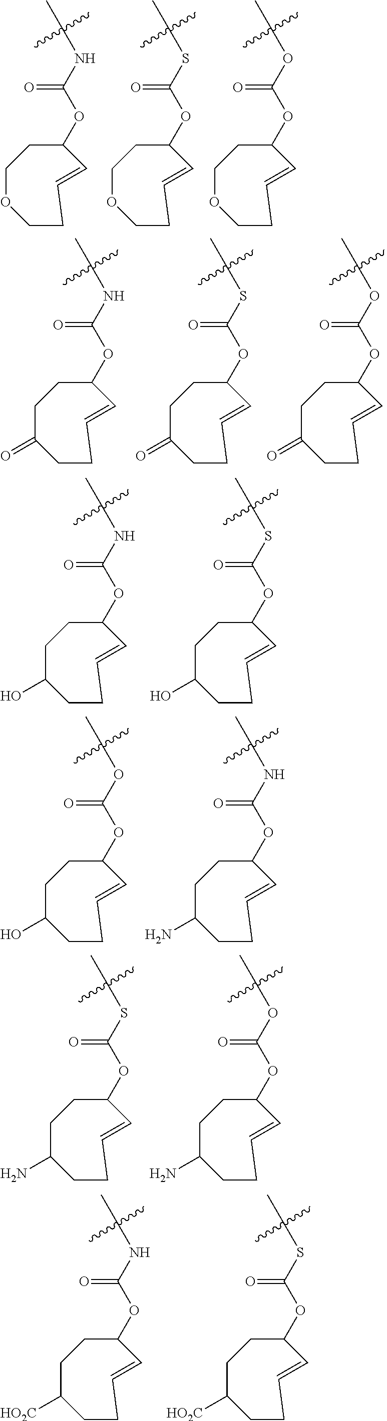

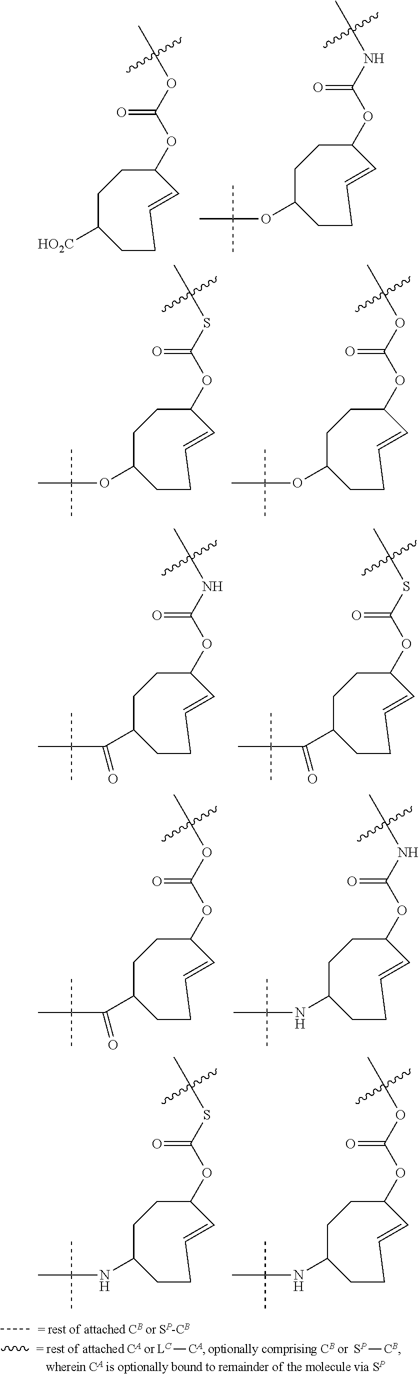

It should be noted that, depending on the choice of nomenclature, the TCO dienophile may also be denoted E-cyclooctene. With reference to the conventional nomenclature, it will be understood that, as a result of substitution on the cyclooctene ring, depending on the location and molecular weight of the substituent, the same cyclooctene isomer may formally become denoted as a Z-isomer. In the present invention, any substituted variants of the invention, whether or not formally “E” or “Z,” or “cis” or “trans” isomers, will be considered derivatives of unsubstituted trans-cyclooctene, or unsubstituted E-cyclooctene. The terms “trans-cyclooctene” (TCO) as well as E-cyclooctene are used interchangeably and are maintained for all dienophiles according to the present invention, also in the event that substituents would formally require the opposite nomenclature. I.e., the invention relates to cyclooctene in which carbon atoms 1 and 6 as numbered below in Formula 4b are in the E (entgegen) or trans position.

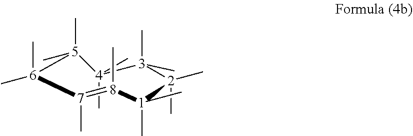

The dienophiles for use in the invention can be synthesized by the skilled person, on the basis of known synthesis routes to cyclooctenes and corresponding hetero atom(s)-containing rings. The skilled person further is aware of the wealth of cyclooctene derivatives that can be synthesized via the ring closing metathesis reaction using Grubbs catalysts. As mentioned above, the TCO possibly includes one or more heteroatoms in the ring. This is as such sufficiently accessible to the skilled person [e.g. WO2016025480]. Reference is made, e.g., to the presence of a thioether in TCO: [Cere et al. J. Org. Chem. 1980, 45, 261]. Also, e.g., an —O—SiR₂—O moiety in TCO: [Prevost et al. J. Am. Chem. Soc. 2009, 131, 14182]. References to TCO syntheses wherein the allylic positioned leaving group (R₄₈) is an ether, ester, carbonate, carbamate or a thiocarbamate are: [Versteegen et al Angew. Chem. Int. Ed. 2018, 57, 10494], and [Steiger et al Chem Comm 2017, 53, 1378]. Exemplary compounds include the following structures, indicated below with literature references. Where a cyclooctene derivative is depicted as a Z-cyclooctene it is conceived that this can be converted to the E-cyclooctene analog.

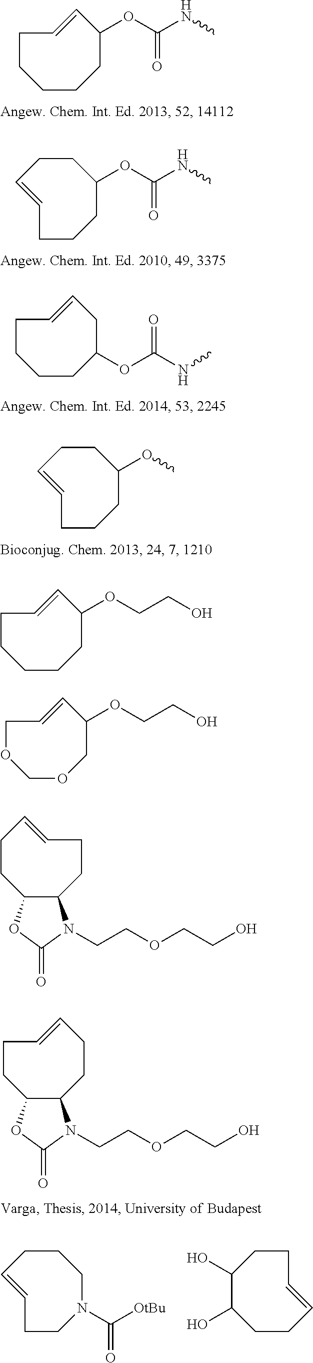

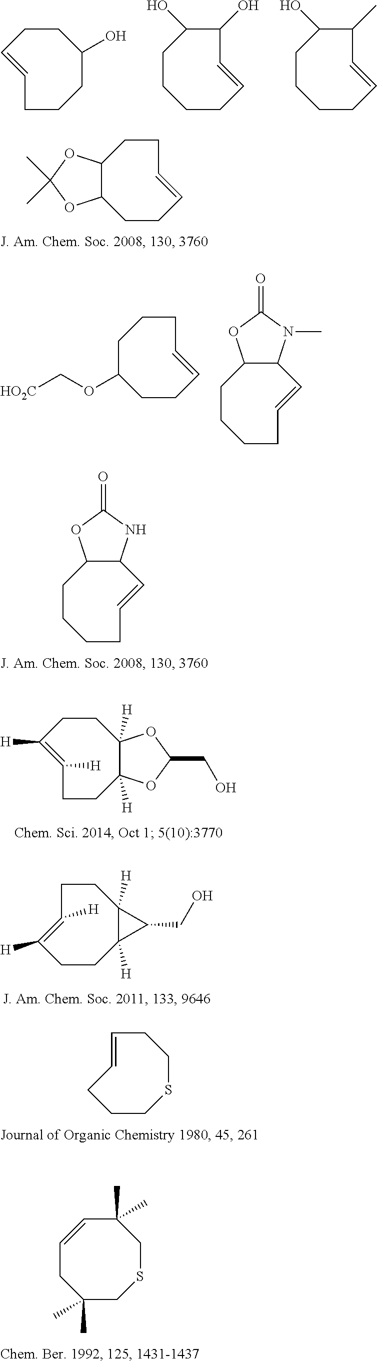

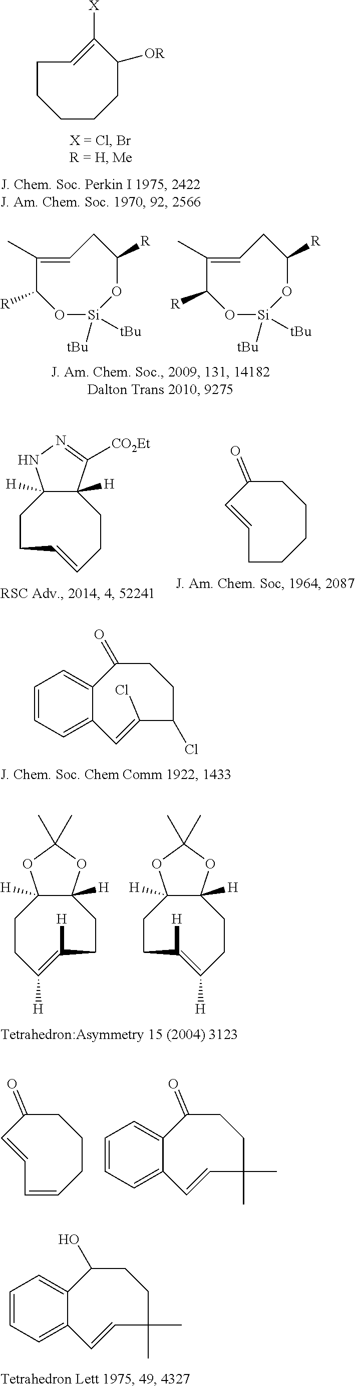

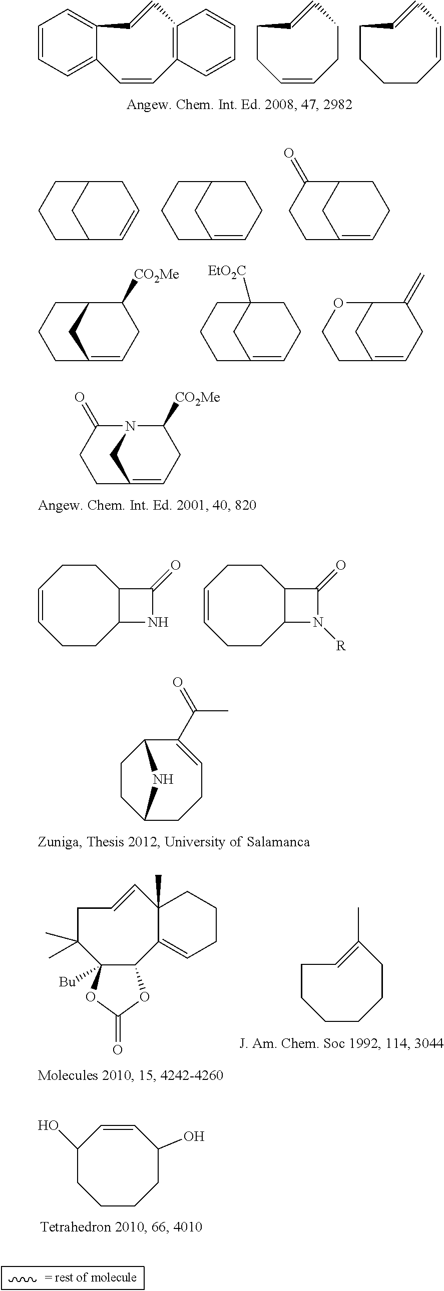

### Section 8—Dienophiles According to Formulae (19)-(22)

In preferred embodiments, the dienophile satisfies Formula (19):

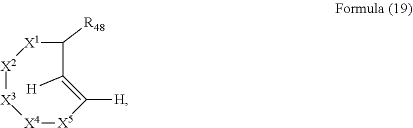

and pharmaceutically acceptable salts thereof,
wherein R₄₈ is selected from the group consisting of —OH,
—OC(O)Cl, —OC(O)O—N-succinimidyl, —OC(O)O-4-nitrophenyl, —OC(O)O-tetrafluorophenyl, —OC(O)O-pentafluorophenyl, —OC(O)—(S^{P})ₖ—C^{A}, —OC(S)—(S^{P})ₖ—C^{A}, —O-(L^{C}((S^{P})ₖC^{A})ₛ((S^{P})ₖ—C^{A})ₛ((S^{P})ᵢ—CB)ⱼ)ᵣ—(S^{P})ₖ—C^{A}, —(S^{P})ₖ—C^{A}, —SC(O)—(S^{P})ₖ—C^{A}, —SC(S)—(S^{P})ₖ—C^{A}, and —S-(L^{C}((S^{P})ₖC^{A})ₛ((S^{P})ₖ—C^{A})ₛ((S^{P})ᵢ—C¹³)ⱼ)ᵣ—(S^{P})ₖ—C^{A}; preferably —OC(O)—(S^{P})ₖ—C^{A}, —OC(S)—(S^{P})ₖ—C^{A}, —O-(L^{C}((S^{P})ₖC^{A})ₛ((S^{P})ₖ—C^{A})ₛ((S^{P})ᵢ—CB)ⱼ)ᵣ—(S^{P})ₖ—C^{A}, and —(S^{P})ₖ—C^{A};
wherein r is an integer in a range of from 0 to 2,
wherein each s is independently 0 or 1,
wherein i is an integer in a range of from 0 to 4,
wherein j is 0 or 1,
wherein each k is independently 0 or 1,
wherein L^{C} is a self-immolative linker, wherein S^{P} is a spacer,
wherein C^{A} denotes a Construct A,
wherein C^{B} denotes a Construct B,
wherein each C^{A} and each C^{B} can be independently selected from the groups defined in Sections 18 and 22,
wherein, when R₄₈ is —OC(O)—(S^{P})ₖC^{A}, —OC(S)—(S^{P})ₖC^{A}, —SC(O)—(S^{P})k-C^{A}, or —SC(S)—(S^{P})ₖ—C^{A}, S^{P} (when k>0) or C^{A} (when k=0) is bound to the —OC(O)—, —OC(S)—, —SC(O)—, or —SC(S)— of R₄₈ via an atom selected from the group consisting of O, C, S, and N, preferably a secondary or a tertiary N, wherein this atom is part of S^{P} or C^{A};
wherein preferably R₄₈ is —OC(O)—(S^{P})ₖC^{A}, and S^{P} (when k>0) or C^{A} (when k=0) is bound to the —OC(O)— of R₄₈ via an atom selected from the group consisting of O, C, S, and N, preferably N, more preferably a secondary or a tertiary N, wherein this atom is part of S^{P} or C^{A};
wherein, when R₄₈ is —O-(L^{C}((S^{P})ₖC^{A})ₛ((S^{P})ₖC^{A})ₛ((S^{P})ᵢ—C^{B})ⱼ)ᵣ—(S^{P})ₖC^{A} or —S-(L^{C}((S^{P})ₖC^{A})ₛ((S^{P})ₖ—C^{A})ₛ((S^{P})ᵢ—C^{B})ⱼ)ᵣ—(S^{P})ₖ—C^{A} and r is O, S^{P} (when k>0) or C^{A} (when k=0) is bound to the —O— or —S— moiety of R₄₈ on the allylic position of the trans-cyclooctene ring of Formula (19) via a group selected from the group consisting of —C(O)—, and —C(S)—, wherein this group is part of S^{P} or C^{A},
wherein, when R₄₈ is —O-(L^{C}((S^{P})ₖC^{A})ₛ((S^{P})ₖC^{A})ₛ((S^{P})ᵢ—C^{B})ⱼ)ᵣ—(S^{P})ₖC^{A} or —S-(L^{C}((S^{P})ₖC^{A})ₛ((S^{P})ₖ—C^{A})ₛ((S^{P})ᵢ—C^{B})ⱼ)ᵣ—(S^{P})ₖ—C^{A} and r is 1, L^{C} is bound to the —O— or —S— moiety on the allylic position of the trans-cyclooctene ring of Formula (19) via a group selected from the group consisting of —C(Y^{C2})Y^{C1}—, and a carbon atom, preferably an aromatic carbon, wherein this group is part of L^{C},
wherein Y^{C1}— is selected from the group consisting of —O—, —S—, and —NR₃₆—,
wherein Y^{C2} is selected from the group consisting of O and S,
wherein, when R₄₈ is —O-(L^{C}(S^{P})ₖC^{A})ₛ((S^{P})ₖC^{A})ₛ((S^{P})ᵢ—C^{B})ⱼ)ᵣ—(S^{P})ₖC^{A}, or —S-(L^{C}((S^{P})ₖC^{A})ₛ((S^{P})ₖ—C^{A})ₛ((S^{P})ᵢ—C^{B})ⱼ)ᵣ—(S^{P})ₖ—C^{A} and r is 1, then S^{P} (when k>0) or C^{A} (when k=0) is bound to L^{C} via a moiety selected from the group consisting of —O—, —S—, and —N—, preferably a secondary or a tertiary N, wherein said moiety is part of S^{P} or C^{A},
wherein, when R₄₈ is —(S^{P})ₖC^{A}, then S^{P} (when k>0) or C^{A} (when k=0) is bound to the allylic position of the trans-cyclooctene of Formula (19) via an —O— or —S— atom, wherein this atom is part of S^{P} or C^{A},
wherein preferably each R₃₆ is independently selected from the group consisting of hydrogen, —(S^{P})ᵢ—C^{B} with i being an integer in a range of from 0 to 4, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo) alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups
wherein for R₃₆ the groups not being hydrogen are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein preferably i is an integer ranging from 0 to 1,
wherein preferably, the R₃₆ groups not being hydrogen are not substituted and do not contain heteroatoms,
wherein X⁵ is —C(R₄₇)₂— or —CHR₄₈, preferably X⁵ is —C(R₄₇)₂—, preferably X⁵ is —CHR₄₈,
wherein each X¹, X², X³, X⁴ is independently selected from the group consisting of —C(R₄₇)₂—, —NR₃₇—, —C(O)—, —O—, such that at most two of X¹, X², X³, X⁴ are not —C(R₄₇)₂—, and with the proviso that no sets consisting of adjacent atoms are present selected from the group consisting of —O—O—, —O—N—, —C(O)—O—, N—N—, and —C(O)—C(O)—,
wherein each R₄₇ is independently selected from the group consisting of hydrogen, —(S^{P})ᵢ—C^{B} with i being an integer in a range of from 0 to 4, —F, —Cl, —Br, —I, —OR₃₇, —N(R₃₇)₂, —SO₃, —PO₃, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(—S)O—R₃₇, C(—S)S—R₃₇, S(O)R₃₇, —S(O)₂R₃₇, NR₃₇S(O)₂R₃₇, —ON(R₃₇)₂, —NR₃₇OR₃₇, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups; wherein preferably each R₄₇ is independently selected from the group consisting of hydrogen, —F, —Cl, —Br, —I, —OH, —NH₂, —SO₃⁻, —PO₃⁻, —NO₂, —CF₃, —SH, —(S^{P})ᵢ—C^{B}, C₁-C₈ alkyl groups, C₂-C₈, alkenyl groups, C₂-C₈, alkynyl groups, C₆-C₁₂ aryl groups, C₂-C₁₂ heteroaryl groups, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups, and C₅-C₁₂ (hetero)arylcycloalkyl groups;
wherein preferably i is an integer ranging from 0 to 1,
wherein the alkyl groups, alkenyl groups, alkynyl groups, aryl, heteroaryl, cycloalkyl groups, cycloalkenyl groups, cycloalkynyl groups, (cyclo)alkyl(hetero)aryl groups, (hetero)aryl(cyclo)alkyl groups, (cyclo)alkenyl(hetero)aryl groups, (hetero)aryl(cyclo)alkenyl groups, (cyclo)alkynyl(hetero)aryl groups, (hetero)aryl(cyclo)alkynyl groups, alkylcycloalkyl groups, cycloalkylalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized,
wherein in some preferred embodiments, the R₄₇ groups not being hydrogen are not substituted,
wherein in some preferred embodiments, the R₄₇ groups not being hydrogen do not contain heteroatoms,
wherein two R₄₇ and/or R₃₇ are optionally comprised in a ring,
wherein two R₄₇ and/or R₃₇ are optionally comprised in a ring so as to form a ring fused to the eight-membered trans-ring,
wherein each R₃₇ is independently selected from the group consisting of hydrogen, —(S^{P})ᵢ—C^{B} with i being an integer in a range of from 0 to 4, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo) alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups;
wherein preferably each R₃₇ is independently selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups, and C₅-C₁₂ (hetero)arylcycloalkyl groups;
wherein preferably i is an integer ranging from 0 to 1,
wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized,
wherein in some preferred embodiments, the R₃₇ groups not being hydrogen are not substituted,
wherein in some preferred embodiments, the R₃₇ groups not being hydrogen do not contain heteroatoms,
wherein in preferred embodiments at most three C^{B} is comprised in the structure of Formula (19), more preferably at most two, most preferably at most one C^{B} is comprised in the structure of Formula (19).
When C^{B} is present in a structure according to any one of Formulae (19), in preferred embodiments C^{B} is bound to the remainder of the molecule via a residue of R₃₂ as defined herein, wherein preferably said residue of R₃₂ equals or is comprised in a Spacer.
A person skilled in the art will understand that “residue of R₃₂” means the conjugation reaction product of R₃₂ with another chemical group so as to form a conjugate between C^{A} and/or C^{B} with the Trigger, L^{C} or S^{P}.

In preferred embodiments, when C^{B} is present in a structure according to Formula (19) C^{B} is bound to the remainder of the molecule via C^{M2} as defined herein, wherein preferably C^{M2} equals or is comprised in a Spacer.

In yet other embodiments, when C^{B} is present in a structure according to Formula (19) C^{B} is bound to the remainder of the molecule via C^{X} as defined herein, wherein preferably C^{X} equals or is comprised in a Spacer.

In some embodiments, moiety C^{X}, C^{M2} and the said residue of R₃₂ are comprised in C^{A} and/or C^{B}.

In preferred embodiments, C^{M2} is selected from the group consisting of amine, amide, thioamide, aminooxy, ether, carbamate, thiocarbamate, urea, thiourea, sulfonamide, and sulfoncarbamate.

In preferred embodiments C^{M2} equals R₁₀ as defined in Section 1.

In preferred embodiments, C^{M2} is selected from the group consisting of:

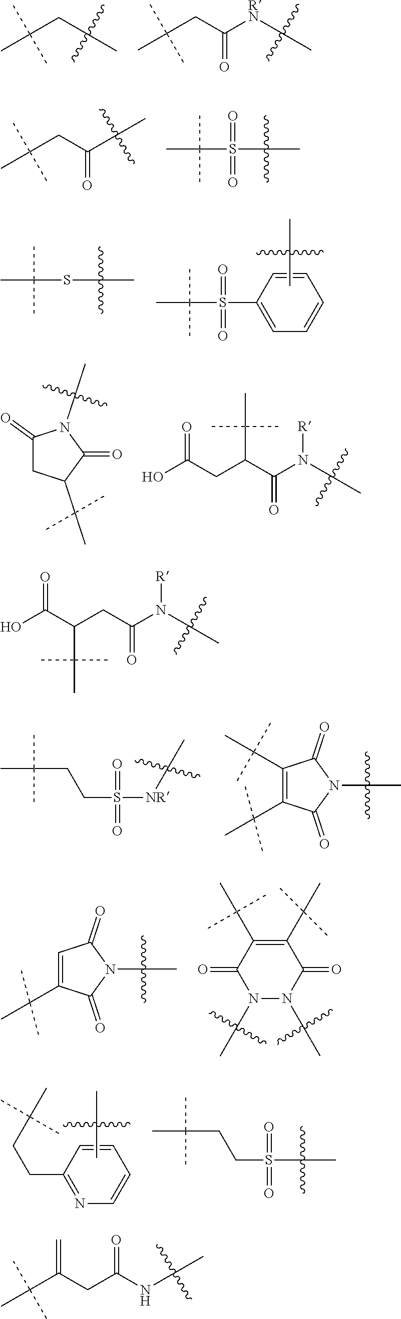

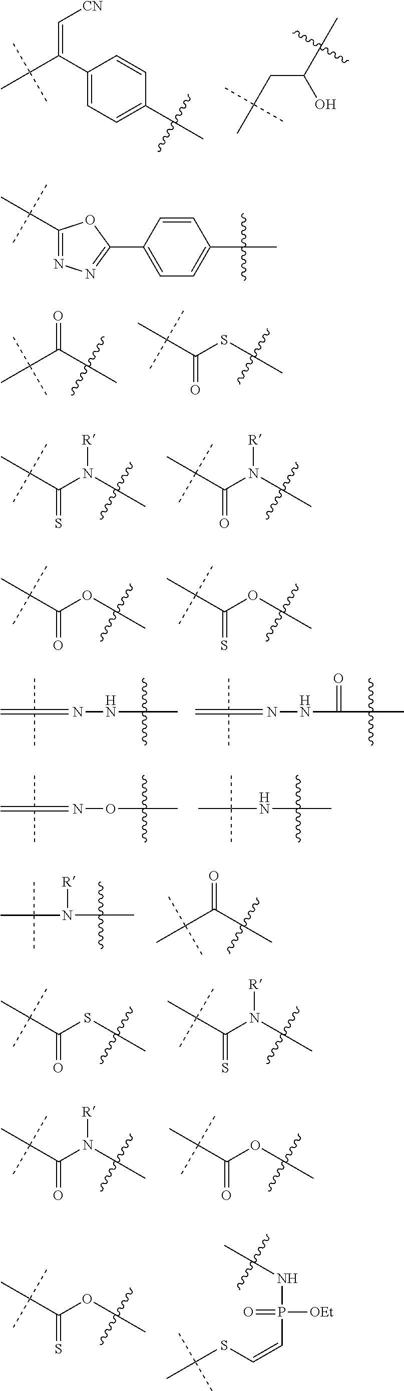

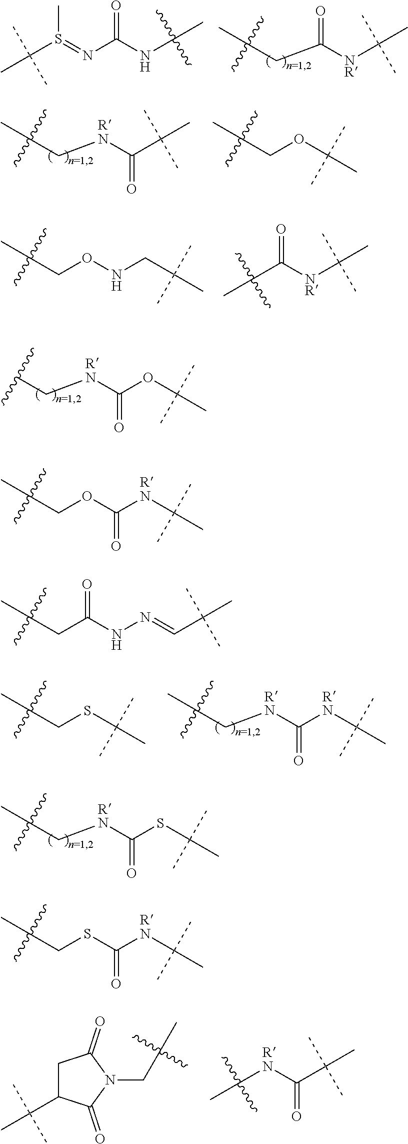

wherein R′ equals R₃₇, wherein the dashed line denotes a bond to or towards C^{B} and the wiggly line denotes a bond to the remaining part of the dienophile. In other embodiments the wiggly line denotes a bond directly to or towards C^{B} and the dashed line denotes a bond to the remaining part of the dienophile.
In preferred embodiments, C^{X} is:

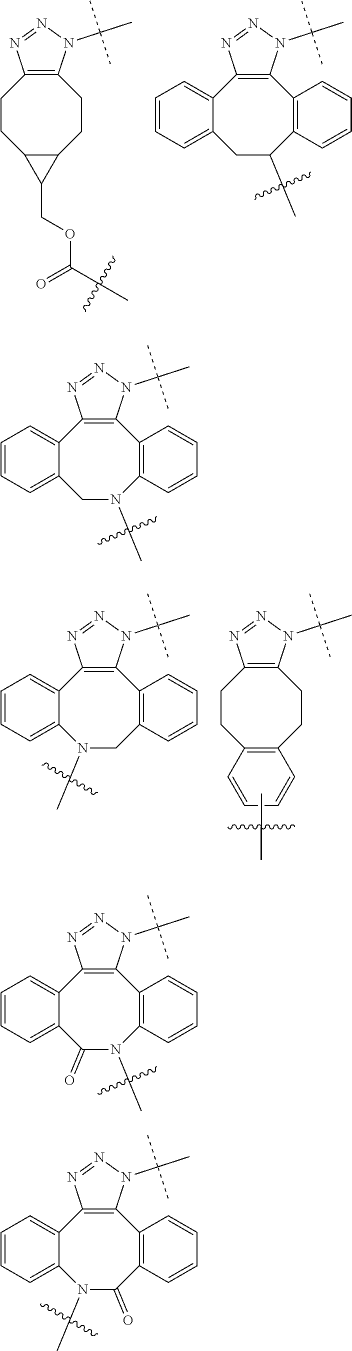

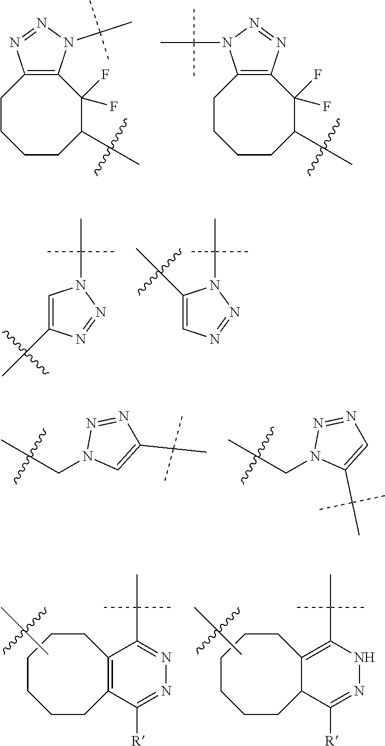

wherein the dashed line denotes a bond to or towards C^{B} and the wiggly line denotes a bond to the remaining part of the dienophile. Herein, R′ is preferably as defined for R₃₇. In other embodiments the wiggly line denotes a bond to or towards C^{B} and the dashed line denotes a bond to the remaining part of the dienophile.
In embodiments where C^{A} is bound to the Trigger via a Spacer S^{P}, then C^{A} can be bound to S^{P} via a residue of R₃₂, or via C^{M2} or via C^{X}, in the same manner as described for C^{B} herein above.
With reference to above schemes with examples of C^{M2} and C^{X}, in some embodiments it is preferred that when C^{A} or C^{B} is a protein, such as an antibody, that the dashed line denotes a bond to or towards C^{A} or C^{B}.
In preferred embodiments, when i is 0, then C^{B} is linked to the remaining part of Formula 19 via a moiety selected from the group consisting of —O—, —C(R⁶)₂—, —NR⁶—, —C(O)—, and —S—, wherein said moieties are part of C^{B}.
In preferred embodiments, when i is at least 1, then C^{B} is linked to S^{P} via a moiety selected from the group consisting of —O—, —C(R⁶)₂—, —NR⁶—, C(O), and —S—, wherein said moieties are part of C^{B}, and S^{P} is linked to the remaining part of Formula 19 via a moiety selected from the group consisting of —O—, —C(R⁶)₂—, —NR⁶—, —C(O)— and —S—, wherein said moieties are part of S^{P}.
In preferred embodiments, when i is at least 1, then S^{P} is linked to the remaining part of L^{C} via a moiety selected from the group consisting of —O—, —C(R⁶)₂—, —NR⁶—, and —S—, wherein said moieties are part of S^{P}.

In a preferred embodiment, at most three R₄₇ in Formula (19) are not H.

In a preferred embodiment, all X in Formula (19) are —C(R₄₇)₂—.

In a preferred embodiment, X¹, X², X³, X⁴ are all —C(R₄₇)₂— and at most 3 of R₄₇ are not H, more preferably at most 2 R₄₇ are not H.

In a preferred embodiment, at most one of X¹, X², X³, X⁴ is not —C(R₄₇)₂— and at most 3 of R₄₇ are not H, more preferably at most 2 R₄₇ are not H.

In a preferred embodiment, two of X², X³, X⁴ together form an amide and at most 3 of R₄₇ are not H, more preferably at most 2 R₄₇ are not H.

In a preferred embodiment, X¹ is C(R₄₇)₂.

In particularly favorable embodiments, R₄₈ is in the axial position.

It is preferred that when two R₄₇ and/or R₃₇ groups are comprised in a ring so as to form a ring fused to the eight-membered trans-ring, that these rings fused to the eight-membered trans-ring are C₃-C₇ cycloalkylene groups and C₄-C₇ cycloalkenylene groups, optionally substituted and containing heteroatoms as described for R₄₇.

In preferred embodiments, each individual C^{B} is linked to the remainder of the structure according to Formula (19) via a moiety that is part of C^{B}

independently selected from the group consisting of —O—, —C(R⁶)₂—, —NR⁶—, —S—,

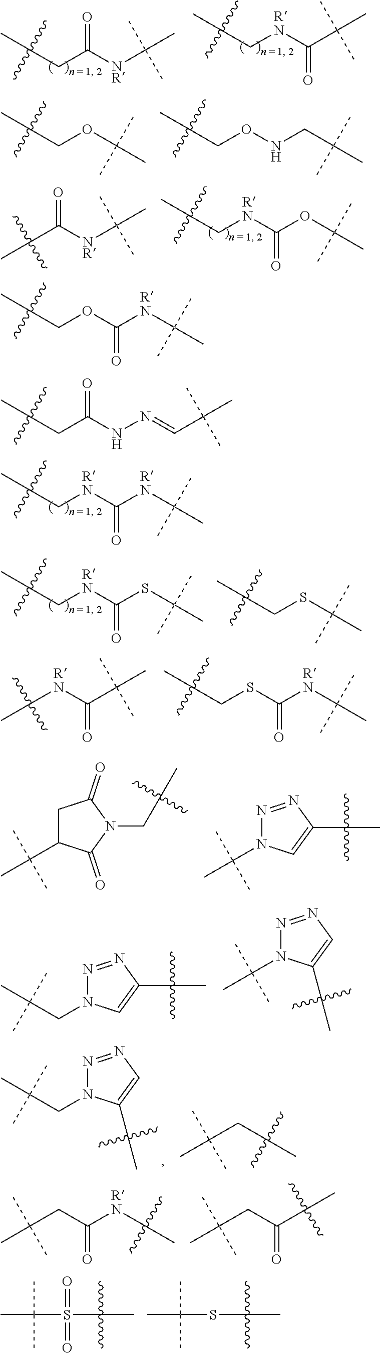

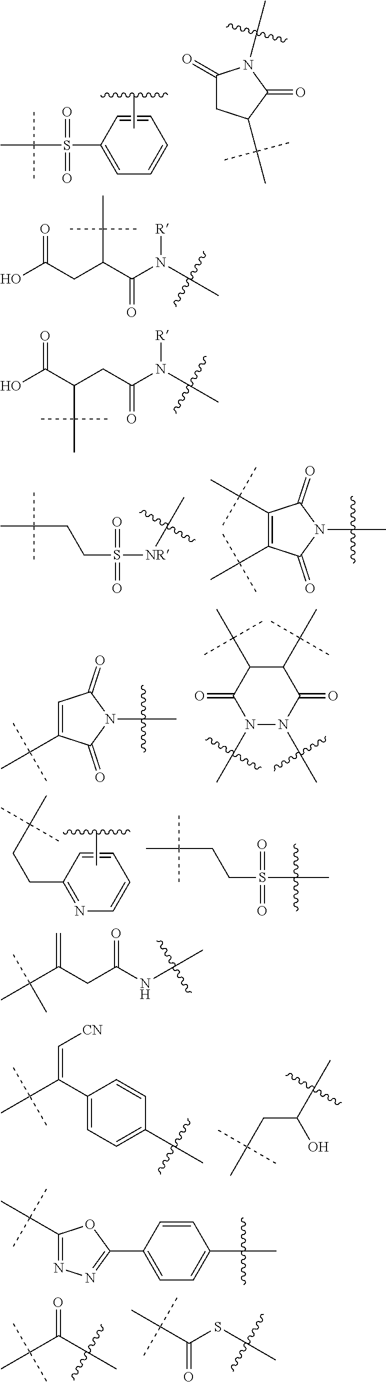

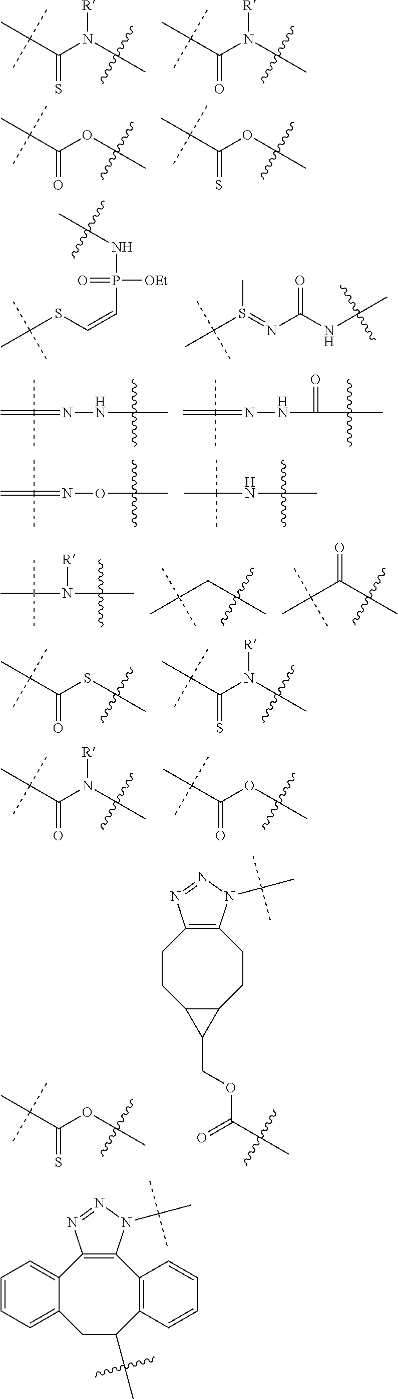

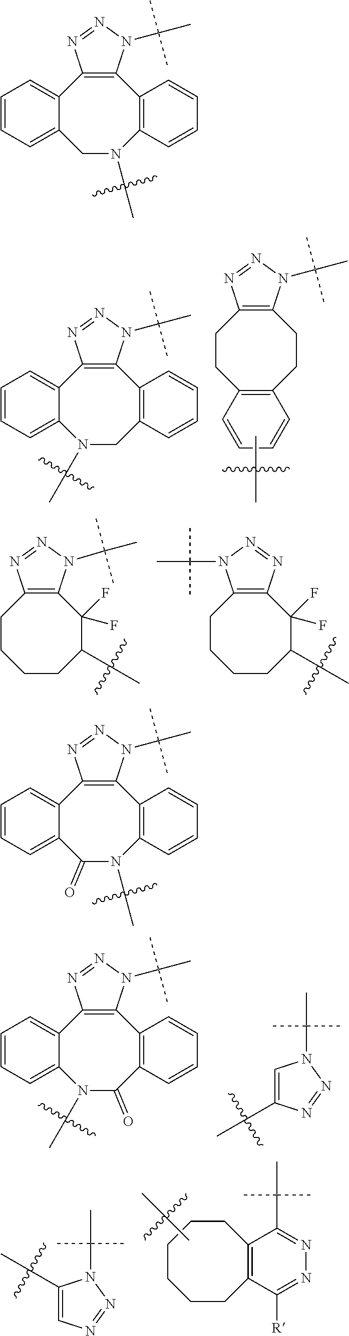

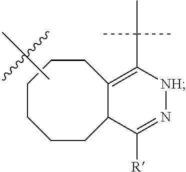

wherein the wiggly line depicts a bond to the remainder of C^{B} or to the remainder of the structure according to Formula (19), and the dashed line depicts either a bond to the remainder of C^{B} when the wiggly line depicts a bond to the remainder of the structure according to Formula (19), or a bond to the remainder of the structure according to Formula (19) when the wiggly line depicts a bond to the remainder of C^{B}. Herein, R′ is preferably as defined for R₃₇.

In preferred embodiments the dienophile satisfies any one of the Formulae (20)-(20m) below:

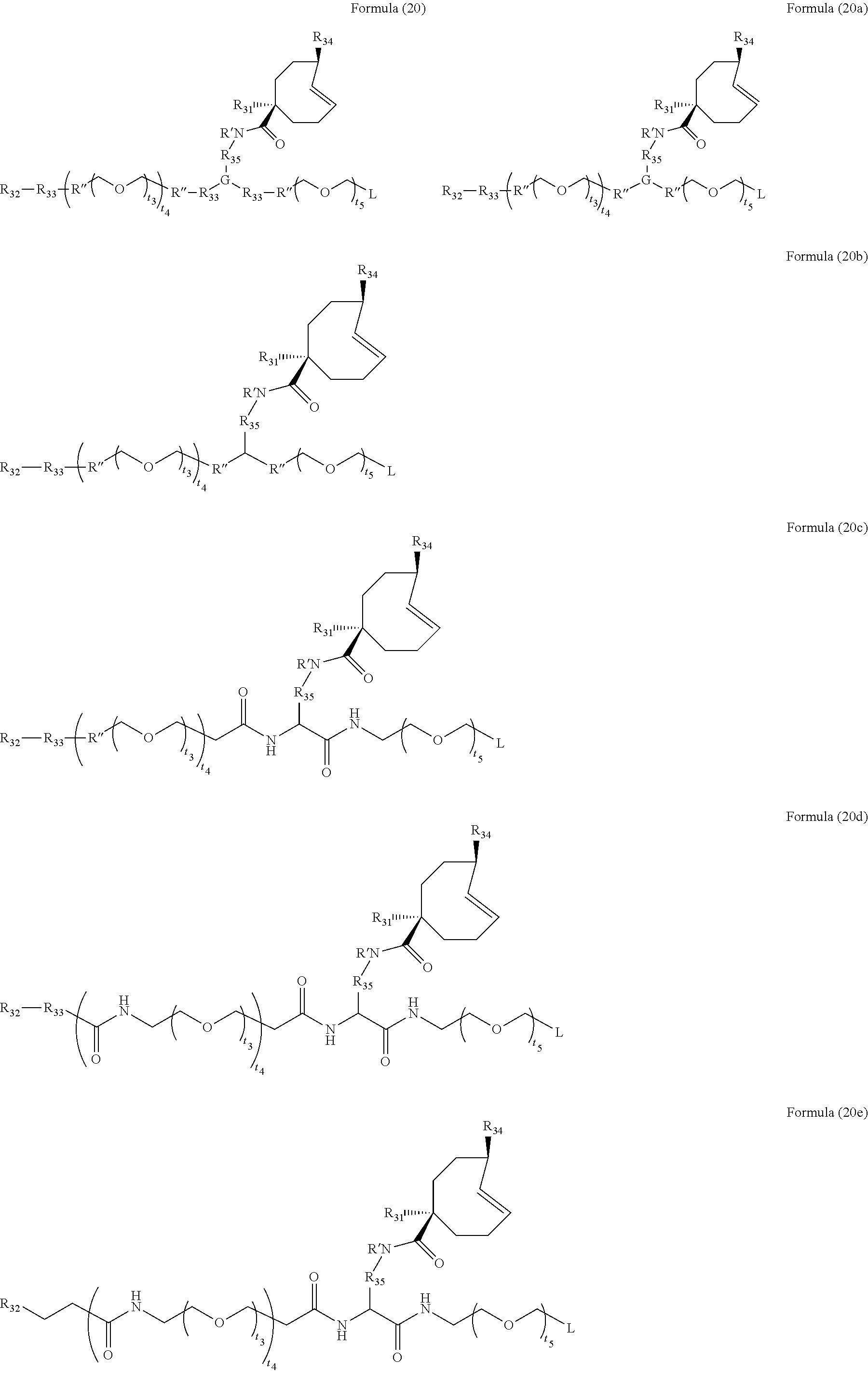

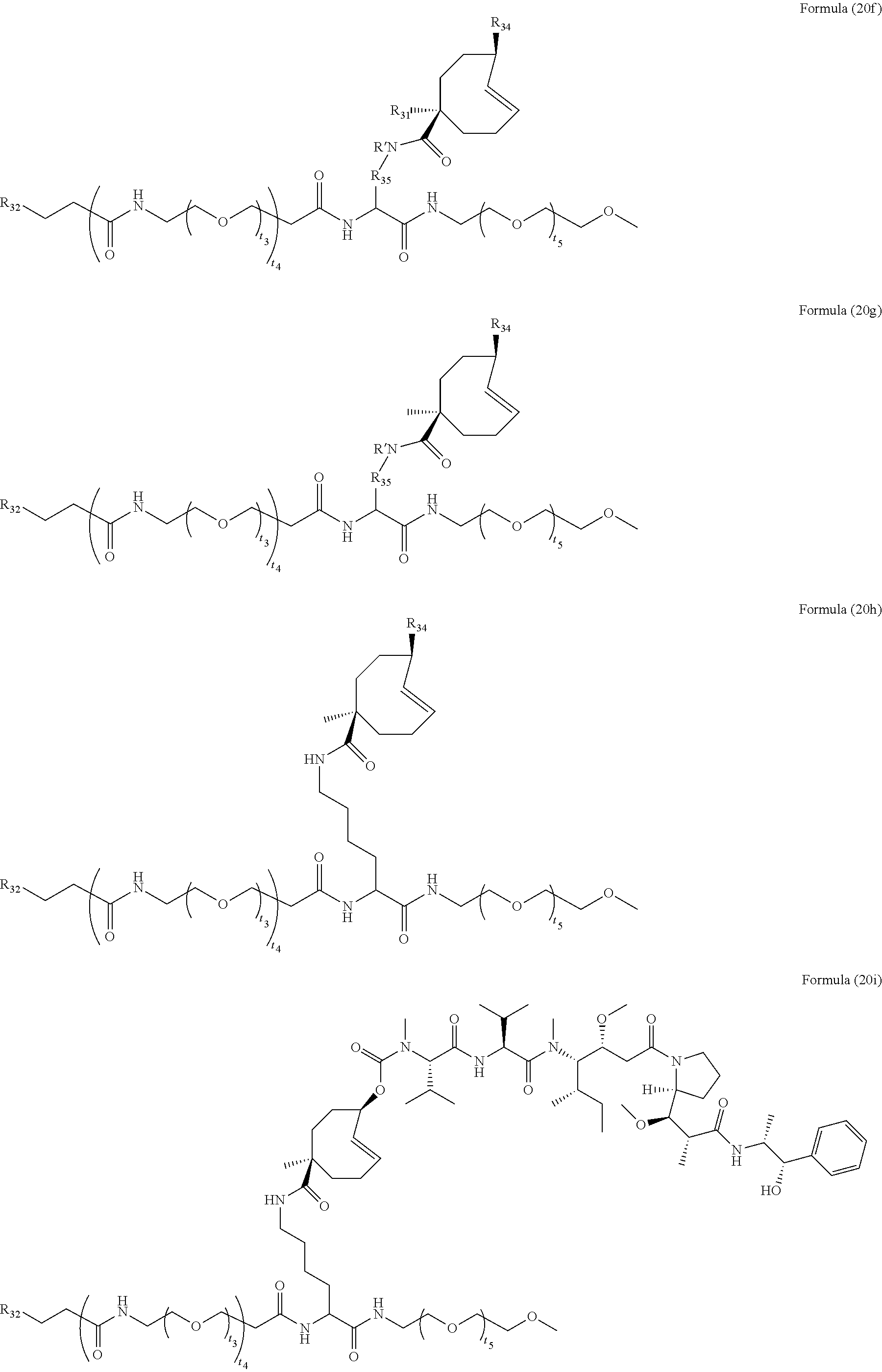

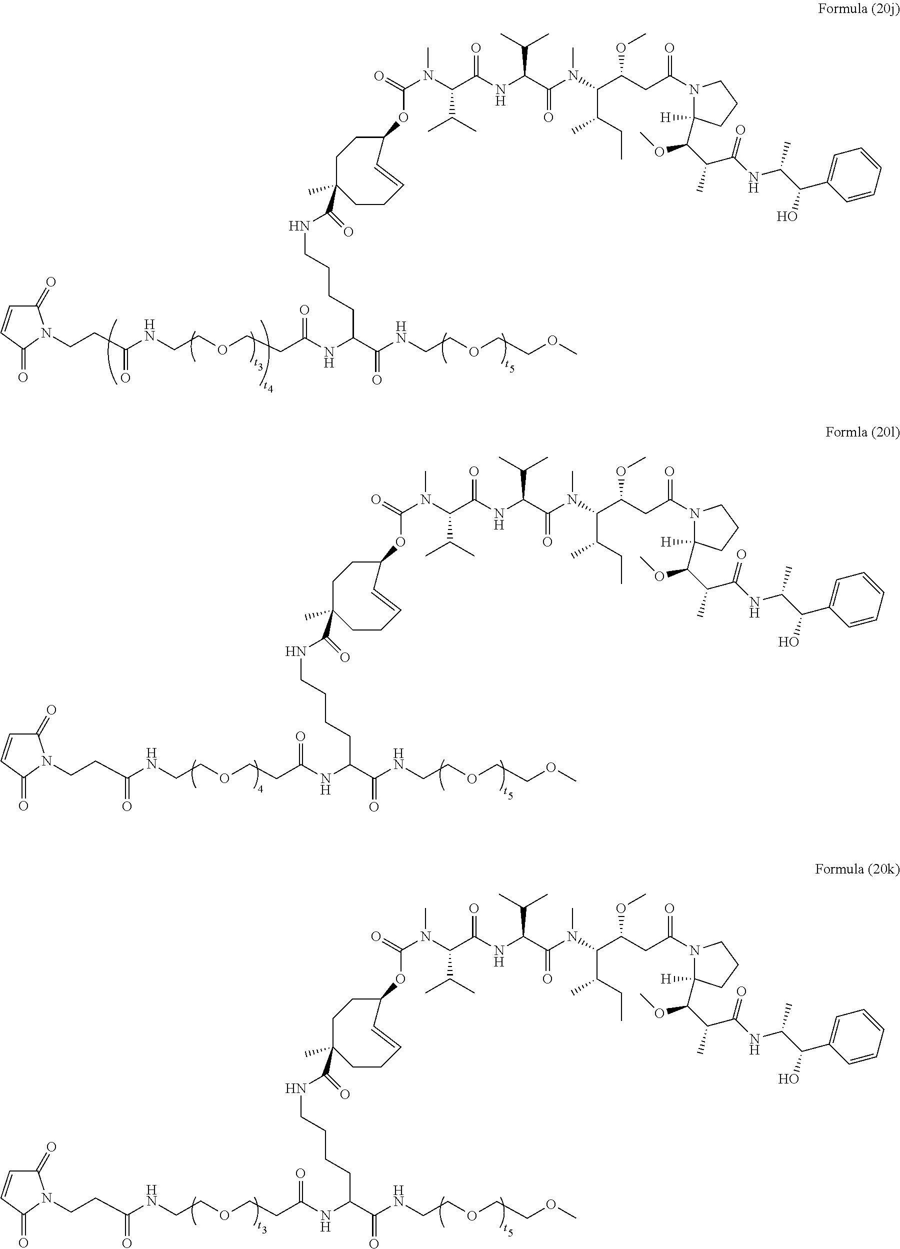

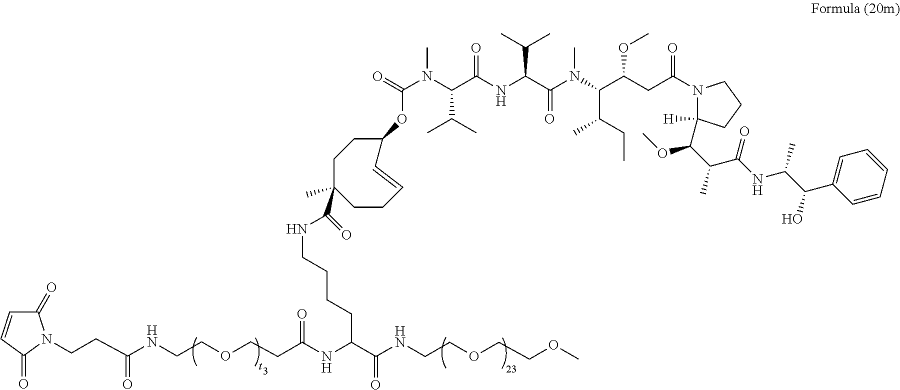

### Formula (21)

In preferred embodiments, the dienophile is comprised in a compound selected from the group consisting of proteins, antibodies, peptoids and peptides, modified with at least one compound according to Formula (20) so as to satisfy Formula (21):

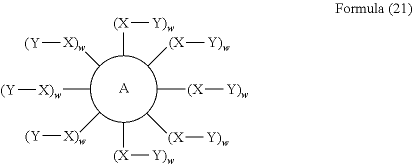

wherein moiety A is selected from the group consisting of proteins, antibodies, peptoids and peptides,
wherein each moiety Y is independently selected from moieties according to Formula (22), wherein at least one moiety Y satisfies said Formula (22):

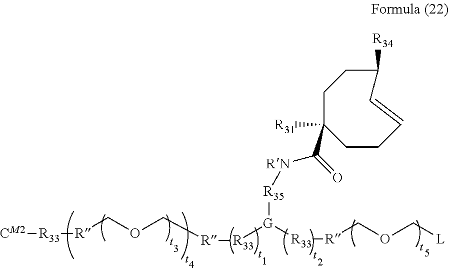

In preferred embodiments, moiety A can be modified with a group according to any one of Formulae (22a), (22b), (22c), (22d), (22e), (22f), (22g), (22h), (22i), (22j), (22k), (22l), and (22m) as disclosed herein.

Preferably, moiety A is modified at 1 to 8 positions, more preferably from 1 to 6 positions, even more preferably at 1 to 4 positions.

In particularly favourable embodiments, moiety A is a diabody according to the sequence listed below in Table 1 as SEQ ID NO:1.

TABLE 1

| Diabody | Diabody sequence (SEQ ID NO: 1) |
|---|---|
| TAG72-binding | SVQLQQSDAELVKPGASVKISCKASGYTFT |
| diabody derived | DHAIHWVKQNPEQGLEWIGYFSPGNDDFKY |
| from the CC49 | NERFKGKATLTADKSSSTAYLQLNSLTSED |
| antibody | SAVYFCTRSLNMAYWGQGTSVTVSSGGGGS |
| | DIVMTQSCSSCPVSVGEKVTLSCKSSQSLL |
| | YSGNQKNYLAWYQQKPGQSPKLLIYWASTR |
| | ESGVPDRFTGSGSGTDFTLSISSVETEDLA |
| | VYYCQQYYSYPLTFGAGTKLVLKR |

In preferred embodiments, the dienophile is comprised in a compound selected from the group consisting of antibodies, proteins, peptoids and peptides comprising at least one moiety M selected from the group consisting of —OH, —NHR′, —CO₂H, —SH, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrille, (oxa)norbornene before modification with a compound according to Formula (20), wherein the compound selected from the group consisting of antibodies, proteins, peptoids and peptides satisfies Formula (21) after modification with at least one compound according to Formula (20);
wherein in Formula (21):
moiety A is selected from the group consisting of antibodies, proteins peptoids and peptides,
wherein each individual w is 0 or 1, wherein at least one w is 1,
wherein each moiety Y is independently selected from moieties according to Formula (22), wherein at least one moiety Y satisfies said Formula (22):
wherein moiety X is part of moiety A and was a moiety M before modification of moiety A,
wherein moiety C^{M2} is part of moiety Y and was a moiety R₃₂ as defined herein before modification of moiety A,
wherein when moiety X is —S— or S—S—, then C^{M2} is selected from the group consisting of

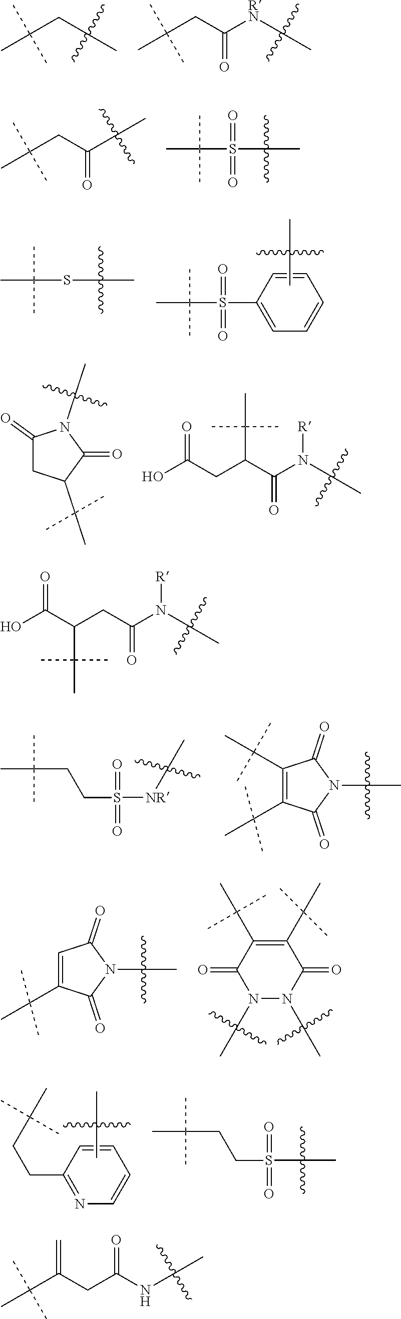

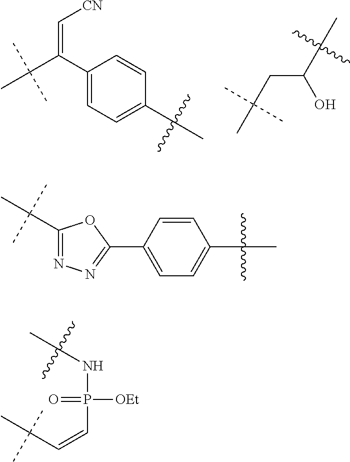

wherein R′ equals R₃₇, wherein the wiggly line denotes a bond to the remaining part of moiety Y, and wherein the dotted line denotes a bond to moiety X,
wherein when moiety X is —SCH₃—, then C^{M2} is preferably

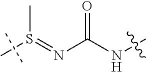

wherein the wiggly line denotes a bond to the remaining part of moiety Y, and wherein the dotted line denotes a bond to moiety X,
wherein when moiety X is —NR′—, then C^{M2} is selected from the group consisting of

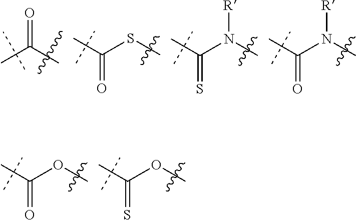

wherein R′ equals R₃₇, wherein the wiggly line denotes a bond to the remaining part of moiety Y, and wherein the dotted line denotes a bond to moiety X,
wherein when moiety X is —C— derived from a moiety M that was —C(O)R′ or —C(O)R′—, then C^{M2} is selected from the group consisting of

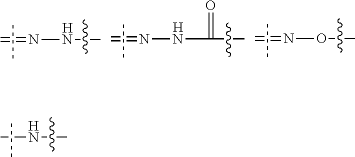

wherein the wiggly line denotes a bond to the remaining part of moiety Y, and wherein the dotted line denotes a bond to moiety X,
wherein when moiety X is —C(O)— derived from a moiety M that was —C(O)OH, then C^{M2} is selected from the group consisting of

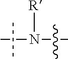

wherein R′ equals R₃₇, wherein the wiggly line denotes a bond to the remaining part of moiety Y, and wherein the dotted line denotes a bond to moiety X,
wherein when moiety X is —O—, then C^{M2} is selected from the group consisting of

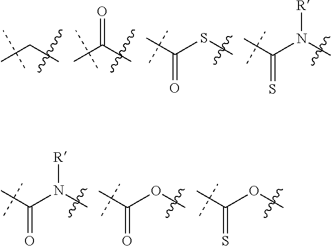

wherein R′ equals R₃₇, wherein the wiggly line denotes a bond to the remaining part of moiety Y, and wherein the dotted line denotes a bond to moiety X,
wherein when moiety X is derived from a moiety M that was —N₃ and that was reacted with an R³² that was comprised an alkyne group, then X and C^{M2} together form a moiety C^{X}, wherein C^{X} comprises a triazole ring,
wherein each C^{X} is independently selected from the group consisting of

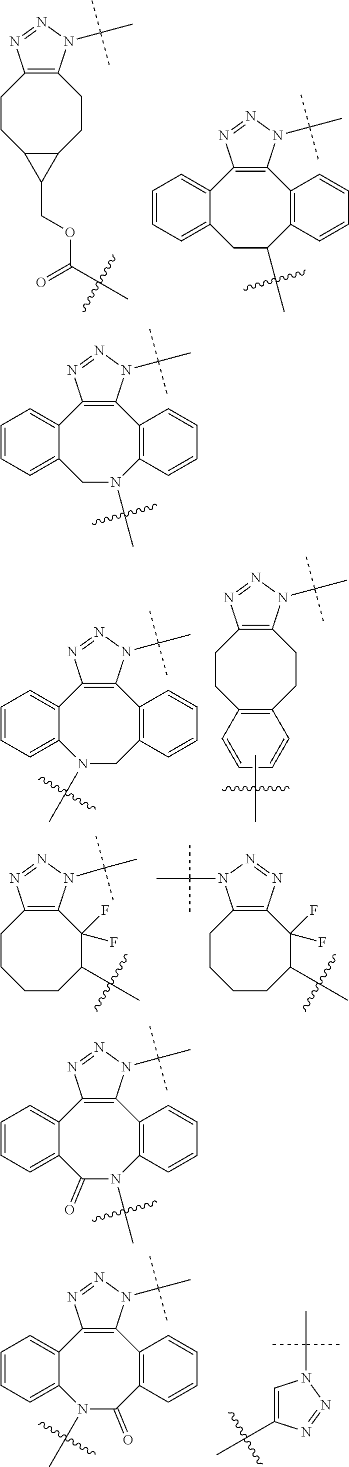

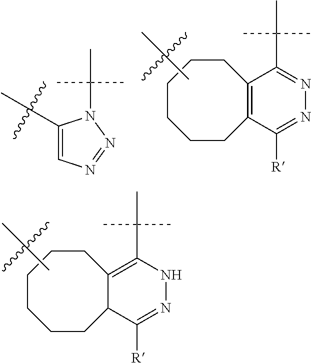

wherein the wiggly line denotes a bond to the remaining part of moiety Y, and wherein the dashed line denotes a bond to moiety X. Herein, R′ is preferably as defined for R₃₇.

### Formula (22)

In preferred embodiments of the invention, the compounds pertaining to Formula (22) can be further specified by any one of the Formulae (22a), (22b), (22c), (22d), (22e), (22f), (22g), (22h), (22i), (22j), (22k), (22l), and (22m) depicted below:
In preferred embodiments, the dienophile satisfies a compound according to Formula (21), wherein moiety A is modified with any one of the compounds depicted in the Formulae below:

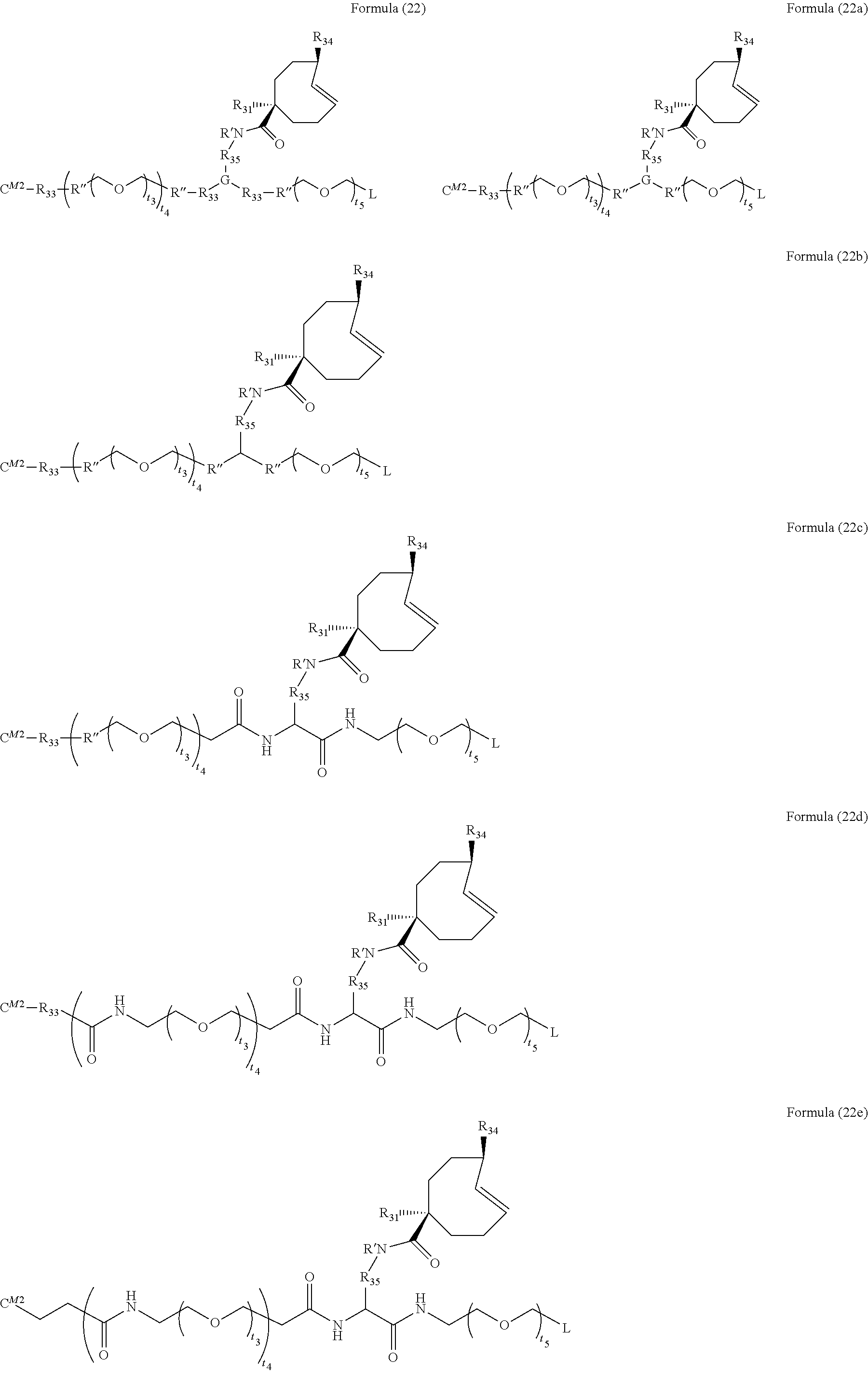

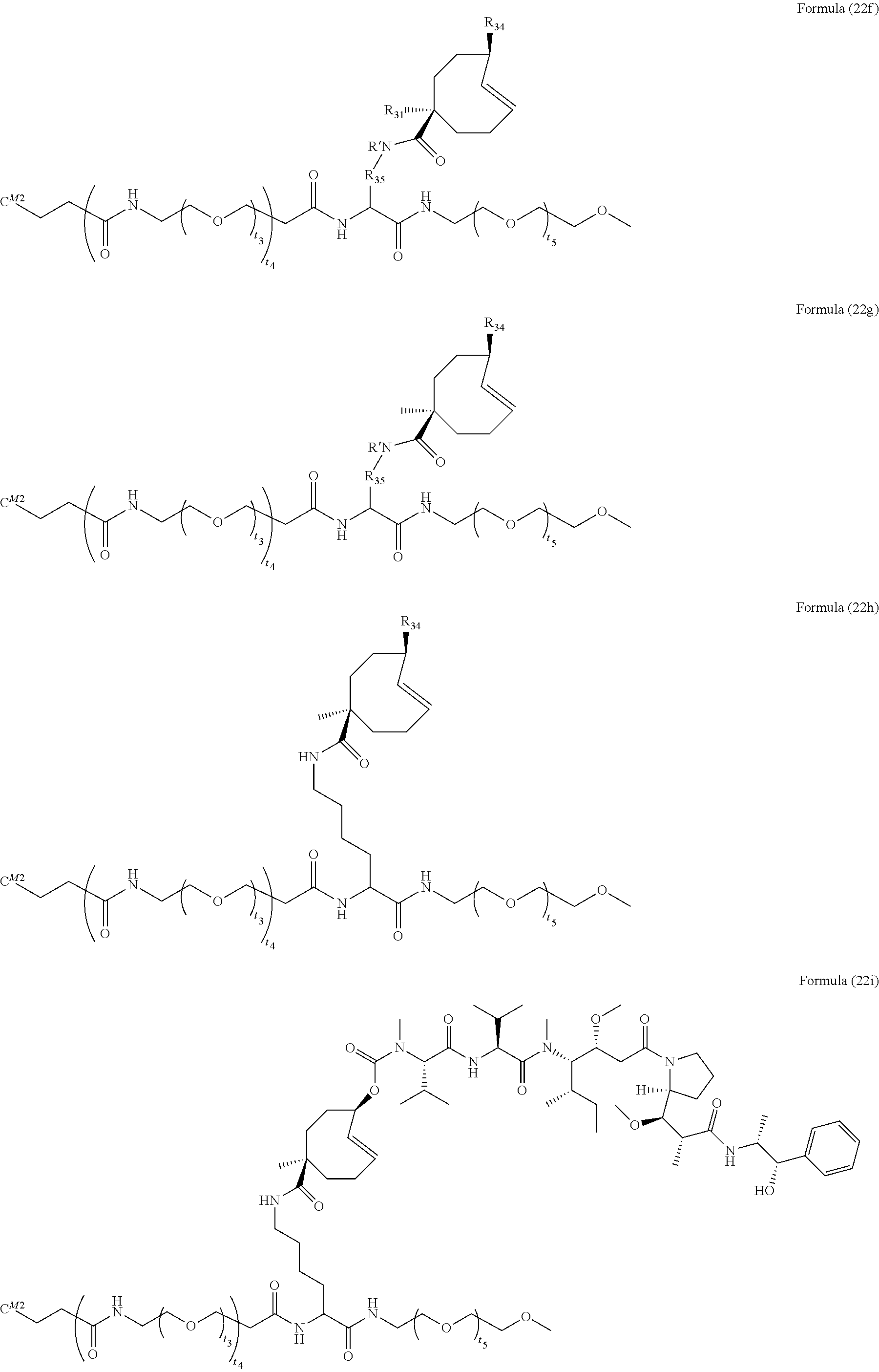

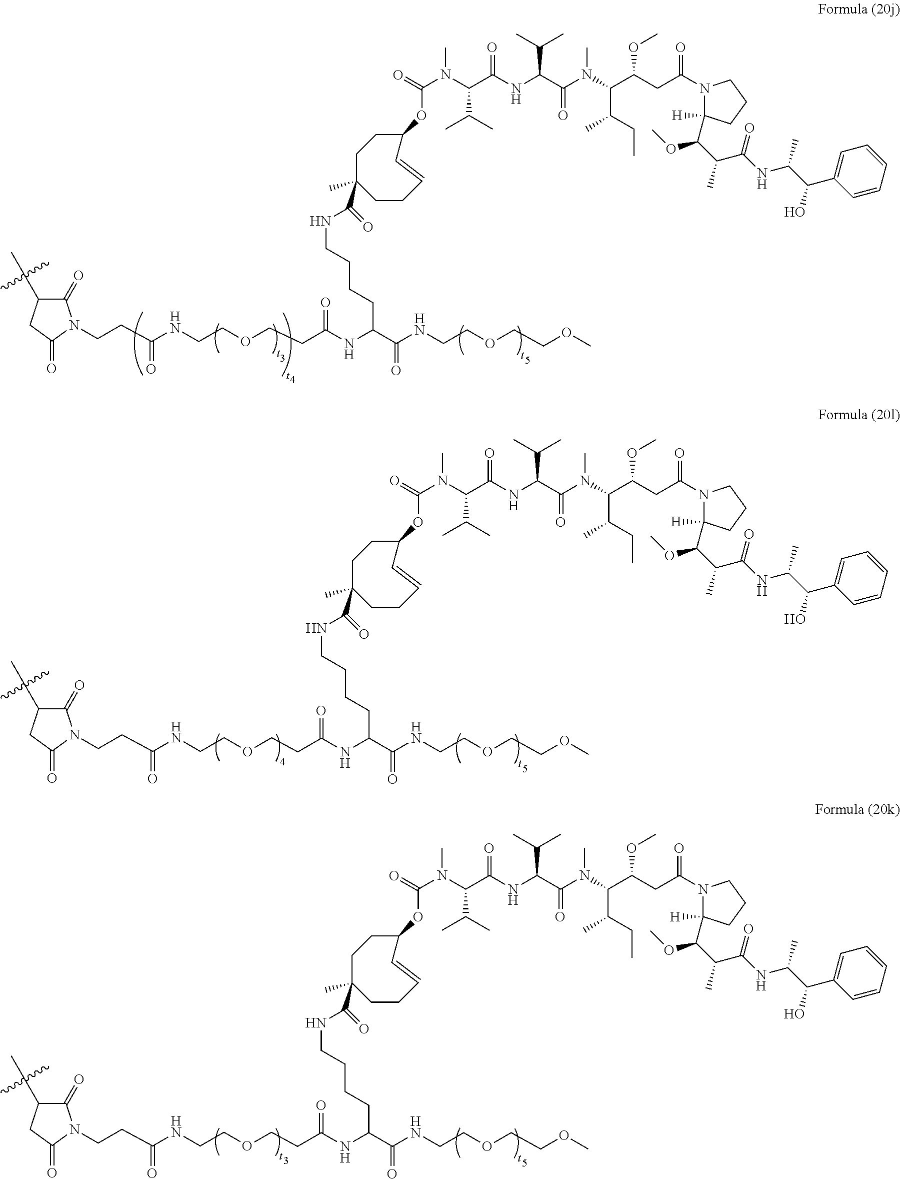

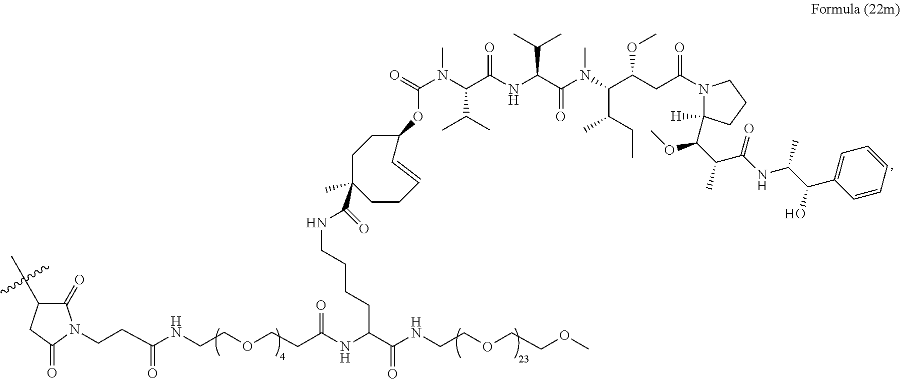

wherein in Formulae (22j), (22k), (22l), (22m), the wiggly line denotes a bond to moiety X of moiety A in Formula (21).

In Formulae (22a), (22b), (22c), (22d), (22e), (22f), (22g), (22h), (22i), (22j), (22k), and (22l), C^{M2}, R′, R″, R₃₁, R₃₃, R₃₄, R₃₅, L, t₁, t₂, t₃, t₄, t₅, and any other variable are as defined in this document for Formula (22).

In Formulae (22j), (22k), and (22l) the wiggly line indicates a bond to moiety X in Formula (21).

It will be understood that the imide moiety (22j), (22k), (22l), and (22m) may hydrolyze in aqueous environments. The hydrolysis products of these compounds, which comprise regioisomers, are understood to be disclosed herein as well.

In a particularly favourable embodiment, in Formula (21) moiety A is a diabody according to SEQ ID NO:1 as disclosed herein, and Y is the compound according to any one of the Formulae (22a), (22b), (22c), (22d), (22e), (22f), (22g), (22h), (22i), (22j), (22k), (22l), and (22m).

Preferably, in Formula (21) moiety A is a diabody according to SEQ ID NO:1 as disclosed herein, and Y is the compound according to the Formula (22m).

More preferably, in Formula (21) moiety A is a diabody according to SEQ ID NO:1 as disclosed herein, and Y is the compound according to the Formula (22m), and in four moieties —(X—Y)_{w} of Formula (21) w is 1, i.e. the diabody according to SEQ ID NO:1 is modified at four positions.

Even more preferably, in Formula (21) moiety A is a diabody according to SEQ ID NO:1 as disclosed herein, and Y is the compound according to the Formula (22m), and in four moieties —(X—Y)_{w} of Formula (21) w is 1, and X in these four moieties —(X—Y)_{w} is a sulphur atom, i.e. S, that is part of a cysteine that is part of the diabody according to SEQ ID NO:1.

### R⁶

In a preferred embodiment, each R⁶ is independently selected from the group consisting of hydrogen, —(S^{P})ᵢ—C^{B} with i being an integer in a range of from 0 to 4, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo) alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups;
wherein preferably i is an integer ranging from 0 to 1,
wherein the R⁶ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.
In a preferred embodiment, R⁶ is selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R⁶ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.
In preferred embodiments, R⁶ is selected from the group consisting of hydrogen, C₁-C₄ alkyl groups, C₂-C₄ alkenyl groups, and C₄₋₆ (hetero)aryl groups, wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In preferred embodiments, R⁶ is selected from the group consisting of hydrogen, C₁-C₃ alkyl groups, C₂-C₃ alkenyl groups, and C₄₋₆ (hetero)aryl groups, wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In preferred embodiments, the R⁶ groups not being hydrogen are not substituted.

In preferred embodiments, the R⁶ groups not being hydrogen do not contain heteroatoms.

In preferred embodiments, the R⁶ groups are hydrogen.

### R⁷

In preferred embodiments, each R⁷ is independently selected from the group consisting of hydrogen, —(S^{P})ᵢ—C^{B} with i being an integer in a range of from 0 to 4, —F, —Cl, —Br, —I, —OR₃₇, —N(R₃₇)₂, —SO₃, —PO₃, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(═S)O—R₃₇, C(═S)S—R₃₇, S(O)R₃₇, —S(O)₂R₃₇, NR₃₇S(O)₂R₃₇, —ON(R₃₇)₂, —NR₃₇OR₃₇, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups; wherein preferably i is an integer ranging from 0 to 1, wherein the alkyl groups, alkenyl groups, alkynyl groups, aryl, heteroaryl, cycloalkyl groups, cycloalkenyl groups, cycloalkynyl groups, (cyclo)alkyl(hetero)aryl groups, (hetero)aryl(cyclo)alkyl groups, (cyclo)alkenyl(hetero)aryl groups, (hetero)aryl(cyclo)alkenyl groups, (cyclo)alkynyl(hetero)aryl groups, (hetero)aryl(cyclo)alkynyl groups, alkylcycloalkyl groups, cycloalkylalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, —NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.
In preferred embodiments, R⁷ is independently selected from the group consisting of hydrogen, —(S^{P})ᵢ—C^{B} with i being an integer in a range of from 0 to 4, —F, —Cl, —Br, —I, —OR₃₇, —N(R₃₇)₂, —SO₃, —PO₃⁻, —NO₂, —CF₃, —SR₃₇, —S(═O)₂N(R₃₇)₂, OC(═O)R₃₇, SC(═O)R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(═S)O—R₃₇, C(═S)S—R₃₇, —S(O)R₃₇, —S(O)₂R₃₇, NR₃₇S(O)₂R₃₇, —ON(R₃₇)₂, —NR₃₇OR₃₇, C₁-C₈ alkyl groups, C₂-C₈, alkenyl groups, C₂-C₈, alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.
wherein preferably i is an integer ranging from 0 to 1,
In preferred embodiments, each R⁷ is independently selected from the group consisting of hydrogen and C₁-C₃ alkyl groups, C₂-C₃ alkenyl groups, and C₄₋₆ (hetero)aryl groups, wherein the alkyl groups, alkenyl groups, and (hetero)aryl groups are optionally substituted with a moiety selected from the group consisting of —Cl, —F, —Br, —I, —OH, —NH₂, ═O, ═NH, —N(CH₃)₂, —S(O)₂CH₃, and —SH, and are optionally interrupted by at most one heteroatom selected from the group consisting of —O—, —S—, —NH—, —P—, and —Si—, wherein the N, S, and P atoms are optionally oxidized, wherein the N atoms are optionally quaternized.

In preferred embodiments, R⁷ is preferably selected from the group consisting of hydrogen, methyl, —CH₂—CH₂—N(CH₃)₂, and —CH₂—CH₂—S(O)₂—CH₃,

In preferred embodiments, R⁷ is hydrogen.

R⁸ and R⁹
R⁸ and R⁹ are as defined for R⁷. In preferred embodiments, at least one or all R⁸ are —H. in preferred embodiments, at least one or all R⁸ are —CH₃. In preferred embodiments, at least one or all R⁹ are —H. In preferred embodiments, at least one or all R⁹ are —CH₃.

### R₃₁

In preferred embodiments, R₃₁ is selected from the group consisting of hydrogen, C₁-C₆ alkyl groups, C₆ aryl groups, C₄-C₅ heteroaryl groups, C₃-C₆ cycloalkyl groups, C₅-C₁₂ alkyl(hetero)aryl groups, C₅-C₁₂ (hetero)arylalkyl groups, C₄-C₁₂ alkylcycloalkyl groups, —N(R′)₂, —OR′, —SR′, —SO₃H, —C(O)OR′, and Si(R′)₃, wherein for R₃₁ the alkyl groups, (hetero)aryl groups, cycloalkyl groups, alkyl(hetero)aryl groups, (hetero)arylalkyl groups, alkylcycloalkyl groups are optionally substituted with a moiety selected from the group consisting of —Cl, —F, —Br, —I, NO₂, SO₃H, PO₃H, —PO₄H₂, —OR′, —N(R′)₂, —CF₃, ═O, ═NR′, —SR′, and optionally contain one or more heteroatoms selected from the group consisting of —O—, —S—, —NR′—, —P—, and —Si—, wherein the N, S, and P atoms are optionally oxidized, wherein the N atoms are optionally quaternized,

In preferred embodiments, R₃₁ is hydrogen. In other preferred embodiments, R₃₁ is —CH₃.

### R₃₂

R₃₂ is a conjugation moiety, which is chemical group that can be used for binding, conjugation or coupling of a Construct, such as Construct-B, or a Spacer, or another molecule or construct of interest. The person skilled in the art is aware of the myriad of strategies that are available for the chemoselective or -unselective or enzymatic coupling or conjugation of one molecule or construct to another.

In preferred embodiments, R₃₂ is a moiety that allows conjugation to a protein comprising natural and/or non-natural amino acids. Moieties suitable for conjugation are known to the skilled person. Conjugation strategies are for example found in [O. Boutureira, G. J. L. Bernardes, Chem. Rev., 2015, 115, 2174-2195].

In particularly favourable embodiments, R₃₂ is selected from the group consisting of N-maleimidyl groups, halogenated N-alkylamido groups, sulfonyloxy N-alkylamido groups, vinyl sulfone groups, (activated) carboxylic acids, benzenesulfonyl halides, ester groups, carbonate groups, sulfonyl halide groups, thiol groups or derivatives thereof, C₂₋₆ alkenyl groups, C₂₋₆ alkynyl groups, C₇₋₁₈, cycloalkynyl groups, C₅₋₁₈, heterocycloalkynyl groups, bicyclo[6.1.0]non-4-yn-9-yl] groups, C₃₋₁₂ cycloalkenyl groups, azido groups, phosphine groups, nitrile oxide groups, nitrone groups, nitrile imine groups, isonitrile groups, diazo groups, ketone groups, (O-alkyl)hydroxylamino groups, hydrazine groups, halogenated N-maleimidyl groups, aryloxymaleimides, dithiophenolmaleimides, bromo- and dibromopyridazinediones, 2,5-dibromohexanediamide groups, alkynone groups, 3-arylpropiolonitrile groups, 1,1-bis(sulfonylmethyl)-methylcarbonyl groups or elimination derivatives thereof, carbonyl halide groups, allenamide groups, 1,2-quinone groups, isothiocyanate groups, isocyanate groups, aldehyde groups, triazine groups, squaric acids, 2-imino-2-methoxyethyl groups, (oxa)norbornene groups, (imino)sydnones, methylsulfonyl phenyloxadiazole groups, aminooxy groups, 2-amino benzamidoxime groups, ethynylphosphonamidates, groups reactive in the Pictet-Spengler ligation and hydrazine-Pictet-Spengler (HIPS) ligation, DNA intercalators, tetrazine groups, and photocrosslinkers.

In preferred embodiments, R₃₂ is an N-maleimidyl group connected to the remaining part of the compound according to any one of Formula (20)-(20e) via the N atom of the N-maleimidyl group.

In other embodiments R₃₂ is selected from the group consisting of, hydroxyl groups, amine groups, halogens, vinyl pyridine groups, disulfide groups, pyridyl disulfide groups, sulfonyloxy groups, mercaptoacetamide groups, anhydride groups, sulfonylated hydroxyacetamido groups, sulfonyl chlorides, thiosemicarbazone, hydrazine carboxylate, and arylhydrazide.

In other embodiments R₃₂ is group that can be connected to another group by means of an enzyme, for example sortase or Tubulin tyrosine ligase.

### R₃₃

In preferred embodiments, each individual R₃₃ is selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized.

In particularly favourable embodiments, each individual R₃₃ is selected from the group consisting of C₁-C₆ alkylene groups, C₂-C₆ alkenylene groups, and C₂-C₆ alkynylene groups, more preferably from the group consisting of C₁-C₃ alkylene groups, C₂-C₃ alkenylene groups, and C₂-C₃ alkynylene groups.

### R₃₄

In preferred embodiments, each individual R₃₄ is selected from the group consisting of —OH, —OC(O)Cl, —OC(O)O—N-succinimidyl, —OC(O)O-4-nitrophenyl, —OC(O)O-tetrafluorophenyl, —OC(O)O-pentafluorophenyl, —OC(O)—C^{A}, —OC(S)—C^{A}, —O-(L^{C}(C^{A})ₛ(C^{A})ₛ((S^{P})ᵢ—C^{B})ⱼ)ᵣ—C^{A}, and —C^{A}, —OC(O)—(S^{P})ₖ—C^{A}, —OC(S)—(S^{P})ₖ—C^{A}, —O-(L^{C}((S^{P})ₖC^{A})ₛ((S^{P})ₖ—C^{A})ₛ((S^{P})ᵢ—C^{B})ⱼ)ᵣ—(S^{P})ₖ—C^{A}, and —(S^{P})ₖ—C^{A}. Therein, r is an integer in range of from 0 to 2, preferably r is 0 or 1, and therein, each s is independently 0 or 1, each k is independently 0 or 1, each i is an integer in a range of from 0 to 4, and j is 0 or 1.

It is preferred that R₃₄ is an axial substituent on the TCO ring.

### R₃₅

In preferred embodiments, each individual R₃₅ is selected from the group consisting of C₁-C₈, alkylene groups, C₂-C₈, alkenylene groups, C₂-C₈, alkynylene groups, C₆ arylene groups, C₄-C₅ heteroarylene groups, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein for the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,

In preferred embodiments, each individual R₃₅ is selected from the group consisting of C₁-C₄ alkylene groups, C₂-C₄ alkenylene groups, C₂-C₄ alkynylene groups, C₆ arylene groups, C₄-C₅ heteroarylene groups, C₃-C₆ cycloalkylene groups, wherein the alkylene groups, alkenylene groups, alkynylene groups, (hetero)arylene groups, and cycloalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,

### R₃₆

In preferred embodiments, R₃₆ is selected from the group consisting of hydrogen, —(S^{P})ᵢ—C^{B} with i being an integer in a range of from 0 to 4, C₁-C₂₄ alkyl groups, C₂-C₂₄alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo) alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo) alkynyl(hetero)aryl groups, C₄-C₂₄ (hetero) aryl(cyclo)alkynyl groups, C₄-C₂₄ alkylcycloalkyl groups, and C₄-C₂₄ cycloalkylalkyl groups.

Preferably each R₃₆ is independently selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups, and C₅-C₁₂ (hetero)arylcycloalkyl groups.

For R₃₆ i preferably is an integer ranging from 0 to 1.
The R₃₆ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.
In preferred embodiments, R₃₆ is selected from the group consisting of hydrogen, —(S^{P})ᵢ—C^{B} with i being an integer in a range of from 0 to 4, C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In preferred embodiments, R₃₆ is selected from the group consisting of hydrogen, C₁-C₃ alkyl groups, C₂-C₃ alkenyl groups, and C₄₋₆ (hetero)aryl groups, wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In preferred embodiments, the R₃₆ groups not being hydrogen are not substituted. In preferred embodiments, the R₃₆ groups not being hydrogen do not contain heteroatoms.

### R₃₇

In preferred embodiments, R₃₇ is selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

In preferred embodiments, R₃₇ is selected from the group consisting of hydrogen, —(S^{P})ᵢ—C^{B}, C₁-C₄ alkyl groups, C₂-C₄ alkenyl groups, C₂-C₄ alkynyl groups, C₆-C₈ aryl, C₂-C₈ heteroaryl, C₃-C₆ cycloalkyl groups, C₅-C₆ cycloalkenyl groups, C₃-C₁₀ alkyl(hetero)aryl groups, C₃-C₁₀ (hetero)arylalkyl groups, C₄-C₈ alkylcycloalkyl groups, C₄-C₈ cycloalkylalkyl groups, C₅-C₁₀ cycloalkyl(hetero)aryl groups and C₅-C₁₀ (hetero)arylcycloalkyl groups, wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized. wherein preferably i is an integer ranging from 0 to 1,

### R₄₇

In preferred embodiments, each R₄₇ is independently selected from the group consisting of hydrogen, —F, —Cl, —Br, —I, —OR₃₇, —N(R₃₇)₂, —SO₃, —PO₃—, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, Q-S)O—R₃₇, C(═S)S—R₃₇, S(O)R₃₇, —S(O)₂R₃₇, NR₃₇S(O)₂R₃₇, —ON(R₃₇)₂, —NR₃₇OR₃₇, —(S^{P})i-C^{B}, C₁-C₈ alkyl groups, C₂-C₈ alkenyl groups, C₂-C₈ alkynyl groups, C₆-C₁₂ aryl groups, C₂-C₁₂ heteroaryl groups, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, —NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.

In preferred embodiments, each R₄₇ is independently selected from the group consisting of hydrogen —F, —Cl, —Br, —I, —OR₃₇, —N(R₃₇)₂, —SO₃, —PO₃⁻, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(═S)O—R₃₇, C(═S)S—R₃₇, S(O)R₃₇, —S(O)₂R₃₇, NR₃₇S(O)₂R₃₇, —ON(R₃₀₂, —NR₃₇OR₃₇, —(S^{P})ᵢ—C^{B}, C₁-C₄ alkyl groups, C₂-C₄ alkenyl groups, C₂-C₄ alkynyl groups, C₆-C₈ aryl groups, C₂-C₈ heteroaryl groups, C₃-C₆ cycloalkyl groups, C₅-C₆ cycloalkenyl groups, C₃-C₁₀ alkyl(hetero)aryl groups, C₃-C₁₀ (hetero)arylalkyl groups, C₄-C₁₀ alkylcycloalkyl groups, C₄-C₁₀ cycloalkylalkyl groups, C₅-C₁₀ cycloalkyl(hetero)aryl groups and C₅-C₁₀ (hetero)arylcycloalkyl groups,

wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.
wherein preferably i is an integer ranging from 0 to 1,

### R′

In preferred embodiments, each R′ is independently defined as for R₃₇.

### R″

In preferred embodiments, each R″ is independently selected from the group consisting of

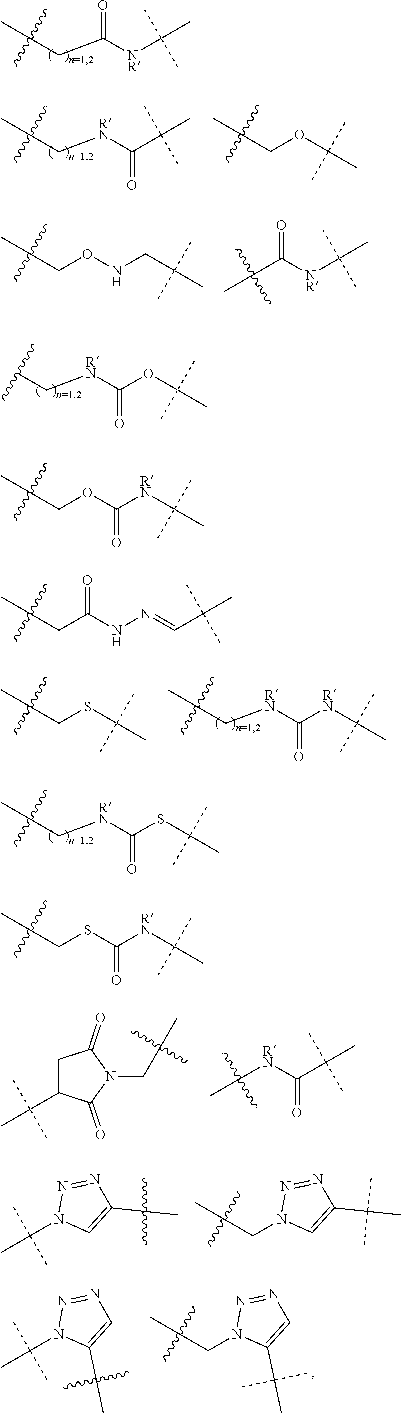

wherein R′ equals R₃₇, wherein the wiggly line depicts a bond to an ethylene glycol group or optionally to the R₃₃ adjacent to R₃₂ when t₄ is 0, and the dashed line depicts a bond to R₃₃ or G.

In preferred embodiments, R″ is —CH₂—C(O)NR′— or —CH₂—NR′C(O)—.

### G

In preferred embodiments, G is selected from the group consisting of CR′, N, C₅-C₆ arenetriyl, C₄-C₅ heteroarenetriyl, C₃-C₆ cycloalkanetriyl, and C₄-C₆ cycloalkenetriyl, wherein the arenetriyl, heteroarenetriyl, cycloalkanetriyl, and cycloalkenetriyl are optionally further substituted with groups selected from the group consisting of —Cl, —F, —Br, —I, —OR′, —N(R′)₂, —SR′, —SO₃H, —PO₃H, —PO₄H₂, —NO₂, —CF₃ and —R₃₁, and optionally contain one or more heteroatoms selected from the group consisting of —O—, —S—, —NR′—, —P—, and —Si—, wherein the N, S, and P atoms are optionally oxidized, wherein the N atoms are optionally quaternized. Preferably, G is CR′.

### L

In preferred embodiments, L is selected from the group consisting of —CH₂—OCH₃, —CH₂—OH, —CH₂—C(O)OH, —C(O)OH. In preferred embodiments, L is preferably —CH₂—OCH₃,

### Moieties M and X

It is understood that when moiety M is modified with a compound according to any one of Formulae (20)-(20e), and M is —OH, —NHR′, or —SH, that it will lose a proton and will become a moiety X that is —O—, —NR′— or —S—, respectively. It is understood that when moiety M is —C(O)OH, that it will lose an —OH upon modification with a compound according to any one of Formulae (20)-(20e), and that the resulting moiety X is —C(O)—. It is understood that when moiety M is —C(O)R′ or —C(O)R′— it will become a moiety X that is —C— upon modification with a compound according to any one of Formulae (20)-(20e).

It is understood that a moiety M that is a —COOH may be derived from the C-terminus of the peptide, protein or peptoid, or from an acidic amino acid residue such as aspartic acid or glutamic acid.

It is understood that moiety M may be derived from non-natural amino acid residues containing —OH, —NHR′, —CO₂H, —SH, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrille, or a (oxa)norbornene.

It is understood that when moiety M is —OH it may be derived from an amino acid residue such as serine, threonine and tyrosine.

It is understood that when moiety M is —SH it may be derived from an amino acid residue such as cysteine.

It is understood that when moiety M is —NHR′ it may be derived from an amino acid residue such as lysine, homolysine, or ornithine.

t₁, t₂, t₃, t₄, t₅
In preferred embodiments, t₁ is 0. In preferred embodiments, t₁ is 1.

In preferred embodiments, t₂ is 0. In preferred embodiments, t₂ is 1.

In preferred embodiments, t₃ is an integer in a range of from 0 to 12. Preferably, t₃ is an integer in a range of from 1 to 10, more preferably in a range of from 2 to 8. In particularly favourable embodiments, t₃ is 4 and y is 1.

In preferred embodiments, t₄ is 0. In preferred embodiments, t₄ is 1.

In preferred embodiments, t₅ is an integer in a range of from 6 to 48, preferably from 15 to 40, more preferably from 17 to 35, even more preferably from 20 to 30, most preferably from 22 to 28. In particularly preferred embodiments, t₅ is 23.

### Section 9—Other Dienophiles

Typically, the dienophile-Construct A conjugate comprises a Construct-A denoted as C^{A} linked, directly or indirectly, to a Trigger moiety denoted as T^{R}, wherein the Trigger moiety is a dienophile. The dienophile, in a broad sense, is an eight-membered non-aromatic cyclic alkenylene moiety (preferably a cyclooctene moiety, and more preferably a trans-cyclooctene moiety). Optionally, the trans-cyclooctene (TCO) moiety comprises at least two exocyclic bonds fixed in substantially the same plane, and/or it optionally comprises at least one substituent in the axial position, and not the equatorial position. The person skilled in organic chemistry will understand that the term “fixed in substantially the same plane” refers to bonding theory according to which bonds are normally considered to be fixed in the same plane. Typical examples of such fixations in the same plane include double bonds and strained fused rings. E.g., the at least two exocyclic bonds can be the two bonds of a double bond to an oxygen (i.e. C═O). The at least two exocyclic bonds can also be single bonds on two adjacent carbon atoms, provided that these bonds together are part of a fused ring (i.e. fused to the TCO ring) that assumes a substantially flat structure, therewith fixing said two single bonds in substantially one and the same plane. Examples of the latter include strained rings such as cyclopropyl and cyclobutyl. Without wishing to be bound by theory, the inventors believe that the presence of at least two exocyclic bonds in the same plane will result in an at least partial flattening of the TCO ring, which can lead to higher reactivity in the retro-Diels-Alder reaction.

In a preferred embodiment, the TCO satisfies the following formula (1a):

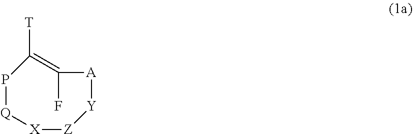

A and P each independently are CR^{a}₂ or CR^{a}X^{D}, provided that at least one is CR^{a}X^{D}. X^{D} is (O—C(O))ₚ-(L^{C})ₙ-(C^{A}), S—C(O)-(L^{C})ₙ-(C^{A}), O—C(S)-(L^{C})ₙ-(C^{A}), S—C(S)-(L^{C})ₙ-(C^{A}), O—S(O)-(L^{C})ₙ-(C^{A}), wherein p=0 or 1, (L^{C})ₙ is an optional linker, with n=0 or 1, preferably linked to T^{R} via S, N, NH, or O, wherein these atoms are part of the linker, which may consist of multiple units arranged linearly and/or branched. C^{A} is one or more therapeutic moieties or drugs, preferably linked via S, N, NH, or O, wherein these atoms are part of the therapeutic moiety.

Preferably, X^{D} is (O—C(O))ₚ-(L^{C})ₙ-(C^{A}), where p=0 or 1, preferably 1, and n=0 or 1.
It is preferred that when C^{A} is bound to T^{R} or L^{C} via NH, this NH is a primary amine (—NH₂) residue from C^{A}, and when C^{A} is bound via N, this N is a secondary amine (—NH—) residue from C^{A}. Similarly, it is preferred that when C^{A} is bound via O or S, said O or S are, respectively, a hydroxyl (—OH) residue or a sulfhydryl (—SH) residue from C^{A}.
It is further preferred that said S, N, NH, or O moieties comprised in C^{A} are bound to an aliphatic or aromatic carbon of C^{A}.
It is preferred that when L^{C} is bound to T^{R} via NH, this NH is a primary amine (—NH₂) residue from L^{C}, and when L^{C} is bound via N, this N is a secondary amine (—NH—) residue from L^{C}. Similarly, it is preferred that when L^{C} is bound via O or S, said O or S are, respectively, a hydroxyl (—OH) residue or a sulfhydryl (—SH) residue from L^{C}.
It is further preferred that said S, N, NH, or O moieties comprised in L^{C} are bound to an aliphatic or aromatic carbon of L^{C}.

Where reference is made in the invention to a linker L^{C} this can be self-immolative or not, or a combination thereof, and which may consist of multiple self-immolative units.

By way of further clarification, if p=0 and n=0, the drug species C^{A} directly constitutes the leaving group of the elimination reaction, and if p=0 and n=1, the self-immolative linker constitutes the leaving group of the elimination. The position and ways of attachment of linkers L^{C} and drugs C^{A} are known to the skilled person (see for example Papot et al, Anti-Cancer Agents in Medicinal Chemistry, 2008, 8, 618-637).

In an interesting embodiment, Y,Z,X,Q each independently are selected from the group consisting of CR^{a}₂, C═CR^{a}₂, C═O, C═S, C═NR^{b}, S, SO, SO₂, O, NR^{b}, and SiR^{c}₂, with at most three of Y, Z, X, and Q being selected from the group consisting of C═CR^{a}₂, C═O, C═S, and C═NR^{b}, wherein two R moieties together may form a ring, and with the proviso that no adjacent pairs of atoms are present selected from the group consisting of O—O, O—NR^{b}, S—NR^{b}, O—S, O—S(O), O—S(O)₂, and S—S, and such that Si is only adjacent to CR^{a}₂ or O.

In a preferred embodiment, the TCO of formula (Ia) is an all-carbon ring. In another preferred embodiment, the TCO of formula (Ia) is a heterocyclic carbon ring, having of one to three oxygen atoms in the ring, and preferably a single oxygen atom.

In another interesting embodiment, one of the bonds PQ, QX, XZ, ZY, YA is part of a fused ring or consists of CR^{a}═CR^{a}, such that two exocyclic bonds are fixed in the same plane, and provided that PQ and YA are not part of an aromatic 5- or 6-membered ring, of a conjugated 7-membered ring, or of CR^{a}═CR^{a}; when not part of a fused ring P and A are independently CR^{a}₂ or CR^{a}X^{D}, provided that at least one is CR^{a}X^{D}; when part of a fused ring P and A are independently CR^{a} or CX^{D}, provided that at least one is CX^{D}; the remaining groups (Y,Z,X,Q) being independently from each other CR^{a}₂, C═CR^{a}₂, C═O, C═S, C═NR^{b}, S, SO, SO₂, O, NR^{b}, SiR^{c}₂, such that at most 1 group is C═CR^{a}₂, C═O, C═S, C═NR^{b}, and no adjacent pairs of atoms are present selected from the group consisting of O—O, O—NR^{b}, S—NR^{b}, O—S, O—S(O), O—S(O)₂, and S—S, and such that Si, if present, is adjacent to CR^{a}₂ or O, and the CR^{a}₂═CR^{a}₂ bond, if present, is adjacent to CR^{a}₂ or C═CR^{a}₂ groups;

T, F each independently denotes H, or a substituent selected from the group consisting of alkyl, F, Cl, Br, or I.

In preferred embodiments fused rings are present that result in two exocyclic bonds being fixed in substantially the same plane. These are selected from fused 3-membered rings, fused 4-membered rings, fused bicyclic 7-membered rings, fused aromatic 5-membered rings, fused aromatic 6-membered rings, and fused planar conjugated 7-membered rings as defined below:

Fused 3-membered rings are:

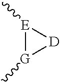

Therein E, G are part of the above mentioned 8-membered ring and can be fused to PQ, QP, QX, XQ, XZ, ZX, ZY, YZ, YA, AY, such that P, A are CR^{a} or CX^{D}, and such that CX^{D} can only be present in A and P.

E-G is CR^{a}—CR^{a} or CR^{a}—CX^{D}, and D is CR^{a}₂, C═O, C═S, C═NR^{b}, NR^{b}, O, S; or E-G is CR^{a}—N or CX^{D}—N, and D is CR^{a}₂, C═O, C═S, C═NR^{b}, NR^{b}O, or S.

Fused 4-membered rings are:

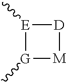

E-G is part of the above mentioned 8-membered ring and can be fused to PQ, QP, QX, XQ, XZ, ZX, ZY, YZ, YA, AY, such that P, A are C, CR^{a} or CX^{D}, and such that CX^{D} can only be present in A and P.

E, G are CR^{a}, CX^{D} or N, and D, M independently from each other are CR^{a}₂, C═O, C═S, C═NR^{b}, C═CR^{a}₂, S, SO, SO₂, O, NR^{b} but no adjacent O—O or S—S groups; or

E-D is C═CR^{a} and G is N, CR^{a}, CX^{D} and M is CR^{a}₂, S, SO, SO₂, O, NR^{b}; or E-D is C═N and G is N, CR^{a}, CX^{D} and M is CR^{a}₂, S, SO, SO₂, O; or

D-M is CR^{a}═CR^{a} and E, G each independently are CR^{a}, CX^{D} or N; or D-M is CR^{a}═N and E is CR^{a}, CX^{D}, N, and G is CR^{a} or CX^{D}; or
E is C, G is CR^{a}, CX^{D} or N, and D, M are CR^{a}₂, S, SO, SO₂, O, NR^{b}, or at most one of C═O, C═S, C═NR^{b}, C═CR^{a}₂, but no adjacent O—O or S—S groups; or E and G are C, and D and M independently from each other are CR^{a}₂, S, SO, SO₂, O, NR^{b} but no adjacent O—O, or S—S groups.
Fused bicyclic 7-membered rings are:

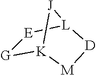

E-G is part of the above mentioned 8-membered ring and can be fused to PQ, QP, QX, XQ, XZ, ZX, ZY, YZ, YA, AY, such that P, A are C, CR^{a} or CX^{D}, and such that CX^{D} can only be present in A and P;

E, G are C, CR^{a}, CX^{D} or N; K, L are CR^{a}; D,M form a CR^{a}═CR^{a} or CR^{a}═N, or D,M independently from each other are CR^{a}₂, C═O, C═S, C═NR^{b}, C═CR^{a}₂, S, SO, SO₂, O, NR^{b} but no adjacent O—O, S—S, N—S groups; J is CR^{a}₂, C═O, C═S, C═NR^{b}, C═CR^{a}₂, S, SO, SO₂, O, NR^{b}; at most 2 N groups; or

E,G are C, CR^{a}, CX^{D}; K is N and L is CR^{a}; D,M form a CR^{a}═CR^{a} bond or D,M independently from each other are CR^{a}₂, C═O, C═S, C═NR^{b}, C═CR^{a}₂, NR^{b} but no adjacent O—O, S—S, N—S groups; J is CR^{a}₂, C═O, C═S, C═NR^{b}, C═CR^{a}₂, S, SO, SO₂, O, NR^{b}; at most 2 N groups; or E,G are C, CR^{a}, CX^{D}; K and L are N; D,M, J independently from each other are CR^{a}₂, C═O, C═S, C═NR^{b}, C═CR^{a}₂ groups;

Fused aromatic 5-membered rings are

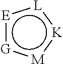

E, G are part of the above mentioned 8-membered ring and can be fused to QX, XQ, XZ, ZX, ZY, YZ.

E and G are C; one of the groups L, K, or M are O, NR^{b}, S and the remaining two groups are independently from each other CR^{a} or N; or E is C and G is N; L, K, M are independently from each other CR^{a} or N.

Fused aromatic 6-membered rings are:

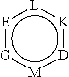

E, G are part of the above mentioned 8-membered ring and can be fused to QX, XQ, XZ, ZX, ZY, YZ.

E,G is C; L, K, D, M are independently from each other CR^{a} or N

Fused planar conjugated 7-membered rings are

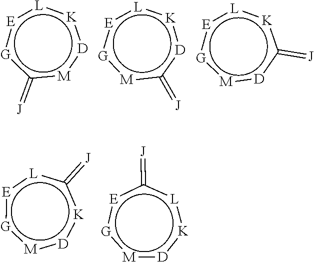

E, G are part of the above mentioned 8-membered ring and can be fused to QX, XQ, XZ, ZX, ZY, YZ

E,G is C; L, K, D, M are CR^{a}; J is S, O, CR^{a}₂, NR^{b}.

R^{a} is as defined for R₄₇, preferably as in section 8.
R^{b} is as defined for R₃₇, preferably as in section 8.
Each R^{c} as above indicated is independently selected from the group consisting of H, alkyl, aryl, O-alkyl, O-aryl, OH;

In all of the above embodiments, optionally one of A, P, Q, Y, X, and Z, or the substituents or fused rings of which they are part, or the self-immolative linker L^{C}, or the drug C^{A}, is bound, optionally via a spacer or spacers S^{P}, to one or more Constructs-B.

The synthesis of TCO's as described above is well available to the skilled person. This expressly also holds for TCO's having one or more heteroatoms in the strained cycloalkene rings. References in this regard include Cere et al. Journal of Organic Chemistry 1980, 45, 261 and Prevost et al. Journal of the American Chemical Society 2009, 131, 14182.

In a preferred embodiment, the trans-cyclooctene moiety satisfies formula (1b):

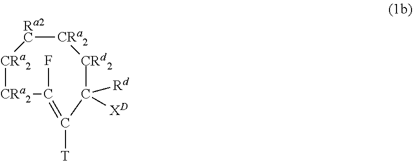

wherein, in addition to the optional presence of at most two exocyclic bonds fixed in the same plane, each R^{a} independently denotes H, or, in at most four instances, a substituent selected from the group consisting of alkyl, aryl, OR′, SR′, S(═O)R′″, S(═O)₂R′″, S(═O)₂NR′R″, Si—R′″, Si—O—R′″, OC(═O)R′″, SC(═O)R′″, OC(═S)R′″, SC(═S)R′″, F, Cl, Br, I, N₃, SO₂H, SO₃H, SO₄H, PO₃H, POOH, NO, NO₂, CN, OCN, SCN, NCO, NCS, CF₃, CF₂—R′, NR′R″, C(═O)R′, C(═S)R′, C(═O)O—R′, C(═S)O—R′, C(═O)S—R′, C(═S)S—R′, C(═O)NR′R″, C(═S)NR′R″, NR′C(═O)—R″, NR′C(═S)—R′″, NR′C(═O)O—R′″, NR′C(═S)O—R″, NR′C(═O)S—R″, NR′C(═S)S—R′″, OC(═O)NR′—R′″, SC(═O)NR′—R″, OC(═S)NR′—R′″, SC(═S)NR′—R″, NR′C(═O)NR″—R″, NR′C(═S)NR″—R″, CR′NR″, with each R′ and each R″ independently being H, aryl or alkyl and R′″ independently being aryl or alkyl;
Each R^{d} as above indicated is independently selected from the group consisting of H, alkyl, aryl, OR′, SR′, S(═O)R′″, S(═O)₂R′″, Si—R′″, Si—O—R″, OC(═O)R′″, SC(═O)R′″, OC(═S)R′″, SC(═S)R′″, F, Cl, Br, I, N₃, SO₂H, SO₃H, PO₃H, NO, NO₂, CN, CF₃, CF₂—R′, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R′″, NR′C(═O)S—R′″, NR′C(═S)S—R′″, NR′C(═O)NR″—R″, NR′C(═S)NR″—R″, CR′NR″, with each R′ and each R″ independently being H, aryl or alkyl and R′″ independently being aryl or alkyl;
wherein R^{d} is as defined for R₄₇, preferably as in Section 8,
wherein two R^{a}>^{d} moieties together may form a ring;
with optionally one R^{a,d} comprised in a linker moiety, optionally via a spacer S^{P}, to a Construct B, and wherein T and F each independently denote H, or a substituent selected from the group consisting of alkyl, F, Cl, Br, and I, and X^{D} is as defined above for formula (Ia).

Preferably, each R^{a} and each R^{d} is selected independently from the group consisting of H, alkyl, O-alkyl, O-aryl, OH, C(═O)NR′R″, NR′C(═O)—R′″, with R′ and R″ each independently being H, aryl or alkyl, and with R′″ independently being alkyl or aryl.

In the foregoing dienophiles, it is preferred that the at least two exocyclic bonds fixed in the same plane are selected from the group consisting of (a) the single bonds of a fused cyclobutyl ring, (b) the hybridized bonds of a fused aromatic ring, (c) an exocyclic double bond to an oxygen, (d) an exocyclic double bond to a carbon, (e) the single bonds of a fused dioxalane ring, (f) the single bonds of a fused cyclopropyl ring.

The TCO, containing one or two X^{D} moieties, may consist of multiple isomers, also comprising the equatorial vs. axial positioning of substituents, such as X^{D}, on the TCO. In this respect, reference is made to Whitham et al. J. Chem. Soc. (C), 1971, 883-896, describing the synthesis and characterization of the equatorial and axial isomers of trans-cyclo-oct-2-en-ol, identified as (1RS, 2RS) and (1SR, 2RS), respectively. In these isomers the OH substituent is either in the equatorial or axial position.

In a preferred embodiment, for prodrug structures where the X^{D} can be either in the axial or the equatorial position, the X^{D} is in the axial position.

Preferred TCO compounds according to this invention are the racemic and enantiomerically pure compounds listed below:

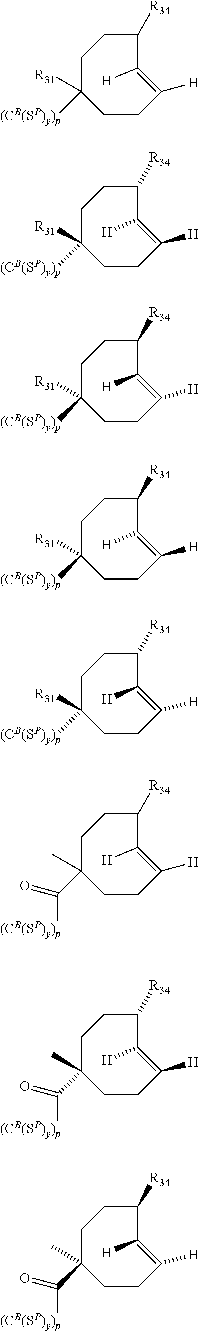

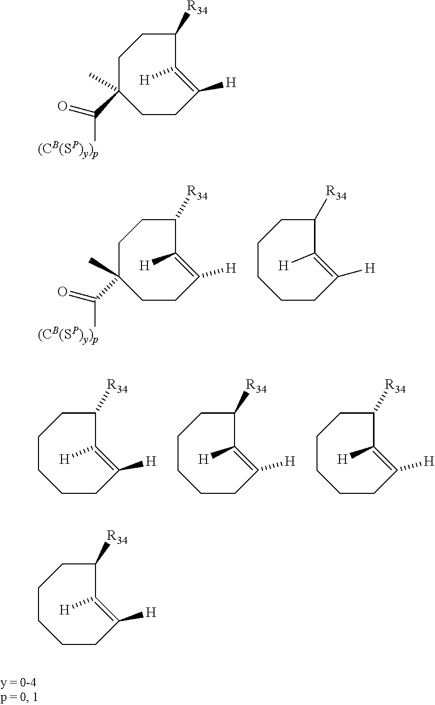

Especially preferred TCO compounds according to this invention are the enantiomerically pure compounds listed below:

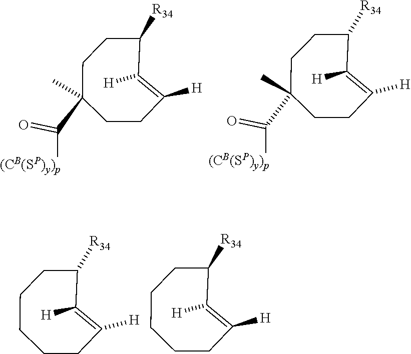

Preferably, R₃₄ is identical to R₄₈ as described herein.
Other preferred TCO compounds are:

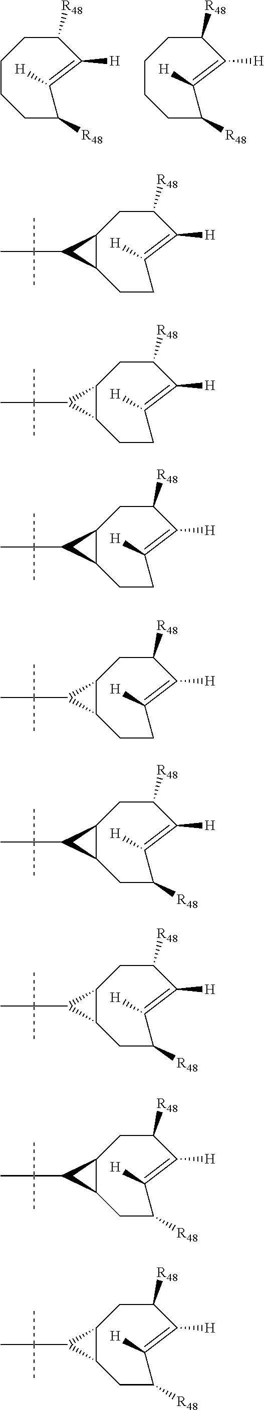

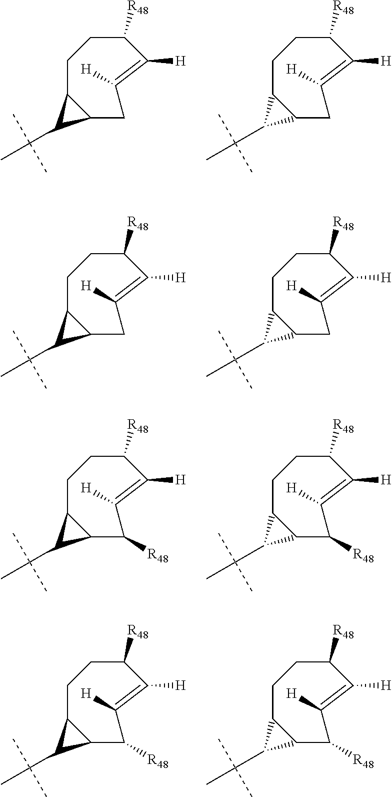

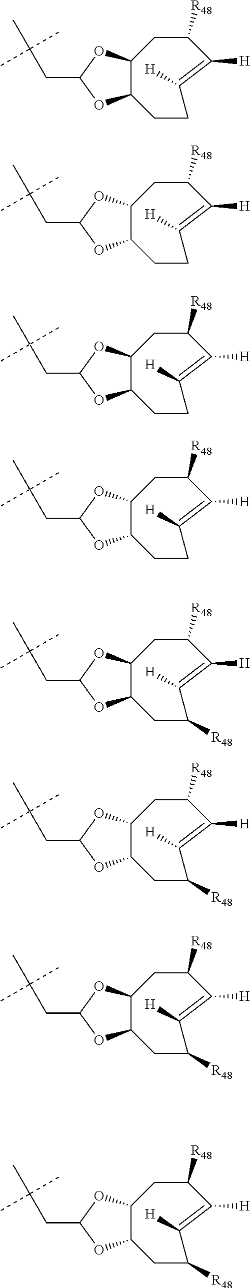

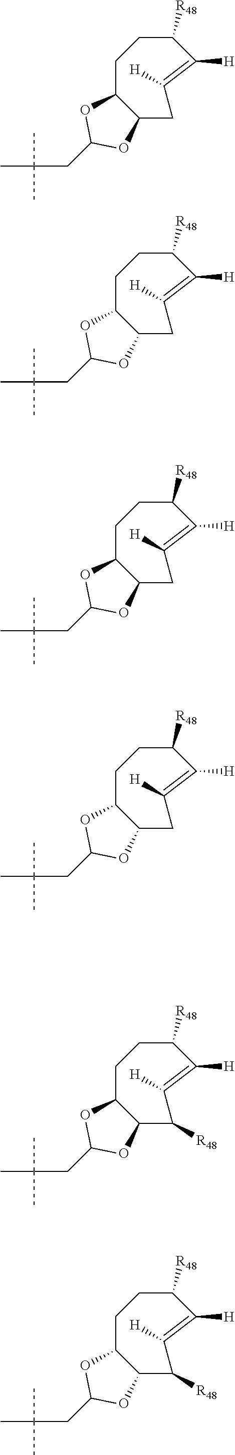

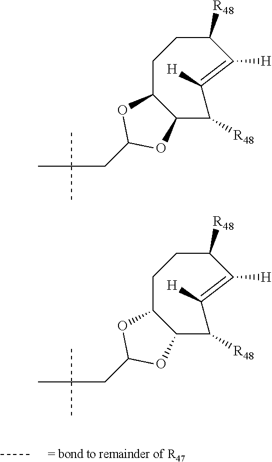

Preferred TCO intermediates to prepare the TCO prodrugs of the invention are listed below. Particularly preferred intermediates from the below are enantiomerically pure compounds A-F, in particular A, D, E, F. A person skilled in the art will understand that compounds E and F still need to be isomerized to E-cyclooctenes, after which the enantiomer with the axial OH can be separated from the enantiomer with the equatorial OH as described by Rossin et al Bioconj. Chem., 2016 27(7):1697-1706.

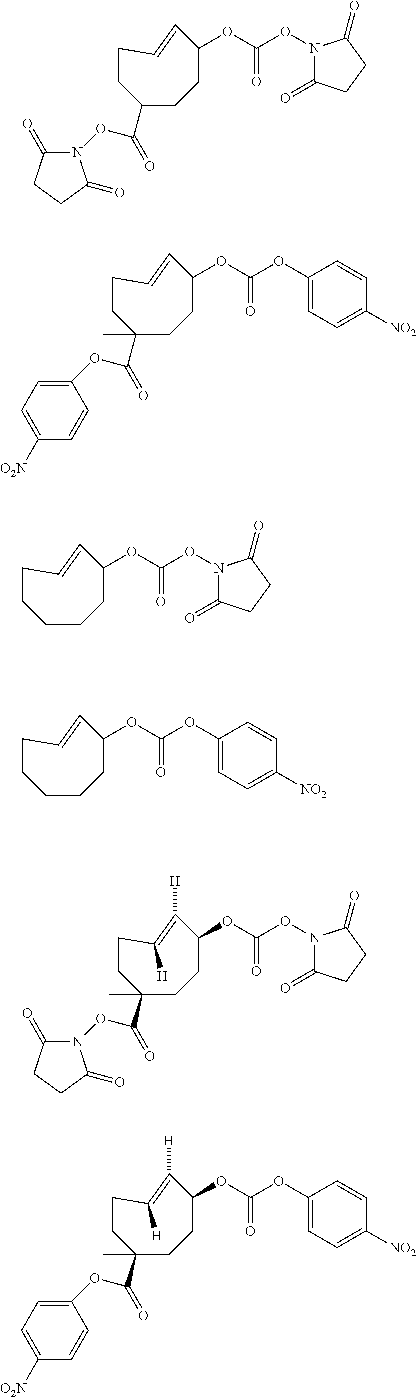

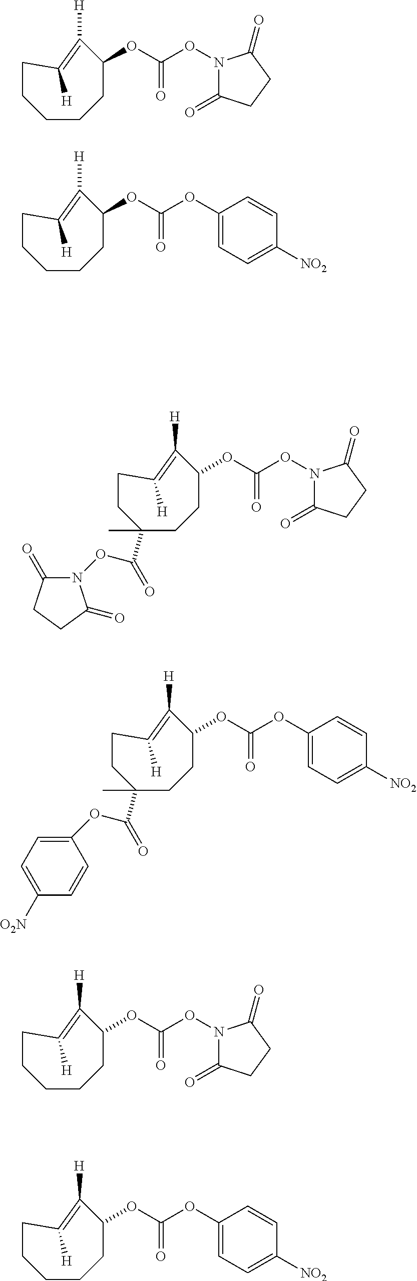

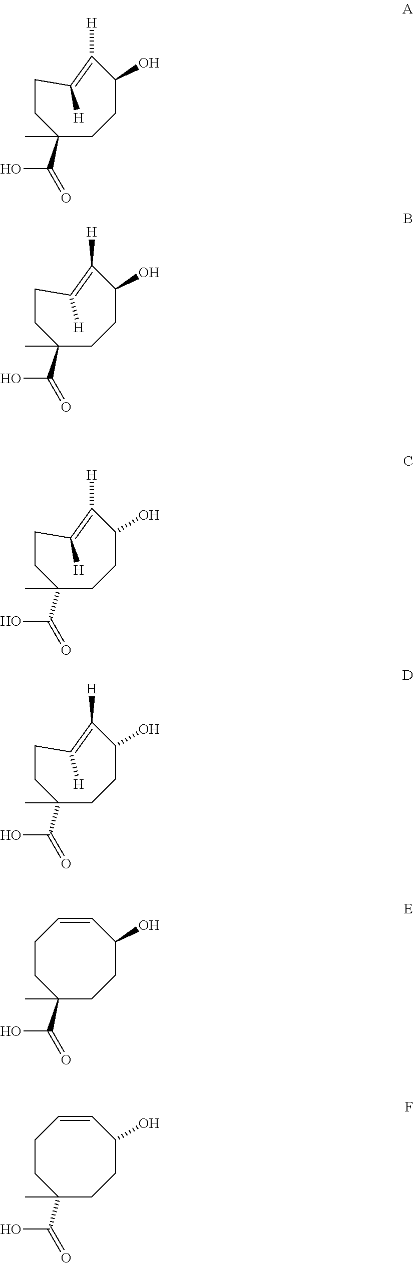

A general synthesis method of a TCO trigger and its corresponding prodrugs is shows directly below. The synthesis method is as reported in Rossin et al Nature Communications 2018, 9, 1484 and Rossin et al Bioconj. Chem., 2016 27(7):1697-1706 with the exception of the conversion of D to F, which now is conducted by mixing D with hydroxide solution in methanol, followed by evaporation and reaction with iodomethane. Please note that for sake of clarity only one of the two enantiomers of E-K is shown. A person skilled in the art will understand that the enantiomers can be separated at various stages in the synthesis using established chiral resolution methods to obtain enantiomerically pure B, E, F, H, for example, such as chiral salts.

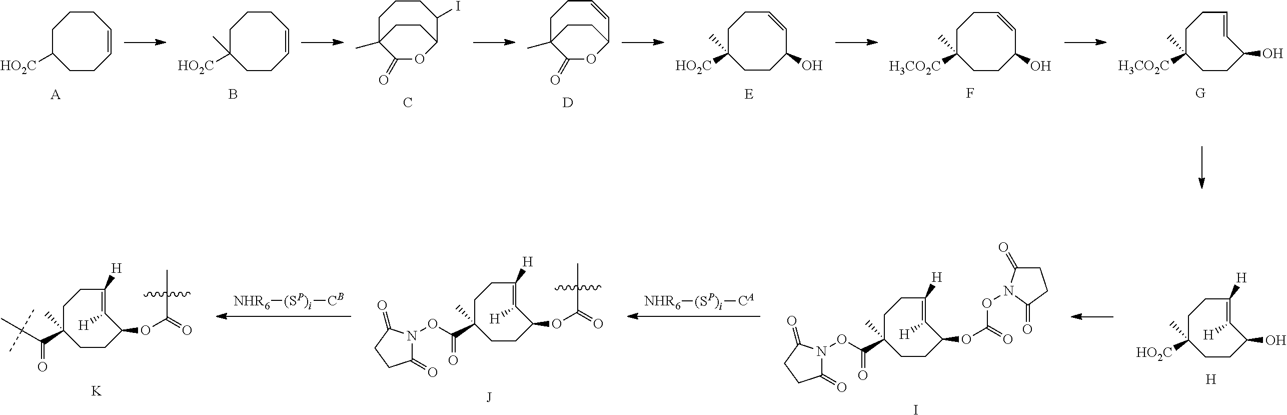

### Section 10—C^{A} and C^{B}

The Constructs A and Constructs B include but are not limited to small molecules, organic molecules, metal coordination compounds, molecules comprising a radionuclide, chelates comprising a radiometal, inorganic molecules, organometallic molecules, biomolecules, polymers, resins, particles (e.g. micro- and nanoparticles), liposomes, micelles, polymersomes, gels, surfaces, cells, biological tissues, and pathogens. Preferably, each C^{A} and C^{B} are independently an organic molecule or an inorganic molecule.

Examples of biomolecules include: carbohydrates, biotin, peptides, peptoids, lipids, proteins, enzymes, oligonucleotides, DNA, RNA, PNA, LNA, aptamers, hormones, toxins, steroids, cytokines, antibodies, antibody fragments (e.g. Fab2, Fab, scFV, diabodies, triabodies, VHH), antibody (fragment) fusions (e.g. bi-specific and trispecific mAb fragments).

In preferred Prodrug embodiments, C^{A} denotes a Construct A that is selected from the group consisting of drugs, targeting agents, and masking moieties. Preferably, Construct A is a drug, preferably a drug as defined herein.

In some preferred Prodrug embodiments, C^{B} denotes a Construct B, wherein said Construct B is selected from the group consisting of masking moieties, drugs, and targeting agents. Preferably, Construct B is selected from the group consisting of masking moieties, and targeting agents.

The Constructs A and Constructs B used in in vitro embodiments include but are not limited to small molecules, organic molecules (including fluorescent dyes), metal coordination compounds, molecules comprising a radionuclide, chelates comprising a radiometal, inorganic molecules, organometallic molecules, biomolecules, drugs, polymers, resins (e.g. polystyrene, agarose), particles (e.g. beads, magnetic beads, gold, silica-based particles and materials, polymers and polymer-based materials, glass, iron oxide particles, micro- and nanoparticles (such as liposomes, polymersomes), gels, surfaces (e.g. glass slides, chips, wavers, gold, metal, silica-based, polymer, plastic, resin), cells, biological tissues, pathogens (viruses, bacteria, fungi, yeast). The Constructs may for example comprise a combination of the aforementioned Constructs.

Construct A and Construct B can also be R₃₂ or a moiety comprising R₃₂, as defined herein, wherein R₃₂ can be used to bind to a further Construct A or B. For example, Construct A can be R₃₂ being a maleimide or photocrosslinker that is bound to the T^{R} via a Spacer S^{P}. The maleimide or photocrosslinker can be used to further conjugate the T^{R} to a protein. In this particular embodiment C^{A} and C^{B} are a biomolecule-binding moiety.

In preferred embodiments, each C^{A} and C^{B} are independently selected from the group consisting of organic molecules, inorganic molecules, organometallic molecules, resins, beads, glass, microparticles, nanoparticles, gels, surfaces, and cells. Preferably, each C^{A} and C^{B} are independently selected from the group consisting of organic molecules, and inorganic molecules.

In preferred embodiments, each C^{A} and C^{B} are independently selected from the group consisting of small molecules, proteins, carbohydrates, peptides, peptoids, oligosaccharides, molecules comprising a radionuclide, fluorescent dyes, inorganic molecules, organometallic molecules, polymers, lipids, oligonucleotides, DNA, RNA, PNA, LNA, drugs, resins, beads, glass, microparticles, nanoparticles, gels, surfaces, and cells.

Preferably, a small molecule is a small organic molecule. Preferably, a small molecule has a molecular weight of at most 2 kDa, more preferably at most 1 kDa, more preferably at most 750 Da, more preferably at most 500 Da, and most preferably at most 300 Da. Preferably, a small molecule has a molecular weight of at least 15 Da, more preferably at least 50 Da, more preferably at least 75 Da, and most preferably at least 100 Da.

In another preferred embodiment, each C^{A} and C^{B} are independently a moiety according to Formula (9) as defined herein.

### Section 11—Construct-Trigger Assemblies and Spacers S^{P}

A Construct-Trigger comprises a conjugate of the Construct or Constructs C^{A} and the Trigger T^{R}. Optionally the Trigger is further linked to Construct or Constructs C^{B}.
The general formula of the Construct-Trigger is shown below in Formula (5a) and (5b). For the avoidance of doubt, as Y^{C} is part of L^{C} and C^{A}, Y^{C} is not separately denoted in Formula (5a) and (5b).

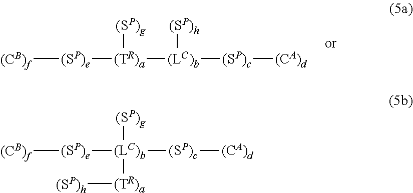

C^{A} is Construct A, C^{B} is Construct B, S^{P} is Spacer; T^{R} is Trigger, and L^{C} is Linker.

b,c,e,f,g,h≥0;a,d≥1. Formula (5a):

c,e,f,g,h≥0;a,b,d≥1. Formula (5b):

In the Trigger-Construct conjugate, the Construct C^{A} and the Trigger T^{R}—the TCO derivative—can be directly linked to each other. They can also be bound to each other via a self-immolative linker L^{C}, which may consist of multiple (self-immolative, or non immolative) units. With reference to Formula 5a and 5b, when L^{C} contains a non immolative unit, this unit equals a Spacer S^{P} and c≥1. It will be understood that the invention encompasses any conceivable manner in which the diene Trigger is attached to the one or more Construct C^{A}. The same holds for the attachment of one or more Construct C^{B} to the Trigger or the linker L^{C}. The same holds for the optional attachment of one or more Spacer S^{P} to the Trigger or the linker L^{C}. Methods of affecting conjugation, e.g. through reactive amino acids such as lysine or cysteine in the case of proteins, are known to the skilled person. Exemplary conjugation methods are outlined in the section on Conjugation herein below.

It will be understood that the Construct C^{A} is linked to the TCO in such a way that the Construct C^{A} is eventually capable of being released after formation of the IEDDA adduct. Generally, this means that the bond between the Construct C^{A} and the TCO, or in the event of a self-immolative Linker L^{C}, the bond between the Linker and the TCO and between the Construct C^{A} and the Linker, should be cleavable. Predominantly, the Construct C^{A} and the optional Linker is linked via a hetero-atom, preferably via O, N, NH, or S. The cleavable bond is preferably selected from the group consisting of carbamate, thiocarbamate, carbonate, ester, ether, thioether, amide, thioester bonds.

It shall be understood that one C^{B} can be modified with more than one Trigger. For example, an antibody can be modified with 4 TCO-drug constructs by conjugation to 4 amino acid residues, wherein C^{A} is drug.

Likewise, it shall be understood that one C^{A} can be modified with more than one Trigger. For example, a protein drug can be masked by conjugation of 4 amino acid residues to 4 TCO-polyethylene glycol constructs, wherein polyethylene glycol is C^{B}.

Furthermore, it shall be understood that one C^{A} can be modified with more than one Trigger, wherein at least one Trigger links to a Targeting Agent, being C^{B}, and at least one Trigger links to a Masking Moiety being C^{B}, wherein C^{A} can be a Drug, preferably a protein.

### Spacers S^{P}

It will be understood that when herein, it is stated that “each individual S^{P} is linked at all ends to the remainder of the structure” this refers to the fact that the spacer S^{P} connects multiple moieties within a structure, and therefore the spacer has multiple ends by definition. The spacer S^{P} may be linked to each individual moiety via different or identical moieties that may be each individually selected. Typically, these linking moieties are to be seen to be part of spacer S^{P} itself. In case the spacer S^{P} links two moieties within a structure, “all ends” should be interpreted as “both ends”. As an example, if the spacer connects a trans-cylooctene moiety to a Construct A, then “the remainder of the molecule” refers to the trans-cylooctene moiety and Construct A, while the connecting moieties between the spacer and the trans-cyclooctene moiety and Construct A (i.e. at both ends) may be individually selected.

In a preferred embodiment, Spacers S^{P} may consist of one or multiple Spacer Units S^{U} arranged linearly and/or branched and may be connected to one or more C^{B} moieties and/or one or more L^{C} or T^{R} moieties. The Spacer may be used to connect C^{B} to one T^{R} (Example A below; with reference to Formula 5a and 5b: f, e, a=1) or more T^{R} (Example B and C below; with reference to Formula 5a and 5b: f, e=1, a≥1), but it can also be used to modulate the properties, e.g. pharmacokinetic properties, of the C^{B}-T^{R}-C^{A} conjugate (Example D below; with reference to Formula 5a and 5b: one or more of c,e,g,h≥1). Thus a Spacer unit does not necessarily connect two entities together, it may also be bound to only one component, e.g. the T^{R} or L^{C}. Alternatively, the Spacer may comprise a Spacer Unit linking C^{B} to T^{R} and in addition may comprise another Spacer Unit that is only bound to the Spacer and serves to modulate the properties of the conjugate (Example F below; with reference to Formula 5a and 5b: e≥1). The Spacer may also consist of two different types of S^{U} constructs, e.g. a PEG linked to a peptide, or a PEG linked to an alkylene moiety (Example E below; with reference to Formula 5a and 5b: e≥1). For the sake of clarity, Example B depicts a S^{U} that is branched by using a multivalent branched S^{U}. Example C depicts a S^{U} that is branched by using a linear S^{U} polymer, such as a peptide, whose side chain residues serve as conjugation groups.

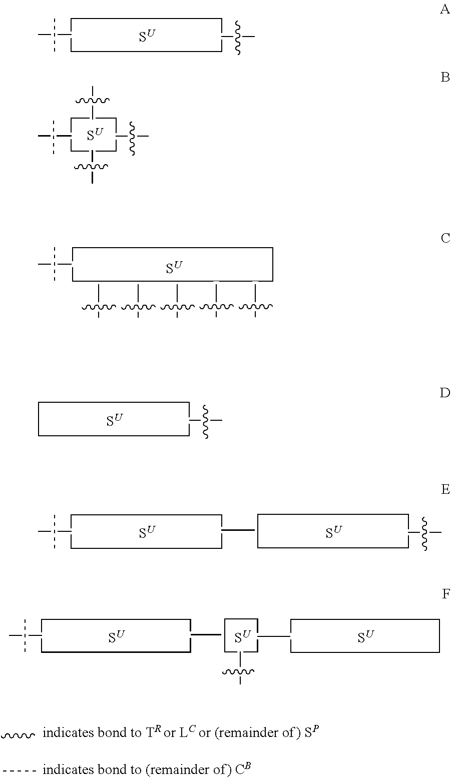

The Spacer may be bound to the Activator in similar designs such as depicted in above examples A-F.
The Spacer Units include but are not limited to amino acids, nucleosides, nucleotides, and biopolymer fragments, such as oligo- or polypeptides, oligo- or polypeptoids, or oligo- or polylactides, or oligo- or poly-carbohydrates, varying from 2 to 200, particularly 2 to 113, preferably 2 to 50, more preferably 2 to 24 and more preferably 2 to 12 repeating units. Exemplary preferred biopolymer S^{U} are peptides.

Yet other examples are alkyl, alkylene, alkenyl, alkenylene, alkynyl, alkynylene, cycloalkyl, cycloalkylene, cycloalkenyl, cycloalkenylene, cycloalkynyl, cycloalkynylene, aryl, arylene, alkylaryl, alkylarylene, arylalkyl, arylalkylene, arylalkenyl, arylalkenylene, arylalkynyl, arylalkynylene, polyethyleneamino, polyamine, which may be substituted or unsubstituted, linear or branched, may contain further cyclic moieties and/or heteroatoms, preferably O, N, and S, more preferably O; wherein In preferred embodiments these example S^{U} comprise at most 50 carbon atoms, more preferably at most 25 carbon atoms, more preferably at most 10 carbon atoms. In some preferred embodiments the S^{U} is independently selected from the group consisting of (CH₂)ᵣ, (C₃-C₈, carbocyclo), O—(CH₂)ᵣ, arylene, (CH₂)ᵣ-arylene, arylene-(CH₂)ᵣ, (CH₂)ᵣ—(C₃-C₈, carbocyclo), (C₃-C₈, carbocyclo)-(CH₂)ᵣ, (C₃-C₈, heterocyclo), (CH₂)ᵣ—(C₃-C₈, heterocyclo), (C₃-C₈, heterocyclo)-(CH₂)ᵣ, —(CH₂)ᵣC(O)NR⁴(CH₂)ᵣ, (CH₂CH₂O)ᵣ, (CH₂CH₂O)ᵣCH₂—(CH₂)ᵣC(O)NR⁴(CH₂ CH₂O)ᵣ, (CH₂)ᵣC(O)NR⁴(CH₂CH₂O)ᵣCH₂, (CH₂CH₂O)ᵣ C(O)NR⁴(CH₂CH₂O)ᵣ, (CH₂CH₂O)ᵣ C(O)NR⁴(CH₂CH₂O)ᵣCH₂, (CH₂CH₂O)ᵣC(O)NR⁴CH₂; wherein r is independently an integer from 1-10, and R⁴ is as defined herein.

Other examples of Spacer Units S^{U} are linear or branched polyalkylene glycols such as polyethylene glycol (PEG) or polypropylene glycol (PPG) chains varying from 2 to 200, particularly 2 to 113, preferably 2 to 50, more preferably 2 to 24 and more preferably 2 to 12 repeating units. It is preferred that when polyalkylene glycols such as PEG and PPG polymers are only bound via one end of the polymer chain, that the other end is terminated with —OCH₃, —OCH₂CH₃, OCH₂CH₂CO₂H.

Other polymeric Spacer Units are polymers and copolymers such as poly-(2-oxazoline), poly(N-(2-hydroxypropyl)methacrylamide) (HPMA), polylactic acid (PLA), polylactic-glycolic acid (PLGA), polyglutamic acid (PG), dextran, polyvinylpyrrolidone (PVP), poly(l-hydroxymethylethylene hydroxymethyl-formal (PHF). Other exemplary polymers are polysaccharides, glycopolysaccharides, glycolipids, polyglycoside, polyacetals, polyketals, polyamides, polyethers, polyesters. Examples of naturally occurring polysaccharides that can be used as S^{U} are cellulose, amylose, dextran, dextrin, levan, fucoidan, carraginan, inulin, pectin, amylopectin, glycogen, lixenan, agarose, hyaluronan, chondroitinsulfate, dermatansulfate, keratansulfate, alginic acid and heparin. In yet other exemplary embodiments, the polymeric S^{U} comprises a copolymer of a polyacetal/polyketal and a hydrophilic polymer selected from the group consisting of polyacrylates, polyvinyl polymers, polyesters, polyorthoesters, polyamides, oligopeptides, polypeptides and derivatives thereof. Exemplary preferred polymeric S^{U} are PEG, HPMA, PLA, PLGA, PVP, PHF, dextran, oligopeptides, and polypeptides.

In some aspects of the invention polymers used in a S^{U} have a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa.

Other exemplary S^{U} are dendrimers, such as poly(propylene imine) (PPI) dendrimers, PAMAM dendrimers, and glycol based dendrimers.

The S^{U} of the invention expressly include but are not limited to conjugates prepared with commercially available cross-linker reagents such as BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-STAB, sulfo-SMCC, sulfo-SMPB, and SVSB, DTME, BMB, BMDB, BMH, BMOE, BM(PEO)₃ and BM(PEO)₄.

To construct a branching Spacer one may use a S^{U} based on one or several natural or non-natural amino acids, amino alcohol, aminoaldehyde, or polyamine residues or combinations thereof that collectively provide the required functionality for branching. For example serine has three functional groups, i.e. acid, amino and hydroxyl groups and may be viewed as a combined amino acid an aminoalcohol residue for purpose of acting as a branching S^{U}. Other exemplary amino acids are lysine and tyrosine.

In preferred embodiments, the Spacer consists of one Spacer Unit, therefore in those cases S^{P} equals S^{U}. In preferred embodiments the Spacer consists of two, three or four Spacer Units.

In some aspects of the S^{P} has a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa. In some aspects of the invention, the S^{P} has a mass of no more than 5000 daltons, no more than 4000 daltons, no more than 3000 daltons, no more than 2000 daltons, no more than 1000 daltons, no more than 800 daltons, no more than 500 daltons, no more than 300 daltons, no more than 200 daltons. In some aspects the S^{P} has a mass from 100 daltons, from 200 daltons, from 300 daltons to 5000 daltons. In some aspects of the S^{P} has a mass from 30, 50, or 100 daltons to 1000 daltons, from about 30, 50, or 100 daltons to 500 daltons.

In preferred embodiments, S^{P} is a spacer selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein for S^{P} the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized.

In preferred embodiments, S^{P} comprises a moiety C^{M2}, C^{X} or a residue of R₃₂, as described herein. In preferred embodiments, said C^{M2}, C^{X} or a residue of R₃₂ connects the S^{P} to C^{B}, C^{A}, L^{C}, or T^{R}. In preferred embodiments, when S^{P} comprises a moiety C^{M2}, C^{X} or a residue of R₃₂, it is coupled to a moiety C^{B} or C^{A} as indicated herein for how compounds according to Formula (21) are coupled to a moiety A according to Formula (20). In that case, C^{B}, for example, is equivalent to moiety A, wherein X is part of C^{B}.

In preferred embodiments, in any Formula as disclosed herein in relation to the invention each individual S^{P} is linked at all ends to the remainder of the structure according to said Formula via moieties that are part of S^{P} and are independently selected from the group consisting of —O—, —C(R⁶)₂—, —NR⁶—, —S—,

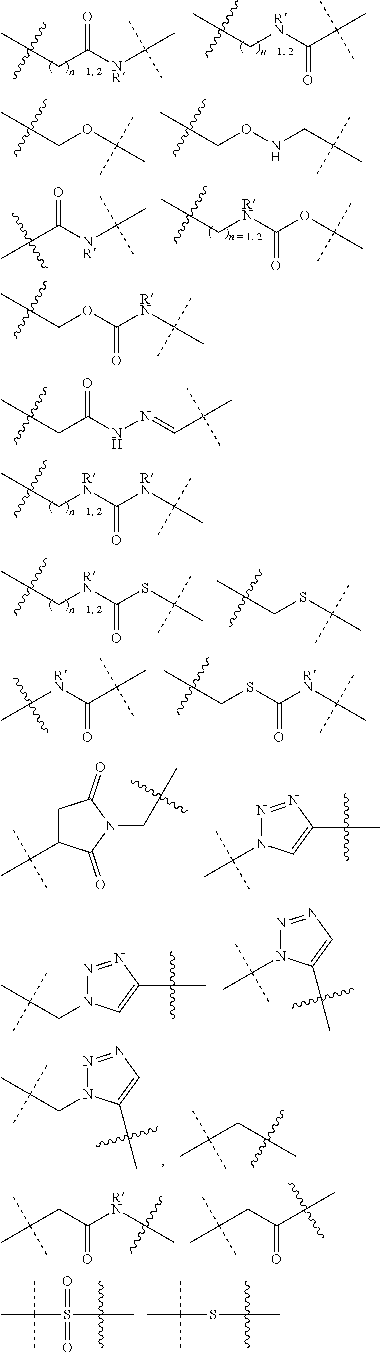

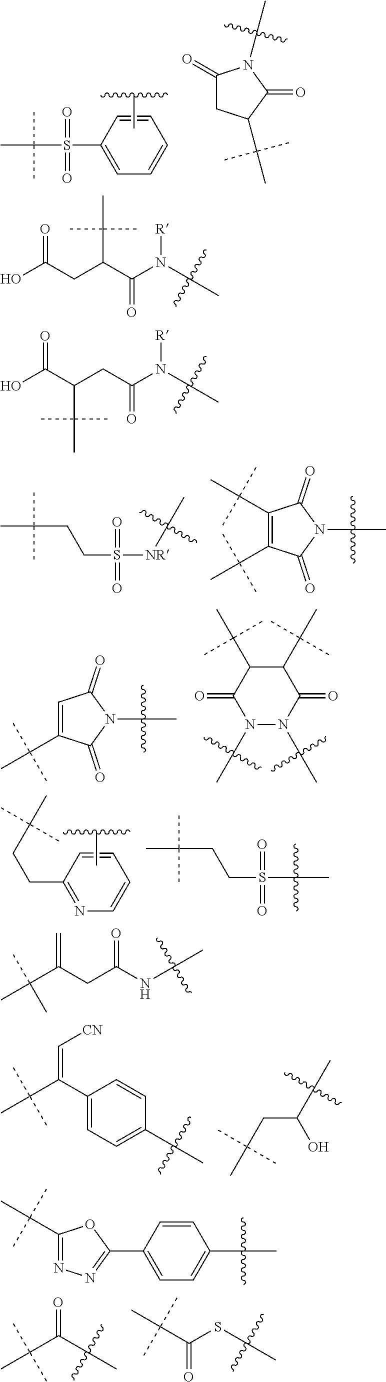

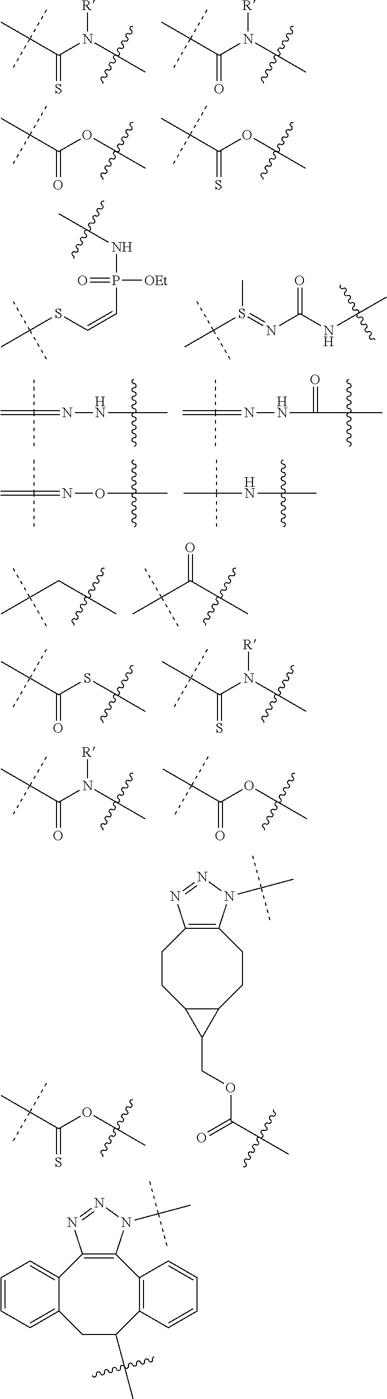

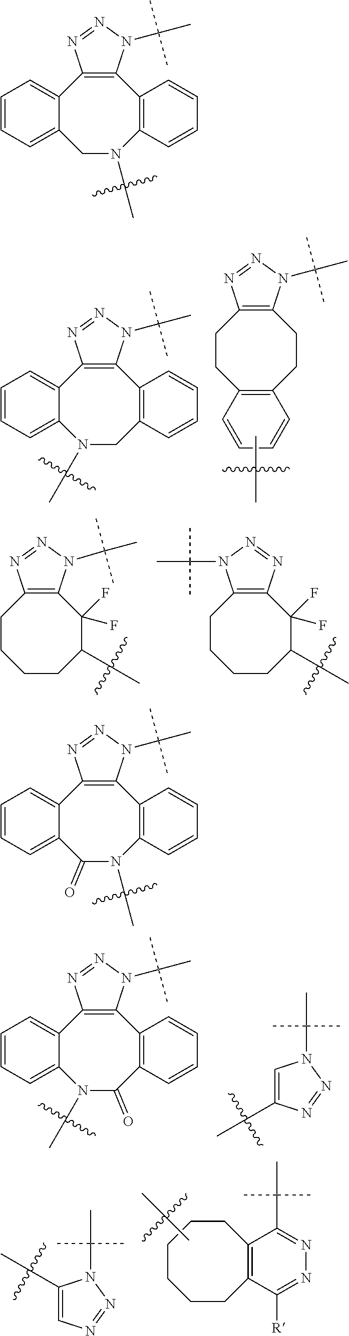

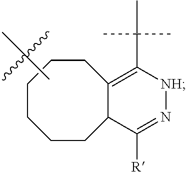

wherein the wiggly line depicts a bond to the remainder of S^{P} or to the remainder of said structure, and the dashed line depicts either a bond to the remainder of S^{P} when the wiggly line depicts a bond to the remainder of said structure, or a bond to the remainder of said structure when the wiggly line depicts a bond to the remainder of S^{P}. Herein, R′ is preferably as defined for R₃₇.
Preferably, R⁶ used in this Section is as defined in Section 8.

### Section 12—Linker L^{C}

L^{C} is an optional self-immolative linker, which may consist of multiple units arranged linearly and/or branched and may release one or more C^{A} moieties. By way of further clarification, if r is 0 the species C^{A} directly constitutes the leaving group of the release reaction, and if r>0, the self-immolative linker L^{C} constitutes the leaving group of the release reaction. The possible L^{C} structures, their use, position and ways of attachment of linkers L^{C}, constructs C^{A} and C^{B}, and the T^{R} are known to the skilled person, see for example [Papot et al., Anticancer Agents Med. Chem., 2008, 8, 618-637]. Nevertheless, preferred but non-limiting examples of self-immolative linkers L^{C} are benzyl-derivatives, such as those drawn below. There are two main self-immolation mechanisms: electron cascade elimination and cyclization-mediated elimination. The preferred example below on the left functions by means of the cascade mechanism, wherein the bond between the allylic carbon of the Trigger and the —O— or —S— attached to said carbon is cleaved, and an electron pair of Y^{C1}, for example an electron pair of NR⁶, shifts into the benzyl moiety resulting in an electron cascade and the formation of 4-hydroxybenzyl alcohol, CO₂ and the liberated C^{A}. The preferred example in the middle functions by means of the cyclization mechanism, wherein cleavage of the bond to the NR⁶ on the side of the Trigger leads to nucleophilic attack of the amine on the carbonyl, forming a 5-ring 1,3-dimethylimidazolidin-2-one and liberating the C^{A}. The preferred example on the right combines both mechanisms, this linker will degrade not only into CO₂ and one unit of 4-hydroxybenzyl alcohol (when Y^{C1} is O), but also into one 1,3-dimethylimidazolidin-2-one unit.

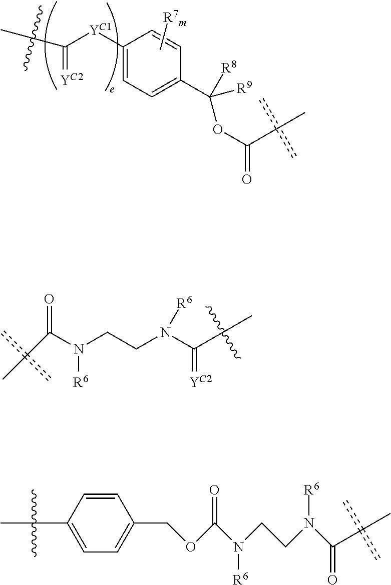

wherein the wiggly line indicates a bond to —O— or —S— on the allylic position of the trans-cyclooctene, and the double dashed line indicates a bond to C^{A}.
By substituting the benzyl groups of aforementioned self-immolative linkers L^{C}, it is possible to tune the rate of release of the construct C^{A}, caused by either steric and/or electronic effects on the cyclization and/or cascade release. Synthetic procedures to prepare such substituted benzyl-derivatives are known to the skilled person (see for example [Greenwald et al, J. Med. Chem., 1999, 42, 3657-3667] and [Thornthwaite et al, Polym. Chem., 2011, 2, 773-790]. Some preferred substituted benzyl-derivatives with different release rates are drawn below.

Self-immolative linkers that undergo cyclization include but are not limited to substituted and unsubstituted aminobutyric acid amide, appropriately substituted bicyclo[2.2.1] and bicyclo[2.2.2] ring system, 2-aminophenylpropionic acid amides, and trimethyl lock-based linkers, see e.g. [Chem. Biol. 1995, 2, 223], [J. Am. Chem. Soc. 1972, 94, 5815], [J. Org. Chem. 1990, 55, 5867], the contents of which are hereby incorporated by reference. Preferably, with an L^{C} that releases C^{A} by means of cyclization, the remainder of C^{A} is bound to L^{C} via an aromatic oxygen of sulfur. It will be understood that e.g. aromatic oxygen means an oxygen that is directly attached to an aromatic group.

Further preferred examples of L^{C} can be found in WO2009017394(A1), U.S. Pat. No. 7,375,078, WO2015038426A1, WO2004043493, Angew. Chem. Int. Ed. 2015, 54, 7492-7509, the contents of which are hereby incorporated by reference.

In preferred embodiments the L^{C} has a mass of no more than 1000 daltons, no more than 500 daltons, no more than 400 daltons, no more than 300 daltons, or from 10, 50 or 100 to 1000 daltons, from 10, 50, 100 to 400 daltons, from 10, 50, 100 to 300 daltons, from 10, 50, 100 to 200 daltons, e.g., 10-1000 daltons, such as 50-500 daltons, such as 100 to 400 daltons.

A person skilled in the art will know that one L^{C} may be connected to another L^{C} that is bound to C^{A}, wherein upon reaction of the Activator with the Trigger T^{R}, L^{C}-L^{C}-C^{A} is released from the T^{R}, leading to self-immolative release of both L^{C} moieties and the C^{A} moiety. With respect to the L^{C} formulas disclosed herein, the L^{C} linking the T^{R} to the other L^{C} then does not release C^{A} but an L^{C} that is bound via Y^{C1} and further links to a C^{A}. The skilled person will acknowledge that this principle also holds for further linkers L^{C} linked to L^{C}, e.g. L^{C}-L^{C}-L^{C}-L^{C}-C^{A}.

In a preferred embodiment, L^{C} is selected from the group consisting of linkers according to Group I, Group II, and Group III, as defined in this Section below.

Linkers according to Group I are

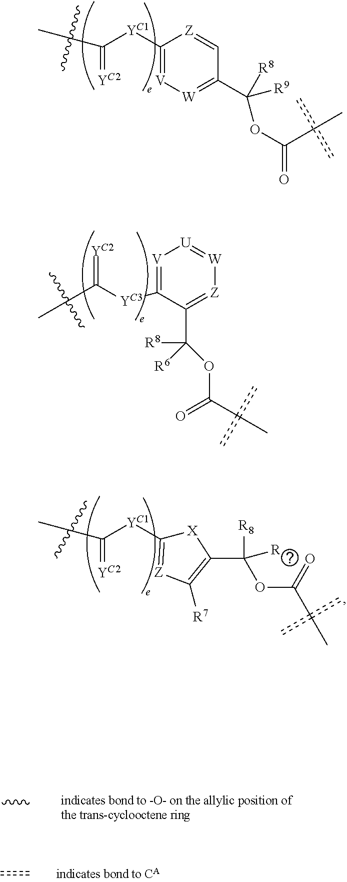

wherein the wiggly line may also indicate a bond to —S— on the allylic position of the trans-cyclooctene, wherein U, V, W, Z are each independently selected from the group consisting of —CR⁷—, and —N—,
wherein e is either 0 or 1,
wherein X is selected from the group consisting of —O—, —S— and —NR⁶—,
wherein preferably each R⁸ and R⁹ are independently selected from the group consisting of hydrogen, C₁-C₄ alkyl groups, C₂-C₄ alkenyl groups, and C₄₋₆ (hetero)aryl groups, wherein for R⁸ and R⁹ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein for linkers according to Group I C^{A} is linked to L^{C} via a moiety selected from the group consisting of —O—, —N—, —C—, and —S—, preferably from the group consisting of secondary amines and tertiary amines, wherein said moieties are part of C^{A}. Preferably, for linkers of Group I both R⁸ and R⁹ are hydrogen.
The linker according to Group II is

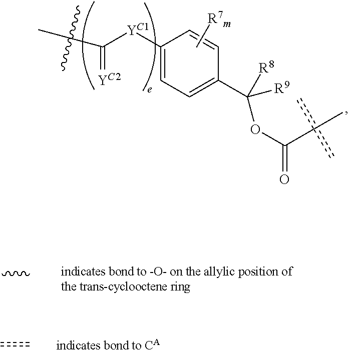

wherein the wiggly line may also indicate a bond to —S— on the allylic position of the trans-cyclooctene, wherein m is an integer between 0 and 2, preferably m is 0,
wherein e is either 0 or 1,
wherein for linkers according to Group II C^{A} is linked to L^{C} via a moiety selected from the group consisting of —O—, —N—, —C—, and —S—, preferably from the group consisting of secondary amines and tertiary amines, wherein said moieties are part of C^{A}. Preferably, for linkers of Group II both R⁸ and R⁹ are hydrogen. Preferably, for linkers of Group II R⁷ is methyl or isopropyl.
Linkers according to Group III are

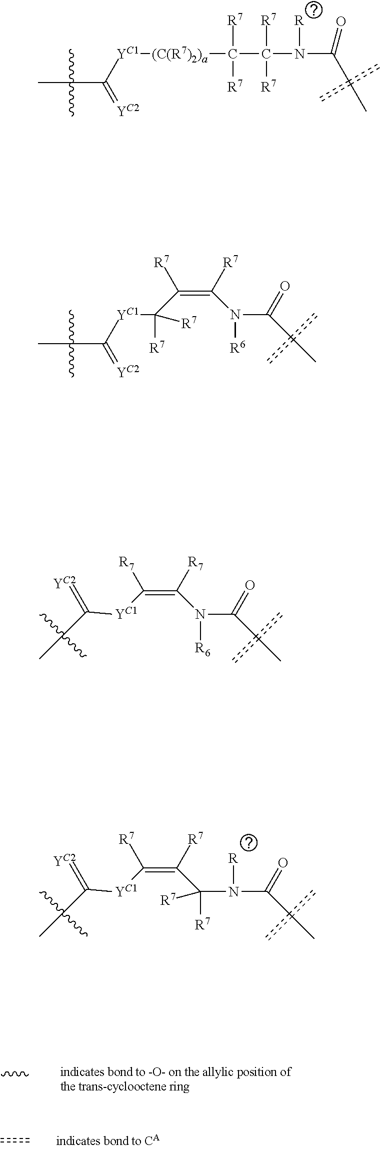

wherein the wiggly line may also indicate a bond to —S— on the allylic position of the trans-cyclooctene, wherein for linkers according to Group III C^{A} is linked to L^{C} via a moiety selected from the group consisting of —O— and —S—, preferably —O— or —S— bound to a C₄₋₆ (hetero)aryl group, wherein said moieties are part of C^{A},
wherein preferably each R⁶ is independently selected from the group consisting of hydrogen, C₁-C₄ alkyl groups, C₂-C₄ alkenyl groups, and C₄₋₆ (hetero)aryl groups,
wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO_{S}H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein preferably each R⁷ is independently selected from the group consisting of hydrogen and C₁-C₃ alkyl groups, C₂-C₃ alkenyl groups, and C₄₋₆ (hetero)aryl groups,
wherein for R⁷ the alkyl groups, alkenyl groups, and (hetero)aryl groups are optionally substituted with a moiety selected from the group consisting of —Cl, —F, —Br, —I, —OH, —NH₂, ═O, ═NH, —N(CH₃)₂, —S(O)₂CH₃, and —SH, and are optionally interrupted by at most one heteroatom selected from the group consisting of —O—, —S—, —NH—, —P—, and —Si—, wherein the N, S, and P atoms are optionally oxidized, wherein the N atoms are optionally quaternized,
wherein R⁷ is preferably selected from the group consisting of hydrogen, methyl, —CH₂—CH₂—N(CH₃)₂, and —CH₂—CH₂—S(O)₂—CH₃.
Preferably, for linkers of Group III, R⁶ is hydrogen. Preferably, for linkers of Group III, R⁶ is methyl.
R⁶, R⁷, R⁸, R⁹ comprised in said Group I, II and III, can optionally also be —(S^{P})—C^{B}.
For all linkers according to Group I and Group II Y^{C1} is selected from the group consisting of —O—, —S—, and preferably —NR⁶—. For all linkers according to Group III, Y^{C1} is —NR⁶—. For all linkers according to Group I, Group II, and Group III, Y^{C2} is selected from the group consisting of O and S, preferably 0.
When r as defined for Formula (19) is two, then the L^{C} attached to the —O— at the allylic position of the trans-cyclooctene is selected from the group consisting of linkers according to Group I and Group II, and the L^{C} between the L^{C} attached to the —O— or —S— at the allylic position of the trans-cyclooctene and C^{A} is selected from Group III, and that the wiggly line in the structures of Group III then denotes a bond to the L^{C} attached to the —O— or —S— at the allylic position of the trans-cyclooctene instead of a bond to the allylic —O— or —S— on the trans-cyclooctene ring, and that the double dashed line in the structures of Groups I and II then denotes a bond to the L^{C} between the L^{C} attached to the —O— or —S— at the allylic position of the trans-cyclooctene and the C^{A} instead of a bond to C^{A}.
In another preferred embodiment, L^{C} is selected from the group consisting of linkers according to Group IV, Group V, Group VI, and Group VII, as defined below in this Section.
Linkers according to Group IV are

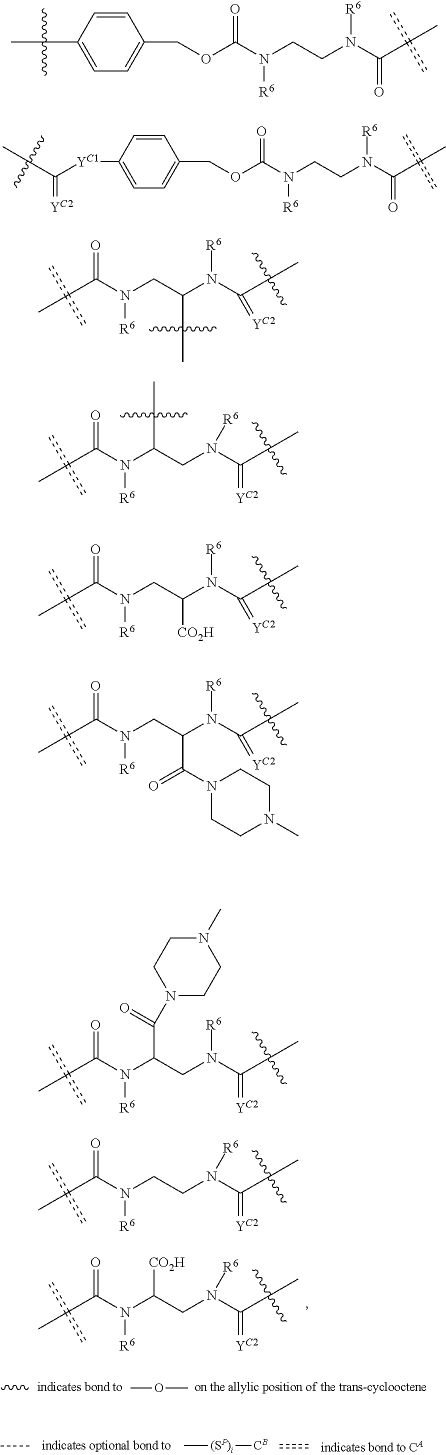

wherein the wiggly line may also indicate a bond to —S— on the allylic position of the trans-cyclooctene, wherein C^{A} is linked to L^{C} via a moiety selected from the group consisting of —O— and —S—, preferably from the group consisting of —O-05-8-arylene- and —S—O₅₋₈-arylene-, wherein said moieties are part of C^{A}.
Linkers according to Group V are

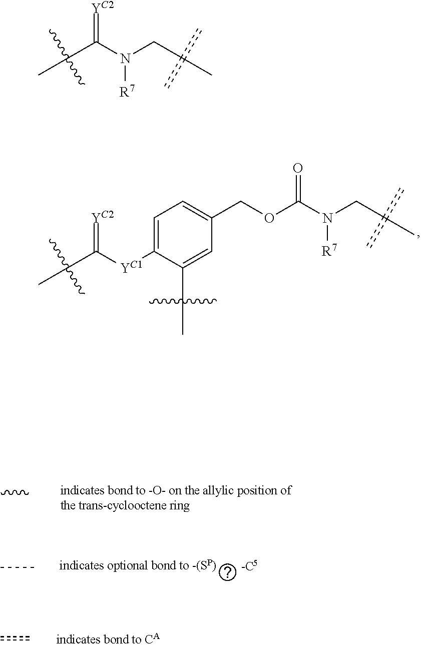

wherein the wiggly line may also indicate a bond to —S— on the allylic position of the trans-cyclooctene, wherein C^{A} is linked to L^{C} via a moiety selected from the group consisting of —O— and —S—, wherein said moieties are part of C^{A}. In the first linker of Group V, R⁷ is preferably —(CH₂)₂—N(CH₃)₂.
Linkers according to Group VI are

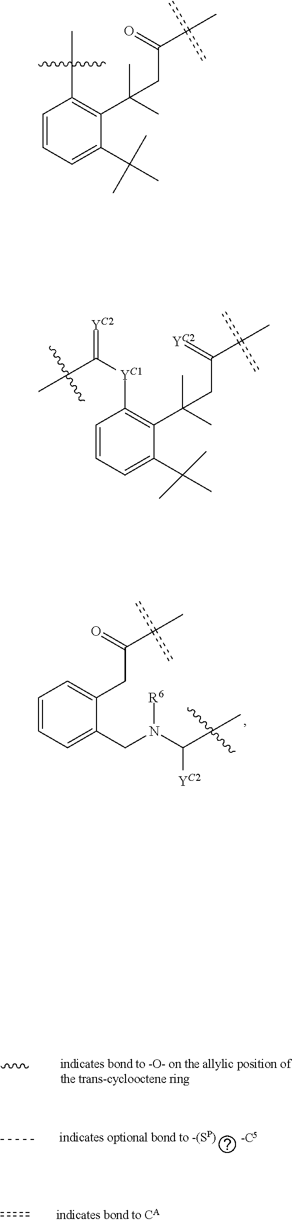

wherein the wiggly line may also indicate a bond to —S— on the allylic position of the trans-cyclooctene, wherein C^{A} is linked to L^{C} via a moiety selected from the group consisting of —O—, —N—, and —S—, preferably a secondary or a tertiary amine, wherein said moieties are part of C^{A}.
Linkers according to Group VII are

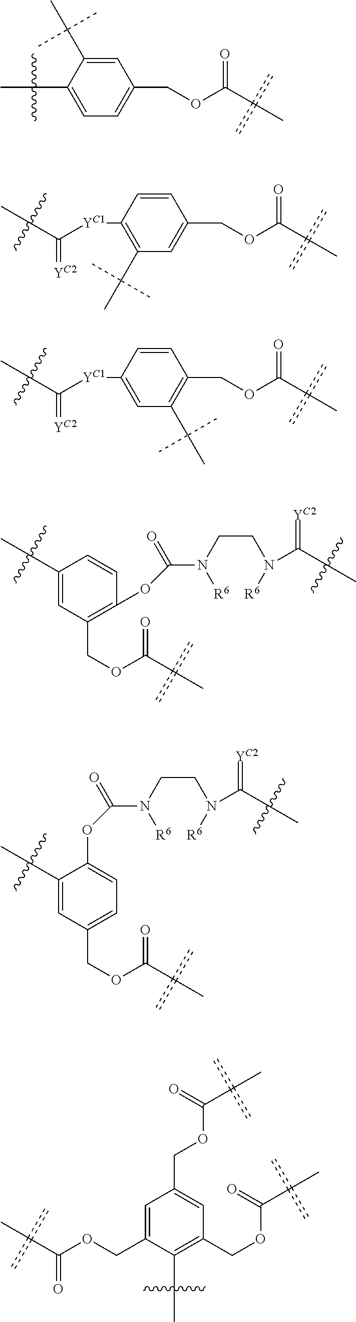

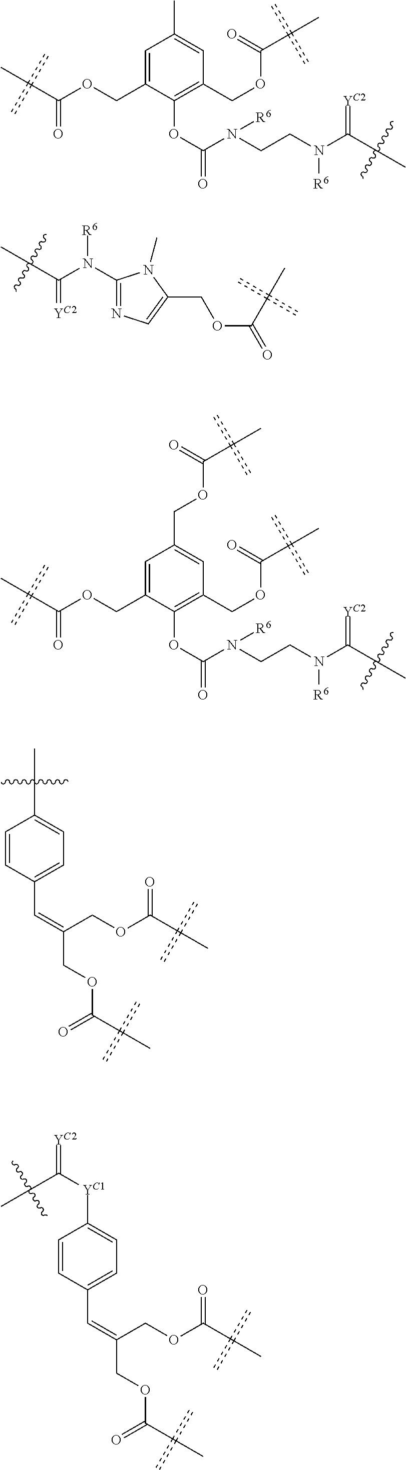

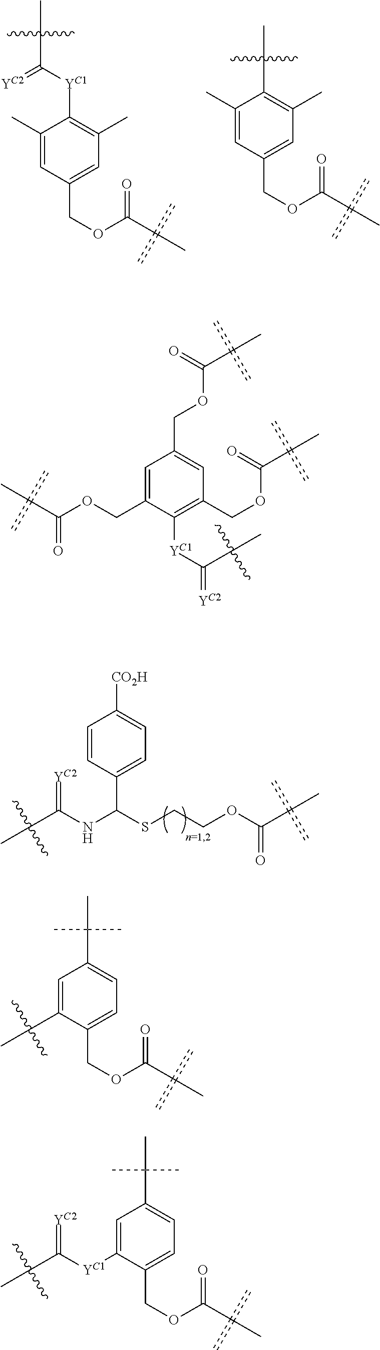

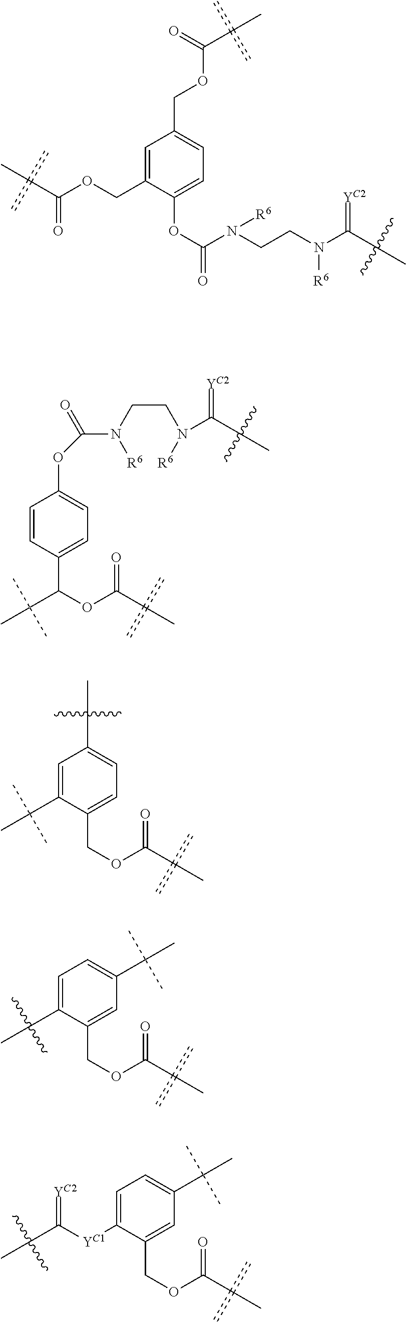

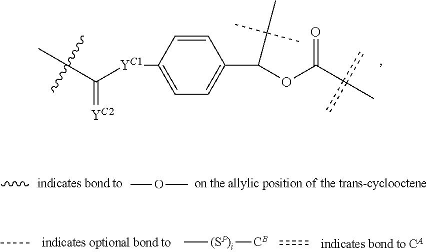

wherein the wiggly line may also indicate a bond to —S— on the allylic position of the trans-cyclooctene, wherein C^{A} is linked to L^{C} via a moiety selected from the group consisting of —O—, —N—, and —S—, preferably from the group consisting of secondary amines and tertiary amines, wherein said moieties are part of C^{A}, wherein when multiple double dashed lines are shown within one L^{C}, each C^{A} moiety is independently selected.
For all linkers according to Group IV, Group V, Group VI, and Group VII, Y^{C1} is selected from the group consisting of —O—, —S—, and —NR⁶—.
For Groups IV-VII preferably R⁶ and R⁷ are as defined in for linkers of Groups I-III; and i and j are as defined for Formula (19).
Preferably, R⁶, R⁷, R⁸, R⁹ used in this Section are as defined in Section 8.
Preferably, R⁶, R⁷, R⁸, R⁹ used in this Section are not substituted.
Preferably, R⁶, R⁷, R⁸, R⁹ used in this Section are hydrogen.

### Section 13—Targeting

### Targeting:

The kits of the invention are very suitable for use in targeted delivery of drugs.

A “primary target” as used in the present invention preferably relates to a target for a targeting agent for therapy. In other embodiments it relates to a target for imaging, theranostics, diagnostics, or in vitro studies. For example, a primary target can be any molecule, which is present in an organism, tissue or cell. Targets include cell surface targets, e.g. receptors, glycoproteins; structural proteins, e.g. amyloid plaques; abundant extracellular targets such as stroma targets, tumor microenvironment targets, extracellular matrix targets such as growth factors, and proteases; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is up regulated or down-regulated in a certain disorder. According to a particular embodiment of the present invention, the primary target is a protein such as a (internalizing or non-internalizing) receptor.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosacharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, fibronectin, VEGF/EGF and VEGF/EGF receptors, TAG72, CEA, CD19, CD20, CD22, CD40, CD45, CD74, CD79, CD105, CD138, CD174, CD227, CD326, CD340, MUC1, MUC16, GPNMB, PSMA, Cripto, Tenascin C, Melanocortin-1 receptor, CD44v6, G250, HLA DR, ED-A, ED-B, TMEFF2, EphB2, EphA2, FAP, Mesothelin, GD2, CAIX, 5T4, matrix metalloproteinase (MMP), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase, MSR1, FAP, CXCR, tumor endothelial marker (TEM), cMET, IGFR, FGFR, GPA33, hCG,

According to a further particular embodiment of the invention, the primary target and targeting agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

In preferred embodiments the Primary Target equals a therapeutic target. It shall be understood that a therapeutic target is the entity that is targeted by the Drug to afford a therapeutic effect.

In preferred embodiments, the Primary Target is blood circulation, and the Targeting Agent is chosen such that a prolonged blood circulation is achieved, for example when the Targeting Agent is polyethyleneglycol.

In preferred embodiments, the Primary Target is an organ. For example, saccharides, in particular hexoses, are known as Targeting Agents for targeting the liver.

### Targeting Agents T^{T}

A Targeting Agent, T^{T}, binds to a Primary Target. In order to allow specific targeting of the above-listed Primary Targets, the Targeting Agent T^{T} can comprise compounds including but not limited to antibodies, antibody derivatives, antibody fragments, antibody (fragment) fusions (e.g. bi-specific and tri-specific mAb fragments or derivatives), proteins, peptides, e.g. octreotide and derivatives, VIP, MSH, LHRH, chemotactic peptides, cell penetrating peptide, membrane translocation moiety, bombesin, elastin, peptide mimetics, organic compounds, inorganic compounds, carbohydrates, monosaccharides, oligosacharides, polysaccharides, oligonucleotides, aptamers, viruses, whole cells, phage, drugs, polymers, liposomes, chemotherapeutic agents, receptor agonists and antagonists, cytokines, hormones, steroids, toxins. Examples of organic compounds envisaged within the context of the present invention are, or are derived from, dyes, compounds targeting CAIX and PSMA, estrogens, e.g. estradiol, androgens, progestins, corticosteroids, methotrexate, folic acid, and cholesterol.

According to a particular embodiment of the present invention, the Primary Target is a receptor and a Targeting Agent is employed, which is capable of specific binding to the Primary Target. Suitable Targeting Agents include but are not limited to, the ligand of such a receptor or a part thereof which still binds to the receptor, e.g. a receptor binding peptide in the case of receptor binding protein ligands. Other examples of Targeting Agents of protein nature include insulin, transferrin, fibrinogen-gamma fragment, thrombospondin, claudin, apolipoprotein E, Affibody molecules such as for example ABY-025, Ankyrin repeat proteins, ankyrin-like repeat proteins, interferons, e.g. alpha, beta, and gamma interferon, interleukins, lymphokines, colony stimulating factors and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin. Alternative examples of targeting agents include DNA, RNA, PNA and LNA which are e.g. complementary to the Primary Target.

Examples of peptides as targeting agents include LHRH receptor targeting peptides, EC-1 peptide, RGD peptides, HER2-targeting peptides, PSMA targeting peptides, somatostatin-targeting peptides, bombesin. Other examples of targeting agents include lipocalins, such as anticalins. One particular embodiment uses Affibodies™ and multimers and derivatives.

In one embodiment antibodies are used as the T^{T}. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as T^{T}, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. For the avoidance of doubt, in the context of this invention the term “antibody” is meant to encompass all of the antibody variations, fragments, derivatives, fusions, analogs and mimetics outlined in this paragraph, unless specified otherwise.

In a preferred embodiment the T^{T} is selected from antibodies and antibody derivatives such as antibody fragments, fragment fusions, proteins, peptides, peptide mimetics, organic molecules, dyes, fluorescent molecules, enzyme substrates.

In a preferred embodiment the T^{T} being an organic molecule has a molecular weight of less than 2000 Da, more preferably less than 1500 Da, more preferably less than 1000 Da, even more preferably less than 500 Da.

In another preferred embodiment the T^{T} is selected from antibody fragments, fragment fusions, and other antibody derivatives that do not contain a Fc domain.

In another embodiment the T^{T} is a polymer and accumulates at the Primary Target by virtue of the EPR effect. Typical polymers used in this embodiment include but are not limited to polyethyleneglycol (PEG), poly(N-(2-hydroxypropyl) methacrylamide) (HPMA), polylactic acid (PLA), polylactic-glycolic acid (PLGA), polyglutamic acid (PG), polyvinylpyrrolidone (PVP), poly(l-hydroxymethylethylene hydroxymethyl-formal (PHF). Other examples are copolymers of a polyacetal/polyketal and a hydrophilic polymer selected from the group consisting of polyacrylates, polyvinyl polymers, polyesters, polyorthoesters, polyamides, oligopeptides, polypeptides and derivatives thereof. Other examples are oligopeptides, polypeptides, glycopolysaccharides, and polysaccharides such as dextran and hyaluronan,

In addition reference is made to [G. Pasut, F. M. Veronese, Prog. Polym. Sci. 2007, 32, 933-961].

According to a further particular embodiment of the invention, the Primary Target and Targeting Agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting Primary Targets with tissue-, cell- or disease-specific expression. For example, the CC49 antibody targets TAG72, the expression of which is limited in normal tissues, but receptors are overexpressed in various solid tumor cell types.

In one embodiment the Targeting Agent specifically binds or complexes with a cell surface molecule, such as a cell surface receptor or antigen, for a given cell population. Following specific binding or complexing of the T^{T} with the receptor, the cell is permissive for uptake of the Prodrug, which then internalizes into the cell. The subsequently administered Activator will then enter the cell and activate the Prodrug, releasing the Drug inside the cell. In another embodiment the Targeting Agent specifically binds or complexes with a cell surface molecule, such as a cell surface receptor or antigen, for a given cell population. Following specific binding or complexing of the T^{T} with the receptor, the cell is not permissive for uptake of the Prodrug. The subsequently administered Activator will then activate the Prodrug on the outside of the cell, after which the released Drug will enter the cell.

As used herein, a T^{T} that “specifically binds or complexes with” or “targets” a cell surface molecule, an extracellular matrix target, or another target, preferentially associates with the target via intermolecular forces. For example, the ligand can preferentially associate with the target with a dissociation constant (K_{d} or K_{D}) of less than about 50 nM, less than about 5 nM, or less than about 500 pM.

In another embodiment the targeting agent T^{T} localizes in the target tissue by means of the EPR effect. An exemplary T^{T} for use in with the EPR effect is a polymer.

In some embodiments the T^{T} can be a cell penetrating moiety, such as cell penetrating peptide. In a preferred embodiment T^{T} can be non-functional that becomes functional upon reaction of the Trigger with the Activator. In a particularly preferred embodiment, said non-functional T^{T} is a portion of a cell penetrating peptide that is bound to another portion of a cell penetrating peptide upon reaction of the Trigger with the Activator. In another preferred embodiment the T^{T}, preferably a cell penetrating peptide, is unmasked upon reaction of the Trigger with the Activator.

In other embodiments, the T^{T} is a polymer, particle, gel, biomolecule or another above listed T^{T} moiety and is locally injected to create a local depot of Prodrug, which can subsequently be activated by the Activator.

In another embodiment the targeting agent T^{T} is a solid material such as but not limited to polymer, metal, ceramic, wherein this solid material is or is comprised in a cartridge, reservoir, depot, wherein preferably said cartridge, reservoir, depot is used for drug release in vivo.

In some embodiments, the targeting agent T^{T} also acts as a Drug, denoted as D^{D}. In a particularly preferred embodiment, the T^{T} acts as a Drug D^{D} by binding the primary target. In other preferred embodiments, the T^{T} acts as a D^{D} after the Trigger has been cleaved.

It is preferred that when a T^{T} is comprised in an embodiment of the invention, it equals C^{B}.

### Section 14—Masking Moieties

In order to avoid the drawbacks of current prodrug activation, it has been proposed to make use of an abiotic, bio-orthogonal chemical reaction to provoke release of the Masking Moiety from the masked Drug. In this type of Prodrug, the Masking Moiety is attached to the Drug via a Trigger, and this Trigger is not activated endogeneously by e.g. an enzyme or a specific pH, but by a controlled administration of the Activator, i.e. a species that reacts with the Trigger moiety in the Prodrug, to induce release of the Masking Moiety or the Drug from the Trigger (or vice versa, release of the Trigger from the Masking Moiety or Drug, however one may view this release process), resulting in activation of the Drug. The previously presented Staudinger approach for this concept, as well as the earlier designs to use the IEDDA for this purpose, has turned out not to work well (vide supra).

In order to better address one or more of the foregoing desires, the present invention provides a kit for the administration and activation of a Prodrug, the kit comprising a Masking Moiety, denoted as M^{M}, linked covalently, directly or indirectly, to a Trigger moiety, which in turn is linked covalently, directly or indirectly, to a Drug, denoted as D^{D}, and an Activator for the Trigger moiety, wherein the Trigger moiety comprises a dienophile satisfying Formula (19) and the Activator comprises a tetrazine satisfying Formula (1).

In another aspect, the invention presents a Prodrug comprising a Masking Moiety, M^{M}, linked, directly or indirectly, to dienophile moiety satisfying above Formula (19).

In yet another aspect, the invention provides a method of modifying a Drug, D^{D}, with one or more Masking Moieties M^{M} affording a Prodrug that can be activated by an abiotic, bio-orthogonal reaction, the method comprising the steps of providing a Masking Moiety and a Drug and chemically linking the Masking Moiety and a Drug to a dienophile moiety satisfying Formula (19).

In a still further aspect, the invention provides a method of treatment wherein a patient suffering from a disease that can be modulated by a drug, is treated by administering, to said patient, a Prodrug comprising a Trigger moiety linked to a Masking Moiety M^{M} and a Drug D^{D}, after activation of which by administration of an Activator, satisfying Formula (1), the Masking Moiety will be released, activating the Drug, wherein the Trigger moiety comprises a dienophile structure satisfying Formula (19).

In a still further aspect, the invention is a compound comprising a dienophile moiety, said moiety comprising a linkage to a Masking Moiety M^{M}, for use in prodrug therapy in an animal or a human being.

In another aspect, the invention is the use of a diene as an Activator for the release, in a physiological environment, of a substance covalently linked to a compound satisfying Formula (19). In connection herewith, the invention also pertains to a diene, for use as an Activator for the release, in a physiological environment, of a substance linked to a compound satisfying Formula (19), and to a method for activating, in a physiological environment, the release of a substance linked to a compound satisfying Formula (19), wherein a tetrazine is used as an Activator.

In another aspect, the invention presents the use of the inverse electron-demand Diels-Alder reaction between a compound satisfying Formula (19) and a dienophile, preferably a trans-cyclooctene, as a chemical tool for the release, in a physiological environment, of a substance administered in a covalently bound form, wherein the substance is bound to a compound satisfying Formula (19).

For the avoidance of doubt, in the context of this invention wherein a M^{M} is removed from an antibody (i.e. Drug) the terms “activatable antibodies” and “Prodrug” mean the same.

For the avoidance of doubt, in the context of this invention wherein a M^{M} is removed from a Drug, the Drug itself can optionally bind to one or more Primary Targets without the use of an additional Targeting Agent T^{T}. In this context, the Primary target is preferably the therapeutic target.

In a preferred embodiment, the Drug comprises a Targeting Agent T^{T} so that the Prodrug can bind a Primary Target. Following activation and M^{M} removal the Drug then binds another Primary Target, which can be a therapeutic target.

In preferred embodiments, the Drug comprises one or more T^{T} moieties, against one or different Primary Targets.

For the avoidance of doubt, in the context of the use of Masking Moieties, Primary target and therapeutic target are used interchangeably.

For the avoidance of doubt, one Drug construct can be modified by more than one Masking Moieties.

In preferred embodiments the activatable antibodies or Prodrugs of this invention are used in the treatment of cancer. In preferred embodiments the activatable antibodies or Prodrugs of this invention are used in the treatment of an autoimmune disease or inflammatory disease such as rheumatoid arthritis. In preferred embodiments the activatable antibodies or Prodrugs of this invention are used in the treatment of a fibrotic disease such as idiopathic pulmonary fibrosis.

Exemplary classes of Primary Targets for activatable antibodies or Prodrugs of this invention include but are not limited to cell surface receptors and secreted proteins (e.g. growth factors), soluble enzymes, structural proteins (e.g. collagen, fibronectin) and the like. In preferred embodiments the Primary Target is an extracellular target. In preferred embodiments, the Primary Target is an intracellular target.

In another embodiment, the drug is a bi- or trispecific antibody derivative that serves to bind to tumor cells and recruit and activate immune effector cells (e.g. T-cells, NK cells), the immune effector cell binding function of which is masked and inactivated by being linked to a dienophile moiety as described above. The latter, again, serving to enable bio-orthogonal chemically activated drug activation.

When D^{D} is C^{B} it is preferred that D^{D} is not attached to remainder of the Prodrug through its antigen-binding domain. Preferably D^{D} is C^{A}.

Masking moieties M^{M} can for example be an antibody, protein, peptide, polymer, polyethylene glycol, polypropylene glycol carbohydrate, aptamers, oligopeptide, oligonucleotide, oligosaccharide, carbohydrate, as well as peptides, peptoids, steroids, organic molecule, or a combination thereof that further shield the bound drug D^{D} or Prodrug. This shielding can be based on e.g. steric hindrance, but it can also be based on a non covalent interaction with the drug D^{D}. Such Masking Moiety may also be used to affect the in vivo properties (e.g. blood clearance; biodistribution, recognition by the immune system) of the drug D^{D} or Prodrug.

In preferred embodiments the Masking Moiety is an albumin binding moiety.
In preferred embodiments, the Masking Moiety equals a Targeting Agent.
In preferred embodiments, the Masking Moiety is bound to a Targeting Agent.
In a preferred embodiment the Drug D^{D}, being C^{A}, is modified with multiple M^{M}, being C^{B}, wherein at least one of the bound M^{M} is T^{T}.
In a preferred embodiment, when C^{A} is D^{D} then D^{D} is not bound to T^{R} via a Spacer S^{P}.
In preferred embodiments the T^{R} can itself act as a Masking Moiety, provided that C^{A} is D^{D}. For the sake of clarity, in these embodiments the size of the T^{R} without the attachment of a M^{M} is sufficient to shield the Drug D^{D} from its Primary Target, which, in this context, is preferably the therapeutic target. The M^{M} of the modified D^{D} can reduce the D^{D}'s ability to bind its target allosterically or sterically.
In specific embodiments, the M^{M} is a peptide and does not comprise more than 50% amino acid sequence similarity to a natural protein-based binding partner of an antibody-based D^{D}.

In preferred embodiments M^{M} is a peptide between 2 and 40 amino acids in length.

In one embodiment the M^{M} reduces the ability of the D^{D} to bind its target such that the dissociation constant of the D^{D} when coupled to the M^{M} towards the target is at least 100 times greater than the dissociation constant towards the target of the D^{D} when not coupled to the M^{M}. In another embodiment, the coupling of the M^{M} to the D^{D} reduces the ability of the D^{D} to bind its target by at least 90%.

In preferred embodiments the M^{M} in the masked D^{D} reduces the ability of the D^{D} to bind the target by at least 50%, by at least 60%, by at least 70%, by at least 75%, by at least 80%, by at least 85%, by at least 90%, by at least 95%, by at least 96%, by at least 97%, by at least 98%, by at least 99%, or by 100%, as compared to the ability of the unmasked D^{D} to bind the target. The reduction in the ability of a D^{D} to bind the target can be determined, for example, by using an in vitro displacement assay, such as for example described for antibody D^{D} in WO2009/025846 and WO2010/081173.

In preferred embodiments the D^{D} comprised in the masked D^{D} is an antibody, which expressly includes full-length antibodies, antigen-binding fragments thereof, antibody derivatives antibody analogs, antibody mimics and fusions of antibodies or antibody derivatives.

In certain embodiments the M^{M} is not a natural binding partner of the antibody. In preferred embodiments, the M^{M} contains no or substantially no homology to any natural binding partner of the antibody. In preferred embodiments the M^{M} is no more than 5%, 10%, 15%, 20%, 25%, 30%, 35%, 40%, 45%, 50%, 55%, 60%, 65%, 70%, 75%, or 80% similar to any natural binding partner of the antibody. In preferred embodiments the M^{M} is no more than 5%, 10%, 15%, 20%, 25%, 30%, 35%, 40%, 45%, 50%, 55%, 60%, 65%, 70%, 75%, or 80% identical to any natural binding partner of the antibody. In preferred embodiments, the M^{M} is no more than 50% identical to any natural binding partner of the antibody. In preferred embodiments, the M^{M} is no more than 25% identical to any natural binding partner of the antibody. In preferred embodiments, the M^{M} is no more than 20% identical to any natural binding partner of the antibody. In preferred embodiments, the M^{M} is no more than 10% identical to any natural binding partner of the antibody.

In the Prodrug, the M^{M} and the Trigger T^{R}—the dienophile derivative—can be directly linked to each other. They can also be bound to each other via a spacer S^{P} or a self-immolative linker L^{C}. It will be understood that the invention encompasses any conceivable manner in which the diene Trigger is attached to the M^{M}. It will be understood that M^{M} is linked to the dienophile in such a way that the M^{M} is eventually capable of being released from the D^{D} after formation of the IEDDA adduct. Generally, this means that the bond between the D^{D} and the dienophile, or in the event of a self-immolative linker L^{C} the bond between the L^{C} and the dienophile and between the D^{D} and the L^{C} should be cleavable. Alternatively, this means that the bond between the M^{M} and the dienophile, or in the event of a self-immolative linker L^{C} the bond between the L^{C} and the dienophile and between the M^{M} and the L^{C} should be cleavable.

In preferred embodiments, the antibody comprised in the masked antibody is a multi-antigen targeting antibody, comprising at least a first antibody or antigen-binding fragment or mimic thereof that binds a first Primary Target and a second antibody or antigen-binding fragment or mimic thereof that binds a second Primary Target. In preferred embodiments, the antibody comprised in the masked antibody is a multi-antigen targeting antibody, comprising a first antibody or antigen-binding fragment or mimic thereof that binds a first Primary Target, a second antibody or antigen-binding fragment or mimic thereof that binds a second Primary Target, and a third antibody or antigen-binding fragment or mimic thereof that binds a third Primary Target. In preferred embodiments, the multi-antigen targeting antibodies bind two or more different Primary Targets. In preferred embodiments, the multi-antigen targeting antibodies bind two or more different epitopes on the same Primary Target. In preferred embodiments the multi-antigen targeting antibodies bind a combination of two or more different targets and two or more different epitopes on the same Primary Target. In preferred embodiments the masked multi-antigen targeting antibodies comprise one M^{M} group, or two or more M^{M} groups. It shall be understood that preferably at least one of the Primary Targets is a therapeutic target.

In preferred embodiments of a multispecific activatable antibody, a scFv can be fused to the carboxyl terminus of the heavy chain of an IgG activatable antibody, to the carboxyl terminus of the light chain of an IgG activatable antibody, or to the carboxyl termini of both light and the heavy chain of an IgG activatable antibody. In preferred embodiments of a multispecific activatable antibody, a scFv can be fused to the amino terminus of the heavy chain of an IgG activatable antibody, to the amino terminus of the light chain of an IgG activatable antibody, or to the amino termini of both light and the heavy chain of an IgG activatable antibody. In preferred embodiments of a multispecific activatable antibody, a scFv can be fused to any combination of one or more carboxyl termini and one or more amino termini of an IgG activatable antibody. Methods of preparing multispecific antibodies are known to the person skilled in the art. In addition reference is made to [Weilde et al., Cancer Genomics & Proteomics 2013, 10, 1-18], [Weidle et al., Seminars in Oncology 2014, 41, 5, 653-660, [Jachimowicz et al., BioDrugs (2014) 28:331-343], the contents of which are hereby incorporated by reference.

In preferred embodiments, a M^{M} linked to a T^{R} is attached to and masks an antigen binding domain of the IgG. In preferred embodiments, a M^{M} linked to a T^{R} is attached to and masks an antigen binding domain of at least one scFv. In preferred embodiments, a M^{M} linked to a T^{R} is attached to and masks an antigen binding domain of the IgG and a M^{M} linked to a T^{R} is attached to and masks an antigen binding domain of at least one scFv.

In preferred embodiments, the M^{M} has a dissociation constant, i.e., dissociation constant at an equilibrium state, K_{d}, for binding to the antibody that is greater than the K_{d} for binding of the antibody to its Primary Target. In preferred embodiments, the M^{M} has a K_{d} for binding to the antibody that is approximately equal to the K_{d} for binding of the antibody to its Primary Target. In preferred embodiments, the M^{M} has a K_{d} for binding to the antibody that is less than the K_{d} for binding of the antibody to its Primary Target. In preferred embodiments, the M^{M} has a K_{d} for binding to the antibody that is no more than 2, 3, 4, 5, 10, 25, 50, 100, 250, 500, or 1,000 fold greater than the K_{d} for binding of the antibody to its Primary Target. In preferred embodiments, the M^{M} has a K_{d} for binding to the antibody that is between 1-5, 2-5, 2-10, 5-10, 5-20, 5-50, 5-100, 10-100, 10-1,000, 20-100, 20-1,000, or 100-1,000 fold greater than the K_{d} for binding of the antibody to its Primary Target.

In preferred embodiments, the M^{M} has an affinity for binding to the antibody that is greater than the affinity of binding of the antibody to its Primary Target. In preferred embodiments, the M^{M} has an affinity for binding to the antibody that is approximately equal to the affinity of binding of the antibody to its Primary Target. In preferred embodiments, the M^{M} has an affinity for binding to the antibody that is less than the affinity of binding of the antibody to its Primary Target. In preferred embodiments, the M^{M} has an affinity for binding to the antibody that is 2, 3, 4, 5, 10, 25, 50, 100, 250, 500, or 1,000 fold less than the affinity of binding of the antibody to its Primary Target. In preferred embodiments, the M^{M} has an affinity of binding to the antibody that is between 1-5, 2-5, 2-10, 5-10, 5-20, 5-50, 5-100, 10-100, 10-1,000, 20-100, 20-1,000, or 100-1,000 fold less than the affinity of binding of the antibody to its Primary Target. In preferred embodiments, the M^{M} has an affinity of binding to the antibody that is 2 to 20 fold less than the affinity of binding of the antibody to its Primary Target.

In preferred embodiments, a M^{M} not covalently linked to the antibody and at equimolar concentration to the antibody does not inhibit the binding of the antibody to its Primary Target. In preferred embodiments, the M^{M} does not interfere of compete with the antibody for binding to the Primary Target when the Prodrug is in a cleaved state.

In preferred embodiments, the antibody has a dissociation constant of about 100 nM or less for binding to its Primary Target.

In preferred embodiments, the antibody has a dissociation constant of about 10 nM or less for binding to its Primary Target.
In preferred embodiments, the antibody has a dissociation constant of about 1 nM or less for binding to its Primary Target.

In preferred embodiments, the coupling of the M^{M} reduces the ability of the antibody to bind its Primary Target such that the dissociation constant (K_{d}) of the antibody when coupled to the M^{M} towards its Primary Target is at least 20 times greater than the K_{d} of the antibody when not coupled to the M^{M} towards its Primary Target.

In preferred embodiments, the coupling of the M^{M} reduces the ability of the antibody to bind its Primary Target such that the K_{d} of the antibody when coupled to the M^{M} towards its Primary Target is at least 40 times greater than the K_{d} of the antibody when not coupled to the M^{M} towards its Primary Target. In preferred embodiments, the coupling of the M^{M} reduces the ability of the antibody to bind its Primary Target such that the K_{d} of the antibody when coupled to the M^{M} towards its Primary Target is at least 100 times greater than the K_{d} of the antibody when not coupled to the M^{M} towards its Primary Target. In preferred embodiments, the coupling of the M^{M} reduces the ability of the antibody to bind its Primary Target such that the K_{d} of the antibody when coupled to the M^{M} towards its Primary Target is at least 1,000 times greater than the K_{d} of the antibody when not coupled to the M^{M} towards its Primary Target. In preferred embodiments, the coupling of the M^{M} reduces the ability of the antibody to bind its Primary Target such that the K_{d} of the antibody when coupled to the M^{M} towards its Primary Target is at least 10,000 times greater than the K_{d} of the antibody when not coupled to the M^{M} towards its Primary

Target.

In preferred embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 100,000 times greater than the K_{d} of the antibody when not coupled to the M^{M} towards its Primary Target.

In preferred embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 1,000,000 times greater than the K_{d} of the antibody when not coupled to the M^{M} towards its Primary Target.
In preferred embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 10,000,000 times greater than the K_{d} of the antibody when not coupled to the M^{M} towards its Primary Target.

Exemplary drugs that can be used in a Prodrug relevant to this invention using Masking Moieties include but are not limited to: antibodies, antibody derivatives, antibody fragments, proteins, aptamers, oligopeptides, oligonucleotides, oligosaccharides, carbohydrates, as well as peptides, peptoids, steroids, toxins, hormones, viruses, whole cells, phage. In preferred embodiments the drugs are low to medium molecular weight compounds, preferably organic compounds (e.g. about 200 to about 2500 Da, preferably about 300 to about 1750 Da, more preferably about 300 to about 1000 Da).

In one embodiment antibodies are used as the Drug. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulins is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, camelid antibodies, minibodies, nanobodies, resurfaced antibodies, humanized antibodies, fully human antibodies, single domain antibody (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which is hereby incorporated by reference. Other embodiments use antibody mimetics as Drug, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. “Antibody fragment” refers to at least a portion of the variable region of the immunoglobulin that binds to its target, i.e. the antigen-binding region. Multimers may be linearly linked or may be branched and may be derived from a single vector or chemically connected, or non-covalently connected. Methods of making above listed constructs are known in the art. For the avoidance of doubt, in the context of this invention the term “antibody” is meant to encompass all of the antibody variations, fragments, derivatives, fusions, analogs and mimetics outlined in this paragraph, unless specified otherwise.

Typical drugs for which the invention is suitable include, but are not limited to: proteins, peptides, oligosacharides, oligonucleotides, monospecific, bispecific and trispecific antibodies and antibody fragment or protein fusions, preferably bispecific and trispecific. In preferred embodiments the activatable antibody or derivative is formulated as part of a pro-Bispecific T Cell Engager (BITE) molecule.

Other embodiments use immunotoxins, which are a fusion or a conjugate between a toxin and an antibody. Typical toxins comprised in an immunotoxins are cholera toxin, ricin A, gelonin, saporin, bouganin, ricin, abrin, diphtheria toxin, Staphylococcal enterotoxin, Bacillus Cyt2Aa1 toxin, Pseudomonas exotoxin PE38, Pseudomonas exotoxin PE38KDEL, granule-associated serine protease granzyme B, human ribonucleases (RNase), or other pro-apoptotic human proteins. Other exemplary cytotoxic human proteins which may be incorporated into fusion constructs are caspase 3, caspase 6, and BH3-interacting domain death agonist (BID). Current immunotoxins have immunogenicity issues and toxicity issues, especially towards vascular endothelial cells. Masking the targeted toxin by a M^{M} such as a PEG or peptide and removing the M^{M} once the masked immunotoxin has bound to its target is expected to greatly reduce the toxicity and immunogenicity problems.

Other embodiments use immunocytokines, which are a fusion or a conjugate between a cytokine and an antibody. Typical cytokines used in cancer therapy include IL-2, IL-7, IL-12, IL-15, IL-21, TNF. A typical cytokine used in autoimmune diseases is the anti-inflammatory IL-10. Masking the targeted cytokine by a M^{M} such as a PEG or peptide and removing the M^{M} once the masked immunocytokine has bound to its target is expected to greatly reduce the toxicity problems. Other embodiments use small to medium sized organic drugs.

In preferred embodiments the unmasked Drug is multispecific and binds to two or more same or different Primary Targets. In preferred embodiments the multispecific Drug comprises one or more (masked) antibodies (also referred to as binding moieties) that are designed to engage immune effector cells. In preferred embodiments the masked multispecific Prodrug comprises one or more (masked) antibodies that are designed to engage leukocytes. In preferred embodiments the masked multispecific Prodrug comprises one or more (masked) antibodies that are designed to engage T cells. In preferred embodiments the masked multispecific Prodrug comprises one or more (masked) antibodies that engage a surface antigen on a leukocyte such as on a T cell, natural killer (NK) cell, a myeloid mononuclear cell, a macrophage and/or another immune effector cell. In preferred embodiments the immune effector cell is a leukocyte, a T cell, a NK cell, or a mononuclear cell.

In an exemplary multispecific masked Prodrug the Prodrug comprises an antibody (i.e. Targeting Agent) for a cancer receptor, e.g. TAG72, a antibody for CD3 on T cells, and an antibody for CD28 on T cells, wherein either the antibody for CD3 or for CD28 or both is masked by a M^{M}. Another example is an activatable antibody that comprises an antibody for a cancer receptor, and an antibody for CD3 on T cells, wherein the antibody for CD3 is masked by a M^{M}. Another example is a Prodrug that has an antibody for a cancer receptor, and an antibody for CD28 on T cells, wherein the antibody for CD28 is masked by a M^{M}. Another example is a Prodrug that has an antibody for a cancer receptor, and an antibody for CD16a on NK cells, wherein the antibody for CD16a is masked by a M^{M}. In yet another embodiment the unmasked Drug binds two different immune cells and optionally in addition a tumor cell. Said multispecific antibody derivatives can for example be prepared by fusing or conjugating antibodies, antibody fragments such as Fab, Fabs, scFv, camel antibody heavy chain fragments and proteins.

In some preferred embodiments the M^{M} reduces the binding of the Drug to Primary Targets, equaling therapeutic targets, selected from CD3, CD28, PD-L1, PD-1, LAG-3, TIGIT, TIM-3, B7H4, Vista, CTLA-4 polysialic acids and corresponding lectins. In other preferred embodiments the M^{M} masks a T-cell agonist, an NK cell agonist, an DC cell agonist.

In preferred embodiments of an immune effector cell engaging masked multispecific Prodrug such as a T-cell engaging multispecific activatable antibody, at least one antibody comprised in the Prodrug is a Targeting Agent and binds a Primary Target that is typically an antigen present on the surface of a tumor cell or other cell type associated with disease, such as, but not limited to, EGFR, erbB2, EpCAM, PD-L1, B7H₃ or CD71 (transferrin receptor), and at least one other antibody comprised in the Prodrug binds Primary Target that is typically a stimulatory or inhibitory antigen present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3 or VISTA. In preferred embodiments it is preferred that the targeted CD3 antigen is CD3e or CD3 epsilon.

One embodiment of the disclosure is a multispecific activatable antibody that includes an antibody Targeting Agent directed to a tumor target and another agonist antibody, the Drug, directed to a co-stimulatory receptor expressed on the surface of an activated T cell or NK cell, wherein the agonist antibody is masked. Examples of co-stimulatory receptors include but are not limited to CD27, CD137, GITR, HVEM, NKG2D, OX40. In this embodiment, once the Prodrug is tumor-bound and activated it would effectively crosslink and activate the T cell or NK cell expressed co-stimulatory receptors in a tumor dependent manner to enhance the activity of T cell or NK cells that are responding to any tumor antigen via their endogenous T cell or NK cell activating receptors. The activation dependent nature of these T cell or NK cell co-stimulatory receptors would focus the activity of the activated multispecific Prodrug to tumor specific T cells without activating all T cells independent of their antigen specificity.

One embodiment of the disclosure is a multispecific activatable antibody targeted to a disease characterized by T cell overstimulation, such as, but not limited to, an autoimmune disease or inflammatory disease microenvironment. Such a Prodrug includes an antibody, for example a IgG or scFv, directed to a target comprising a surface antigen expressed in a tissue targeted by a T cell in autoimmune or inflammatory disease and an antibody, for example IgG or scFv, directed to an inhibitory receptor expressed on the surface of a T cell or NK cell, wherein the T cell or NK cell inhibitory antibody is masked. Examples of inhibitory receptors include but are not limited to BTLA, CTLA-4, LAG3, PD-1, TIGIT, TIM3, and NK-expressed KIRs. Examples of a tissue antigen targeted by T cells in autoimmune disease include but are not limited to a surface antigen expressed on myelin or nerve cells in multiple sclerosis or a surface antigen expressed on pancreatic islet cells in Type 1 diabetes. In this embodiment, the Prodrug localizes at the tissue under autoimmune attack or inflammation, is activated by the Activator and co-engages the T-cell or NK cell inhibitory receptor to suppress the activity of autoreactive T cells responding to any disease tissue targeted antigens via their endogenous TCR or activating receptors.

Other non-limiting exemplary Primary Targets for the binding moieties comprised in Drugs of this invention are listed in the patent WO2015/013671, the contents of which are hereby incorporated by reference.

In another embodiment, the Drug is a masked vaccine, which can be unmasked at a desired time and/or selected location in the body, for example subcutaneously and/or in the proximity of lymph nodes. In another embodiment, the Drug is a masked antigen, e.g. a masked peptide, which optionally is present in a Major Histocompatibility Complex (MHC) and which can be unmasked at a desired time and/or selected location in the body, for example subcutaneously and/or in the proximity of lymph nodes.

The Prodrug may further comprise another linked drug, which is released upon target binding, either by proteases, pH, thiols, or by catabolism. Examples are provided in the review on Antibody-drug conjugates in [Polakis, Pharmacol. Rev. 2016, 68, 3-19]. The invention further contemplates that the Prodrug can induce antibody-dependent cellular toxicity (ADCC) or complement dependent cytotoxicity (CDC) upon unmasking of one or more moieties of the Prodrug. The invention also contemplates that the Prodrug can induce antibody-dependent cellular toxicity (ADCC) or complement dependent cytotoxicity (CDC) independent of unmasking of one or more moieties of the Prodrug.

Some embodiments use as said additional drug antiproliferative/antitumor agents, antibiotics, cytokines, anti-inflammatory agents, anti-viral agents, antihypertensive agents, chemosensitizing, radiosensitizing agents, DNA damaging agents, anti-metabolites, natural products and their analogs.

It is preferred that the Drug is a protein or an antibody.

### Section 15—Administration of a Prodrug

When administering the Prodrug (as further defined in the sections below) and the Activator to a living system, such as an animal or human, in preferred embodiments the Prodrug is administered first, and it will take a certain time period before the Prodrug has reached the Primary Target. This time period may differ from one application to the other and may be minutes, days or weeks. After the time period of choice has elapsed, the Activator is administered, will find and react with the Prodrug and will thus activate the Prodrug and/or afford Drug release at the Primary Target. In some preferred embodiments, the time interval between the administration of the Prodrug and the Activator is between 10 minutes and 4 weeks. In some preferred embodiments, the time interval between the administration of the Prodrug and the Activator is between 1 hour and 2 weeks, preferably between 1 and 168 hours, more preferably between 1 and 120 hours, even more preferably between 1 and 96 hours, most preferably between 3 and 72 hours.

The compounds and combinations of the invention can be administered via different routes including but not limited to intravenous or subcutaneous injection, intraperitoneal, local injection, oral administration, rectal administration and inhalation. Formulations suitable for these different types of administrations are known to the skilled person. Prodrugs or Activators according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include for example saline, buffered saline, dextrose, water, glycerol, ethanol, and combinations thereof. The formulation should suit the mode of administration.

It will be understood that the chemical entities administered, viz. the Prodrug and the Activator, can be in a modified form that does not alter the chemical functionality of said chemical entity, such as salts, hydrates, or solvates thereof.

After administration of the Prodrug, and before the administration of the Activator, it is preferred to remove excess Prodrug by means of a Clearing Agent in cases when Prodrug activation in circulation is undesired and when natural Prodrug clearance is insufficient. A Clearing Agent is an agent, compound, or moiety that is administered to a subject for the purpose of binding to, or complexing with, an administered agent (in this case the Prodrug) of which excess is to be removed from circulation. The Clearing Agent is capable of being directed to removal from circulation. The latter is generally achieved through liver receptor-based mechanisms, although other ways of secretion from circulation exist, as are known to the skilled person. In the invention, the Clearing Agent for removing circulating Prodrug, preferably comprises a dienophile moiety, e.g. as discussed above, capable of reacting to the tetrazine moiety of the Prodrug.

In other preferred embodiments the Activator is administered first, followed by the Prodrug, wherein the time interval between the administration of the two components ranges from 1 minute to 12 weeks, preferably 1 minute to 2 weeks, preferably from 10 minutes to 3 days.

In preferred embodiments, the Prodrug and Activator are administered at the same time. either as two separate administrations or as a co-administration.

In yet another embodiment, the Prodrug and Activator are reacted with one another prior to administration and the resulting reaction mixture is then administered, wherein the time interval between start of the reaction and the administration varies from 1 minute to 3 days, preferably 1 minute to 1 day, more preferably from 1 minute to 3 hours.

### Section 16—Therapeutic Use

In preferred embodiments, the compounds, combinations, and kits are for use as a medicament. Alternatively, the compounds, combinations, and kits are used in a method for treating patients, said method comprising administering the compounds comprised in the compounds, combinations, and kits to a subject.

### Section 17—the Inverse Electron-Demand Diels-Alder Reaction (IEDDA)

The established IEDDA conjugation chemistry generally involves a pair of reactants that comprise, as one reactant (i.e. one Bio-orthogonal Reactive Group), a suitable diene, such as a derivative of tetrazine, e.g. an electron-deficient tetrazine and, as the other reactant (i.e. the other Bio-orthogonal Reactive Group), a suitable dienophile, such as a trans-cyclooctene (TCO). The exceptionally fast reaction of (substituted) tetrazines, in particular electron-deficient tetrazines, with a TCO moiety results in an intermediate that rearranges to a dihydropyridazine Diels-Alder adduct by eliminating N₂ as the sole by-product. The initially formed 4,5-dihydropyridazine product may tautomerize to a 1,4- or a 2,5-dihydropyridazine product, especially in aqueous environments. Below a reaction scheme is given for a [4+2] IEDDA reaction between (3,6)-di-(2-pyridyl)-s-tetrazine diene and a trans-cyclooctene dienophile, followed by a retro Diels Alder reaction in which the product and dinitrogen is formed. Because the trans-cyclooctene derivative does not contain electron withdrawing groups as in the classical Diels Alder reaction, this type of Diels Alder reaction is distinguished from the classical one, and frequently referred to as an “inverse-electron-demand Diels Alder (IEDDA) reaction”. In the following text the sequence of both reaction steps, i.e. the initial Diels-Alder cyclo-addition (typically an inverse electron-demand Diels Alder cyclo-addition) and the subsequent retro Diels Alder reaction will be referred to in shorthand as the “inverse electron-demand Diels Alder reaction” or “inverse electron-demand Diels Alder conjugation” or “IEDDA”. The product of the reaction is then the IEDDA adduct or conjugate. This is illustrated in Scheme 1 below.

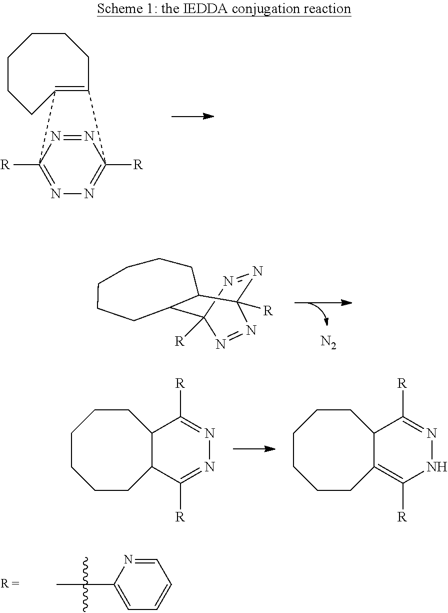

The two reactive species are abiotic and do not undergo fast metabolism or side reactions in vitro or in vivo. They are bio-orthogonal, e.g. they selectively react with each other in physiologic media. Thus, the compounds and the method of the invention can be used in a living organism. Moreover, the reactive groups are relatively small and can be introduced in biological samples or living organisms without significantly altering the size of biomolecules therein. References on the inverse electron demand Diels Alder reaction, and the behavior of the pair of reactive species include: [Thalhammer et al., Tetrahedron Lett., 1990, 31, 47, 6851-6854], [Wijnen et al., J. Org. Chem., 1996, 61, 2001-2005], [Blackman et al., J. Am. Chem. Soc., 2008, 130, 41, 13518-19], Rossin et al., Angew. Chem. Int. Ed. 2010, 49, 3375], [Devaraj et al., Angew. Chem. Int. Ed. 2009, 48, 7013], [Devaraj et al., Angew. Chem. Int. Ed., 2009, 48, 1-5].

### The IEDDA Pyridazine Elimination Reaction

Below, the dienophile, a TCO, that is comprised in combinations and kits of the invention may be referred to as a “Trigger”. The dienophile is connected at the allylic position to a Construct-A. Moreover, tetrazines that are used in the IEDDA pyridazine elimination reaction may be referred to as “Activators”. The term Construct-A in this invention is used to indicate any substance, carrier, biological or chemical group, of which it is desired to have it first in a bound (or masked) state, and being able to provoke release from that state.

The inventors previously demonstrated that the dihydropyridazine product derived from a tetrazine (the Activator) and a TCO containing a carbamate-linked drug (doxorubicin, the Construct-A) at the allylic position is prone to eliminate CO₂ and the amine-containing drug, eventually affording aromatic pyridazine.

Without wishing to be bound by theory, the inventors believe that the Activator provokes Construct-A release via a cascade mechanism within the IEDDA adduct, i.e. the dihydropyridazine. The cascade mechanism can be a simple one step reaction, or it can be comprised in multiple steps that involves one or more intermediate structures. These intermediates may be stable for some time or may immediately degrade to the thermodynamic end-product or to the next intermediate structure. In any case, whether it be a simple or a multistep process, the result of the cascade mechanism is that the Construct-A gets released from the IEDDA adduct. Without wishing to be bound by theory, the design of the diene is such that the distribution of electrons within the IEDDA adduct is unfavorable, so that a rearrangement of these electrons must occur. This situation initiates the cascade mechanism, and it therefore induces the release of the Construct-A. Specifically, and without wishing to be bound by theory, the inventors believe that the NH moiety comprised in the various dihydropyridazine tautomers, such as the 1,4-dihydropyridazine tautomer, of the IEDDA adduct can initiate an electron cascade reaction, a concerted or consecutive shift of electrons over several bonds, leading to release of the Construct-A. Occurrence of the cascade reaction in and/or Construct-A release from the Trigger is not efficient or cannot take place prior to the IEDDA reaction, as the Trigger-Construct-A conjugate itself is relatively stable as such. The cascade can only take place after the Activator and the Trigger-Construct conjugate have reacted and have been assembled in the IEDDA adduct.

With reference to Scheme 2 below, and without wishing to be bound by theory, the inventors believe that the pyridazine elimination occurs from the 1,4-dihydropyridazine tautomer 4. Upon formation of the 4,5-dihydropyridazine 3, tautomerization affords intermediates 4 and 7, of which the 2,5-dihydropyridazine 7 cannot eliminate the C^{A}. Instead it can slowly convert into aromatic 8, which also cannot eliminate C^{A} or it can tautomerize back to intermediate 3. Upon formation of 4 the C^{A} is eliminated near instantaneously, affording free C^{A} 8 as an amine, and pyridazine elimination products 5 and 6. This elimination reaction has been shown to work equally well in the cleavage of carbonates, esters and ethers from the TCO trigger. The Trigger in Scheme 2 is also optionally bound to a Construct-B (C^{B}), which in this case cannot release from the Trigger. Thereby Construct A can be separated from Construct B by means of the IEDDA pyridazine elimination.

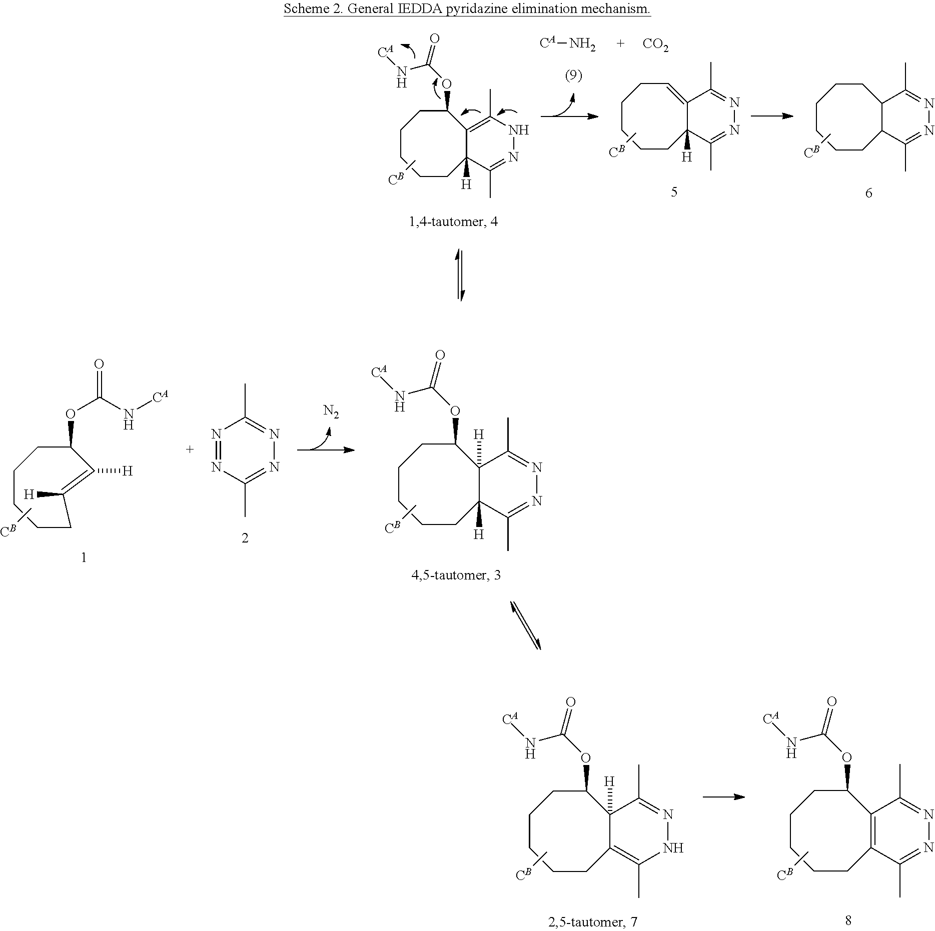

In preferred embodiments, the dienophile trigger moiety used in the present invention comprises a trans-cyclooctene ring. Herein, this eight-membered ring moiety will be defined as a trans-cyclooctene moiety, for the sake of legibility, or abbreviated as “TCO” moiety. It will be understood that the essence resides in the possibility of the eight-membered ring to act as a dienophile and to be released from its conjugated Construct-A upon reaction.

The tetrazines of the invention and dienophiles are capable of reacting in an inverse electron-demand Diels-Alder reaction (IEDDA). IEDDA reaction of the Trigger with the Activator leads to release of the Construct-A through an electron-cascade-based elimination, termed the “pyridazine elimination”. When an Activator reacts with a Trigger capable of eliminating Construct-A, the combined process of reaction and Construct-A elimination is termed the “IEDDA pyridazine elimination”.

This invention provides an Activator that reacts with a Construct-A-conjugated Trigger, resulting in the cleavage of the Trigger from the Construct-A. In one prominent embodiment this results in the cleavage of Construct-A from Construct-B. In another embodiment the Trigger cleavage results in cleavage of one Construct A from another Construct A, as the dienophile Trigger of Formula 19 can comprise two allylic positioned Constructs-A, wherein one or both can release from the Trigger upon reaction with a diene. In another embodiment, Trigger cleavage results in the cleavage of one or more Construct-A from one or more Construct-B. Construct-B is the Construct that is bound to the dienophile, and cannot be released from the dienophile, unless it is bound to the allylic position via a spacer or self-imolative linker that also binds Construct-A. In preferred embodiments, the Trigger is used as a reversible covalent bond between two molecular species.

Scheme 4a below is a general scheme of Construct release according to this invention, wherein the Construct being released is termed Construct-A (C^{A}), and wherein another Construct, Construct-B (C^{B}) can optionally be bound to the dienophile, but not via the allylic position, wherein Construct-B cannot be released from the dienophile.

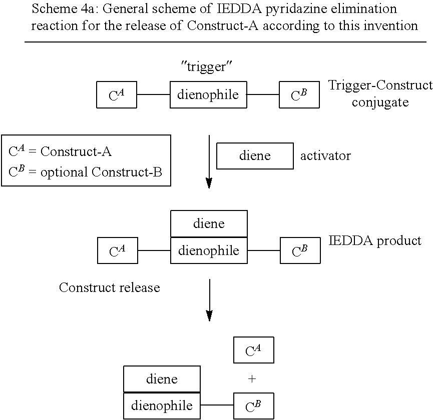

Scheme 4b below is a general scheme of Construct release according to another embodiment of this invention, wherein Construct-B (C^{B}) is bound to the dienophile via a spacer or self-imolative linker that also binds Construct-A and, wherein when the spacer or self-immolative linker is released from the allylic position then Construct-B and Construct A are released from the Trigger and from each other.

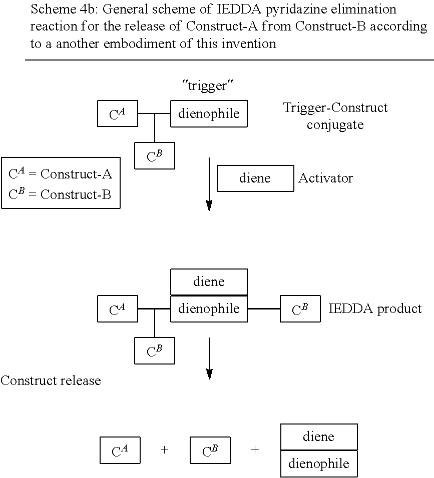

Scheme 4c below is a general scheme of Construct release according to another embodiment of this invention, wherein the Trigger is linked to two allylic positioned Construct-A's, and wherein one or both Constructs-A's can be released from the Trigger, in any case resulting in cleavage of one Construct-A from the other Construct-A.

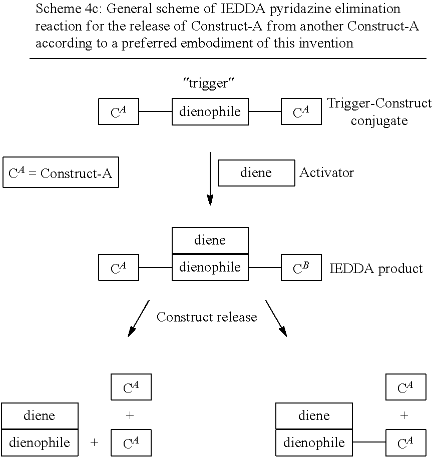

The Construct release occurs through a powerful, abiotic, bio-orthogonal reaction of the dienenophile (Trigger) with the diene (Activator), viz. the aforementioned IEDDA. The masked or bound Construct is a Construct-dienenophile conjugate. Possibly the Construct-A is linked to one or more additional Constructs A linked via a self-immolative linker. It will be understood that in Scheme 3 in the IEDDA adduct as well as in the end product after release, the indicated dienophile group and the indicated diene group are the residues of, respectively, the dienophile and diene groups after these groups have been converted in the IEDDA reaction.

The invention provides, in one aspect, the use of a tetrazine as an Activator for the release, in a chemical, biological, or physiological environment, of a Construct linked to a TCO. In connection herewith, the invention also pertains to a tetrazine as an Activator for the release, in a chemical, biological, or physiological environment, of a substance linked to a TCO. The fact that the reaction is bio-orthogonal, and that many structural options exist for the reaction pairs, will be clear to the skilled person. E.g., the IEDDA reaction is known in the art of bioconjugation, diagnostics, pre-targeted medicine. Reference is made to, e.g., WO 2010/119382, WO 2010/119389, and WO 2010/051530. Whilst the invention presents an entirely different use of the reaction, it will be understood that the various structural possibilities available for the IEDDA reaction pairs as used in e.g. pre-targeting, are also available in the field of the present invention.

Other than is the case with e.g. medicinally active substances, where the in vitro or in vivo action is often changed with minor structural changes, the present invention first and foremost requires the right chemical reactivity combined with sufficient stability for the intended application. Thus, the possible structures extend to those of which the skilled person is familiar with that these are reactive as dienophiles.

### The TCO Trigger:

In a preferred embodiment, the dienophile Trigger moiety used in the present invention comprises a trans-cyclooctene ring, and particularly refers to a structure satisfying Formula (19), the ring optionally including one or more hetero-atoms. The skilled person is familiar with the fact that the dienophile activity is not necessarily dependent on the presence of all carbon atoms in the ring, since also heterocyclic monoalkenylene eight-membered rings are known to possess dienophile activity.

Thus, in general, the invention is not limited to strictly trans-cyclooctene. The person skilled in organic chemistry will be aware that other eight-membered ring-based dienophiles exist, which comprise the same endocyclic double bond as the trans-cyclooctene, but which may have one or more heteroatoms elsewhere in the ring. I.e., the invention generally pertains to eight-membered non-aromatic cyclic alkene moieties, preferably a cyclooctene moiety, and more preferably a trans-cyclooctene moiety.

Trans-cyclooctene or E-cyclooctene derivatives are very suitable as Triggers, especially considering their high reactivity. Optionally, the trans-cyclooctene (TCO) moiety comprises at least two exocyclic bonds fixed in substantially the same plane, and/or it optionally comprises at least one substituent in the axial position, and not the equatorial position. The person skilled in organic chemistry will understand that the term “fixed in substantially the same plane” refers to bonding theory according to which bonds are normally considered to be fixed in the same plane. Typical examples of such fixations in the same plane include double bonds and strained fused rings. E.g., the at least two exocyclic bonds can be the two bonds of a double bond to an oxygen (i.e. C═O). The at least two exocyclic bonds can also be single bonds on two adjacent carbon atoms, provided that these bonds together are part of a fused ring (i.e. fused to the TCO ring) that assumes a substantially flat structure, therewith fixing said two single bonds in substantially one and the same plane. Examples of the latter include strained rings such as cyclopropyl and cyclobutyl. Without wishing to be bound by theory, the inventors believe that the presence of at least two exocyclic bonds in the same plane will result in an at least partial flattening of the TCO ring, which can lead to higher reactivity in the IEDDA reaction. A background reference providing further guidance is WO 2013/153254.

### Tetrazine

The compound according to the invention may herein be referred to as “Activator”. The tetrazine typically reacts with the other Bio-orthogonal Reactive Group, that is a dienophile (vide supra). The diene of the Activator is selected so as to be capable of reacting with the dienophile of the TCO by undergoing a Diels-Alder cycloaddition followed by a retro Diels-Alder reaction, giving the IEDDA adduct. This intermediate adduct then releases the Construct-A, where this release can be caused by various circumstances or conditions that relate to the specific molecular structure of the IEDDA adduct.

Synthesis routes to tetrazines in general are readily available to the skilled person, based on standard knowledge in the art. References to tetrazine synthesis routes include for example Lions et al, J. Org. Chem., 1965, 30, 318-319; Horwitz et al, J. Am. Chem. Soc., 1958, 80, 3155-3159; Hapiot et al, New J. Chem., 2004, 28, 387-392, Kaim et al, Z. Naturforsch., 1995, 50b, 123-127; Yang et al., Angew. Chem. 2012, 124, 5312-5315; Mao et al., Angew. Chem. Int. Ed. 2019, 58, 1106-1109; Qu et al. Angew. Chem. Int. Ed. 2018, 57, 12057-12061; Selvaraj et al., Tetrahedron Lett. 2014, 55, 4795-4797; Fan et al., Angew. Chem. Int. Ed. 2016, 55, 14046-14050.

### Section 18—Prodrug

A Prodrug is a conjugate of the Drug and the TCO and comprises a Drug that is capable of increased therapeutic action after release of Construct-A from the TCO. Such a Prodrug may optionally have specificity for disease targets.

With reference for Formula 19, Section 8, each Construct A and each Construct B are independently selected from the group consisting of drugs, targeting agents and masking moieties, provided that at least one Drug is comprised in the structure of Formula (19),

In a preferred embodiment Construct A is a Drug D^{D}.
In a preferred embodiment, when C^{B} is a targeting agent or a masking moiety, then C^{A} is a D^{D}.
In a preferred embodiment, when C^{B} is a D^{D}, then C^{A} is a masking moiety or a targeting agent.
In a preferred embodiment, when C^{A} is D^{D} then D^{D} is not bound to T^{R} or L^{C} via a Spacer S^{P}.
In a preferred embodiment at most one C^{B} is comprised in the structure of Formula (19),
In preferred embodiments Trigger cleavage results in the cleavage of one C^{A} from one C^{B}.

In another embodiment the Trigger cleavage results in cleavage of one C^{A} from another C^{A}, when the dienophile Trigger of Formula 19 links to two allylic positioned C^{A} (with reference to Formula 19: X⁵ is —CHR₄₈) or when one L^{C} is bound to two C^{A} moieties, wherein one or both C^{A} can release from the Trigger upon reaction with a diene, and wherein one C^{A} is a T^{T} or M^{M} and the other is the Drug. Optionally, one or more C^{B} can be additionally present and can independently be T^{T}/M^{M} or Drug.

In some embodiments Trigger cleavage results in the cleavage of one C^{A} from two or more C^{B}. In some embodiments Trigger cleavage results in the cleavage of one C^{B} from two or more C^{A}. In preferred embodiments, the Trigger is bound to only one C^{A} and one C^{B}. In other preferred embodiments, the Trigger is bound to two C^{A} moieties and no C^{B} moieties. In other preferred embodiments, the dienophile does not comprise a C^{B} moiety.

In some embodiments wherein a D^{D} is to be released from a T^{T} or M^{M}, to ensure efficient cleavage of the D^{D} from T^{T} or M^{M}, if there are multiple C^{B} moieties bound to the Trigger via X¹-X⁵ then all these C^{B} moieties are collectively either T^{T}/M^{M} or D^{D}. If there is one or more C^{B} bound via X¹-X⁵ and one C^{B} bound via L^{C} then the C^{B} bound via L^{C} can be selected independently from the C^{B} moieties bound to X¹-X⁵.

If there are multiple C^{A} moieties bound to the Trigger, either through substitution of both allylic positions in the dienophile (with reference to Formula 19: X⁵ is —CHR₄₈), or through binding of multiple C^{A} moieties via one linker L^{C} (with reference to Formula 19: r is 1 or 2), or both, each C^{A} can independently be T^{T}/M^{M} or Drug.

In a preferred embodiment, C^{B} is not comprised in X⁵.

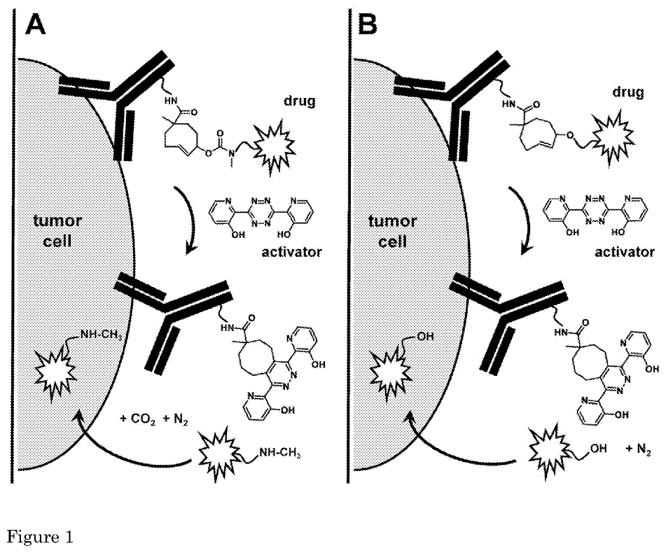

In a preferred embodiment the targeted Prodrug is an Antibody-Drug Conjugate (ADC). Activation of the Prodrug by the IEDDA pyridazine elimination of the TCO with the Activator leads to release of the Drug (FIG. 1). In a preferred embodiment the Drugs comprised in said ADC are low to medium molecular weight compounds, preferably organic compounds (e.g. about 200 to about 2500 Da, preferably about 300 to about 1750 Da, more preferably about 300 to about 1000 Da).

It is desirable to be able to activate targeted Prodrugs such as ADCs selectively and predictably at the target site without being dependent on homogenous penetration and targeting, and on endogenous activation parameters (e.g. pH, enzymes) which may vary en route to and within the target, and from indication to indication and from patient to patient. The use of a biocompatible chemical reaction that does not rely on endogenous activation mechanisms for selective Prodrug activation would represent a powerful new tool in cancer therapy. It would expand the scope to cancer-related receptors and extracellular matrix targets that do not afford efficient internalization of the ADC and therefore cannot be addressed with the current ADC approaches. In addition, extraneous and selective activation of Prodrugs when and where required leads to enhanced control over Prodrug activation, intracellularly and extracellularly. Finally this approach would maximize the bystander effect, allowing more efficient Drug permeation throughout the tumor tissue.

Other areas that would benefit from an effective prodrug approach are protein-based therapies and immunotherapy, for example bispecific T-cell engaging antibody constructs, which act on cancer by binding cancer cells and by engaging the immune system [Trends in Biotechnology 2015, 33, 2, 65]. Antibody constructs containing an active T-cell binding site suffer from peripheral T-cell binding. This not only prevents the conjugate from getting to the tumor but can also lead to cytokine storms and T-cell depletion. Photo-activatable anti-T-cell antibodies, i.e. T-cell directed Prodrugs, in which the anti-T-cell activity is only restored when and where it is required (i.e. after tumor localization via the tumor binding arm), following irradiation with UV light, has been used to overcome these problems [Thompson et al., Biochem. Biophys. Res. Commun. 366 (2008) 526-531]. However, light based activation is limited to regions in the body where light can penetrate, and is not easily amendable to treating systemic disease such as metastatic cancer.

Other proteins that could benefit from a Prodrug approach are immunotoxins and immunocytokines which suffer from respectively immunogenicity and general toxicity.

Hydrophilic polymers (such as polyethylene glycol, peptide and proteins have been used as cleavable masking moieties of various substrates, such as proteins, drugs and liposomes, in order to reduce their systemic activity. However, the used cleavage strategies were biological (pH, thiol, enzyme), as used in the ADC field, with the same drawbacks

In order to avoid the drawbacks of current prodrug activation, this invention makes use of an abiotic, bio-orthogonal chemical reaction to provoke release of the Drug from the Prodrug, such as an ADC. In this type of ADC, in a preferred embodiment, the Drug is attached to the antibody (or another type of Targeting Agent) via a Trigger, and this Trigger is not activated endogeneously by e.g. an enzyme or a specific pH, but by a controlled administration of the Activator, i.e. a species that reacts with the Trigger moiety in the ADC, to induce release of the Drug from the Trigger (or vice versa, release of the Trigger from the Drug, however one may view this release process) (FIG. 1).

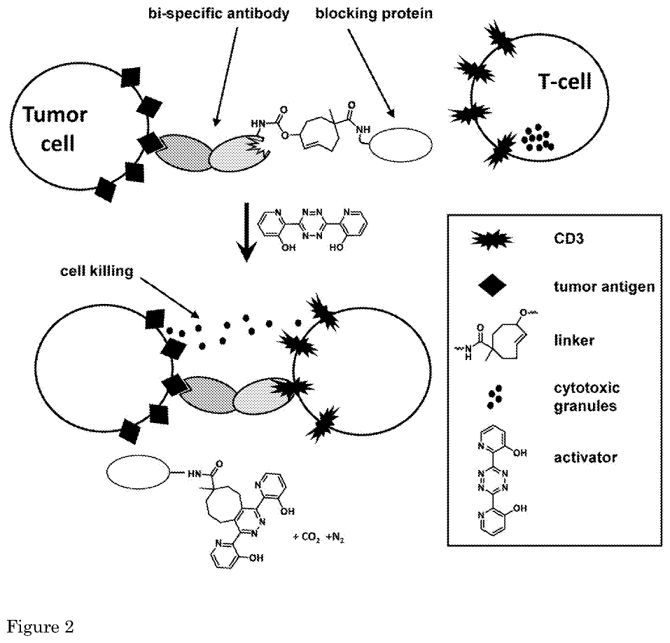

In another preferred embodiment, the Prodrug comprises a Drug bound via the trigger to a Masking Moiety. Administration of the Activator, induces release of the Drug from the Masking Moiety, resulting in activation of the Drug. In a particular embodiment, a protein with specificity for a tumor target is fused to a protein with specificity for the CD3 receptor on T-cells, wherein the CD3 binding domain is masked by conjugation of a cysteine near the domain to a Trigger comprising a Masking Moiety. Following tumor binding of the masked bispecific protein, the Activator is administered leading to unmasking of the CD3 domain and the binding to T-cells (FIG. 2).

In a preferred embodiment, the present invention provides a kit for the administration and activation of a Prodrug, the kit comprising a Drug, denoted as C^{A}, linked directly, or indirectly through a linker L^{C}, to a Trigger moiety T^{R}, wherein T^{R} or L^{C} is bound to a Construct-B, C^{B}, that is Targeting Agent T^{T} or a Masking Moiety M^{M}, and an Activator for the Trigger moiety, wherein the Trigger moiety comprises a dienophile and the Activator comprises a diene, the dienophile satisfying Formula (19).

In preferred embodiments, C^{B} is the Drug and C^{A} is a targeting agent or a masking moiety.

In yet another aspect, the invention provides a method of modifying a Drug compound into a Prodrug that can be triggered by an abiotic, bio-orthogonal reaction, the method comprising the steps of providing a Drug and chemically linking the Drug to a TCO moiety satisfying Formula (19).

In a still further aspect, the invention provides a method of treatment wherein a patient suffering from a disease that can be modulated by a Drug, is treated by administering, to said patient, a Prodrug comprising a Drug, a Trigger moiety and a Targeting agent after activation of which by administration of an Activator the Drug will be released, wherein the Trigger moiety comprises a structure satisfying Formula (19).

In a still further aspect, the invention is a compound comprising a TCO moiety, said moiety comprising a linkage to a Drug, for use in Prodrug therapy in an animal or a human being.

In another aspect, the invention is the use of a tetrazine as an Activator for the release, in a physiological environment, of a substance covalently linked to a compound satisfying Formula (19). In connection herewith, the invention also pertains to a tetrazine for use as an Activator for the release, in a physiological environment, of a substance linked to a compound satisfying Formula (19), and to a method for activating, in a physiological environment, the release of a substance linked to a compound satisfying Formula (19), wherein a tetrazine is used as an Activator.

In preferred embodiments a Prodrug is a conjugate of the Drug and the Trigger and thus comprises a Drug that is capable of increased therapeutic action after its release from the Trigger. In embodiments where the Prodrug is targeted to a Primary Target, as is the case with for example Antibody Drug Conjugates, the Prodrug can comprise a Targeting agent T^{T}, which is bound to either the Trigger or the L^{C}.

According to a further particular embodiment of the invention, the Prodrug is selected so as to target and or address a disease, such as cancer, an inflammation, an autoimmune disease, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme.

According to one embodiment, the Prodrug and/or the Activator can be, but are not limited to, multimeric compounds, comprising a plurality of Drugs and/or bioorthogonal reactive moieties. These multimeric compounds can be polymers, dendrimers, liposomes, polymer particles, or other polymeric constructs.

It is preferred that the optional L^{C} comprised in the Prodrug is self-immolative, affording traceless release of the Drug.

It shall be understood that one T^{T}, being C^{A} or C^{B}, can be modified with more than one Trigger. For example, an antibody can be modified with four TCO-Drug constructs by conjugation to four amino acid residues, wherein preferably when C^{B} is the T^{T} then C^{A} is a Drug, and wherein when C^{A} is the T^{T}, then C^{B} or another C^{A} is a Drug.

It shall be understood that one D^{D}, being C^{A} or C^{B}, can be modified with more than one Trigger. For example, an protein drug can be modified with multiple TCO-M^{M} constructs by conjugation to multiple amino acid residues, wherein preferably when C^{B} is the M^{M} then C^{A} is a Drug, and wherein when C^{A} is the M^{M}, then C^{B} or another C^{A} is a Drug.

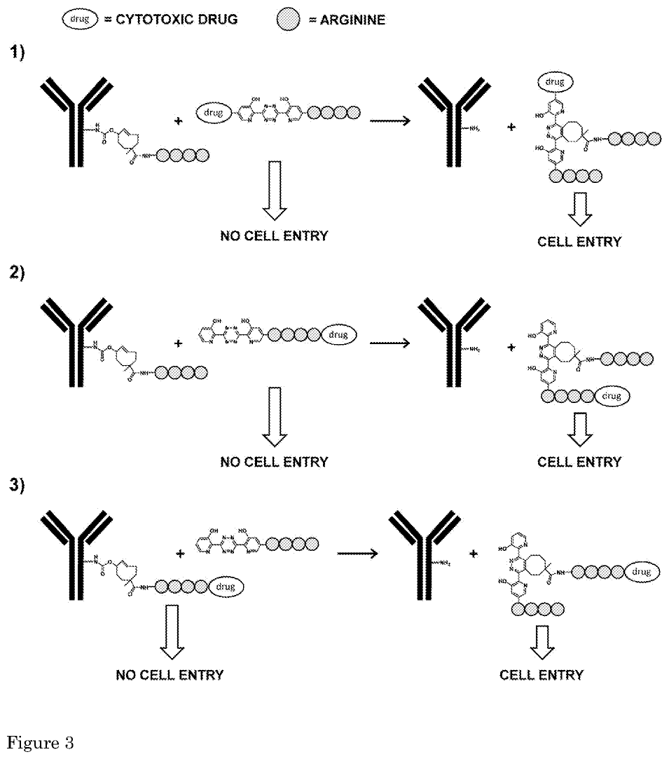

In other embodiments the IEDDA pyridazine elimination with the compounds of this invention is used to generate a cell penetrating drug at the target site in vivo. With reference to FIG. 3, in one particular embodiment, a cell penetrating peptide (CPP) containing 8 sequential arginine residues is assembled out of 2 peptides containing 4 arginine residues, which as a tetramer do not exhibit cell penetration [Bode et al., Chem. Sci., 2019, 10, 701]. In FIG. 3 panel 1, a tumor-targeted antibody containing a click-cleavable linker bound to a tetra-arginine moiety (a T^{T}) is reacted with systemically adminstered tetrazine containing a drug and another tetra-arginine moiety (a T^{T}). Reaction leads to formation of a drug linked to 8 arginine residues that can then penentrate surrounding cells. In FIG. 3 panel 2, shows the same concept with the drug bound via a different position. In FIG. 3, panel 3, the tumor-targeted antibody containing a click-cleavable linker bound to a tetra-arginine moiety (a T^{T}), which is further linked to a Drug. This Antibody drug conjugate is reacted with systemically administered tetrazine containing another tetra-arginine moiety (a T^{T}). Reaction leads to formation and liberation of a drug linked to 8 arginine residues that can then penentrate surrounding cells.

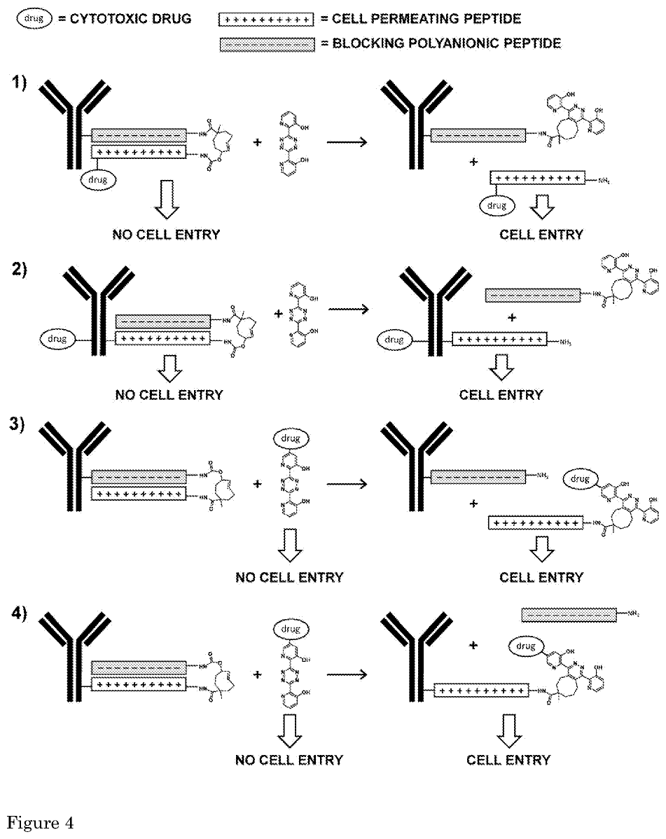

In other embodiments the IEDDA pyridazine elimination with the compounds of this invention is used to unmask a cell penetrating peptide leading to cell penetration of a drug at the target site in vivo. With reference to FIG. 4, in one particular embodiment, a polycationic cell penetrating peptide (CPP) containing 10 sequential arginine residues is masked by a polyanionic polyglutamate peptide containing 10 glutamate residues and which is linked via the TCO linker to the polyarginine linker. Reference is made to [Duijnhoven et al., J Nucl Med 2011, 52, 279]. In FIG. 4, panel 1, the tumor-targeted antibody containing a polyanionic peptide linked to click-cleavable TCO linker which in turn is bound to a poly-arginine peptide which is further linked to a Drug is reacted with systemically adminstered tetrazine leading to liberation of a drug linked to the polyarginine peptide that can then penentrate surrounding cells. In panel 2, a tumor-bound antibody-drug conjugate (ADC) which in addition is modified with a masked poly-arginine peptide is reacted with systemically adminstered tetrazine leading to unmasking of poly-arginine peptide and internalization of the ADC. In panel 3, a tumor-bound antibody modified with a masked poly-arginine peptide is reacted with systemically adminstered tetrazine-drug conjugate leading to unmasking and release of the poly-arginine peptide and its concomitant conjugation to the tetrazine-drug, leading to internalization of said drug. In panel 4, a tumor-bound antibody modified with a masked poly-arginine peptide is reacted with systemically adminstered tetrazine-drug conjugate leading to unmasking the poly-arginine peptide and the concomitant conjugation to the tetrazine-drug to the antibody, leading to internalization of the insitu formed ADC. In some preferred embodiments the polyanionic and polycationic peptides have a length of 8 amino acids. In some preferred embodiments wherein this invention is used to unmask a cell penetrating peptide leading to cell penetration of a drug at the target site in vivo, the Drug is or comprises a therapeutic radioactive moiety.

In other embodiments the IEDDA pyridazine elimination with the compounds of this invention is used to assemble a Drug in vivo. In an exemplary embodiment, and in a similar approach as with the above used CPP) a Drug being a prodrug is C^{B} and T^{T} is C^{A}. After binding to a Primary Target, an Activator that is bound to a Drug being a prodrug is adminstered and reacts with the Trigger on the Primary Target. This results in the concomittant conjugation of both prodrug moieties to one another, forming an active Drug, and the release of the Drug from the T^{T}.

In other embodiments the Prodrug comprises a Drug bound via T^{R} to a polymer, a gel, a solid material (e.g. of an implantable drug depot) or a biomolecule reactive moiety such as R³², and this Prodrug is directly administered into/near the region of interest (e.g. a tumor), resulting in local immobilization of the Prodrug, which can subsequently be activated by local or systemic administration of the Activator. Conversely, in other embodiments, the Activator is bound to a R⁸⁷ being a polymer, a gel, a solid material (e.g. of an implantable drug depot) or a biomolecule reactive moiety such as R³², and is directly administered into/near the region of interest (e.g. a tumor), resulting in local immobilization of the Activator, which can locally activate the subsequently (locally or systemically) administered Prodrug. In another embodiment, the Activator, bound to a Targeting Agent, is systemically adminstered and allowed to bind the Primary Target, after which a non-targeted or targeted Prodrug is adminstered systemically and is activated at the Primary Target. In some embodiments the Prodrug consists of two constructs, which are the same or different Drugs D^{D}, and one or both D^{D} are activated upon cleavage of T^{R}. In some embodiments, the Prodrug comprises two different D^{D}, with one D^{D} functioning as a targeting agent T^{T} in addition to being a D^{D}. In a particularly preferred embodiment, one of or both D^{D} are activated upon cleavage of T^{R}. In other embodiments, the Activator used to activate a targeted bound

Prodrug, e.g. a tumor-bound ADC, comprises a Drug, for example a therapeutic radioactive moiety (e.g. 177-Lu-DOTA chelate complex conjugated to the tetrazine) to augment the therapeutic effect of the released drug. For example, therapeutic radiation (e.g. beta emission) is known to stimulate an anticancer immune response. By releasing an anticancer drug and at the same time activating the immune system a greater anticancer effect can be obtained. In preferred embodiments the released Drug is a cytotoxic molecule, to be combined with therapeutic radiation (low or high dose). In preferred embodiments the released Drug is a radiation sensitizer, to be combined with therapeutic radiation (low or high dose), In other embodiments the released Drug is an immune modulator (e.g. a TLR agonist, or a cytokine) to be combined with therapeutic radiation (low or high dose).

In another embodiment the Prodrug comprises a T^{T} bound via the T^{R} to a Drug, wherein the Drug is or comprises a therapeutic radioactive moiety, and wherein, optionally, release of the radioactive moiety at the target site, e.g. a tumor, may result in deeper and more homogeneous penetration and distribution of the radioactivity than when the radioactive moiety remains bound to T^{T}.

### Section 18a—Drug Deactivation

In other embodiments the IEDDA pyridazine elimination with the compounds of this invention is used to deactivate a Drug in vivo. In these embodiments a Drug is allowed to circulate in vivo and to exert its therapeutic action, and after an optimal interval, the Drug is deactivated by administration of a tetrazine, resulting in cleavage of the Drug and its deactivation, reducing undesired side effects. Exemplary Drugs that can be deactivated in this manner include biomolecules, wherein one (bio)molecule is linked to another (bio)molecule via the Trigger (as C^{A} or C^{B}) and this (bio)molecule-(bio)molecule conjugate is therapeutically active whereas the cleaved biomolecules are not. Exemplary biomolecule conjugates are protein-protein, peptide-peptide, protein-peptide, protein-organic molecule, organic molecule-organic molecule conjugates. In a preferred embodiment, the drug deactivation is spatially controlled instead of or in addition to temporally controlled. For example, the Activator can be administered specifically to a location of the body where a systemically administered Drug is not desired to work, e.g. a particular organ, while the Drug remains active in other locations/systemically. Conversely the Activator can be administered systemically to deactivate a Drug in circulation, while maintaining Drug activity in a particular location, e.g. an organ or a site of disease. In one embodiment, the Drug is an immunesuppressant used in the context of organ transplantation and which is desired to work near the organ to prevent transplant rejection, but is desired to be inactive in other areas.
In other embodiments, the Drug D^{D} is active when bound to the Trigger T^{R}, and is deactivated upon the IEDDA pyridazine elimination with a tetrazine. In some embodiments the Prodrug consists of two constructs, which are the same or different Drugs D^{D}, and one or both D^{D} are deactivated upon cleavage of T^{R}. In a preferred embodiment, administration of a T^{T}-T^{R}-D^{D} conjugate leads to accumulation and therapeutic activity of the Drug in the target tissue, after which a tetrazine is adminstered at a desired time and/or location to cleave D^{D} from T^{T} resulting in a reduction of the therapeutic or toxic effect at the Primary Target.

### Section 19—Drugs

Drugs that can be used in a Prodrug relevant to this invention are pharmaceutically active compounds.

In a preferred embodiment the pharmaceutically active compound is selected from the group consisting of cytotoxins, antiproliferative/antitumor agents, antiviral agents, antibiotics, anti-inflammatory agents, chemosensitizing agents, radiosensitizing agents, immunomodulators, immunosuppressants, immunostimulants, anti-angiogenic factors, enzyme inhibitors.

In preferred embodiments these pharmaceutically active compounds are selected from the group consisting of antibodies, antibody derivatives, antibody fragments, proteins, aptamers, oligopeptides, oligonucleotides, oligosaccharides, carbohydrates, as well as peptides, peptoids, steroids, toxins, hormones, cytokines, chemokines

In preferred embodiments these drugs are low to medium molecular weight compounds, preferably organic compounds (e.g. about 200 to about 2500 Da, preferably about 300 to about 1750 Da, more preferably about 300 to about 1000 Da).

Exemplary cytotoxic drug types for use as conjugates to the TCO and to be released upon IEDDA reaction with the Activator, for example for use in cancer therapy, include but are not limited to DNA damaging agents, DNA crosslinkers, DNA binders, DNA alkylators, DNA intercalators, DNA cleavers, microtubule stabilizing and destabilizing agents, topoisomerases inhibitors, radiation sensitizers, anti-metabolites, natural products and their analogs, peptides, oligonucleotides, enzyme inhibitors such as dihydrofolate reductase inhibitors and thymidylate synthase inhibitors.

Examples include but are not limited to colchinine, vinca alkaloids, anthracyclines (e.g. doxorubicin, epirubicin, idarubicin, daunorubicin), camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, deBouganin, dactinomycines, CC1065 and its analogs, duocarmycins, maytansines, maytansinoids, dolastatins, auristatins, pyrrolobenzodiazepines and dimers (PBDs), indolinobenzodiazepines and dimers, pyridinobenzodiazepines and dimers, mitomycins (e.g. mitomycin C, mitomycin A, caminomycin), melphalan, leurosine, leurosideine, actinomycin, tallysomycin, lexitropsins, bleomycins, podophyllotoxins, etoposide, etoposide phosphate, staurosporin, esperamicin, the pteridine family of drugs, SN-38 and its analogs, platinum-based drugs, cytotoxic nucleosides.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, and RNA polymerase inhibitors.

Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. Suitable auristatins are also described in U.S. Publication Nos. 2003/0083263, 2011/0020343, and 2011/0070248; PCT Application Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety.

Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497. Other examples include mertansine and ansamitocin.

Pyrrolobenzodiazepines (PBDs), which expressly include dimers and analogs, include but are not limited to those described in [Denny, Exp. Opin. Ther. Patents, 10(4):459-474 (2000)], [Hartley et al., Expert Opin Investig Drugs. 2011, 20(6):733-44], Antonow et al., Chem Rev. 2011, 111(4), 2815-64]. Exemplary indolinobenzodiazepines are described in literature. Exemplary pyridinobenzodiazepines are described in literature.

Calicheamicins include, e.g. enediynes, esperamicin, and those described in U.S. Pat. Nos. 5,714,586 and 5,739,116

Examples of duocarmycins and analogs include CC1065, duocarmycin SA, duocarmycin A, duocarmycin B1, duocarmycin B2, duocarmycin C1, duocarmycin C2, duocarmycin D, DU-86, KW-2189, adozelesin, bizelesin, carzelesin, seco-adozelesin, CPI, CBI. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety.

Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety.

Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety.

Exemplary cryptophycin compounds are described in U.S. Pat. Nos. 6,680,311 and 6,747,021; the disclosures of which are incorporated herein by reference in their entirety.

Exemplary platinum compounds include cisplatin, carboplatin, oxaliplatin, iproplatin, ormaplatin, tetraplatin.

Exemplary DNA binding or alkylating drugs include CC-1065 and its analogs, anthracyclines, calicheamicins, dactinomycines, mitromycines, pyrrolobenzodiazepines, indolinobenzodiazepines, pyridinobenzodiazepines and the like.

Exemplary microtubule stabilizing and destabilizing agents include taxane compounds, such as paclitaxel, docetaxel, tesetaxel, and carbazitaxel; maytansinoids, auristatins and analogs thereof, vinca alkaloid derivatives, epothilones and cryptophycins.

Exemplary topoisomerase inhibitors include camptothecin and camptothecin derivatives, camptothecin analogs and non-natural camptothecins, such as, for example, CPT-11, SN-38, topotecan, 9-aminocamptothecin, rubitecan, gimatecan, karenitecin, silatecan, lurtotecan, exatecan, diflometotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med Chem., 30:1774 (1987).

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure.

Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MLN8237; PLK inhibitors such as, for example, BI 2536, BI6727, GSK461364, ON-01910; and KSP inhibitors such as, for example, SB 743921, SB 715992, MK-0731, AZD8477, AZ3146 and ARRY-520.

Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors.

Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,944 and include GDC-0973, GSK1120212, MSC1936369B, AS703026, RO5126766 and RO4987655, PD0325901, AZD6244, AZD8330 and GDC-0973.

Exemplary B-raf inhibitors include CDC-0879, PLX-4032, and SB590885.

Exemplary B p38 MAPK inhibitors include BIRB 796, LY2228820 and SB 202190.

Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib.

Exemplary protein chaperon inhibitors include HSP90 inhibitors. Exemplary inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA).

Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461.

Exemplary Wnt/Hedgehog signalling pathway inhibitors include vismodegib, cyclopamine and XAV-939.

Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin.

Exemplary immunemodulators are APRIL, cytokines, including IL-2, IL-7, IL-10, IL-12, IL-15, IL-21, TNF, interferon gamma, GMCSF, NDV-GMCSF, and agonists and antagonists of STING, agonists and antagonists of TLRs including TLR1/2, TLR3, TLR4, TLR7/8, TLR9, TLR12, agonists and antagonists of GITR, CD3, CD28, CD40, CD74, CTLA4, OX40, PD1, PDL1, RIG, MDA-5, NLRP1, NLRP3, AIM2, IDO, MEK, cGAS, and CD25, NKG2A.

Other exemplary drugs include puromycins, topetecan, rhizoxin, echinomycin, combretastatin, netropsin, estramustine, cemadotin, discodermolide, eleutherobin, mitoxantrone, pyrrolobenzimidazoles (PBI), gamma-interferon, Thialanostatin (A) and analogs, CDK11, immunotoxins, comprising e.g. ricin A, diphtheria toxin, cholera toxin.

In exemplary embodiments of the invention, the drug moiety is a mytomycin compound, a vinca alkaloid compound, taxol or an analogue, an anthracycline compound, a calicheamicin compound, a maytansinoid compound, an auristatin compound, a duocarmycin compound, SN38 or an analogue, a pyrrolobenzodiazepine compound, a indolinobenzodiazepine compound, a pyridinobenzodiazepine compound, a tubulysin compound, a non-natural camptothecin compound, a DNA binding drug, a kinase inhibitor, a MEK inhibitor, a KSP inhibitor, a P13 kinase inhibitor, a topoisomerase inhibitor, or analogues thereof.

In one preferred embodiment the drug is a non-natural camptothecin compound, vinca alkaloid, kinase inhibitor, (e.g. P13 kinase inhibitor: GDC-0941 and PI-103), MEK inhibitor, KSP inhibitor, RNA polymerase inhibitor, PARP inhibitor, docetaxel, paclitaxel, doxorubicin, dolastatin, calicheamicins, SN38, pyrrolobenzodiazepines, pyridinobenzodiazepines, indolinobenzodiazepines, DNA binding drugs, maytansinoids DM1 and DM4, auristatin MMAE, CC1065 and its analogs, camptothecin and its analogs, SN-38 and its analogs.

In another preferred embodiment the drug is selected from DNA binding drugs and microtubule agents, including pyrrolobenzodiazepines, indolinobenzodiazepines, pyridinobenzodiazepines, maytansinoids, maytansines, auristatins, tubulysins, duocarmycins, anthracyclines, taxanes.

In another preferred embodiment the drug is selected from colchinine, vinca alkaloids, tubulysins, irinotecans, an inhibitory peptide, amanitin and deBouganin.

In another preferred embodiment the drug is a radioactive moiety, said moiety comprising a radioactive isotope for radiation therapy. A radionuclide used for therapy is preferably an isotope selected from the group consisting of ²⁴Na, ³²P, ³³P, ⁴⁷Sc, ⁵⁹Fe, ⁶⁷Cu, ⁷⁶As, ⁷⁷As, ⁸⁰Br, ⁸²Br, ⁸⁹Sr, ⁹⁰Nb, ⁹⁰Y, ¹⁰³Ru, ¹⁰⁵Rh, ¹⁰⁹Pd, ¹¹¹Ag, ¹¹¹In, ¹²¹Sn, ¹²⁷Te, ¹³¹I, ¹⁴⁰La, ¹⁴¹Ce, ¹⁴²Pr, ¹⁴³Pr, ¹⁴⁴Pr, ¹⁴⁹Pm, ¹⁴⁹Tb, ¹⁵¹Pm, ¹⁵³Sm, ¹⁵⁹Gd, ¹⁶¹Tb, ¹⁶⁵Dy, ¹⁶⁶Dy, ¹⁶⁶Ho, ¹⁶⁹Er, ¹⁷²Tm, ¹⁷⁵Yb, ¹⁷⁷Lu, ¹⁸⁶Re, ¹⁸⁸Re, ¹⁹⁸Au, ¹⁹⁹Au, ²¹¹At, ²¹¹Bi ²¹²Bi, ²¹²Pb, ²¹³Bi, ²¹⁴Bi, ²²³Ra, ²²⁴Ra, ²²⁵Ac, and ²²⁷Th

When the radioactive moiety is intended to comprise a metal, such as ¹⁷⁷Lu, such radiometal is preferably provided in the form of a chelate. In such a case the radioactive moiety preferably comprises a structural moiety capable of forming a coordination complex with such a metal. A good example hereof are macroyclic lanthanide(III) chelates derived from 1,4,7,10-tetraazacyclododecane-1,4,7,10-tetraacetic acid (H₄dota).

In a preferred embodiment, the structural moiety capable of forming a coordination complex with such a metal is a chelating moiety selected from the group consisting of DTPA (diethylenetriaminepentaacetic acid), DOTA (1,4,7,10-tetraazacyclododecane-N,N′,N″,N″-tetraacetic acid), NOTA (1,4,7-triazacyclononane-N,N′,N″-triacetic acid), TETA (1,4,8,11-tetraazacyclotetradecane-N,N′,N″,N′-tetraacetic acid), OTTA (N1-(p-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide), and HYNIC (hydrazinonicotinamide), DOTAM, TACN, sarcophagine, and 3,4-HOPO-based chelators.

In other embodiments the radioactive moiety comprises a prosthetic group (i.e. a phenol) that is bound by a non-metal radionuclide, such as ¹³¹I. In another embodiment, a combination of two or more different drugs are used.

In preferred embodiments the released Drug is itself a prodrug designed to release a further drug.

In preferred embodiments the released Drug is itself a prodrug designed to be coupled to another moiety, such as another prodrug) to form an active Drug.

In preferred embodiments the Drug has increased therapeutic efficacy after reaction of the Trigger with the Activator.

In some embodiments the Drug has reduced therapeutic efficacy after reaction of the Trigger with the Activator.

Drugs optionally include a (portion of a) membrane translocation moiety (e.g. adamantine, poly-lysine/arginine, TAT, human lactoferrin) and/or a targeting agent (against e.g. a tumor cell receptor) optionally linked through a stable or labile linker. Exemplary references include: Trends in Biochemical Sciences, 2015. 40, 12, 749; J. Am. Chem. Soc. 2015, 137, 12153-12160; Pharmaceutical Research, 2007, 24, 11, 1977.

It will further be understood that, in addition to one or more targeting agents (or C^{B}) that may be attached to the Trigger or Linker L^{C} a targeting agent T^{T} may optionally be attached to a drug, optionally via a spacer S^{P}. In some embodiments, the targeting efficacy of said T^{T} attached to a drug is increased after reaction of the Trigger with an Activator, wherein preferably the Drug is C^{B}, wherein preferably the Activator comprises a T^{T}, wherein optionally the targeting efficacy of said T^{T} comprised in the Activator is increased after reaction of the Trigger with an Activator.

Alternatively, it will be further understood that the targeting agent (or C^{B}) may comprise one or more additional drugs which are bound to the targeting agent by other types of linkers, e.g. cleavable by proteases, pH, thiols, or by catabolism.

The invention further contemplates that when a targeting agent is a suitably chosen antibody or antibody derivative that such targeting agent can induce antibody-dependent cellular toxicity (ADCC) or complement dependent cytotoxicity (CDC).

Several drugs may be replaced by an imageable label to measure drug targeting and release.

It will be understood that chemical modifications may also be made to the desired compound in order to make reactions of that compound more convenient for purposes of preparing conjugates of the invention.

Drugs containing an amine functional group for coupling to the TCO include mitomycin-C, mitomycin-A, daunorubicin, doxorubicin, aminopterin, actinomycin, bleomycin, 9-amino camptothecin, N8-acetyl spermidine, 1-(2 chloroethyl)1,2-dimethanesulfonyl hydrazide, tallysomycin, cytarabine, dolastatins (including auristatins) and derivatives thereof.

Drugs containing a hydroxyl function group for coupling to the TCO include etoposide, camptothecin, taxol, esperamicin, 1,8-dihydroxy-bicyclo[7.3.1]trideca-4-9-diene-2,6-diyne-13-one (U.S. Pat. No. 5,198,560), podophyllotoxin, anguidine, vincristine, vinblastine, morpholine-doxorubicin, n-(5,5-diacetoxy-pentyl)doxorubicin, and derivatives thereof.

Drugs containing a sulfhydryl functional group for coupling to the TCO include esperamicin and 6-mecaptopurine, and derivatives thereof.

It will be understood that the drugs can optionally be attached to the TCO derivative through a self-immolative linker L^{C}, or a combination thereof, and which may consist of multiple (self-immolative, or non immolative) units.

Several drugs may be replaced by an imageable label to measure drug targeting and release.

According to a further particular embodiment of the invention, the Prodrug is selected so as to target and or address a disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme.

In the Prodrug, the Construct-A and the TCO derivative can be directly linked to each other. They can also be bound to each other via a linker or a self-immolative linker L^{C}. It will be understood that the invention encompasses any conceivable manner in which the dienophile TCO is attached to the Contruct-A. In preferred embodiments Construct-A is a Drug. Methods of affecting conjugation to these drugs, e.g. through reactive amino acids such as lysine or cysteine in the case of proteins, are known to the skilled person.

### Section 21—Log P

In preferred embodiments, the compounds of the invention have a Log P value of 3.0 or lower, preferably 2.0 or lower, more preferably 1.0 or lower, most preferably 0.0 or lower.

In another preferred embodiment the Log P of compounds of the invention have a value in a range of from 2.0 and −2.0, more preferably in a range of from 1.0 and −1.0.

In embodiments where it is required that the Activator has an extracellular volume of distribution it is preferred that the Log P of the Activator is at most 2, preferably at most 1, more preferably at most 0, even more preferably at most −1.

In embodiments where it is required that the Activator has an intracellular volume of distribution it is preferred that the Log P of the Activator is at least −1, preferably at least 0, more preferably at least 1, even more preferably at least 2.

### Section 22—In Vitro Applications

The invention provides a tool for the controlled release of a bound substance (bound to any carrier or any other chemical group) for a diverse range of applications, including but not limited to drug delivery, chemical biology, diagnostics, organic, protein, and material chemistry, radiochemistry, capture and release resins, biological and chemical sensors, surface patterning or modification, cell and tissue culture and biomolecule manipulation (e.g. conjugation, crosslinking, trapping, releasing).

Particularly, the release can be in a chemically complex environment, including organic synthesis and material synthesis, biological surroundings, which include the synthesis and handling conditions of biomolecules. This includes the presence of diverse chemical functionalities, organic solvents, aqueous solvents biological media and tissues and cell lysates. The invention preferably relates to in vitro release at ambient temperature.

The invention provides, in one aspect, the use of a tetrazine as an Activator for the release, in a chemical, biological, or physiological environment, of a construct linked to a trans-cyclooctene. The term Construct in this invention is used to indicate any substance, carrier, or chemical group, of which it is desired to have it first in a bound (or masked) state, and being able to provoke release from that state. The Construct may be present in the form of two or more Constructs, linked via a self-immolative linker.

The invention includes in one aspect a Trigger which functions as a protective or masking group for use in organic, peptide, protein, bio-, surface, solid-phase chemistry.

The invention includes in one aspect a Trigger which functions as a cleavable mask or linker for use in organic and bioorganic synthesis, materials science, chemical biology, diagnostics, and medicine. The invention can be used for the manipulation of small molecules, peptides, proteins, oligonucleotides, polymers, glycans, nanoparticles, and on surfaces (e.g., glass slides, gold, resins). Further examples include: compound library synthesis, protein engineering, functional proteomics, activity-based protein profiling, target guided synthesis of enzyme inhibitors, chemical remodeling of cell surfaces, tracking of metabolite analogues, and imaging tagged biomolecules in live cells.

Therein, when used as a mask, with reference to formula 5a and 5b: f is preferably 0. Therein, when used as a linker, with reference to formula 5a and 5b: f is preferably 1.

Selective removal of the Trigger via reaction with the Activator unmasks the construct. Exemplary chemical moieties that can be protected and selectively deprotected include, but are not limited to, amines, thiols, hydroxyl, carboxylic acid, aminooxy groups.

Furthermore, the Activator may be conjugated to a resin, especially solid-phase synthesis resins, such as polystyrene, or to a bead. Thus circumventing the need for solution phase purification after the deprotection step. Therein, with reference to Formula 1, R₈₇ is a polymer, resin or a bead.

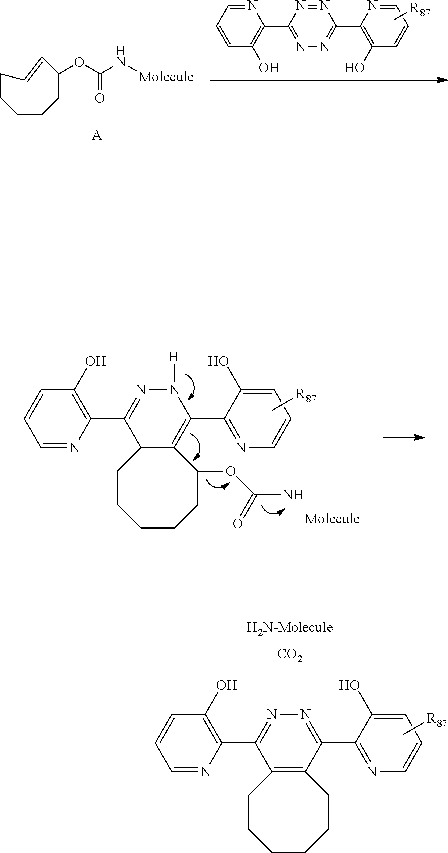

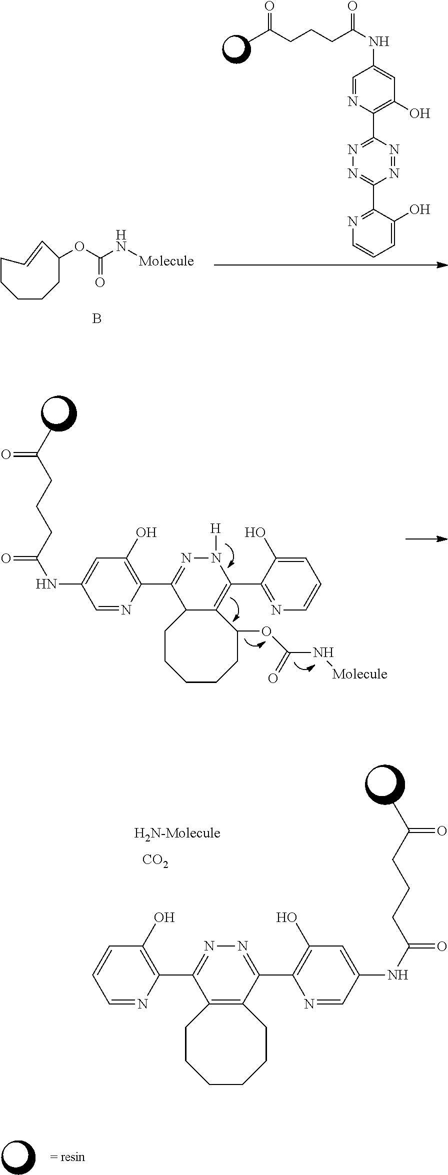

In the case of biomolecules such as proteins the Trigger can be introduced after the protein has been formed or during protein synthesis by means of genetically incorporating a Trigger-modified aminoacid. There have been many studies demonstrating the incorporation of unnatural aminoacids in proteins, including TCO and tetrazine (e.g. Chalker et al. Acc Chem Res, Vol. 44, No. 9, 2011, 730-741). In this way one can for example conduct bioconjugation chemistry elsewhere on the molecule, after which the TCO-masked amino acid can be unveiled and selectively manipulated. This so-called “Tag-and-Modify” approach thus allows for multiple and different post-translational modifications on a single protein and can be extended to controlled assembly and fragmentation of complex materials, proteins, cells, tissues. Examples of masked aminoacid derivatives, which can be used in such as approach are shown below. Compound A is the aminoacid cysteine of which the thiol functionality is masked by TCO. After release of TCO the thiol can be used in conjugation reactions. In addition to achieving selectivity, the ability to mask and unmask a thiol enables stabilization of thiol containing proteins against undesired oxidation and disulfide formation. Compound B is aminoacid lysine conjugated via its e-amine moiety to a TCO. Compound C is the aminoacid lysine conjugated via its e-amine moiety to a TCO-masked aminooxy functionality. After unmasking this aminooxy can be selectively conjugated to aldehyde and ketone derivatives. Compound D is amino acid serine conjugated via its hydroxyl moiety to TCO. Compound E is amino acid glutamic acid conjugated via its γ-carboxylate moiety to TCO.

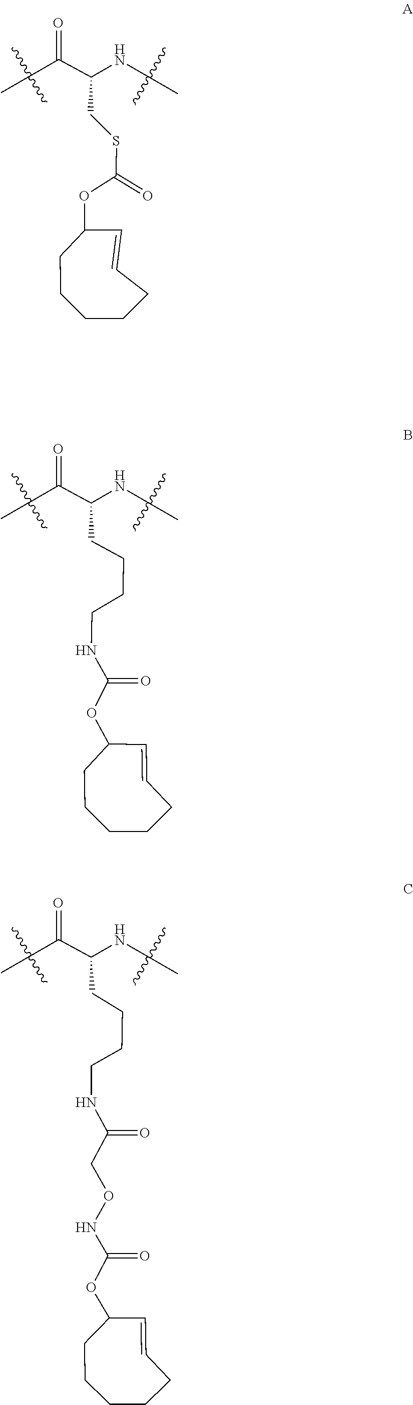 [SMILES: *N[C@H](CSC(=O)OC1/C=C/CCCCC1)C(*)=O ; *N[C@H](CCCCNC(=O)OC1/C=C/CCCCC1)C(*)=O ; *N[C@H](CCCCNC(=O)CONC(=O)OC1/C=C/CCCCC1)C(*)=O]

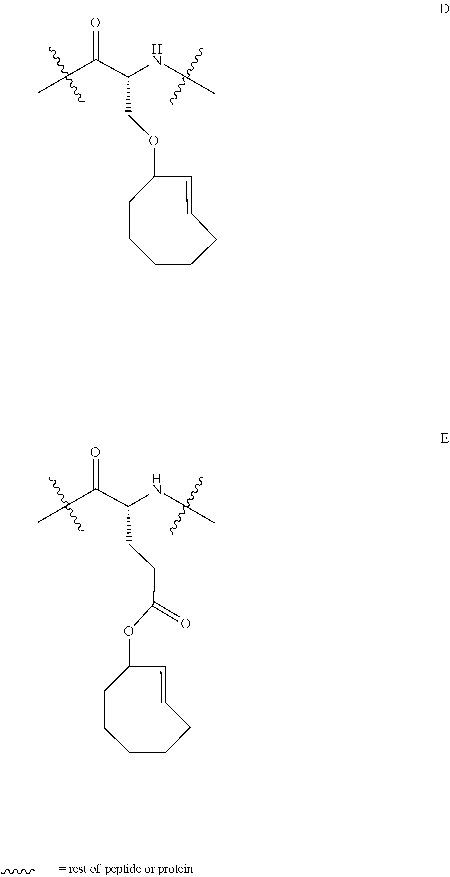

In a similar embodiment depicted below the TCO mask is used to stabilize protein formulations in e.g. stock solutions to prevent aggregation and precipitation. For this purpose the TCO is functionalized with C^{B} being an hydrophilic moiety such as a PEG (or e.g. a carbohydrate moiety), and one or multiple TCO-C^{B} groups are conjugated to the protein (being C^{A}) via e.g. lysine residues. At the time of use, the protein solution is contacted with the Activator yielding the unmasked parent protein C^{A}. Also here it may be advantageous to use an Activator that is conjugated to solid phase synthesis resins or a bead, thus circumventing the need for solution phase purification after the unmasking step.

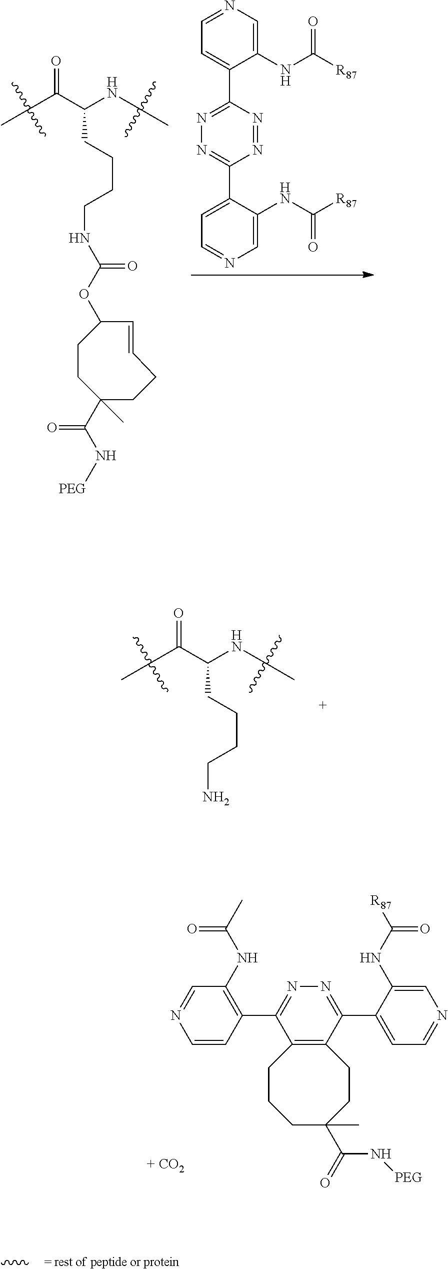

In another embodiment the Trigger is used as a chemically cleavable mask in the patterning or etching of surfaces with application in e.g. spatially controlled cell and tissue culture, or for (e.g. protein, DNA) microarray assembly. Selective removal of the mask reveals e.g. free amine or thiol moieties (comprised in C^{A}), which can be used for further modifications, such as the conjugation of cell adhering peptides in the case surfaces for cell culture. For example, the TCO can be used as cleavable linker between a surface or surface-coated gel and cell-interacting moieties, such as integrin binders, for use in cell culture. After cell culture on this surface or in this 3D the cells can be removed from the surface or the surface-coated gel by mild cleavage of the TCO, instead of resorting to harsh trypsinization, or physical force.

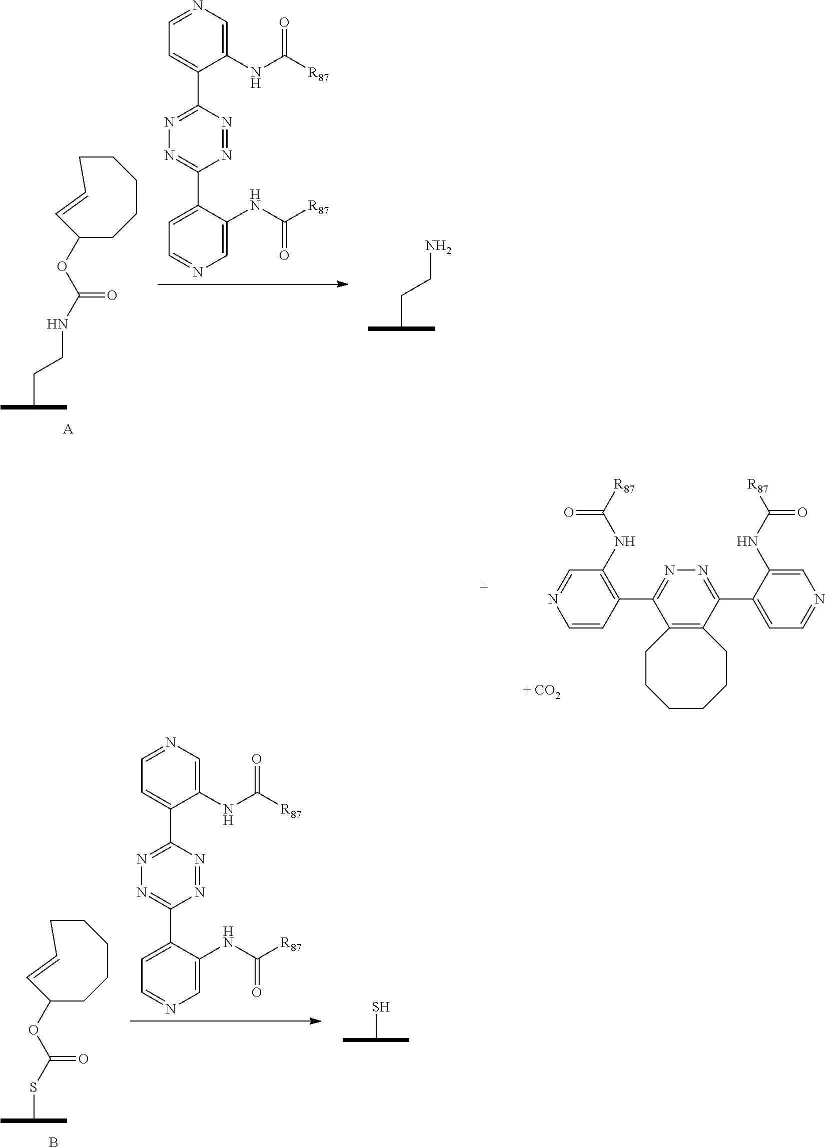

In an alternative embodiment the TCO mask is used to spatially and or temporally control the action of biomolecules in vitro or in vivo. For example, the action of a particular enzyme in an in vitro assay can be controlled by using an enzyme that has been deactivated through conjugation to one or more TCO masks, followed by contacting the enzyme with the Activator followed by release of the TCO mask and, affording the parent, active enzyme (C^{A}). Reference is made to [Li et al. Nat. Chem. Biol., 2014, 10, 1003-1005; Zhang et al. ACS Central Sci. 2016, 2, 5, 325-31]. Another example, useful in chemical biology, is the TCO-protection of certain aminoacid residues in a protein against enzymatic action such as phosphorylation by kinase, allowing spatial and temporal control over the phosphorylation after Activator addition. Alternatively, phospho aminoacids in phosphoproteins can be masked by TCO to be revealed at desired time by use of the Activator, as shown in a similar approach using light activatable masks by Rothman et al (2005) J. Am. Chem. Soc, 127, 847.

In another aspect of the invention the Trigger is used as a cleavable linker in “catch and release” systems, such as those used in chemical biology. Therein, with reference to Formula 5a and 5b: f is preferably 1.

One application of these linkers is in the purification of proteins tagged with a biotinylated Activity Based Probes (ABP). Biotinylated ABPs are often used for enrichment of captured enzymes, for instance, by pull-down with streptavidin-coated beads. The main disadvantages of this approach, however, are that the conditions to liberate the captured proteins from the beads are harsh (boiling of the sample, all or not in the presence of unmodified biotin) and that, beside the target proteins, both endogenously biotinylated proteins and (denatured) streptavidin can end up in the sample. In addition the presence of the biotin complicates MS/MS analysis. Furthermore, in another application, this concept can be used to capture and release whole cells with e.g. antibodies conjugated to e.g. a bead or solid support for example for the purpose of further analysis with FACS, requiring healthy intact cells. Several groups have developed linker systems that can be incorporated in the ABP, or alternatively in a bioorthogonal reagent for two-step ABPP, and that can be cleaved in a chemoselective manner after affinity pull-down. Examples include the disulfide, diazobenzene, and bisaryl hydrazine cleavable linkers (Willems L. I. et al. (2011) Acc. Chem. Res. 44, 718-729). However, these linkers have a limited bioorthogonality. In below scheme several embodiment examples are shown of the use of the Trigger for this application. In addition to the enhanced bioorthogonality this method also offers the opportunity to introduce a new label or affinity tag or to preserve a synthetic handle for further modification, through the binding of the tetrazine activator to the TCO.

Example A1 depicts the capturing of an enzyme by a ABP conjugated to biotin via a TCO linker. The complex is subsequently bound and isolated by streptavidin coated beads, after which the linker is cleaved by the Activator, and the enzyme, comprising the ABP linked to the IEDDA residue is released. In Example A2, the same concept is used with a reversed Trigger, leading to traceless release of the ABP-enzyme complex. Example B depicts an analogous 2 step ABP approach where the enzyme is captured by an ABP functionalized with an azide moiety. The complex is subsequently reacted with a cyclooctyne moiety, which is linked to a biotin via a TCO linker. It has been shown in Weissleder Angewandte Chemie 2011 that the TCO/tetrazine pair can be used orthogonally to and in the presence of the azide-octyn pair. In Example C the cyclooctyne-TCO-biotin probe approach described in B) is used to capture a specific protein, which has metabolically incorporated an azide-modified aminoacid. Example D depicts the capturing of cells using magnetic beads conjugated to antibodies via the Trigger. After binding and isolation using magnetic action, the cells are detached under mild conditions by adding the Activator. Example E depicts an alternative to the general approach in Examples A-D where the TCO combines the function of the biotin tag with the function of releasable linker. In this example, target cells are first bound by TCO-modified antibodies, followed by addition of tetrazine-coated beads. Suitably chosen tetrazine-TCO pairs will give a release with a half life of >2 h and given the required reaction time of <10 minutes thus allow enough time for isolation of the bead-cell complex before the complex releases the cell automatically through release of the linker.

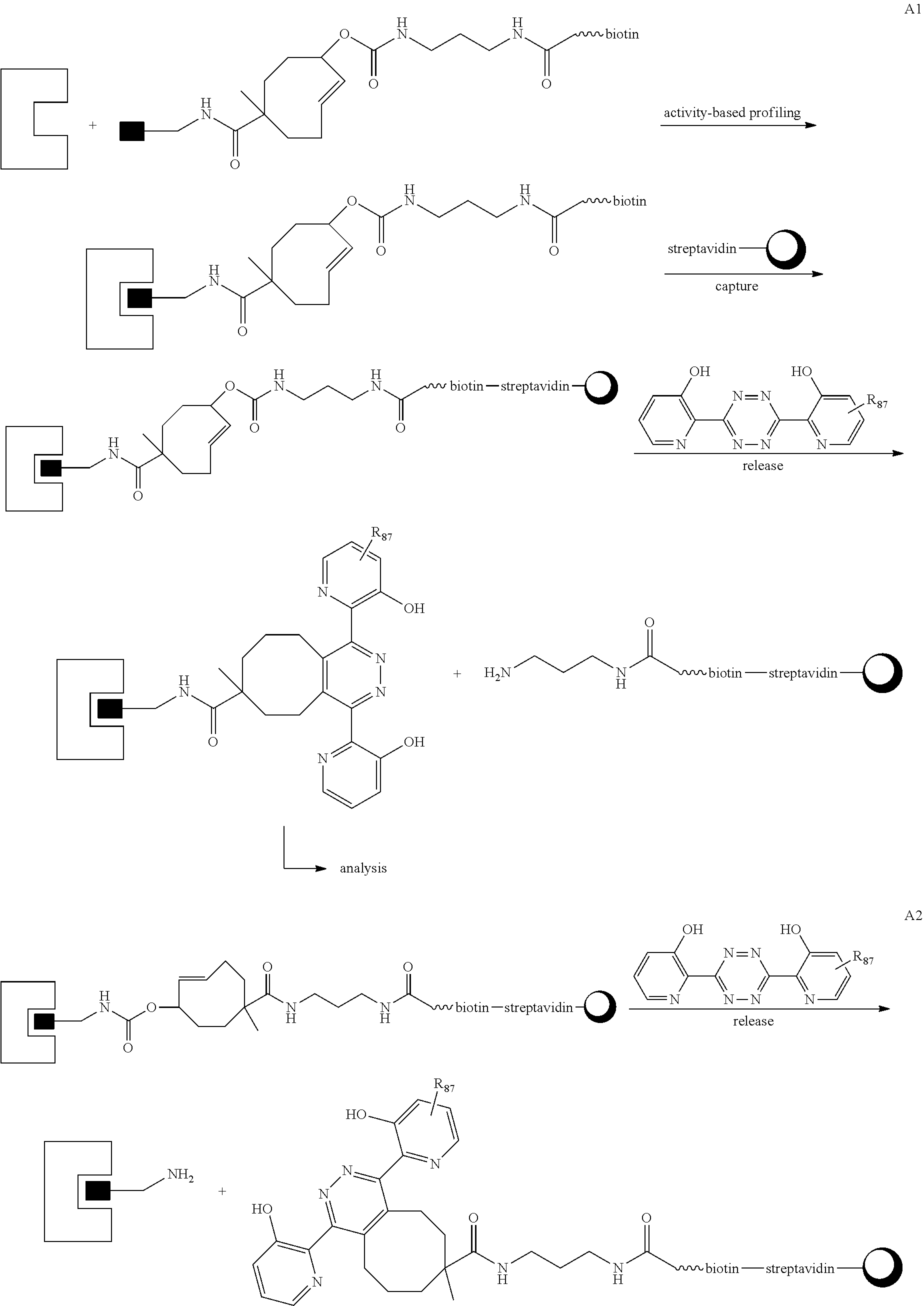

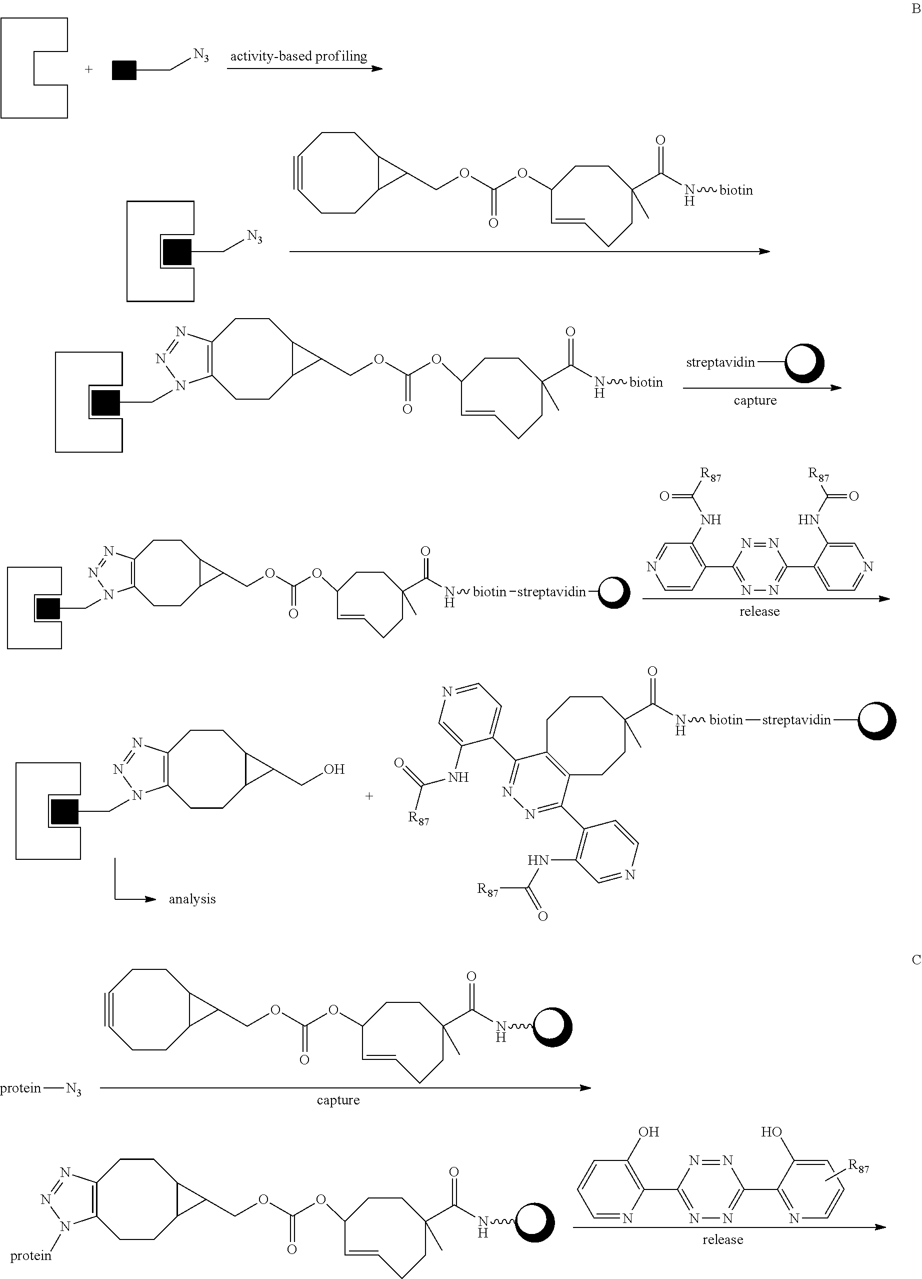

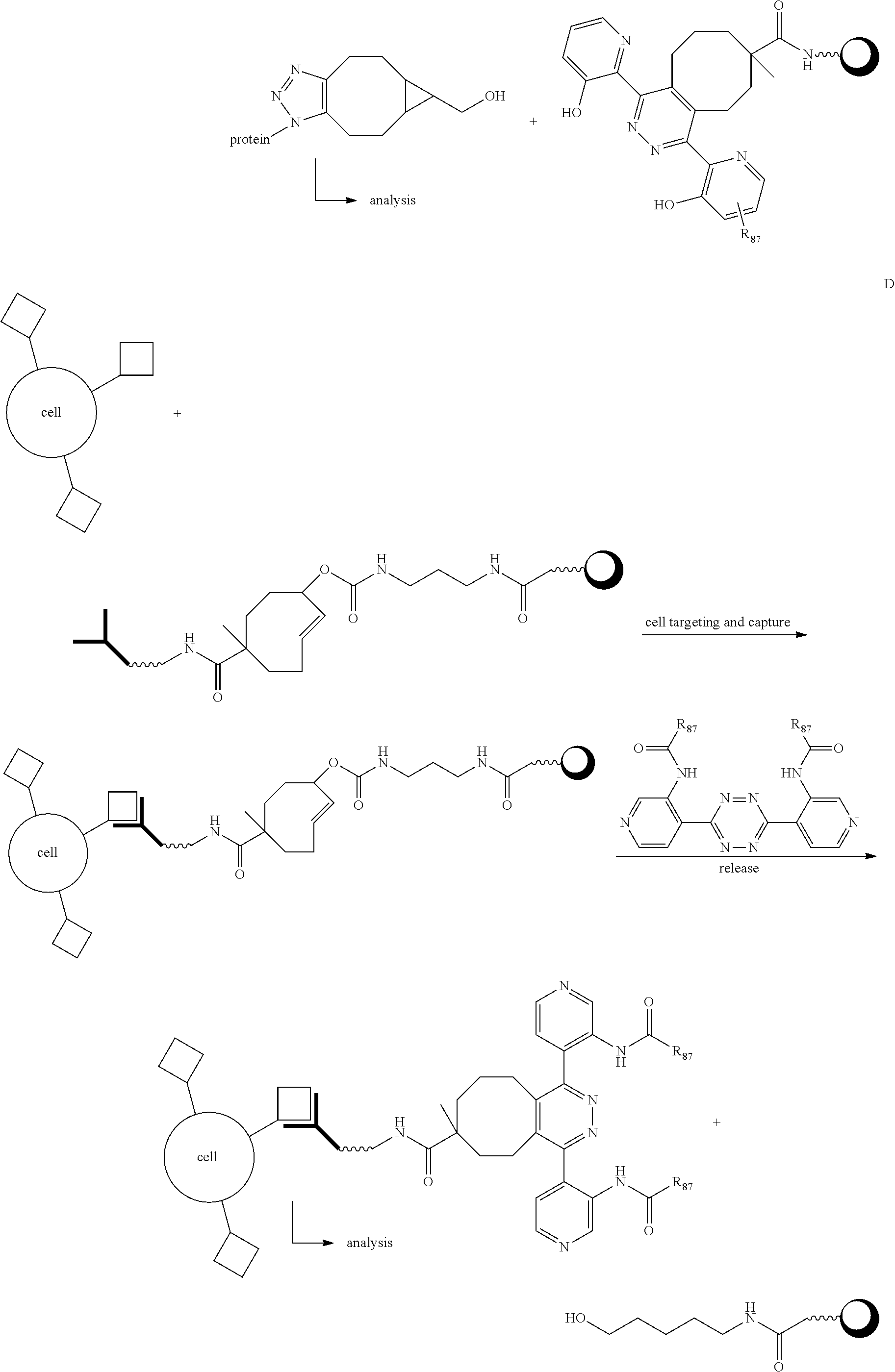

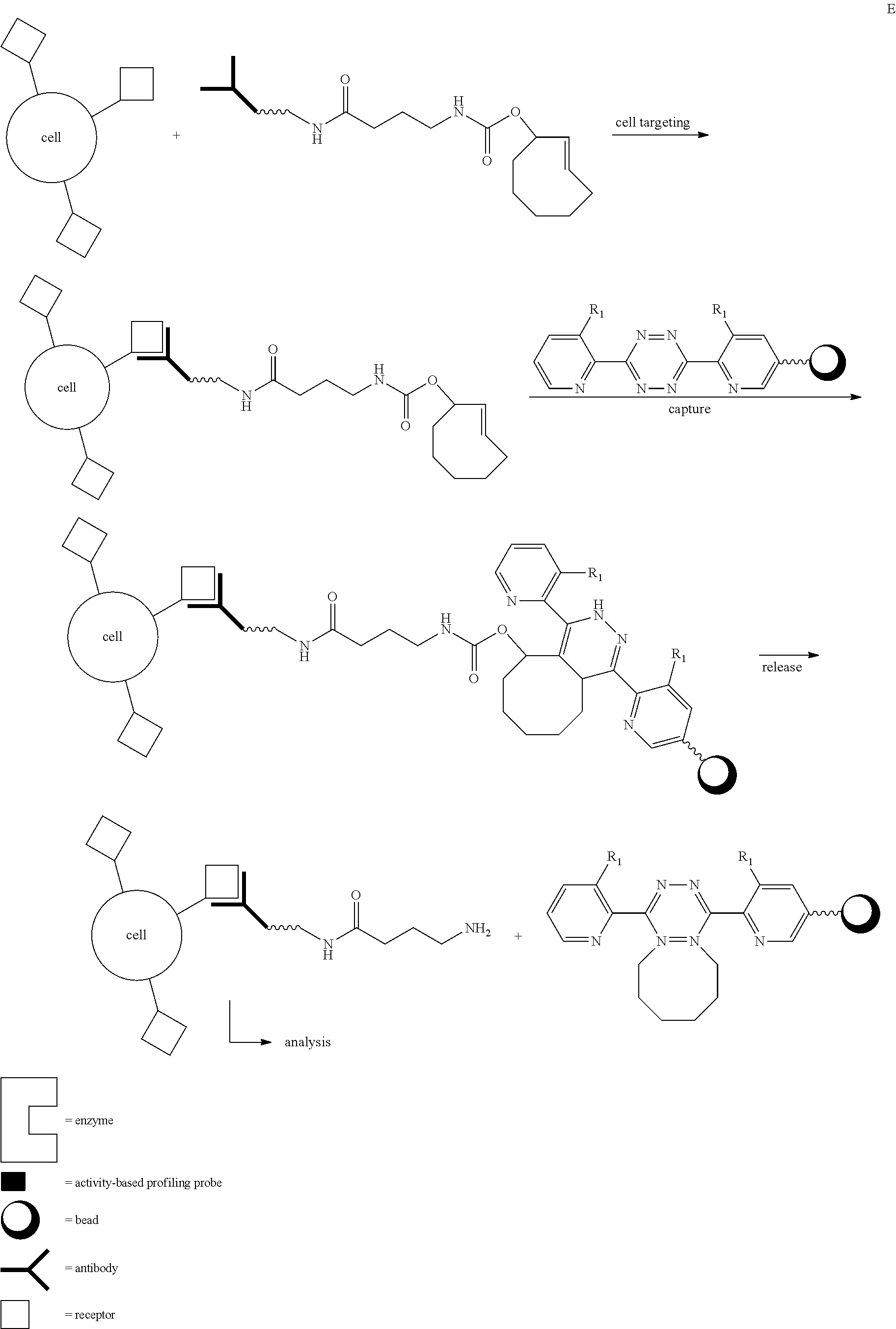

An alternative aspect of the invention comprises the Trigger as a chemoselective cleavable linker between a solid support and a solid support-bound substance. Therein, with reference to formula 5a and 5b: f is preferably 1.

In one embodiment, the Trigger is used as cleavable linker in solid phase synthesis. Solid-phase synthesis methods have been used in organic synthesis over the last decades, first for peptides, then for oligonucleotides, followed by other organic molecules. This development was accompanied by the development of various cleavable linkers for the detachment of molecules from the solid support resin. Examples include linkers that can be cleaved by acids, bases, fluoride ion, or photochemical reactions (Maruta et al. Tetrahedron Letters 47 (2006) 2147-2150; Shabat et al. Chem. Eur. J 2004, 10, 2626). An alternative bioorthogonal approach may expand the scope of compatible functionalities. Here a solid phase synthesis resin, such as polystyrene, is functionalized with TCO Triggers upon which the molecule of interest, for example a peptide, is synthesized. After the synthesis is complete, the Activator is added which reacts with the Trigger releasing the product (e.g. peptide) from the resin-bound Trigger into solution.

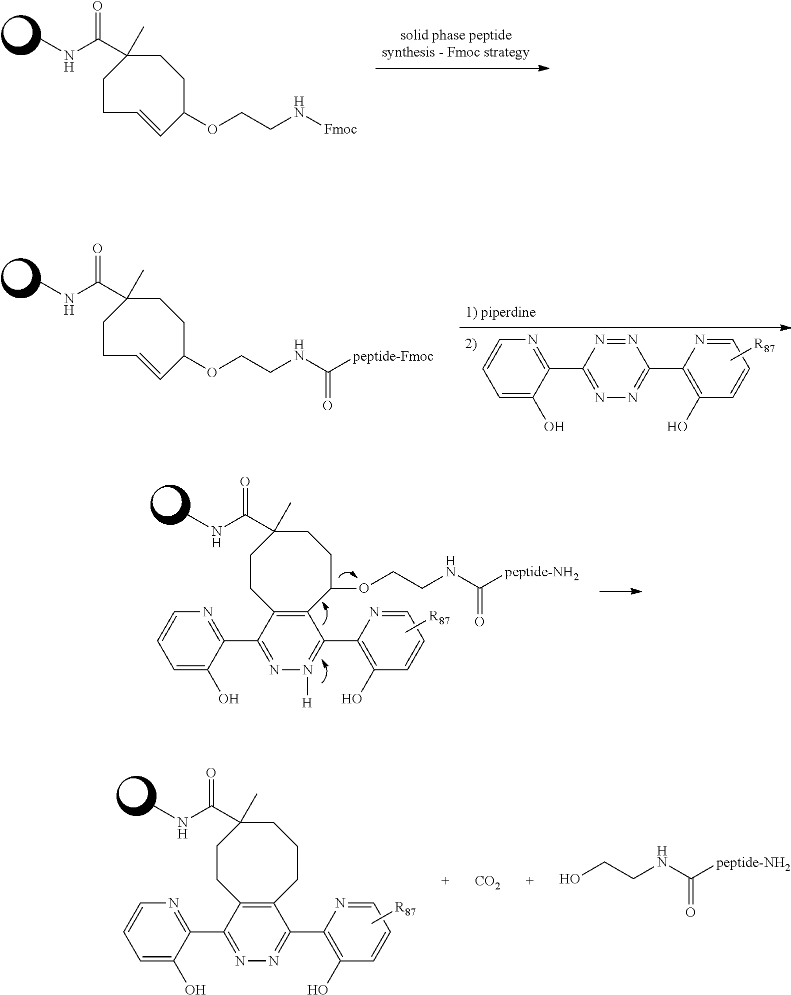

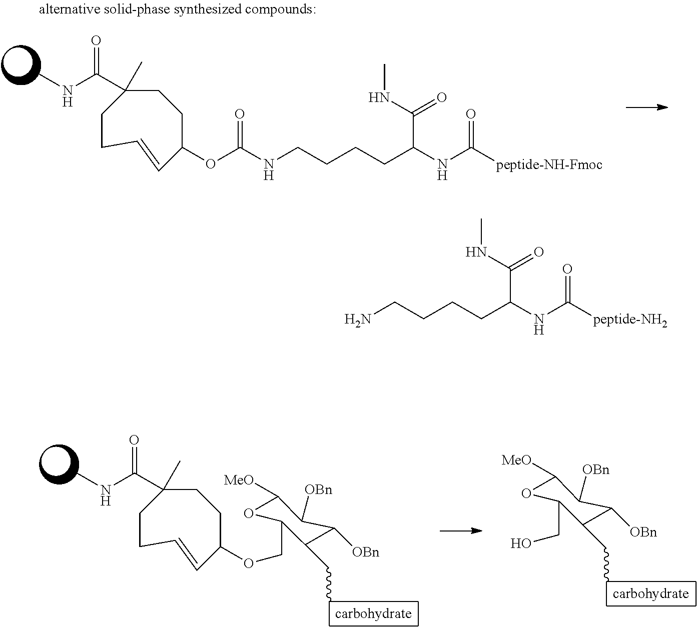

An alternative embodiment, shown directly below, comprises the selective release and activation of a surface-bound chemical reagent in a cartridge or a lab on a chip device.

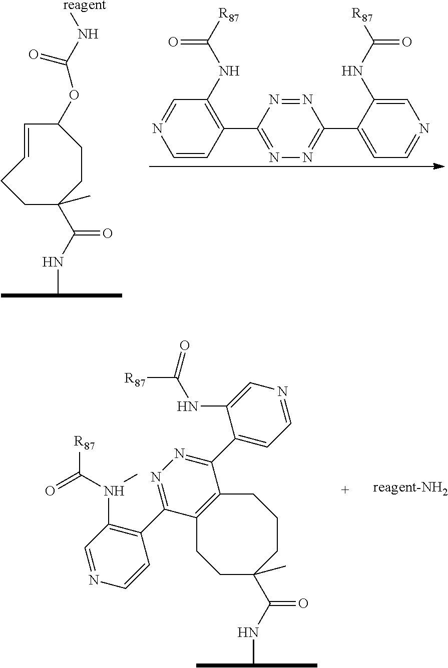

In yet another aspect the Trigger functions as a cleavable linker for reversible biomolecule crosslinking, and/or and immobilization, followed by release. Applications in chemical biology include a) the use of two proteins linked together via TCO, and their studying their action in e.g. a cellular environment before and after TCO cleavage; b) cleavage of a protein-TCO-targeting agent conjugate in cells to release the protein from a particular subcellular domain; c) cleavage of a protein-targeting agent-TCO conjugate in cells to target the protein to a particular subcellular domain (see Lim Acc Chem Res 2011).

In another embodiment, the CA is a masked antigen, e.g. a masked peptide comprising a peptide linked to a mask via the Trigger, which optionally is present in a Major Histocompatibility Complex (MHC), and which can be unmasked in vitro at a desired time.

In another embodiment, the C^{A} is DNA or RNA and C^{A} is linked via the Trigger to a transfection agent (i.e. C^{B}), designed to deliver the DNA or RNA into a cell in vivo or in vitro. Upon transfection, an activator is administered that tracelessly releases the DNA or RNA from the transfection agent.

In one embodiment the Trigger is used as cleavable linker in a biomolecule biotinylation agent for application in biomolecule detection, isolation and purification. Thus, use is made of biomolecule-reactive Trigger-biotin conjugates for the cleavable (reversible) attachment of biotin to peptides, proteins (for example cell surface proteins), glycoproteins, DNA and other biomolecules. The cleavable linker allows mild detachment of the bound biomolecule after affinity purifying biotinylated proteins using immobilized avidin or streptavidin. With reference to the scheme directly below, Probe A is useful for biotinilation of lysine residues in proteins. With R═SO₃Na the agent will remain charged and in the extracellular space and is especially useful for labeling cell membrane proteins. Probe B is an aminooxy-biotin reagent and probe C is a hydrazide-biotin reagent and as such B and C are useful for biotinylating glycoproteins and other molecules that have oxidizable polysaccharides groups. Compounds D-F are a photoactivatable reagent that enables biotinylation of nucleic acids and other molecules that do not have readily available amine or sulfhydryl groups for coupling. When exposed to strong ultraviolet or visible light, the aryl azide group of D-F converts to a reactive nitrene that readily reacts to form covalent bonds with a variety of chemical groups, such as nucleic acids. Compound G enables simple and efficient reversible biotinylation of antibodies, cysteine-containing peptides and other thiol-containing molecules. Compound H is a reagent that enables proteins to be temporarily labeled at sulfhydryl sites for later photo-induced covalent attachment and transfer of biotinylation to an interacting protein, thereby tagging the previously unknown interacting protein(s) for affinity purification, detection, analysis (e.g. mass spectrometry, electrophoresis or sequencing).

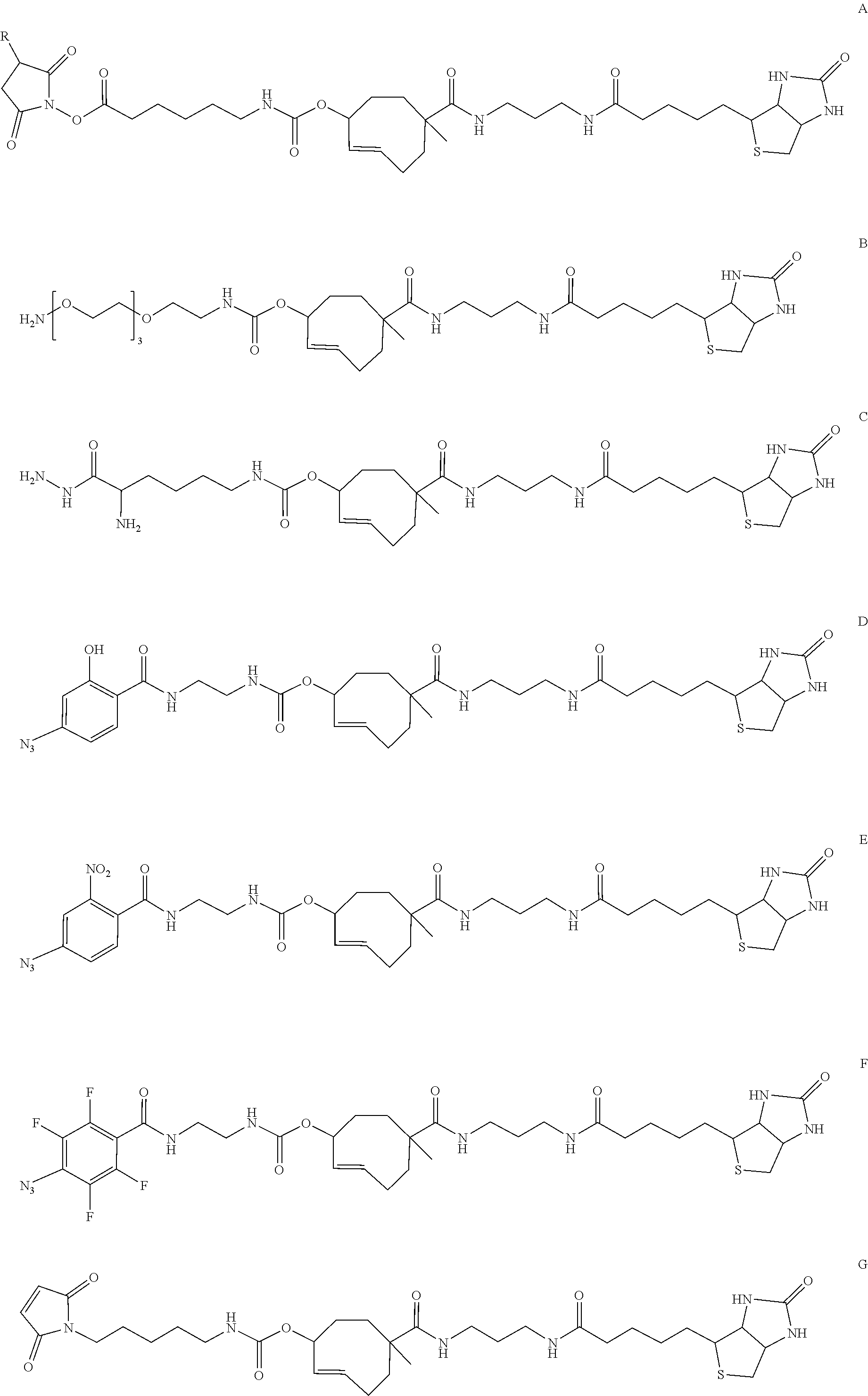

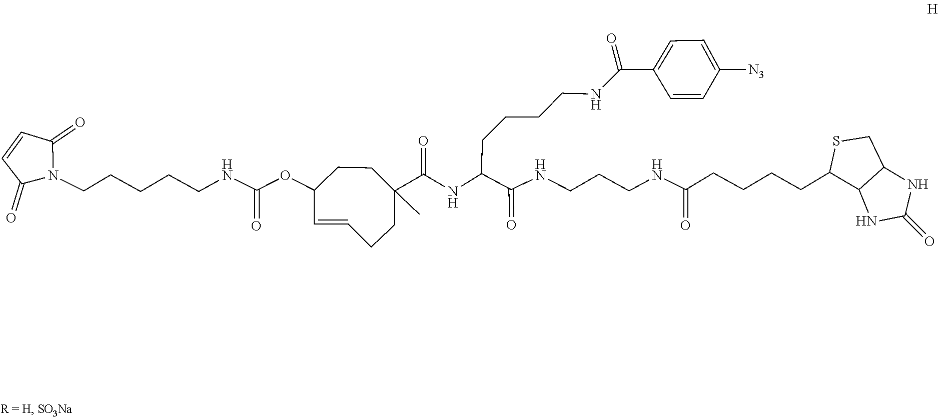 [SMILES: CC1(C(=O)NC(CCCCNC(=O)c2ccc(N=[N+]=[N-])cc2)C(=O)NCCCNC(=O)CCCCC2SCC3NC(=O)NC32)CC/C=C/C(OC(=O)NCCCCCN2C(=O)C=CC2=O)CC1]

Analogously, the linkers directly below can be used for protein labeling and capture using tetrazine coated beads or resin. The tetrazine moieties will react with the TCO functionalized biomolecule allowing isolation, followed by automatic biomolecule release. Suitably chosen tetrazine-TCO pairs will give a release with a half life of >2 h and given the required reaction time of <10 minutes thus allow enough time for isolation of the complex before the complex releases the biomolecule automatically through release of the linker.

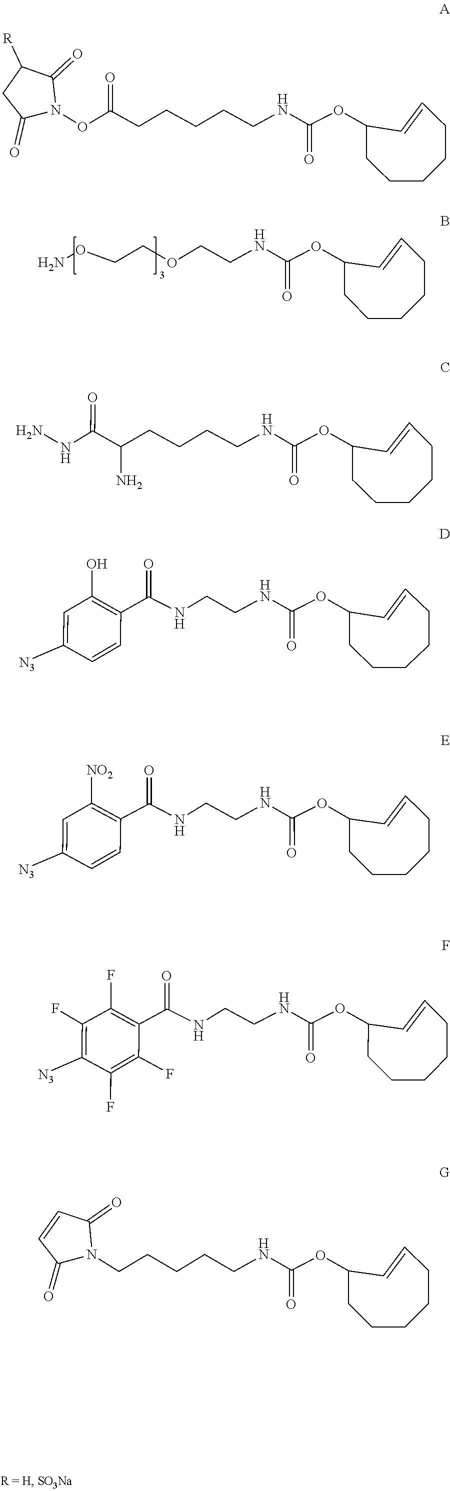

In another embodiment the Trigger is used as cleavable linker in a crosslinker between two biomolecules for e.g. application in biomolecule detection, immobilization, isolation and purification, in particular with respect to biomolecule interactions. Examples include crosslinking of cell surface proteins prior to cell lysis and immunoprecipitation, fixing of protein interactions to allow identification of weak or transient protein interactions, fixing of tissues for immunostaining, 1-step bioconjugations, and immobilization of proteins onto eg amine-coated surfaces. Thus, use is made of biomolecule-reactive bifunctional crosslinkers containing a cleavable Trigger for the cleavable (reversible) attachment of biomolecules such as peptides, glycoproteins, DNA to one another. Such as linker can for example be used in a “shotgun” approach to capture interaction complexes. When using lysine reactive moieties, the reagent will crosslink any and all interacting molecules whose respective lysine residues come within the spacer length of the crosslinker. Subsequently, a particular interaction complex is detected after crosslinking and (usually) cell lysis by immunoprecipitation or by administering a specific antibody or other probe for one of the target molecules in the complex. With reference to the scheme directly below, compound A is an homobifunctional lysine-reactive crosslinker, useful for the crosslinking of for example two proteins. Compound B is a cleavable heterobifunctional amine-reactive photocrosslinker, useful with molecules where no amine residue is available or accessible (even DNA, polysaccharides and other molecules). These heterobifunctional linkers enable “two-step” reactions in which “bait” proteins can be labeled, added to a cell and light-activated to crosslink at the desired time (e.g., upon cell stimulation when the interaction of interested is presumed to occur), followed by isolation and mild cleavage through the Trigger. Compound C is a cleavable homobifunctional thiol reactive crosslinker for covalent but reversible conjugation between e.g. proteins or peptide cysteines. Compound D is a cleavable heterobifunctional thiol and amine reactive crosslinker for covalent but reversible conjugation between e.g. proteins or peptide cysteines and lysines. Compound E is a cleavable modification reagent for aminoacids, allowing a temporary change in protein charge.

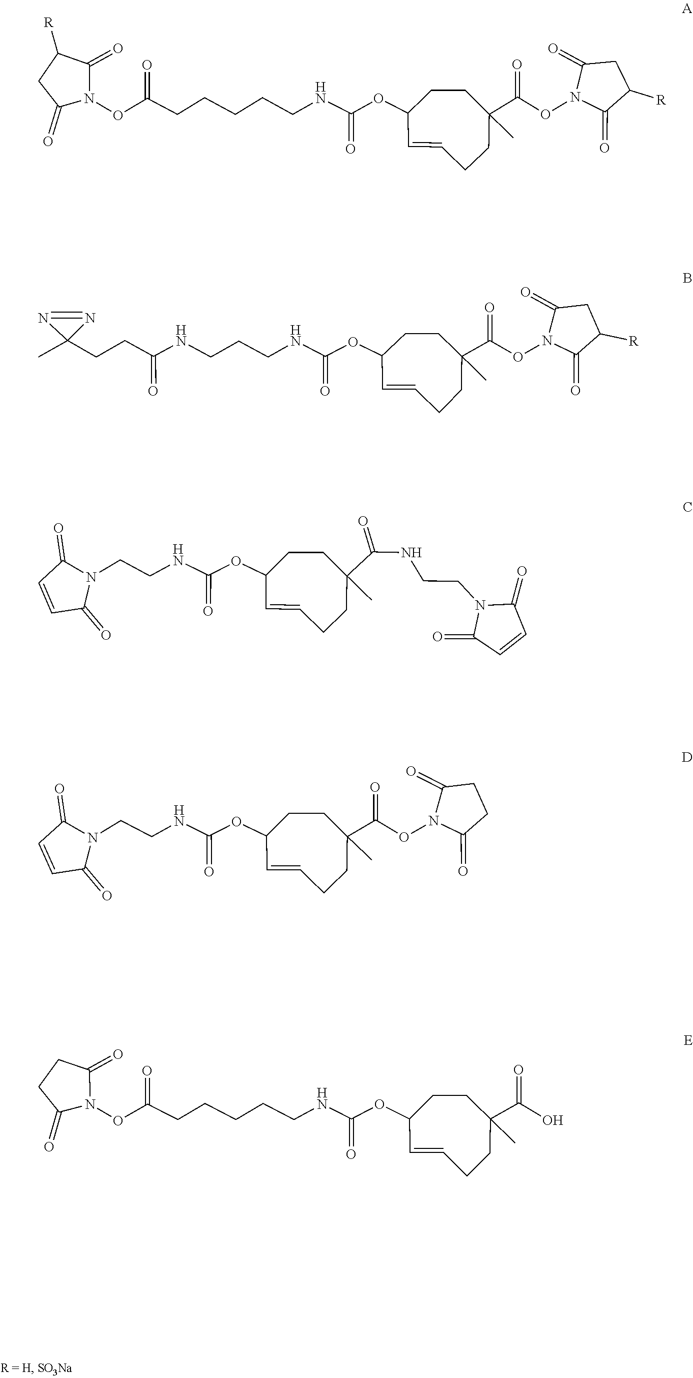

In another aspect the Trigger is used as in a radiolabeling kit, as shown in the scheme directly below. Use is made of a bead linked to a ¹⁸F-labeled TCO, and a tetrazine-peptide derivative is added, which reacts with the TCO affording ¹⁸F-TCO-tetrazine-peptide. With reference to formula 5, f is preferably 1. In a different approach, a peptide is bound to the bead via the TCO and a ¹⁸F-tetrazie derivative is added which reacts with the TCO affording a ¹⁸F-tetrazine-TCO-peptide.

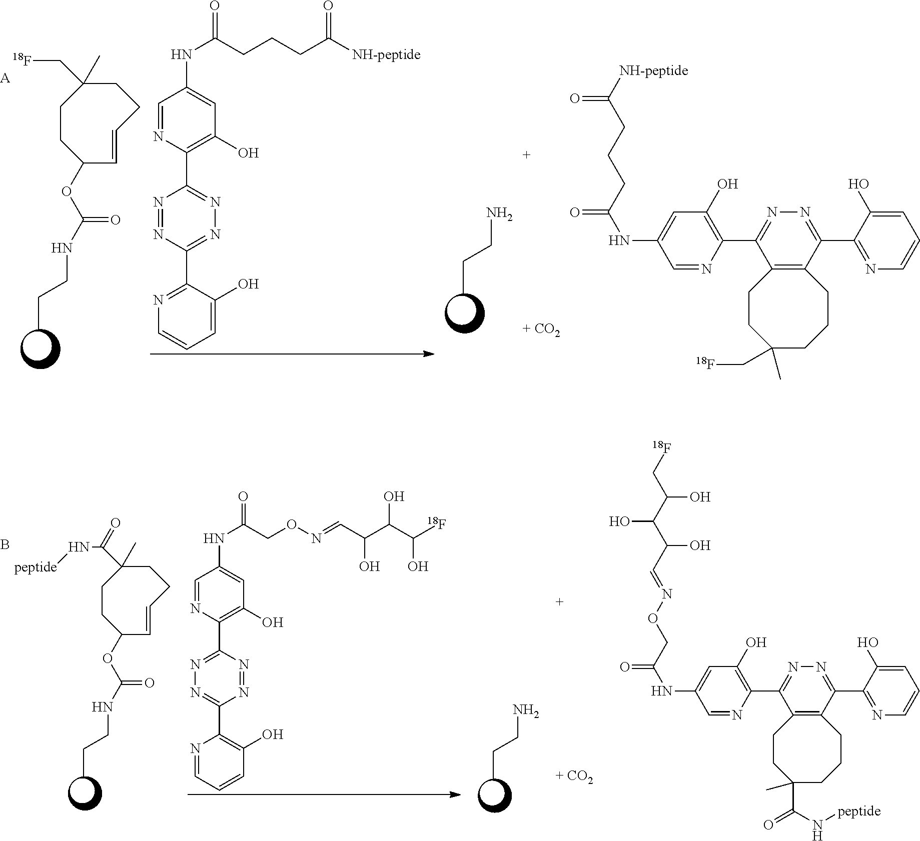

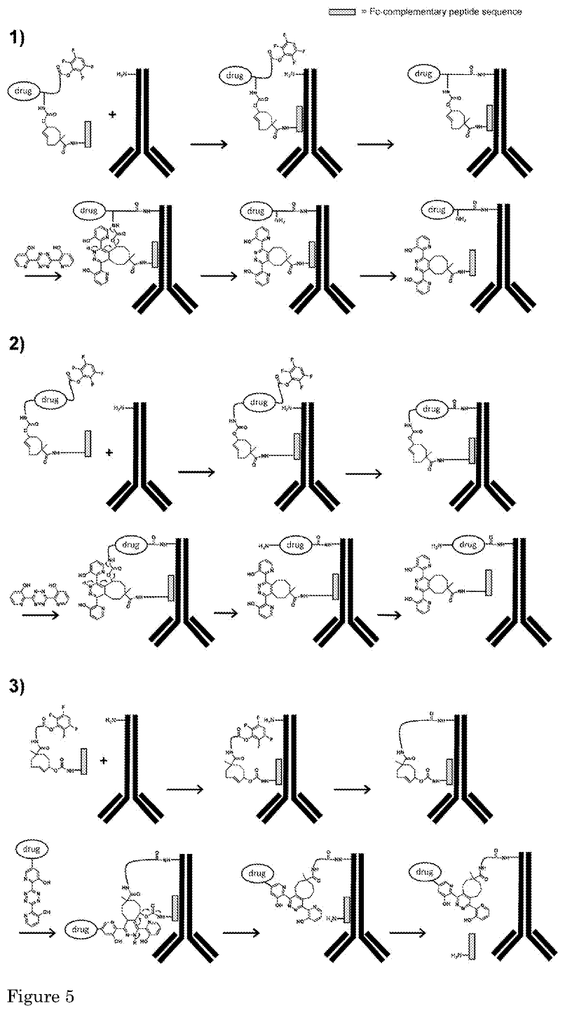

In another embodiment, the compounds of the invention are used for site specific antibody conjugation. Reference is made to FIG. 5. In panel 1, a peptide that binds to a specific region on the antibody near a specific conjugatable amino acid residue (in this case a lysine), is modified with a click-cleavable TCO linker that further links to a Drug (to be conjugated) and an active ester (e.g. TFP ester) for lysine conjugation. Upon binding of the peptide to the protein the TFP ester will react with the lysine residue due to its proximity. Subsequently, the TCO linker is cleaved by tetrazine, the peptide is washed off the antibody, leading to a site specifically conjugated antibody-drug conjugate. Panel 2 shows the same approach wherein the Drug links the TCO to the active ester. In panel 3, the peptide is modified with a click-cleavable TCO linker that further links to the active ester (e.g. TFP ester) for lysine conjugation. Upon binding of the peptide to the protein and lysine conjugation, a tetrazine-drug conjugate is added which leads to concomitant cleavage of the peptide and conjugation of the drug.

In another aspect the Trigger is used in a diagnostic kit, see directly below. With reference to formula 5, f is preferably 1. Immobilized (e.g. in a 96-well plate) TCO conjugated to one or more quenched fluorophores is contacted with a sample containing an unknown amount of tetrazine moieties. These tetrazines have been previously incorporated in a biomolecule as amino acid residue in a metabolic engineering experiment, or alternatively the sample contains e.g. tetrazine-based pesticide. Reaction of the tetrazine with the TCO effects release and dequenching of the fluorophores, allowing readout via e.g. UV absorption.

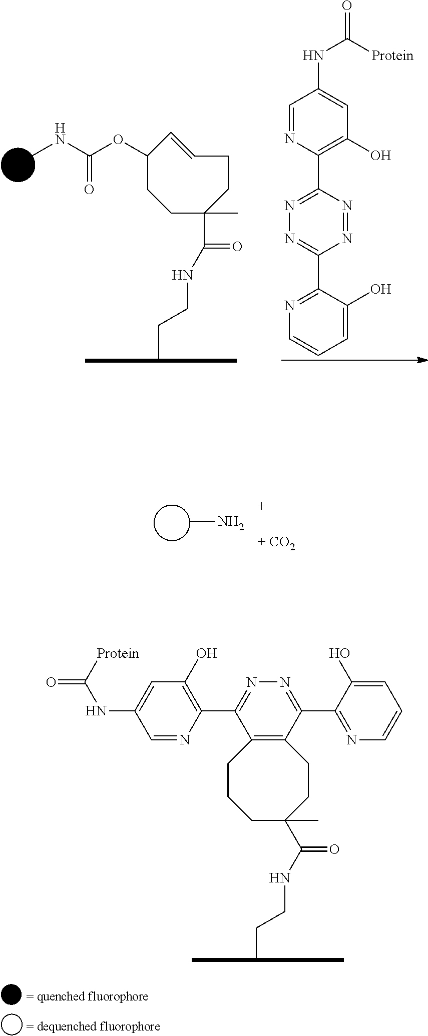

The Constructs A and Constructs B used in in vitro embodiments include but are not limited to small molecules, organic molecules (including fluorescent dyes), metal coordination compounds, molecules comprising a radionuclide, chelates comprising a radiometal, inorganic molecules, organometallic molecules, biomolecules, drugs, polymers, resins (e.g. polystyrene, agarose), particles (e.g. beads, magnetic beads, gold, silica-based particles and materials, polymers and polymer-based materials, glass, iron oxide particles, micro- and nanoparticles (such as liposomes, polymersomes), gels, surfaces (e.g. glass slides, chips, wavers, gold, metal, silica-based, polymer, plastic, resin), cells, biological tissues, pathogens (viruses, bacteria, fungi, yeast). The Constructs may for example comprise a combination of the aforementioned Constructs.

Examples of biomolecules include: carbohydrates, biotin, peptides, peptoids, lipids, proteins, enzymes, oligonucleotides, DNA, RNA, PNA, LNA, aptamers, hormones, toxins, steroids, cytokines, antibodies, antibody fragments (e.g. Fab2, Fab, scFV, diabodies, triabodies, VHH), antibody (fragment) fusions (e.g. bi-specific and trispecific mAb fragments).

Construct A and Construct B can also be R₃₂ or a moiety comprising R₃₂, as defined herein, wherein R₃₂ can be used to bind to a further Construct A or B. For example, Construct A can be R₃₂ being a maleimide or photocrosslinker that is bound to the T^{R} via a Spacer S^{P}. The maleimide or photocrosslinker can be used to further conjugate the T^{R} to a protein. In this embodiment C^{A} and C^{B} are a biomolecule-binding moiety.

In preferred embodiments C^{A} and C^{B} are bound to the T^{R} via a Spacer S^{P}, wherein the S^{P} is or comprises C^{M2}, C^{X} or a residue of R₃₂.

In a preferred embodiment the Construct A is a biomolecule.

In another preferred embodiment the C^{A} is a biomolecule and C^{B} is selected from the group of polymer, resin, particle, solid support

In another preferred embodiment the C^{B} is a biomolecule and C^{A} is selected from the group of polymer, resin, particle, gel, surface

In some embodiments of the invention, C^{A} and/or C^{B} equals an R₃₂ group and this or these groups function as biomolecule binding moieties. Preferred R₃₂ groups for use as biomolecule binding moieties include but are not limited to biotin, carboxylic acids and their activated esters such as N-hydroxysuccinimide ester and para-nitrophenyl ester, isocyanate, isothiocyanate, N-maleimide groups, bromoacetamide and iodoacetamides, azido groups, alkynyl groups such as (hetero)cycloalkynyl group and terminal alkynyl groups, aminooxy groups, hydrazinyl groups, and photoreactive groups.

In some embodiments of the invention, C^{A} and C^{B} equal R₃₂ and these moieties are bound to different locations on the same biomolecule, resulting in a cycle.

In another preferred embodiment the C^{A} is a R₃₂ that is a biomolecule binding moiety, and C^{B} is a R₃₂ that binds to a polymer, resin, particle or a solid support.

In another preferred embodiment the C^{B} is a R₃₂ that is a biomolecule binding moiety and C^{A} is a R₃₂ that binds to a polymer, resin, particle or a solid support.

In another embodiment, either C^{A} or C^{B} is or comprises biotin.

In another embodiment the Trigger cleavage results in cleavage of one C^{A} from another C^{A}, when the dienophile Trigger of formula 19 links to two allylic positioned C^{A} (with reference to Formula 19: X⁵ is —CHR₄₈) or when one L^{C} is bound to two C^{A} moieties, wherein one or both C^{A} can release from the Trigger upon reaction with a diene. Optionally, one or more C^{B} can be additionally present.

In some embodiments Trigger cleavage results in the cleavage of one C^{A} from two or more C^{B}. In some embodiments Trigger cleavage results in the cleavage of one C^{B} from two or more C^{A}. In preferred embodiments, the Trigger is bound to only one C^{A} and one C^{B}. In other preferred embodiments, the Trigger is bound to two C^{A} moieties and no C^{B} moieties. In some embodiments wherein a C^{A} is to be released from the Trigger but not C^{B}, then it is preferred that C^{B} is not bound via L^{C}. In a preferred embodiment, only one surface (being C^{A} or C^{B}) is bound to the Trigger

In a preferred embodiment, the Activator comprises R₈₇.

In a preferred embodiment, the Activator may be conjugated to surfaces, gels, resins, especially solid phase synthesis resins, such as polystyrene, Janda gel and the like.

In a preferred embodiment, the Activator can comprise a biomolecule, a Drug or a Targeting Agent.

In a preferred embodiment, Activators may optionally comprise a membrane translocation moiety (e.g. adamantine, poly-lysine/arginine, TAT, human lactoferrin). Exemplary references regarding such moieties include: Trends in Biochemical Sciences, 2015. 40, 12, 749; J. Am. Chem. Soc. 2015, 137, 12153-12160; Pharmaceutical Research, 2007, 24, 11, 1977.

In a preferred embodiment, the Activator does not comprise R₈₇.

Suitable Spacers S^{P}, for use in a Trigger conjugate of this invention are listed in the section Spacers (vide supra). In some embodiments the Spacer has at most 50 carbon atoms, more preferably at most 25 carbon atoms, more preferably at most 10 carbon atoms. Other preferred Spacers are PEG and PPG polymers, and oligopeptoids, preferably ranging from 2 to 50 repeating units, more preferably from 2 to 24 repeating units, more preferably from 2 to 12 repeating units.

It will be understood that all the embodiments described herein, regardless of whether they are described in the same Section, can be combined. All references mentioned herein are incorporated by reference in their entirety. The invention is illustrated below with non-limiting examples.

### Section 23—Examples

### Example 1—Materials and Methods

All reagents, chemicals, materials and solvents were obtained from commercial sources and were used as received, including nitrile starting compounds that not have been described. All solvents were of AR quality. Monomethyl auristatin E (MMAE) and deuterated (d₈) MMAE were purchased from MedChemExpress. Sodium [¹²⁵I]iodide solutions were purchased from PerkinElmer. [¹¹¹In]Indium chloride solution was purchased from Curium. The Bolton-Hunter reagent (N-succinimidyl-3-[4-hydroxyphenyl]propionate, SHPP) and Zeba desalting spin columns (7 kDa MW cut-off, 0.5 mL) were purchased from Pierce Protein Research (Thermo Fisher Scientific). Mouse plasma was purchased from Innovative Research. 29-Amino-3,6,9,12,15,18,21,24,27-nonaoxanonacosan-l-ol was purchased from PurePEG. 3,6-Dimethyl-1,2,4,5-tetrazine and (E)-cyclooct-2-en-1-yl (4-nitrophenyl) carbonate were prepared according to literature procedures [Versteegen et al., Angew. Chem. Int. Ed. 2013, 52, 14112-14116]. The anti-TAG72 diabody (AVP04-58) was produced, functionalized with PEG-TCO-MMAE groups and characterized as reported [Rossin et al. Nature Communications 2018, 9, 1484], yielding an ADC AVP0458-TCO-MMAE with DAR=4. Analytical thin layer chromatography was performed on Kieselgel F-254 precoated silica plates. Column chromatography was carried out on Screening Devices B. V. silica gel (flash: 40-63 pm mesh and normal: 60-200 pm mesh). ¹H-NMR and ¹³C-NMR spectra were recorded on a Bruker Avance III HD (400 MHz for ¹H-NMR and 100 MHz for ¹³C-NMR) spectrometer at 298 K. Chemical shifts are reported in ppm downfield from TMS at rt. Abbreviations used for splitting patterns are s=singlet, d=doublet, dd=double doublet, t=triplet, q=quartet, m=multiplet and br=broad. HPLC-PDA/MS was performed using a Shimadzu LC-10 AD VP series HPLC coupled to a diode array detector (Finnigan Surveyor PDA Plus detector, Thermo Electron Corporation) and an Ion-Trap (LCQ Fleet, Thermo Scientific). HPLC-analyses were performed using a Alltech Alltima HP C₁₈ 3μ column using an injection volume of 1-4 pL, a flow rate of 0.2 mL min⁻¹ and typically a gradient (5% to 100% in 10 min, held at 100% for a further 3 min) of MeCN in H₂O (both containing 0.1% formic acid) at 298 K. Quantitative HPLC-SIM-MS was performed on a Shimadzu Nexera-i LC-2040C 3D HPLC equipped with a Shimadzu LCMS-2020 mass spectrometer.

Size exclusion chromatography (SEC) was performed on an Akta system (GE Healthcare Life Science) equipped with a BioSep SEC-s3000 column (Phenomenex). Radio-HPLC was performed on an Agilent 1100 system, equipped with a Gabi radioactive detector (Raytest). The samples were loaded on an Alltima C18 column (4.6×150 mm, 5p), which was eluted at 1 mL min-1 with a linear gradient of water (A) and acetonitrile (B) containing 0.1% v/v % TFA (4 min at 20% B followed by an increase to 70% B in 11 min). Radio-ITLC was performed on ITLC-SG strips (Varian Inc.) eluted with 200 mM EDTA in saline solution (¹¹¹In) or a 1:1 mixture of methanol/ethyl acetate (¹²⁵I-labeling). In these conditions the radiolabeled products remain at the base while unbound ¹¹¹In and [¹²⁵I]I-SHPP migrates with an R_{f} of 0.7-0.9. SDS-PAGE was performed on a Mini-PROTEAN Tetra Cell system using 4-20% precast Mini-PROTEAN TGX gels and Precision Plus Protein All Blue Prestained protein standards (BioRad Laboratories). The radioactivity distribution on ITLC strips and SDS-PAGE gels was monitored with a Typhoon FLA 7000 phosphor imager (GE Healthcare Life Science) using the AIDA software.

### Example 2—Synthesis of Tetrazines

Control tetrazine 2.19 was purchased from commercial sources. Control tetrazines 2.17, 2.18 and 2.20 were prepared according to literature procedures [Rossin et al., Angew Chem Int Ed 2010, 49, 3375-3378; Versteegen et al., Angew Chem Int Ed 2013, 52/53, 14112-14116].

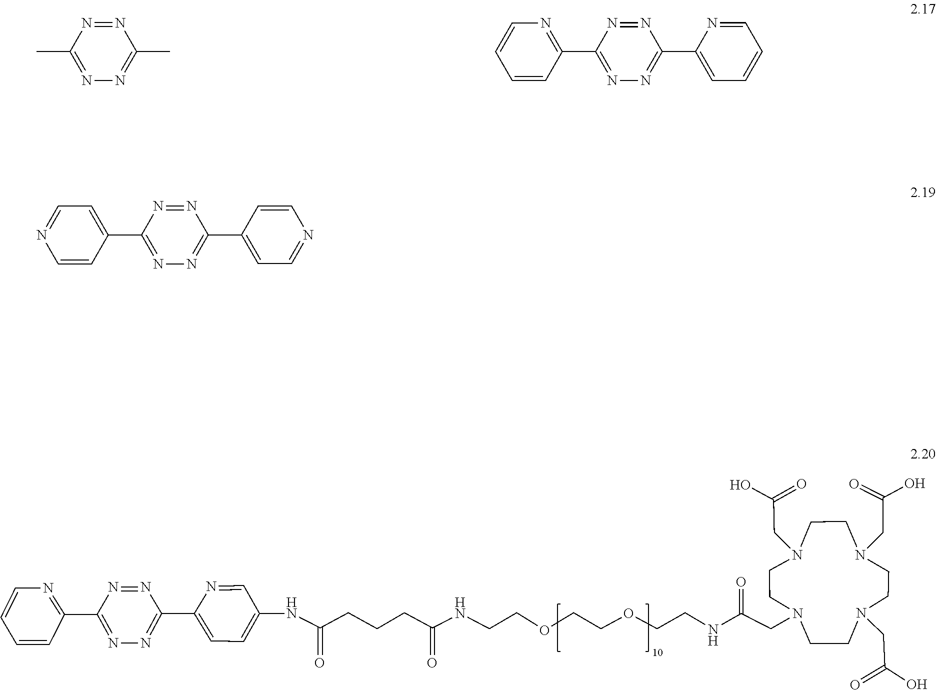

### 2,2′-(1,2,4,5-Tetrazine-3,6-diyl)bis(pyridin-3-amine) (2.1)

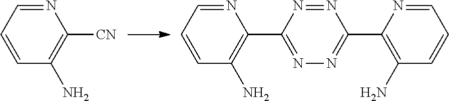 [SMILES: N#Cc1ncccc1N ; Nc1cccnc1-c1nnc(-c2ncccc2N)nn1]

3-Aminopicolinonitrile (125 mg, 1.0 mmol) and hydrazine hydrate (300 μL, 5.0 mmol, 5 eq) were stirred at 100° C. for 20 h. Cold water (2 mL) was added and the suspension was stirred at rt for 5 min. Filtration, washing of the solid with cold water and cold ethanol (both 5×2 mL) and drying in vacuo yielded the intermediate [2H]-TZ (38 mg, 0.14 mmol, 27%) as an orange solid. To the [2H]-TZ and PhI(OAc)₂ (75 mg, 0.23 mmol, 1.6 eq) dichloromethane (1 mL) was added and the suspension was stirred at rt for 3 h. In time a color change occurred from orange to red. The suspension was filtrated, the solid was washed with dichloromethane (5×1 mL) and dried in vacuo yielding pure 2.1 (31 mg, 0.12 mmol, 22% overall) as a red solid. ¹H-NMR (DMSO-d6): δ=8.13 (dd, 2H, ArH), 7.36 (2dd, 4H, ArH), 6.98 (br s, 4H, NH₂) ppm. ¹³C-NMR (DMSO-d6): δ=162.8, 146.6, 138.3, 129.5, 126.9, 124.5 ppm. ESI-MS: m/z Calc. for C₁₂H₁₀N₈ 266.10; Obs. [M+H]⁺267.08, [2M+H]⁺532.92, [2M+Na]⁺555.00.

### N,N′-(2,2′-(1,2,4,5-Tetrazine-3,6-diyl)bis(pyridine-3,2-diyl))diacetamide (2.2)

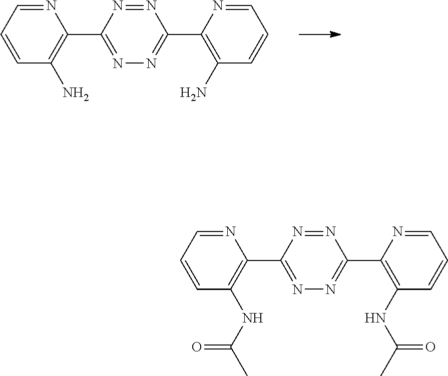 [SMILES: Nc1cccnc1-c1nnc(-c2ncccc2N)nn1 ; CC(=O)Nc1cccnc1-c1nnc(-c2ncccc2NC(C)=O)nn1]

2.1 (12 mg, 45 μmol) was suspended in acetic anhydride (0.5 mL) and the suspension was heated at 50° C. for 3 d. The mixture was precipitated in diethyl ether (6 mL) and the solution was decanted. The solid was washed with diethyl ether (2 mL) and the solution was decanted, after which the wash step was repeated. Next, the solid was triturated with water (2 mL), the mixture was centrifuged at 12.7 krpm for 1 min and the solution was decanted. The solid was subsequently dissolved in methanol (1 mL), after which non-dissolved impurities were removed by filtration. The filtrate was evaporated to dryness and the obtained residue was triturated with water (2 mL). After centrifugation at 12.7 krpm for 1 min and decantation, the solid was dried in vacuo yielding 2.2 (0.75 mg, 2.1 μmol, 5%) as a purple-red solid. ESI-MS: m/z Calc. for C₁₆H₁₄N₈O₂ 350.12; Obs. [M+H]⁺351.17, [2M+Na]⁺722.92.

### 4,4′-(1,2,4,5-Tetrazine-3,6-diyl)bis(pyridin-3-amine) (2.3)

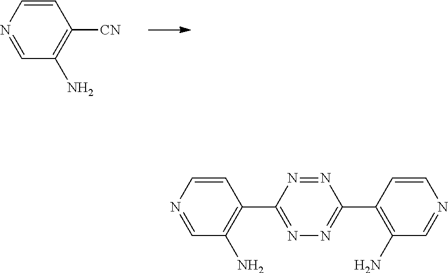 [SMILES: N#Cc1ccncc1N ; Nc1cnccc1-c1nnc(-c2ccncc2N)nn1]

3-Aminoisonicotinonitrile (200 mg, 1.6 mmol), S (26 mg, 0.8 mmol, 0.5 eq) and hydrazine hydrate (460 μL, 8.1 mmol, 5 eq) were stirred at 100° C. for 16 h. Cold water (2 mL) was added and the suspension was stirred at rt for 5 min. The suspension was filtrated and the solid was washed with cold water and cold ethanol (both 5×4 mL). Trituration in ethanol (20 mL) at 50° C. followed by filtration, washing of the solid with ethanol (5×4 mL) and drying in vacuo yielded the intermediate [2H]-TZ (144 mg, 0.54 mmol, 67%) as an orange solid. The [2H]-TZ was stirred in DMSO (5 mL) at 40° C. while bubbling through 02. In time a color change occurred from orange to red. After stirring for 16 h the reaction mixture was added dropwise to water (70 mL) and the resulting suspension was filtrated. The solid was washed with water, ethanol and dichloromethane (all 5×4 mL) and dried in vacuo yielding pure 2.3 (130 mg, 0.49 mmol, 61% overall) as a red solid. ¹H-NMR (DMSO-d6): δ=8.40 (br s, 2H, ArH), 8.20 (d, 2H, ArH), 7.95 (br d, 2H, ArH), 7.16 (br s, 4H, NH₂) ppm. ¹³C-NMR (DMSO-d6): δ=162.7, 144.3 (br), 141.3, 135.9, 120.8 (br), 115.9 ppm. ESI-MS: m/z Calc. for C₁₂H₁₀N₈ 266.10; Obs. [M+H]⁺267.17.

### N,N′-(4,4′-(1,2,4,5-Tetrazine-3,6-diyl)bis(pyridine-4,3-diyl))diacetamide (2.4)

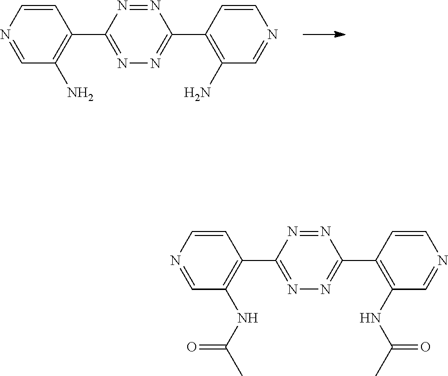 [SMILES: Nc1cnccc1-c1nnc(-c2ccncc2N)nn1 ; CC(=O)Nc1cnccc1-c1nnc(-c2ccncc2NC(C)=O)nn1]

2.3 (22 mg, 83 μmol) was suspended in acetic anhydride (0.5 mL) and the suspension was heated at 50° C. for 3 d. The mixture was precipitated in diethyl ether (6 mL) and the solution was decanted. Next, the solid was triturated with water (2 mL), the mixture was centrifuged at 12.7 krpm for 1 min and the solution was decanted. This trituration-centrifugation procedure was repeated using methanol and 2-propanol (both 2 mL). The resulting solid was dried in vacuo yielding pure 2.4 (12 mg, 34 μmol, 41%) as a purple-red solid. ¹H-NMR (DMSO-d6): 45=10.35 (s, 2H, NH), 9.01 (s, 2H, ArH), 8.67 (d, 2H, ArH), 8.02 (d, 2H, ArH), 2.00 (s, 6H, CH₃) ppm. ¹³C-NMR (DMSO-d6): 45=168.7, 164.1, 146.4, 146.1, 132.4, 131.4, 123.7, 23.3 ppm. ESI-MS: m/z Calc. for C₁₆H₁₄N₈O₂ 350.12; Obs. [M+H]⁺351.17.

### 2,2′-(1,2,4,5-Tetrazine-3,6-diyl)diisonicotinic acid (2.5)

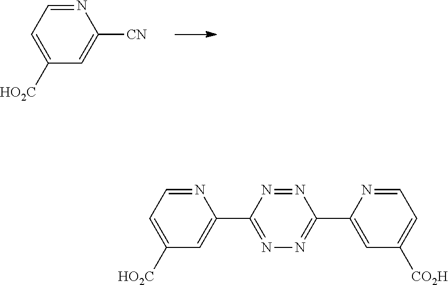 [SMILES: N#Cc1cc(C(=O)O)ccn1 ; O=C(O)c1ccnc(-c2nnc(-c3cc(C(=O)O)ccn3)nn2)c1]

2-Cyanoisonicotinic acid (450 mg, 2.9 mmol) and hydrazine hydrate (810 μL, 14.3 mmol, 5 eq) were stirred at 100° C. for 1 h. The resulting suspension was filtrated and the solid was washed with ethanol and acetone (both 5×4 mL). Trituration in acetone (25 mL) at 50° C. followed by filtration, washing of the solid with acetone (5×2 mL) and drying in vacuo yielded the intermediate [2H]-TZ (224 mg, 0.69 mmol, 48%) as an orange solid. The [2H]-TZ was suspended in acetic acid (12 mL) and concentrated HNO₃ (3 drops) was added, causing an immediate color change from orange to purple. The suspension was stirred at rt for 15 min and filtrated. The solid was washed with acetic acid, acetone and diethyl ether (all 5×4 mL) and dried in vacuo yielding pure 2.5 (224 mg, 0.69 mmol, 48% overall) as a purple solid. ¹H-NMR (DMSO-d6): δ=14.08 (br s, 2H, COOK), 9.16 (d, 2H, ArH), 8.99 (s, 2H, ArH), 8.16 (d, 2H, ArH) ppm.

### 4,4′-(1,2,4,5-Tetrazine-3,6-diyl)dipicolinic acid (2.6)

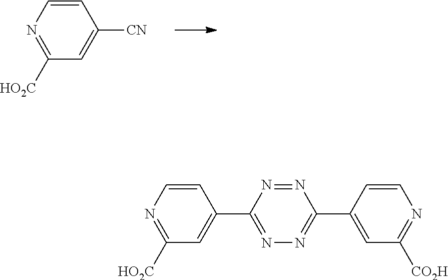 [SMILES: N#Cc1ccnc(C(=O)O)c1 ; O=C(O)c1cc(-c2nnc(-c3ccnc(C(=O)O)c3)nn2)ccn1]

4-Cyanopicolinic acid (100 mg, 0.68 mmol), Zn(OTf)₂ (15 mg, 42 μmol, 0.06 eq), ethanol (300 pL) and hydrazine hydrate (300 μL, 5.3 mmol, 8 eq) were stirred at 60° C. for 24 h. Ethanol (2 mL) was added and the suspension was stirred at rt for 5 min. The suspension was filtrated and the solid was washed with ethanol (5×2 mL). Trituration in methanol (8 mL) at 50° C. followed by filtration, washing of the solid with methanol (5×2 mL) and drying in vacuo yielded the intermediate [2H]-TZ (57 mg, 0.17 mmol, 52%) as an orange solid. The [2H]-TZ was stirred in 1 M HCl (4 mL) and NaNO₂ (52 mg, 0.74 mmol, 4 eq) in water (200 μL) was added dropwise causing an immediate color change from orange to pink. After stirring at rt for 20 min the suspension was filtrated, the solid was washed with 1 M HCl, ethanol and acetone (all 5×2 mL) and dried in vacuo. This yielded pure 2.6 (30 mg, 93 μmol, 27% overall) as a pink solid. ¹H-NMR (DMSO-d6): δ=13.65 (br s, 2H, COOH), 9.10 (d, 2H, ArH), 9.05 (s, 2H, ArH), 8.70 (d, 2H, ArH) ppm. ESI-MS: m/z Calc. for C₁₄H₈N₆O₄ 324.06; Obs. [M+H]⁺325.17.

### 2,2′-(1,2,4,5-Tetrazine-3,6-diyl)bis(pyridin-3-ol) (2.7)

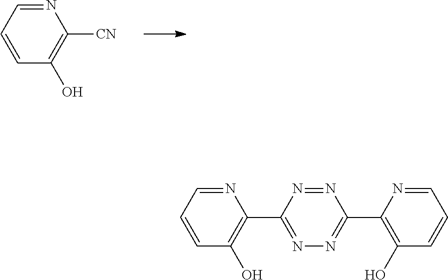 [SMILES: N#Cc1ncccc1O ; Oc1cccnc1-c1nnc(-c2ncccc2O)nn1]

3-Hydroxypicolinonitrile (100 mg, 0.82 mmol) and hydrazine hydrate (280 μL, 4.9 mmol, 6 eq) were stirred at 90° C. for 2 h. Ethanol (4 mL) was added and the suspension was stirred at rt for 5 min. The suspension was filtrated and the solid was washed with ethanol (5×2 mL). Drying of the solid in vacuo yielded pure intermediate [2H]-TZ (59 mg, 0.22 mmol, 54%) as a yellow solid. The [2H]-TZ was suspended in acetic acid (6 mL) and NaNO₂ (75 mg, 1.1 mmol, 5 eq) in water (500 μL) was added dropwise. The suspension was stirred at rt for 1 h during which a clear red solution was obtained and, eventually, a red precipitate arose. Chloroform and water (both 40 mL) were added and the layers were separated. The aqueous layer was extracted with chloroform (2×20 mL) and the combined organic layers were dried using Na₂SO₄. After filtration, the filtrate was evaporated to dryness yielding pure 2.7 (55 mg, 0.21 mmol, 50% overall) as a red solid. ¹H-NMR (DMSO-d6): δ=10.74 (br s, 2H, OH), 8.38 (m, 2H, ArH), 7.57 (m, 4H, ArH) ppm. ¹³C-NMR (DMSO-d6): δ=164.3, 154.3, 141.4, 137.5, 127.5, 125.3 ppm. ESI-MS: m/z Calc. for C₁₂H₈N₆O₂ 268.07; Obs. [M+H]+269.17, [M+Na]⁺291.25.

### 4,4′-(1,2,4,5-Tetrazine-3,6-diyl)bis(pyridin-3-ol) (2.8)

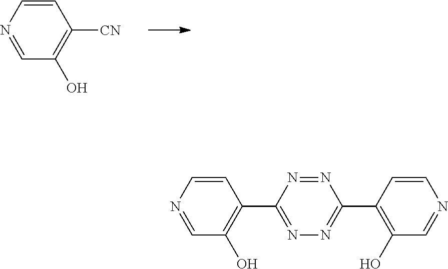 [SMILES: N#Cc1ccncc1O ; Oc1cnccc1-c1nnc(-c2ccncc2O)nn1]

3-Hydroxyisonicotinonitrile (62 mg, 0.52 mmol), Zn(OTf)₂ (10 mg, 28 μmol, 0.05 eq), ethanol (80 μL) and hydrazine hydrate (300 μL, 5.3 mmol, 10 eq) were stirred at 60° C. for 21 h. After removal of the volatiles in vacuo the solid was stirred in methanol (4 mL) for 5 min at rt. The suspension was filtrated, the solid was washed with methanol (5×2 mL) and dried in vacuo yielding impure intermediate [2H]-TZ as an orange solid. The [2H]-TZ was stirred in acetic acid (4 mL) and NaNO₂ (32 mg, 0.45 mmol) in water (200 μL) was added dropwise causing an immediate color change from orange to red. After stirring at rt for 1 h the suspension was filtrated, the solid was washed with ethanol, water and ethanol (all 5×2 mL) and dried in vacuo. This yielded 2.8 (10 mg, 37 μmol, 14% overall) as a red solid. ¹H-NMR (DMSO-d6): δ=10.89 (br, 2H, OH), 8.56 (s, 2H, ArH), 8.37 (d, 2H, ArH), 8.04 (d, 2H, ArH) ppm. ESI-MS: m/z Calc. for C₁₂H₈N₆O₂ 268.07; Obs. [M+H]+269.17.

### Di-t-butyl ((2,2′-(1,2,4,5-tetrazine-3,6-diyl)bis(pyridine-4,2-diyl))bis(methylene))dicarbamate (2.9)

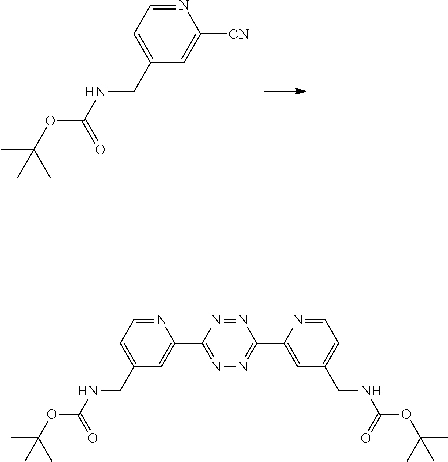 [SMILES: CC(C)(C)OC(=O)NCc1ccnc(C#N)c1 ; CC(C)(C)OC(=O)NCc1ccnc(-c2nnc(-c3cc(CNC(=O)OC(C)(C)C)ccn3)nn2)c1]

t-Butyl ((2-cyanopyridin-4-yl)methyl)carbamate (355 mg, 1.5 mmol), 3-mercaptopropionic acid (135 μL, 1.5 mmol, 1 eq), ethanol (570 μL) and hydrazine hydrate (570 μL, 10 mmol, 6.6 eq) were stirred at 60° C. for 43 h. The resulting orange paste was dissolved in chloroform (100 mL) and the organic layer was washed with water (40 mL). The aqueous phase was extracted with chloroform (20 mL) and the combined organic layers were dried with Na₂SO₄. Filtration and removal of the solvent in vacuo yielded slightly impure intermediate [2H]-TZ as an orange solid. The [2H]-TZ was dissolved in chloroform (5 mL) and PhI(OAc)₂ (372 mg, 1.1 mmol, ˜1.5 eq) was added as a solid. The solution was stirred at rt for 2 h during which a purple precipitate arose. The suspension was filtrated and the solid was dried in vacuo yielding pure 2.9 (270 mg, 0.55 mmol, 72% overall) as a purple solid. ¹H-NMR (CDCl₃/MeOD 9:1): δ=8.89 (d, 2H, ArH), 8.67 (s, 2H, ArH), 7.53 (d, 2H, ArH), 5.67 (br t, 2H, NH), 4.51 (d, 4H, CH₂), 1.50 (s, 18, CH₃) ppm. ¹³C-NMR (CDCl₃/MeOD 9:1): δ=163.8, 156.4, 151.0, 150.9, 150.0, 125.1, 122.7, 80.4, 43.4, 28.4 ppm. ESI-MS: m/z Calc. for C₂₄H₃₀N₈O₄ 494.24; Obs. [M+H]⁺495.25, [2M+H]⁺989.08.

### (2,2′-(1,2,4,5-Tetrazine-3,6-diyl)bis(pyridine-4,2-diyl))dimethanamine, TFA Salt (2.10)

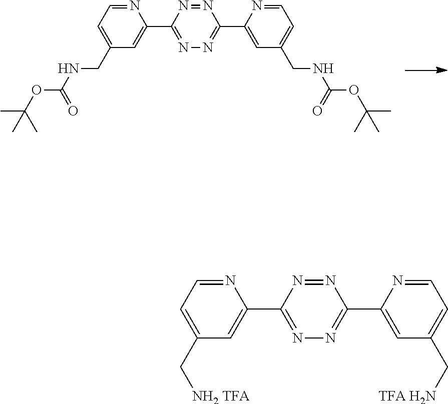

2.9 (50 mg, 0.10 mmol) was dissolved in chloroform/TFA 1:1 (4 mL) and the solution was stirred under Ar at rt for 1 h. The volatiles were removed in vacuo and the solid was flushed with chloroform (4 mL) and methanol (2×4 mL). The solid was dried in vacuo yielding pure 2.10 (50 mg, 96 μmol, 95%) as a red solid. ¹H-NMR (DMSO-d6): δ=9.01 (d, 2H, ArH), 8.78 (s, 2H, ArH), 8.50 (br s, 6H, NH₃⁺), 7.80 (d, 2H, ArH), 4.34 (s, 4H, CH₂) ppm. ¹³C-NMR (DMSO-d6): δ=163.3, 158.5 (q), 151.0, 150.4, 144.9, 126.2, 123.8, 117.1 (q), 41.2 ppm. ¹⁹F-NMR (DMSO-d6): δ=−73.5 ppm. ESI-MS: m/z Calc. for C₁₈H₁₆F₆N₈O₄ 522.12; Obs. [M−2TFA+H]⁺295.25.

### Di-t-butyl ((4,4′-(1,2,4,5-tetrazine-3,6-diyl)bis(pyridine-4,2-diyl))bis(methylene))dicarbamate (2.11)

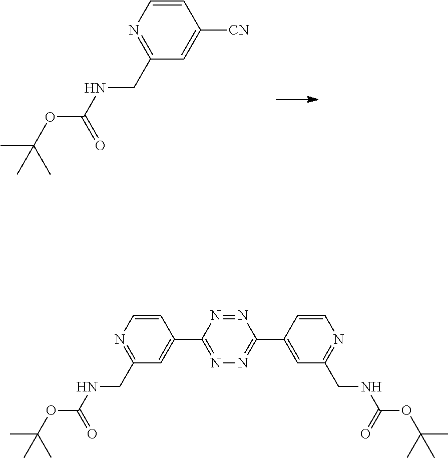 [SMILES: CC(C)(C)OC(=O)NCc1cc(C#N)ccn1 ; CC(C)(C)OC(=O)NCc1cc(-c2nnc(-c3ccnc(CNC(=O)OC(C)(C)C)c3)nn2)ccn1]

t-Butyl ((4-cyanopyridin-2-yl)methyl)carbamate (99 mg, 0.42 mmol), Zn(OTf)₂ (8.2 mg, 23 μmol, 0.05 eq), ethanol (150 μL) and hydrazine hydrate (150 μL, 2.6 mmol, 6.2 eq) were stirred at 60° C. for 15 h. 3-Mercaptopropionic acid (30 μL, 0.34 mmol, 0.8 eq) and hydrazine hydrate (100 μL, 1.7 mmol, 4 eq) were added and the solution was stirred at 60° C. for 23 h, resulting in a much improved conversion. Chloroform (20 mL) and water (20 mL) were added and the layers were separated. The aqueous phase was extracted with chloroform (2×10 mL) and the combined organic layers were dried with Na₂SO₄. After filtration, the filtrate was evaporated to dryness yielding impure intermediate [2H]-TZ as an orange solid. The [2H]-TZ was dissolved in dichloromethane (3 mL) and PhI(OAc)₂ (80 mg, 0.24 mmol) was added as a solid. The solution was stirred at rt for 1 h during which a color change occurred from orange to red. Since the conversion was low (based on HPLC-PDA/MS), PhI(OAc)₂ (100 mg, 0.30 mmol) was added as a solid and the solution was stirred at rt for 2 h. Column chromatography (flash SiO₂) using an elution gradient of 10% to 60% EtOAc in CHCl₃ and, in a second chromatography step (normal SiO₂), 60% acetone in heptane yielded pure 2.11 (12 mg, 24 μmol, 11% overall) as a pink solid. ¹H-NMR (CDCl₃): δ=8.88 (d, 2H, ArH), 8.50 (s, 2H, ArH), 8.40 (d, 2H, ArH), 5.60 (br, 2H, NH), 4.65 (d, 4H, CH₂), 1.50 (s, 18, CH₃) ppm. ¹³C-NMR (CDCl₃): δ=163.9, 159.9, 156.2, 150.8, 139.5, 120.1, 119.4, 80.0, 46.1, 28.6 ppm. ESI-MS: m/z Calc. for C₂₄H₃₀N₈O₄ 494.24; Obs. [M+H]+495.17, [2M+Na]⁺1011.17.

### (4,4′-(1,2,4,5-Tetrazine-3,6-diyl)bis(pyridine-4,2-diyl))dimethanamine, TFA Salt (2.12)

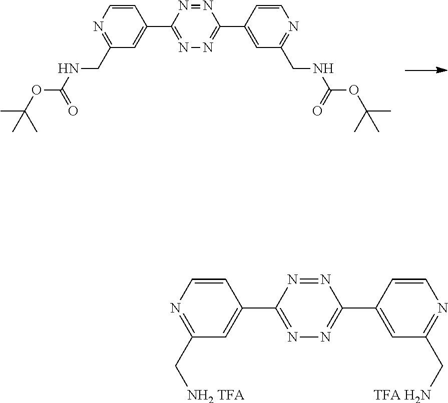

2.11 (12 mg, 24 μmol) was dissolved in chloroform/TFA 1:1 (2 mL) and the solution was stirred under Ar at rt for 1 h. The volatiles were removed in vacuo and the solid was flushed with chloroform (2 mL). The solid was dried in vacuo yielding pure 2.12 (12 mg, 23 μmol, 95%) as a red solid. ¹H-NMR (MeOD): δ=8.98 (d, 2H, ArH), 8.65 (s, 2H, ArH), 8.57 (d, 2H, ArH), 4.50 (s, 4H, CH₂) ppm. ¹³C-NMR (MeOD): δ=164.9, 155.5, 152.0, 142.2, 122.5, 121.3, 44.2 ppm. ¹⁹F-NMR (MeOD): δ=−77.5 ppm. ESI-MS: m/z Calc. for C₁₈H₁₆F₆N₈O₄ 522.12; Obs. [M-2TFA+H]⁺295.17.

### 5,5′-((2,2′-(1,2,4,5-Tetrazine-3,6-diyl)bis(pyridine-3,2-diyl))bis(azanediyl))bis(5-oxopentanoic acid) (2.13)

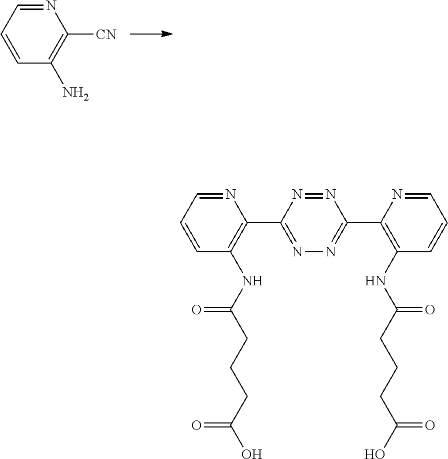 [SMILES: N#Cc1ncccc1N ; O=C(O)CCCC(=O)Nc1cccnc1-c1nnc(-c2ncccc2NC(=O)CCCC(=O)O)nn1]

3-Aminopicolinonitrile (0.62 g, 5.2 mmol), 3-mercaptopropionic acid (0.46 mL, 5.2 mmol, 1 eq) and hydrazine hydrate (1.8 mL, 32 mmol, 6 eq) were stirred at 90° C. for 18 h. Cold water (2 mL) was added and the suspension was stirred at rt for 5 min. Filtration, washing of the solid with cold water and cold ethanol (both 5×2 mL) and drying in vacuo yielded the intermediate [2H]-TZ (0.66 g, 2.4 mmol, 94%) as an orange solid. [2H]-TZ (200 mg, 0.75 mmol) and glutaric anhydride (0.9 g, 7.5 mmol, 10 eq) were suspended in dry THF (3 mL). Upon stirring at 52° C. the suspension cleared and the mixture was stirred at 52° C. for 19 h. The obtained suspension was filtrated and the resulting solid was washed with diethyl ether (3×4 mL). The solid was transferred to a round-bottom flask and the solvent was removed in vacuo. The compound was suspended in acetic acid (8 mL) and concentrated nitric acid (6 drops) was added dropwise (CAUTION: toxic fumes). The initially orange suspension turned red in seconds and the mixture was stirred at rt for 30 min. The suspension was filtrated and the resulting solid was washed with acetic acid (3×4 mL) and diethyl ether (3×4 mL). The solid was dried in vacuo at 35° C. for 2 h yielding pure 2.13 (327 mg, 0.66 mmol, 89%) as a red solid. ¹H-NMR (DMSO-d6): δ=10.15 (s, 2H, NH), 8.65 (dd, 2H, ArH), 8.32 (dd, 2H, ArH), 7.71 (dd, 2H, ArH), 2.27 (t, 4H, CH₂), 2.22 (t, 4H, CH₂), 1.70 (qn, 4H, CH₂CH₂CH₂) ppm. ¹³C-NMR (DMSO-d6): δ=174.1, 171.0, 164.5, 145.8, 142.2, 134.5, 132.0, 126.1, 35.0, 32.8, 20.1 ppm. ESI-MS: m/z Calc. for C₂₂H₂₂N₈O₆ 494.17; Obs. [M+H]⁺495.33, λₘₐₓ=240, 328 and 525 nm.

### N¹,N^{1′}-(2,2′-(1,2,4,5-Tetrazine-3,6-diyl)bis(pyridine-3,2-diyl))bis(N⁵-(29-hydroxy-3,6,9,12,15,18,21,24,27-nonaoxanonacosyl)glutaramide) (2.14)

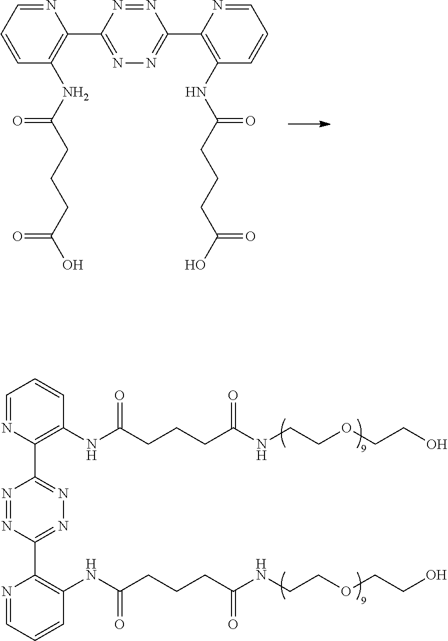

2.13 (32.7 mg, 66 μmol) and 29-amino-3,6,9,12,15,18,21,24,27-nonaoxanonacosan-1-ol (64.1 mg, 0.14 mmol, 2.1 eq) were suspended in DMF (0.6 mL). A solution of PyBOP (92 mg, 0.17 mmol, 2.6 eq) in DMF (0.5 mL) and N-methylmorpholine (44 jut, 0.40 mmol, 6 eq) were added. The mixture was stirred at rt for 2 h during which the suspension eventually cleared. Column chromatography (flash SiO₂) using an elution gradient of 8% to 16% methanol in chloroform yielded impure product. The compound was dissolved in chloroform (60 mL, with some added methanol) and washed with water/brine 1:1 (30 mL). The organic layer was dried with Na₂SO₄, the mixture was filtrated and the solution was evaporated to dryness. This yielded pure 2.14 (57 mg, 41 μmol, 63%) as a red sticky solid. 2.14 should be stored at −80° C. to avoid decomposition by hydrolysis. ¹H-NMR (DMSO-d6): δ=10.16 (s, 2H, ArNH), 8.65 (dd, 2H, ArH), 8.33 (dd, 2H, ArH), 7.79 (t, 2H, NHCH₂), 7.71 (dd, 2H, ArH), 4.54 (t, 2H, OH), 3.53-3.33 (m, 76H, OCH₂), 3.16 (m, 4H, NHCH₂), 2.23 (t, 4H, CH₂), 2.07 (t, 4H, CH₂), 1.70 (qn, 4H, CH₂CH₂CH₂) ppm. ESI-MS: m/z Calc. for C₆₂H₁₀₄N₁₀O₂₄ 1372.72; Obs. [2M+H]⁺687.58, [M+H]⁺1373.83, λₘₐₓ=242 and 524 nm.

### 5,5′-((4,4′-(1,2,4,5-Tetrazine-3,6-diyl)bis(pyridine-4,3-diyl))bis(azanediyl))bis(5-oxopentanoic acid) (2.15)

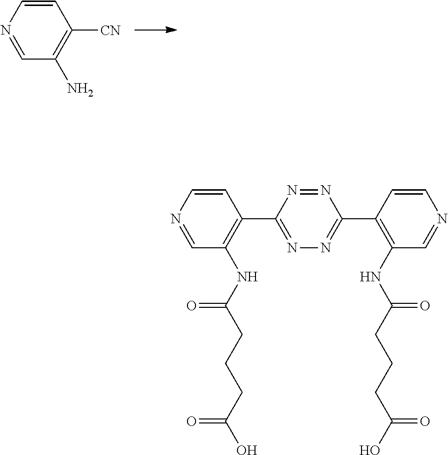 [SMILES: N#Cc1ccncc1N ; O=C(O)CCCC(=O)Nc1cnccc1-c1nnc(-c2ccncc2NC(=O)CCCC(=O)O)nn1]

3-Aminoisonicotinonitrile (200 mg, 1.6 mmol), S (26 mg, 0.8 mmol, 0.5 eq) and hydrazine hydrate (0.46 mL, 8.1 mmol, 5 eq) were stirred at 100° C. for 16 h. Cold water (2 mL) was added and the suspension was stirred at rt for 5 min. The suspension was filtrated and the solid was washed with cold water and cold ethanol (both 5×4 mL). Trituration of the solid in ethanol (20 mL) at 50° C. for 15 min was followed by filtration. The solid was washed with ethanol (5×4 mL) and dried in vacuo yielding the intermediate [2H]-TZ (144 mg, 0.54 mmol, 67%) as an orange solid. The [2H]-TZ (200 mg, 0.75 mmol) and glutaric anhydride (0.9 g, 7.5 mmol, 10 eq) were suspended in dry THF (2 mL) and the mixture was stirred under an Ar atmosphere at 52° C. for 14 d. In time, the reaction mixture turned into a paste and additional THF was occasionally added to ensure that stirring continued. After ¹H-NMR confirmed close to full conversion the paste was transferred to a round-bottom flask and the solvent was removed in vacuo. The compound was suspended in acetic acid (20 mL) and concentrated nitric acid (0.75 mL) was added dropwise (CAUTION: toxic fumes). The initially orange suspension turned red in seconds and the mixture was stirred at rt for 30 min. The suspension was filtrated and the resulting solid was washed with acetic acid (3×4 mL). The solid was thoroughly broken up using a spatula, washed with diethyl ether (3×4 mL) and dried in vacuo at 30° C. for 1 h. The dry solid was powdered using a spatula and stirred in methanol (6 mL) at rt for 30 min. The suspension was filtrated and the resulting solid was washed with methanol (2×8 mL) and diethyl ether (5×8 mL). The solid was dried in vacuo at 30° C. for 1 h yielding 2.15 (313 mg, 0.63 mmol, 85%) as a pink solid. ¹H-NMR (DMSO-d6): δ=10.41 (s, 2H, NH), 9.05 (s, 2H, ArH), 8.67 (d, 2H, ArH), 8.04 (d, 2H, ArH), 2.33 (t, 4H, CH₂), 2.24 (t, 4H, CH₂), 1.72 (qn, 4H, CH₂CH₂CH₂) ppm. ¹³C-NMR (DMSO-d6): δ=174.1, 171.2, 164.0, 145.1, 144.8, 132.9, 131.9, 124.3, 35.0, 32.8, 20.1 ppm. ESI-MS: m/z Calc. for C₂₂H₂₂N₈O₆ 494.17; Obs. [M+H]⁺495.33, λₘₐₓ=240, 328 and 525 nm.

### N¹,N^{1′}-(4,4′-(1,2,4,5-Tetrazine-3,6-diyl)bis(pyridine-4,3-diyl))bis(N⁵-(29-hydroxy-3,6,9,12,15,18,21,24,27-nonaoxanonacosyl)glutaramide) (2.16)

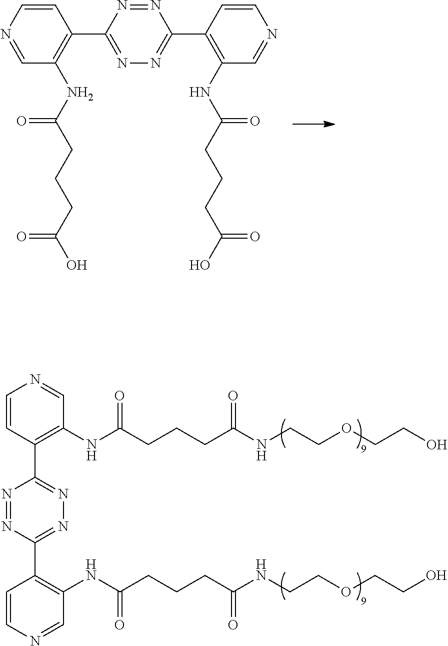

2.15 (27.5 mg, 56 μmol) and 29-amino-3,6,9,12,15,18,21,24,27-nonaoxanonacosan-1-ol (53.5 mg, 0.12 mmol, 2.1 eq) were suspended in DMF (0.5 mL). A solution of PyBOP (75 mg, 0.14 mmol, 2.5 eq) in DMF (0.4 mL) and N-methylmorpholine (37 jut, 0.33 mmol, 6 eq) were added. The mixture was stirred at rt for 90 min during which the suspension eventually cleared. Column chromatography (flash SiO₂) using an elution gradient of 10% to 20% methanol in chloroform yielded impure product. The compound was dissolved in chloroform (40 mL, with some added methanol) and washed with water/brine 1:1 (25 mL). The organic layer was dried with Na₂SO₄, the mixture was filtrated and the solution was evaporated to dryness. This yielded pure 2.16 (36 mg, 56 μmol, 47%) as a red sticky solid. 2.16 should be stored at −80° C. to avoid decomposition by hydrolysis. ¹H-NMR (DMSO-d6): δ=10.35 (s, 2H, ArNH), 9.05 (s, 2H, ArH), 8.66 (d, 2H, ArH), 8.02 (d, 2H, ArH), 7.80 (t, 2H, NHCH₂), 4.54 (t, 2H, OH), 3.53-3.34 (m, 76H, OCH₂), 3.17 (q, 4H, NHCH₂), 2.29 (t, 4H, CH₂), 2.09 (t, 4H, CH₂), 1.72 (qn, 4H, CH₂CH₂CH₂) ppm. ¹³C-NMR (CDCl₃): δ=172.56, 171.54, 163.89, 145.73, 145.29, 133.81, 128.16, 122.44, 72.35, 70.5-69.7 (m), 61.50, 39.18, 36.56, 34.95, 21.30 ppm. ESI-MS: m/z Calc. for C₆₂H₁₀₄N₁₀O₂₄ 1372.72; Obs. [2M+H]⁺687.75, [M+H]⁺1373.75, λₘₐₓ=237 and 526 nm.

### TZ 2.15 Conjugate with Ser-Ala-Asn-OH Peptide (2.21)

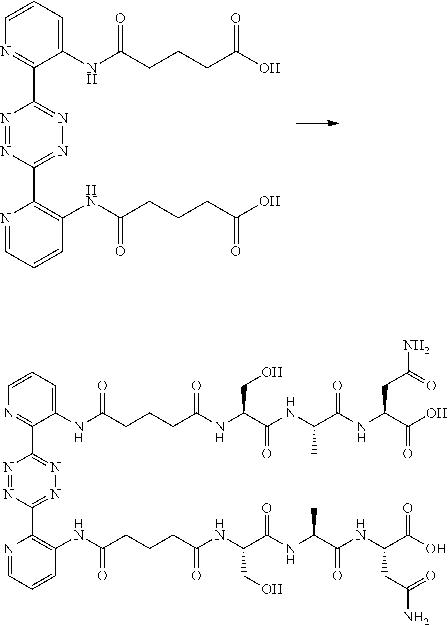 [SMILES: O=C(O)CCCC(=O)Nc1cccnc1-c1nnc(-c2ncccc2NC(=O)CCCC(=O)O)nn1 ; C[C@H](NC(=O)[C@H](CO)NC(=O)CCCC(=O)Nc1cccnc1-c1nnc(-c2ncccc2NC(=O)CCCC(=O)N[C@@H](CO)C(=O)N[C@@H](C)C(=O)N[C@@H](CC(N)=O)C(=O)O)nn1)C(=O)N[C@@H](CC(N)=O)C(=O)O]

2.15 is treated with PyBOP (2 eq) and DiPEA (10 eq) in dry DMSO for 30 min prior to addition of peptide H-Ser-Ala-Asn-OH ((2S)-2-[(2S)-2-[(2S)-2-amino-3-hydroxy propanamido]propanamido]-3-carbamoylpropanoic acid) (2.5 eq). After stirring for 60 min, acidification, preparative HPLC (a H₂O/MeCN gradient with 0.1% TFA) and lyophilization, the title compound is isolated.

### Example 3—Synthesis TCO Compounds

TCO derivatives 3.7, 3.8, 3.9, 3.10, 3.11 were prepared according to literature procedures [Versteegen et al., Angew Chem Int Ed 2013, 53, 14112; Rossin et al., Bioconjug Chem 2016, 27, 1697; Versteegen et al., Angew Chem Int Ed 2018, 57, 1094].

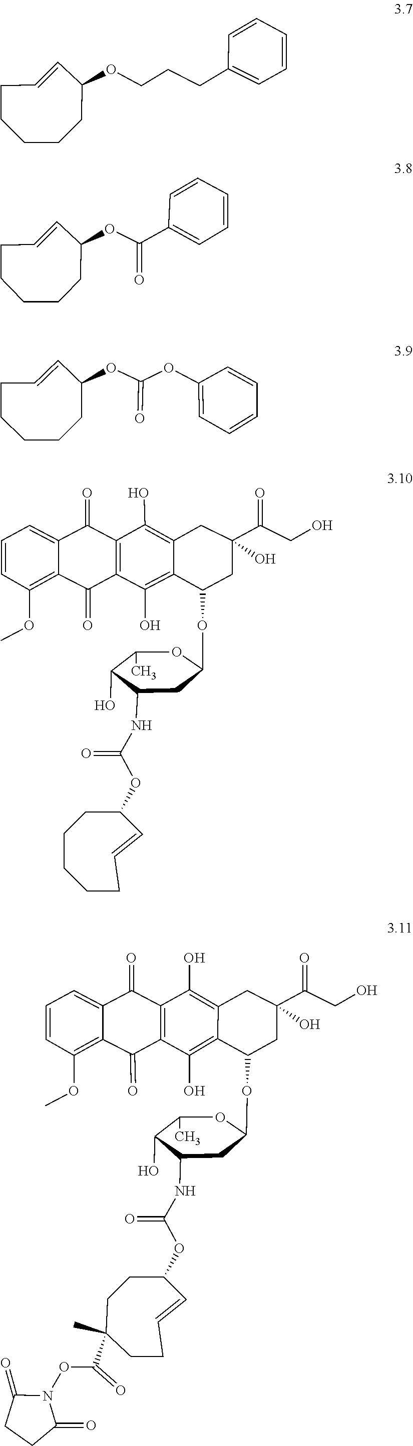

### Synthesis of TCO Linker Used in Diabody ADC (AVP0458-TCO-MMAE)

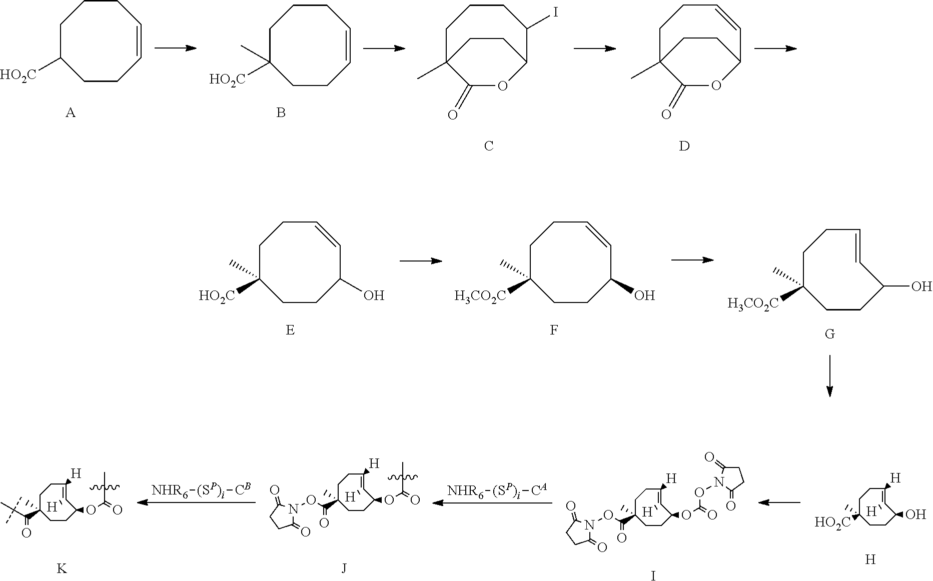

The axial TCO linker (I) used in diabody ADC was prepared as reported [Rossin et al Nature Communications 2018, 9, 1484], with the exception of the conversion of D to F. A solution of the lactone D (23.85 g, 143.7 mmol) in 150 mL methanol and potassium hydroxide (12.53 g, 190 mmol) was heated under reflux for 4 h. Ethyl acetate (20 mL) was added and reflux was continued for 30 minutes. The solution was rotary evaporated. 100 mL toluene was added to the residue, and the mixture was once more rotary evaporated at 60 C. 100 mL DMF was added to the residue and the mixture was rotary evaporated in order to remove the last traces of methanol and toluene. The suspension was cooled in ice, and iodomethane (45 g, 317 mmol) was added over a 20 min period. The mixture was stirred overnight, and the resulting solution was poured into 200 mL water and 200 mL TBME. The layers were separated and the organic layers was washed with 3×50 mL water. The successive aqueous layers were extracted with 150 and 100 mL TBME. Drying and rotary evaporation yielded the hydroxy-ester F, which can be used as such in the irradiation step.

### NHS-TCO-mPEG₁₂₀ (TCO-Masking Moiety) (3.1)

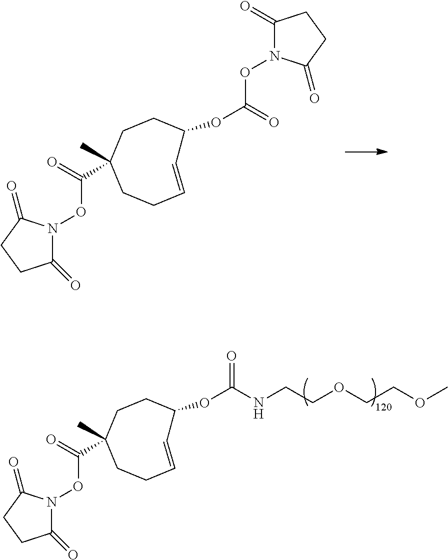

The TCO Trigger I (5.1 mg, 12 μmol, axial isomer) was treated with mPEG₁₂₀-amine (48 mg, 9 μmol, MW 5 kDa) and DiPEA (4.2 μL, 24 μmol) in CH₂Cl₂ for 16 hours. The reaction mixture was precipitated in Et₂O:heptanes, and again in Et₂O prior to drying under N₂ and lyophilization to yield 3.1 as a white fluffy powder in 42% yield (27 mg, 5 μmol). ¹H NMR (400 MHz, CHCl₃): δ 5.88-5.96 (m, 1H), 5.64 (dd, J₁=16.7, J₂=2.0 Hz, 1H), 5.38 (m, 1H), 5.20 (s, 1H), 3.98-4.07 (m, 6H), 3.81-3.83 (m, 3H), 3.63-3.67 (bs, 510H), 3.54-3.57 (m, 6H), 2.83 (s, 4H), 2.27-2.39 (m, 3H), 1.85-2.10 (m, 4H), 1.27 (s, 3H) ppm.

### (E)-Cyclooct-2-en-1-yl dimethylcarbamate (Axial Isomer) (3.2)

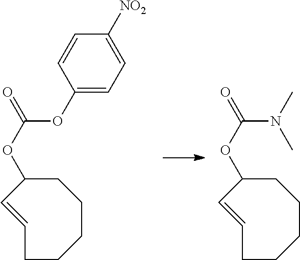 [SMILES: O=C(Oc1ccc([N+](=O)[O-])cc1)OC1/C=C/CCCCC1 ; CN(C)C(=O)OC1/C=C/CCCCC1]

(E)-cyclooct-2-en-1-yl (4-nitrophenyl) carbonate (axial isomer) (109 mg, 0.374 mmol) was dissolved in THF (2 mL), and a solution of dimethylamine (0.468 ml 2 M in THF) was added. After 1 h the solution was evaporated to dryness and the residue was dissolved in chloroform (5 mL), and washed with subsequently 1 M aqueous citric acid (2×2 mL), and 1 M aqueous NaOH (3×2 mL). The organic layer was dried over sodium sulfate, filtered and evaporated to dryness to yield 3.2 as a colorless oil (69 mg, 93%). ¹H-NMR (CDCl₃): δ=5.80 (m, 1H), 5.55 (dd, 1H), 5.36 (d, 1H), 2.97 (s, 3H), 2.93 (s, 3H), 2.47 (m, 1H), 2.01 (m, 3H), 1.79 (m, 1H), 1.68 (m, 2H), 1.43 (m, 1H), 1.08 (m, 1H), 0.82 (m, 1H) ppm. ¹³C-NMR (CDCl₃): δ=155.81, 131.70, 131.63, 74.35, 40.85, 36.04, 35.90, 30.33, 29.16, 24.18 ppm. ESI-MS: m/z Calc. for C₁₁H₁₉NO₂ 197.14; Obs. [M+H]⁺198.17, λₘₐₓ<200 nm.

### (1R,4E)-Cyclooct-4-en-1-yl 2,5-dioxopyrrolidin-1-yl carbonate (3.3)

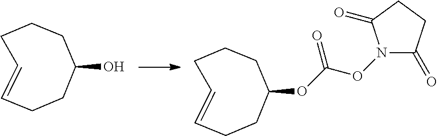

(1R,4E)-cyclooct-4-en-1-ol (TCO-5-OH, axial isomer) (0.20 g, 1.6 mmol) was treated with DSC (0.81 g, 3.16 mmol), DMAP (0.22 g, 1.8 mmol) and DiPEA (1.4 mL, 4.9 mmol) in dry MeCN for 16 h at rt. The reaction mixture was partitioned between EtOAc and 2M HCl followed by washing of the organics with saturated NaHCO₃. After drying over Na₂SO₄ and concentration, compound 3.3 was isolated after silica gel column chromatography (EtOAc:heptanes, 1:9 to 3:7). Yield: 36% (0.15 g, 0.56 mmol).

### 3-({[(1R,4E)-Cyclooct-4-en-1-yloxy]carbonyl}amino)-2-sulfopropanoic acid (3.4)

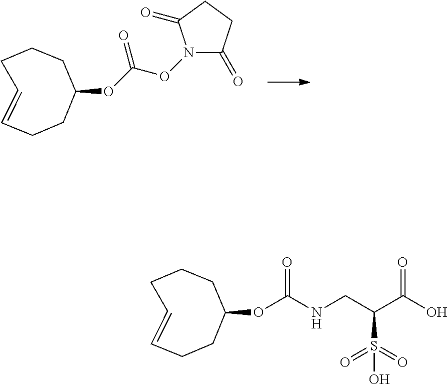

3.3 (0.15g, 0.56 mmol) in MeCN was added to 3-amino-2-sulfopropanoic acid (0.14 g, 0.84 mmol) in sat. NaHCO₃. After stirring for 1 h, the MeCN was removed in vacuo. Following acidification, preparative HPLC (a H₂O/MeCN gradient with 0.1% TFA) and lyophilization, 3.4 was isolated in 74% yield (0.13 g, 0.42 mmol). ESI-MS: m/z calc for C₁₂H₁₉NO₇S 321.09; Obs. [M−H]⁺320.20.

### 1-({2-[2-(2-Aminoethoxy)ethoxy]ethyl}carbamoyl)-2-({[(1R,4E)-cyclooct-4-en-1-yloxy]carbonyl}amino)ethane-1-sulfonic acid (3.5)

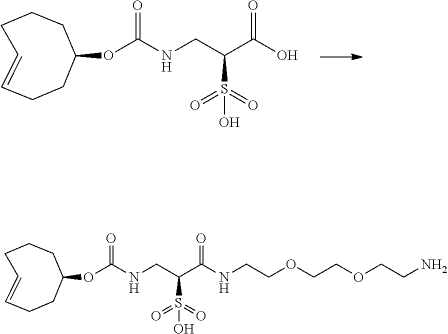

PyBOP (0.28 g, 0.54 mmol) was added to a solution of 3.4 (0.13 g, 0.42 mmol) and amino-PEG₂-amine (2.47 g, 16.6 mmol) in MeCN and the reaction mixture was stirred for 1 hour at rt. Following acidification with aqueous citric acid, preparative HPLC (a H₂O/MeCN gradient with 0.1% TFA) and lyophilization, 3.5 was isolated in 71% yield (0.17 g, 0.30 mmol) as stick solid. ESI-MS: m/z calc for C₁₈H₃₃N₃O₈S 451.20; Obs. [M+H]⁺452.12.

### Maleimide-TCO-TCO Linker (3.6)

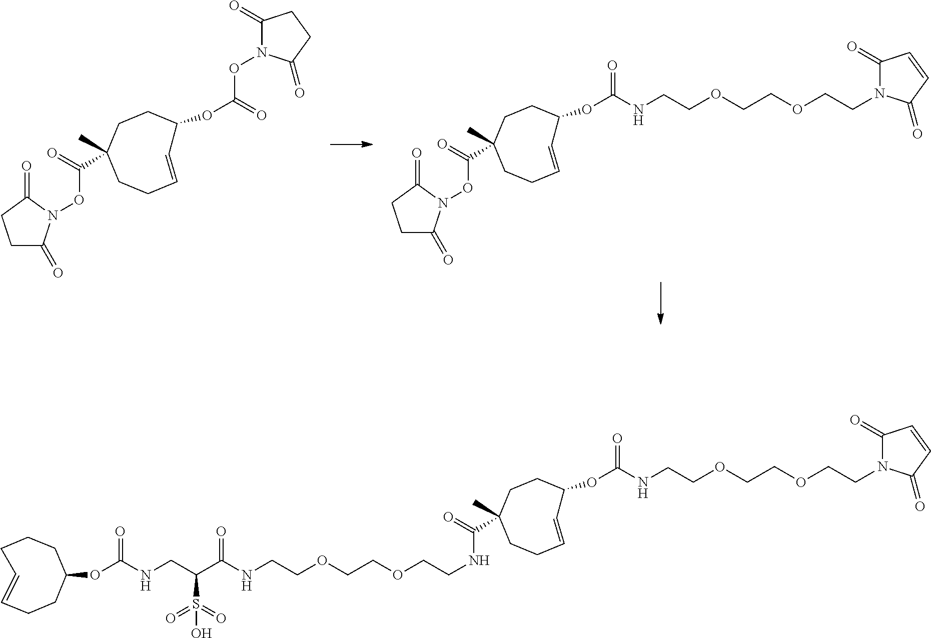

The TCO linker I (10 mg, 22.6 μmol, axial isomer) was treated with the TFA salt of 1-{2-[2-(2-aminoethoxy) ethoxy]ethyl}-2,5-dihydro-1H-pyrrole-2,5-dione (7.7 mg, 22.6 μmol), and DiPEA (11.9 μL, 67.8 μmol) in DMSO for 3 h. 3.5 (15.3 mg, 27.1 μmol) and DiPEA (11.9 μL, 67.8 μmol) were added and the reaction mixture was stirred for 16 h at rt. Preparative HPLC (a H₂O/MeCN gradient with 0.1% TFA) and lyophilization, yielding 3.6 in 9.3% yield (1.8 mg, 2.1 μmol). ESI-MS: m/z calc for C₃₉H₆₁N₅O₁₅S 871.39; Obs. [M+H]⁺872.16.

### (Z)-9-Azabicyclo[6.2.0]dec-4-en-10-one (3.12)

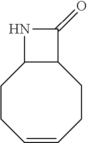 [SMILES: O=C1NC2CC/C=C\CCC12]

To 275 mL cyclooctadiene (2.25 mol), 300 mL DCM and 8.0 g sodium carbonate (75.5 mmol) on ice was added chlorosulfonyl isocyanate (101.9 g, 0.72 mol) over 1 hour at 4-6° C. Following 4 days of stirring, the mixture was poured gradually (in 20 min) into a mechanically stirred mixture of 150 g Na₂HPO₄ (0.843 mol), 150 g sodium sulfite (1.19 mol) and 1 kg ice/150 mL DCM. The mixture was put in an ice-bath and after stirring rapidly for 15 min, 65.5 g NaHCO₃ (0.780 mol) was added in portions over 1 h. After stirring for an hour, H₂O was added. The organic phase was washed with H₂O and the combined H₂O layers were extracted with EtOAc. Drying and concentration of the organic layers gave a residue that, after 4 days of resting, was decanted, treated with hept and filtered to yield 3.12 (20.26 g). ¹H-NMR (300 MHz, CDCl₃): δ 6.0 (bs, 1H), 5.68 (m, 2H), 3.8 (m, 1H), 3.3 (m, 1H), 2.4 (m, 2H), 2.0 (m, 6H) ppm.

Additional crude material was by from the decanted liquid and after treating the combined H₂O layers obtained by repeating the workup steps followed purified by silica column purification (hept/EtOAc). Combined yield 55.63 g (0.368 mol, 51%).

### Methyl (1R,4Z,8S)-8-aminocyclooct-4-ene-1-carboxylate HCl (3.13)

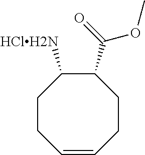

To a cooled solution of 3.12 (55.63 g, 0.368 mol) in 300 mL MeOH was added thionyl chloride (68 mL, 0.937 mol) over 5. After overnight stirring at rt and concentration, the resulting solidifying oil was stirred for 2 hr with TBME (200 mL). 3.13 was isolated after filtration and washing with TBME. Yield: 54.10 g (0.246 mol, 67%). ¹H-NMR (300 MHz, CDCl₃): δ 5.75-5.58 (m, 2H), 3.79 (s+m, 4H), 3.24 (t, 1H), 2.65 (m, 1H), 2.5-1.7 (m, 7H) ppm.

### Methyl (1R,4Z,8S)-8-((tert-butoxycarbonyl)amino)cyclooct-4-ene-1-carboxylate (3.14)

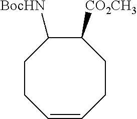 [SMILES: COC(=O)[C@H]1CC/C=C\CCC1NC(=O)OC(C)(C)C]

To 3.13 (17.78 g, 80.9 mmol) and NEt₃ (17.3 g, 171.3 mmol) in 25 mL DCM and 100 mL toluene on ice was added Boc-anhydride (20.1 g, 92.2 mmol) over 30 min. After stirring for 3 days at rt, 50 ml H₂O was then added and the mixture was stirred for 15 min. The layers were separated and the upper layer was washed with 40 mL H₂O. The successive aq. layers were extracted with 50 mL toluene. Drying and concentrating gave 23.07 g brown oil, (81.4 mmol). ¹H-NMR (300 MHz, CDCl₃): δ 5.8-5.55 (m, 2H), 5.05 (m, 1H), 4.15 (m, 1H), 3.72 (s, 3H), 2.85 (m, 1H), 2.45 (m, 1H), 2.4-1.5 (m, 7H), 1.43 (s, 9H) ppm.

### Methyl (rel-1S,8S,Z)-8-((tert-butoxycarbonyl)amino)cyclooct-4-ene-1-carboxylate (3.15)

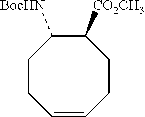 [SMILES: COC(=O)[C@H]1CC/C=C\CC[C@@H]1NC(=O)OC(C)(C)C]

To 3.14 in 75 mL MeOH on ice was added 46 g 25 wt % sodium methoxide in MeOH slowly followed by stirring overnight and concentration. TBME, ice and H₂O were added to the residue followed by separation and additional washing of the upper layer with H₂O. TBME, 15 g citric acid and ice was added to the first H₂O layer. The layers were separated and the upper layer was washed with the second H₂O layer. Drying and rotary evaporation followed by chromatography on silica (hept/EtOAc/NEt₃) gave the separated cis and trans isomers (NHBoc vs CO₂CH₃). This cis material was treated and purified again to yield a second crop of trans isomer material, to give a combined yield of 10.64 g (37.5 mmol, 46%). ¹H-NMR (300 MHz, CDCl₃): δ 5.7 (m, 2H), 4.63 (m, 1H), 4.0 (m, 1H), 3.66 (s, 3H), 2.75 (m, 1H), 2.5-1.5 (m, 8H), 1.41 (s, 9H) ppm.

### (rel-1S,8S,Z)-8-((tert-Butoxycarbonyl)amino)cyclooct-4-ene-1-carboxylic acid (3.16)

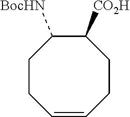 [SMILES: CC(C)(C)OC(=O)N[C@H]1CC/C=C\CC[C@@H]1C(=O)O]

To 3.15 (10.64 g, 37.5 mmol) and 11.0 g potassium carbonate in 50 mL H₂O and was added 50 mL MeOH over a few min and the mixture was stirred at 30° C. over the 3 days, then heated at 62° C. for 22 hr to give a clear solution. Most of the MeOH was removed by rotary evaporation and the remaining solution was washed with 2×50 mL toluene. The combined H₂O layers were cooled in ice, 100 mL toluene was added and the mixture was slowly acidified with 11 g citric acid. The layers were separated and the H₂O layer was extracted with toluene. Drying and rotary evaporation gave 10.3 g of the acid. The NMR showed multiple broad signals (at δ 6.6, 5.7, 5.15, 4.9, 4.2, 4.1, 3.9, 2.9, 2.8, 2.7, 2.5-1.4 ppm).

### tert-Butyl N-[(1S,2S,4Z,6R)-8-oxo-7-oxabicyclo[4.2.2]dec-4-en-2-yl]carbamate (3.17)

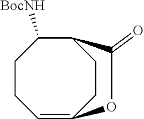

3.16 (31.56, 117.2 mmol) and 36.0 g NaHCO₃ (429 mmol) in 200 mL DCM and 120 ml H₂O was stirred well for 1 h, then cooled in ice. A total amount of 150.4 mmol potassium iodide and 36.00 g iodine (0.142 mol) was added over 90 min and the mixture was stirred for 3 days. After dilution with DCM and H₂O, 6.0 g sodium sulfite was added slowly to make the mixture colorless. The organic layer was washed with H₂O. The combined H₂O layers were extracted with DCM. Drying and concentrating gave a residue (40.9 g), which was dissolved in 200 mL toluene. 23.08 g DBU (151.6 mmol) was added, and the mixture was warmed for 18 hr at 70° C. After cooling, toluene and ice were added. The organic layer was washed with H₂O and the successive aq. layers were extracted with toluene. Drying and concentrating gave 3.17 as a viscous oil (18.5 g). ¹H-NMR (300 MHz, CDCl₃): δ 5.83 (m, 1H), 5.62 (m, 1H), 5.10 (m, 1H), 4.76 (broad, 1H), 4.27 (broad, 1H), 3.50 (broad, 1H), 2.73 (broad, 1H), 2.17 (m, 1H), 2.1-1.4 (m, 4H), 1.43 and 1.42 (2s, 9H) ppm.

### Methyl (rel-1S,2S,6R,Z)-2-((tert-butoxycarbonyl)amino)-6-hydroxy cyclooct-4-ene-1-carboxylate (3.18)

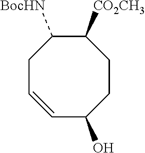 [SMILES: COC(=O)[C@H]1CC[C@@H](O)/C=C\C[C@@H]1NC(=O)OC(C)(C)C]

To cooled 3.17 (9.47 g, 35.42 mmol) in 30 mL THF and 10 mL MeOH was added 110 mg Na (4.8 mmol) in 20 mL MeOH and the solution was stirred overnight (Note: esterification is accompanied by partial epimerization when more than 1 eq. of base is used). The solution was poured into 100 mL water and 100 mL toluene. The layers were separated and the aq. layer was extracted with toluene. The toluene layers were washed water, dried and concentrated to give 10.3 g of 3.18. ¹H-NMR (300 MHz, CDCl₃): δ 5.7 (m, 1H), 5.55 (m, 1H), 4.6 (m, 2H), 3.65 (s, 3H), 2.55 (m, 2H), 2.3 (m, 1H), 2.1-1.5 (m, 5H), 1.41 (s, 9H) ppm.

### Methyl (rel-1S,2S,6R,Z)-6-hydroxy-2-(((2-(trimethylsilyl)ethoxy)carbonyl) amino)cyclooct-4-ene-1-carboxylate (3.19)

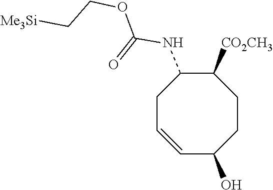 [SMILES: COC(=O)[C@H]1CC[C@@H](O)/C=C\C[C@@H]1NC(=O)OCC[Si](C)(C)C]

To cooled 3.18 (2.25 g, 7.52) in 30 mL DCM was added 6.28 g TFA (55.1 mmol) in 5 mL DCM over 20 min. The solution was stirred for 6 hr, reaching rt after 2 hr followed by concentration (4.40 g). It was dissolved in 30 mL DCM, 5.0 g NEt₃ was added (49.5 mmol) and the solution was cooled. 2.05 g N-[2-(trimethylsilyl)ethoxycarbonyloxy]succinimide (7.91 mmol) was added and the solution was stirred overnight. The solution was diluted with DCM and washed with H₂O. The successive aq. layers were extracted with DCM. Drying and rotavapping gave 3.43 g residue, which was chromatographed on silica (hept/EtOAc) Yield: 1.63 g (4.75 mmol, 63%). ¹H-NMR (300 MHz, CDCl₃): δ 5.75 (m, 1H), 5.58 (m, 1H), 4.66 (d, 1H), 4.6 (m, 1H), 4.14 (bt, 2H), 3.65 (s, 3H), 2.55 (m, 2H), 2.3 (m, 1H), 2.1-1.5 (m, 5H), 0.95 (bt, 2H), 0.03 (s, 9H) ppm.

### Methyl (1S,2S,4E,6R)-6-hydroxy-2-({[2-(trimethylsilyl)ethoxy]carbonyl}amino)cyclooct-4-ene-1-carboxylate (3.20)

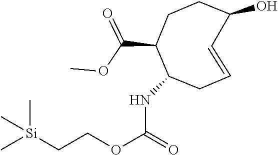

3.19 (1.63 g, 4.75 mmol) and 1.65 g methyl benzoate were irradiated in a 4/1 mixture of hepts and EtOAc, while flushing the solution continuously through a 10 g column of 10% silver nitrate on silica. After a total irradiation time of 23 h, the solution contained no starting compound anymore. The column was eluted with 75 mL TBME, then with 75 ml TBME/5% MeOH. Each eluate was stirred with (the same) 25 mL 15 wt % ammonia. The layers were separated and the organic layer was washed with 15 mL H₂O, then dried and rotary evaporated. NMR indicated that the material (0.85 g) was the axial-TCO product. ¹H-NMR (300 MHz, CDCl₃): δ 5.86 (m, 1H), 5.52 (d, 1H), 4.68 (bs, 2H), 4.12 (bt, 2H), 3.94 (m, 1H), 3.67 (m) and 3.62 (s) (4H), 2.95 (m, 1H), 2.2 (m, 2H), 1.85 (m, 2H), 1.4 (m, 2H), 0.95 (m, 2H), 0.03 (s, 9H) ppm.

### Methyl (1S,2S,4E,6R)-6-{[(4-nitrophenoxy)carbonyl]oxy}-2-({[2-(trimethylsilyl)ethoxy] carbonyl}amino)cyclooct-4-ene-1-carboxylate (3.21)

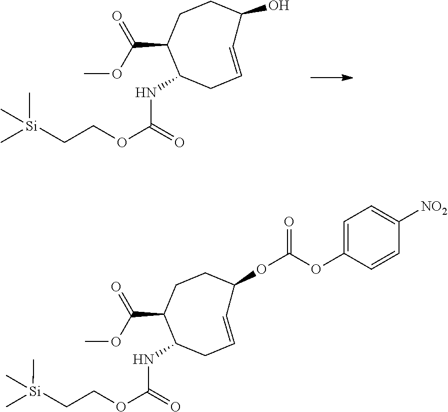

3.20 (140 mg; 0.408 mmol) was dissolved in CHCl₃ (10 mL), and DMAP (199 mg; 1.63 mmol) was added. The solution was cooled to 0° C., and 4-nitrophenyl chloroformate (123 mg; 0.612 mmol) was added. The mixture was stirred at 0° C. under an atmosphere of Ar for 30 min, and then washed with 0.5 M aq. citric acid solution (2 times 10 mL). The organic layer was isolated, dried over Na₂SO₄, filtered, and evaporated to dryness. The crude product was purified by column chromatography on silica gel, using an elution gradient of 5% to 30% EtOAc in n-hept. This yielded 3.21 as a white solid (60 mg). ¹H-NMR (CDCl₃): δ 8.28 (d, J=9.1 Hz, 2H), 7.39 (d, J=9.1 Hz, 2H), 5.86 (ddd, J=15.5, 11.6, 3.7 Hz, 1H), 5.53 (d, J=12.8 Hz, 1H), 5.50 (s, 1H), 4.72 (d, J=10.3 Hz, 1H), 4.13 (t, J=8.4 Hz, 2H), 3.98 (m, 10H), 3.65 (s, 2H), 3.00 (dt, J=11.5, 4.3 Hz, 1H), 2.33-2.11 (br.m, 3H), 2.00 (m, 1H), 1.66 (m, 2H), 0.95 (m, 2H), 0.03 (s, 9H) ppm. ¹³C-NMR (CDCl₃): δ 13C NMR (101 MHz, CDCl₃) δ 175.82, 155.41, 155.30, 151.46, 145.42, 130.42, 127.72, 125.31, 121.72, 77.50, 53.80, 51.90, 36.04, 25.52, 17.71, −1.49 ppm.

### Methyl (1S,2S,4E,6R)-6-((dimethylcarbamoyl)oxy)-2-(((2-(trimethyl silyl)ethoxy)carbonyl) amino)cyclooct-4-ene-1-carboxylate (3.22)

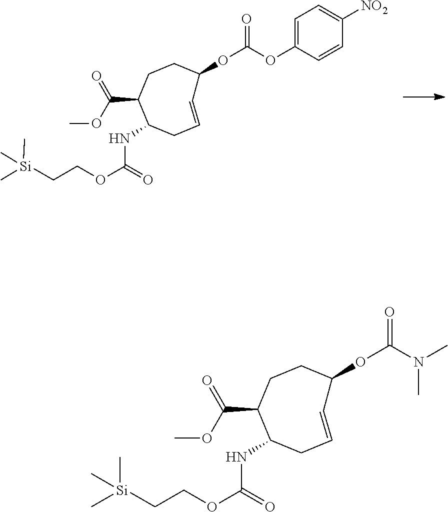

3.21 (30 mg; 0.059 mmol) was dissolved in THF (2 mL). A solution of dimethylamine in THF (0.074 mL 2 M; 0.147 mmol) was added and the mixture was stirred at 20° C. for 30 min. The mixture was evaporated to dryness, dissolved in CHCl₃ (15 mL) and washed with subsequently 0.5 M aq. citric acid solution (2 times 10 mL), and 1 M aq. NaOH (2 times 10 mL). The organic layer was isolated, dried over Na₂SO₄, filtered, and evaporated to dryness to yield 3.22 as a colorless oil (29 mg). ¹H-NMR (CDCl₃): δ 5.68 (ddd, J=15.8, 11.4, 3.8 Hz, 1H), 5.50 (dd, J=16.4, 2.5 Hz, 1H), 5.43 (d, J=2.7 Hz, 1H), 4.74 (m, 1H), 4.12 (t, J=8.5 Hz, 2H), 3.96 (m, 1H), 3.63 (s, 2H), 2.93 (t, J=5.5 Hz, 6H), 2.20 (m, 2H), 2.05 (ddd, J=13.7, 5.6, 2.1 Hz, 1H), 1.92 (ddd, J=15.6, 5.6, 2.2 Hz, 1H), 1.57 (m, 2H), 0.95 (m, 2H), 0.06 (s, 9H) ppm. ¹³C-NMR (CDCl₃): δ 176.12, 155.33, 132.73, 126.04, 125.47, 73.25, 63.13, 58.02, 54.05, 51.74, 42.72, 36.38, 35.81, 30.28, 25.69, 17.67, −1.51 ppm.

### Methyl (1S,2S,4E,6R)-2-amino-6-((dimethylcarbamoyl)oxy)cyclooct-4-ene-1-carboxylate (3.23)

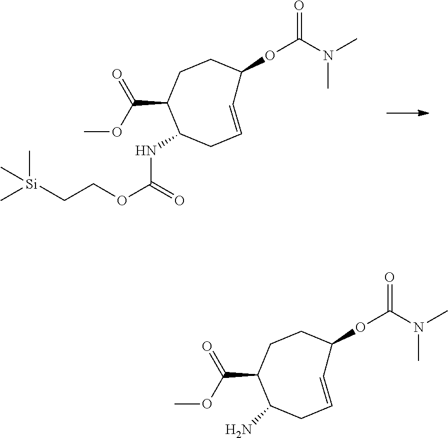

3.22 (7.25 mg; 0.0175 mmol) was dissolved in MeCN (0.5 mL), and potassium fluoride (4.1 mg; 0.070 mmol) was added, followed by tetrabutylammonium fluoride (0.053 mL of a 1 M solution in THF; 0.053 mmol). The mixture was heated to 45° C. for 18 h, and then evaporated to dryness. The crude product was dissolved in CHCl₃ (1.5 mL), and washed with 0.1 M aq. sodium carbonate (3 times 1 mL). The organic layer was isolated, dried over Na₂SO₄, filtered, and evaporated to dryness to yield 3.23 as a mixture of isomers as a colorless oil (4 mg). ¹H-NMR (CDCl₃): δ 5.72 (ddd, J=16.0, 11.6, 3.8 Hz, 1H), 5.50 (dd, J=16.4, 2.5 Hz, 1H), 5.42 (d, J=3.0 Hz, 1H), 3.68 (s, 3H), 3.18 (td, J=10.1, 4.7 Hz, 1H), 2.93 (s, 6H), 2.85 (m, 1H), 2.03 (m, 2H), 1.67 (m, 2H), 1.48 (m, 4H) ppm. ¹³C-NMR (CDCl₃): δ 177.67, 155.44, 132.17, 127.19, 73.46, 59.79, 56.41, 51.60, 44.63, 37.07, 36.40, 35.83, 26.03 ppm.

### N-(But-3-en-1-yl)-N-(3,3-diethoxypropyl)-2,2,2-trifluoroacetamide (3.24)

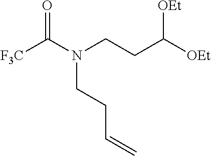 [SMILES: C=CCCN(CCC(OCC)OCC)C(=O)C(F)(F)F]

3,3-diethoxypropan-1-amine (11.1 g, 75.4 mmol), 4-bromo-1-butene (10.8 g, 80.0 mmol) and 24.0 g potassium carbonate (173.9 mmol) in 50 mL DMF was stirred for 45 min at rt, then for 3 hr at 60° C. Following concentration, the residue was diluted with TBME, filtered, and the solid was washed with TBME. 10.05 g DIPEA (77.9 mmol) was added, the solution was cooled and TFA anhydride (16.94 g, 80.66 mmol) was added in 10 min. The solution was stirred for 3 hr at rt, then 12.0 g NaHCO₃ was added, followed by 75 g ice. The mixture was stirred for 10 min, and the organic layer was washed with 50 mL H₂O. The successive aq. layers were extracted with 100 mL TBME. Drying and rotary evaporation gave 19.0 g residue, which was chromatographed (hept/EtOAc), to yield 12.11 g (40.7 mmol, 54% over 2 steps, sufficiently pure for the next step). ¹H-NMR (300 MHz, CDCl₃): δ 5.75 (m, 1H), 5.1 (m, 2H), 4.5 (m, 1H), 3.65 (m, 2H), 3.45 (m, 6H), 2.35 (q, 2H), 1.90 (m, 2H), 1.20 (t, 6H) ppm. ¹⁹F-NMR (282 MHz, CDCl₃): δ −69.2, −69.0 ppm.

### N-(But-3-en-1-yl)-2,2,2-trifluoro-N-(3-oxopropyl)acetamide (3.25)

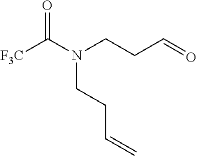

To 3.24 (16.0 g) in 40 mL acetic acid was added 10 mL H₂O and the solution was stirred for 3 days. After removal of some EtOH. It was then stirred for 3 d at 30° C., a few mL H₂O were added, and the solution was partially evaporated. The remainder was diluted with H₂O, then extracted with toluene. The successive toluene layers were washed with H₂O, then dried and rotary evaporated to give 7.95 g product (35.6 mmol, 47% over two steps). ¹H-NMR (300 MHz, CDCl₃): δ 9.64 (2s, 1H), 5.6 (m, 1H), 4.98 (m, 2H), 3.57 (m, 2H), 3.36 (m, 2H), 2.73 (m, 2H), 2.23 (m, 2H) ppm. ¹³C-NMR (75 MHz, CDCl₃): δ 199.7, 198.8, 157 (m), 133.9, 133.1, 117.9, 117.5, 116.2 (d), 47.7, 47.6, 46.4, 42.9, 41.3, 41.1, 33.0 ppm.

### 5-(N-(But-3-en-1-O-2,2,2-trifluoroacetamido)pent-1-en-3-yl 2,2,2-trifluoroacetate (3.26)

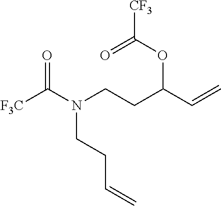 [SMILES: C=CCCN(CCC(C=C)OC(=O)C(F)(F)F)C(=O)C(F)(F)F]

To 3.25 (4.65 g, 20.8 mmol) in 75 mL THF was added 20 mL 1.6 N vinylmagnesium bromide (32 mmol) at −60 to −70° C. over 15 min. The mixture was allowed to warm to 0° C. and then the light-yellow solution was poured into 16 g ammonium chloride in 100 mL H₂O and 100 mL TBME. The layers were separated and the aq. layer was extracted with TBME. Drying and rotary evaporation gave 5.84 g residue. The trifluoroacetyl group appeared to switch partially to the OH (also on standing), therefore it was converted into the bis-TFA compound.

Trifluoroacetic anhydride (4.8 mL, 35 mmol) was added dropwise to a solution of the crude product obtained above in DIPEA (10 mL, 58 mmol) and DCM (100 mL) at 0° C. The mixture was stirred at this temp for 2 h, then rotary evaporated. The residue was purified by column chromatography (EtOAc/het) to give 0.83 g of 3.26. ¹H-NMR (300 MHz, CDCl₃): δ 5.8 (m), 5.4 (m) and 5.1 (m) (7H), 3.4 (m, 4H), 2.36 (q, 2H), 2.09 (q, 2H) ppm. ¹³C-NMR (75 MHz, CDCl₃): δ 133.8, 133.0, 132.9, 132.5, 120.3, 120.0, 118.3, 117.8, 77.1, 76.6, 47.4, 47.4, 46.5, 43.4, 43.4, 43.3, 33.1, 32.7, 31.2, 30.8 ppm. ¹⁹F-NMR (282 MHz, CDCl₃): δ −75.3, −75.2, −69.3, −69.0 ppm.

### (Z)-1-(2,2,2-Trifluoroacetyl)-1,2,3,4,7,8-hexahydroazocin-4-yl 2,2,2-trifluoroacetate (3.27)

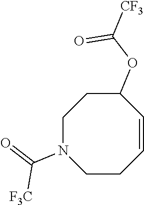 [SMILES: O=C(OC1/C=C\CCN(C(=O)C(F)(F)F)CC1)C(F)(F)F]

A solution of 3.26 (2.3 g, 6.62 mmol) in DCM (700 mL) was degassed and put under N₂. Grubbs 2^{nd} generation catalyst (282 mg, 0.33 mmol) was added and the mixture was stirred at 45° C. for 18h and then at rt for 20h under N₂. The solution was rotary evaporated and the residue was purified by column chromatography on silica, using hept/DCM 1/2 to DCM. ¹H-NMR (300 MHz, CDCl₃): δ 5.9 (m, 1H), 5.7 (m, 2H), 3.75 (m, 2H), 3.4 (m, 2H), 2.45 (m, 3H), 2.0 (m, 1H) ppm. ¹⁹F-NMR (282 MHz, CDCl₃): δ −75.2, −68.8 ppm.

### 2-(Trimethylsilyl)ethyl (Z)-4-hydroxy-3,4,7,8-tetrahydroazocine-1(2H)-carboxylate (3.28)

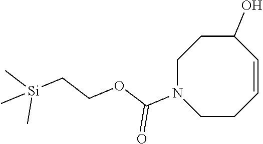 [SMILES: C[Si](C)(C)CCOC(=O)N1CC/C=C\C(O)CC1]

3.27 (1.4 g, 4.39 mmol) was heated under reflux for 2 hr with 550 mg NaOH (13.75 mmol), 5 mL H₂O and 25 mL MeOH. The MeOH was removed by rotary evaporation and 50 mL DCM and Na₂SO₄ were added to the residue. Filtration, repetition (2×) and rotary evaporation gave 600 mg of the aminol intermediate (4.72 mmol) as a solidifying oil. ¹H-NMR (300 MHz, CDCl₃): δ 5.65 (m, 2H), 4.67 (m, 1H), 2.93 (2t, 1H), 2.8 (m, 2H), 2.63 (dt, 1H), 2.3 (m, 1H), 2.15 (m, 1H), 1.95 (m, 1H), 1.6 (m, 1H) ppm.

This aminol was dissolved in 20 mL DCM, 1.24 g (12.3 mmol) NEt₃ was added, followed by 1.38 g N-[2-(trimethylsilyl)ethoxy carbonyloxy]succinimide (5.32 mmol). The solution was stirred overnight, and concentrated prior to addition of 75 mL toluene and 20 mL H₂O. The layers were separated and the upper layer was washed with H₂O. The successive aq. layers were extracted with toluene. Drying and rotary evaporation yielded a solidifying oil, which was chromatographed on silica (hept/EtOAc). Yield: 1.11 g (4.09 mmol). ¹H-NMR (300 MHz, CDCl₃, 2 rotational isomers in a ca. 1.5/1 ratio): δ 5.75 (m) and 5.65 (m) (2H), 4.5 (m, 1H), 4.18 (m, 2H), 3.9 (2t) and 3.8 (2t) (1H), 3.5 (m, 1H), 2.9 (m, 2H), 2.6-2.0 (m, 3H), 1.58 (m, 1H), 1.0 (m, 2H), 0.0 (s, 9H) ppm. ¹³C-NMR (75 MHz, CDCl₃): δ 157, 156, 136.3, 136.1, 127.3, 126.7, 67.5, 67.5, 63.5, 63.4, 49.2, 46.2, 45.4, 36.5, 35.6, 28.2, 27.8, 17.9, 17.8, −1.4, −1.5 ppm.

### 2-(Trimethylsilyl)ethyl (E)-4-hydroxy-3,4,7,8-tetrahydroazocine-1(2H)-carboxylate (3.29)

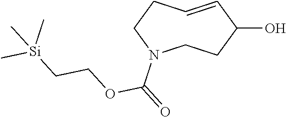 [SMILES: C[Si](C)(C)CCOC(=O)N1CC/C=C/C(O)CC1]

3.28 (1.11 g, 4.09 mmol) and 1.36 g methyl benzoate (10 mmol) in 4:1 hept/EtOAc was irradiated, the irradiated solution being continuously flushed through a column with 8 g 10% silver nitrate (4.70 mmol) on silica for 10 h. The column was flushed with TBME, TBME/5% MeOH and TBME/20% MeOH. All fractions, as well as the column material, were stirred for 10 min with 15 mL 25% ammonia/10 mL H₂O. The layers were separated, the upper layer was dried and concentrated yielding material (total 710 mg) consisting of 3.29 as a mixture of two isomers (largely the axial-TCO, which has a broad singlet at 4.67), and some minor impurities. ¹H-NMR (300 MHz, CDCl₃, positions of relevant signals): δ 5.9 (m), 5.55 (dd), 4.67 (bs), 4.2 (m), 3.6 (m), 2.6 (m), 2.5-2.0 (broad m), 1.9-1.6 (m), 1.3 (m), 1.1-0.8 (m), 0.0 (s) ppm.

### 2-(Trimethylsilyl)ethyl (S,E)-4-(((4-nitrophenoxy)carbonyl)oxy)-3,4,7,8-tetrahydroazocine-1(2H)-carboxylate (3.30)

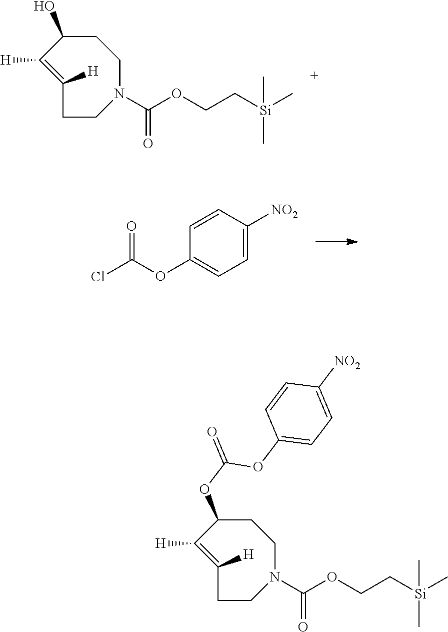

3.29 (115 mg; 0.424 mmol) was dissolved in CHCl₃ (10 mL), and DMAP (207 mg; 1.69 mmol) was added. The solution was cooled to 0° C., and 4-nitrophenyl chloroformate (128 mg; 0.636 mmol) was added. The mixture was stirred at 0° C. under an atmosphere of Ar for 1 h, and then washed with 0.5 M aq. citric acid solution (2×10 mL). The organic layer was isolated, dried over Na₂SO₄, filtered, and evaporated to dryness. The crude product was purified by column chromatography on silica gel, using an elution gradient of 5% to 25% EtOAc in n-hept. This yielded the pure isomers with the carbonate group in axial position (56 mg of a colorless oil), or equatorial position (36 mg of a colorless oil). axial isomer: ¹H-NMR (CDCl₃): δ 8.29 (d, J=9.3 Hz, 2H), 7.41 (d, J=9.3 Hz, 2H), 5.90 (m, 1H), 5.54 (m, 1H), 5.51 (s, 1H), 4.28 (m, 1H), 4.18 (m, 2H), 3.70 (m, 1H), 2.87-2.55 (br.m, 3H), 2.40 (m, 2H), 2.09 (t, J=15.0 Hz, 1H), 1.03 (m, 2H), 0.06 (s, 9H) ppm.

equatorial isomer: ¹H-NMR (CDCl₃): δ 8.29 (d, J=9.3 Hz, 2H), 7.40 (d, J=9.3 Hz, 2H), 5.82 (m, 1H), 5.63 (m, 1H), 5.28 (m, 1H), 4.37-4.22 (m, 1H), 4.27-4.12 (m, 2H), 3.85 (m, 1H), 2.67 (m, 2H), 2.55-2.26 (br.m, 4H), 1.05 (m, 2H), 0.06 (s, 9H) ppm.

### 2-(Trimethylsilyl)ethyl (S,E)-4-((dimethylcarbamoyl)oxy)-3,4,7,8-tetrahydroazocine-1(2H)-carboxylate (3.31)

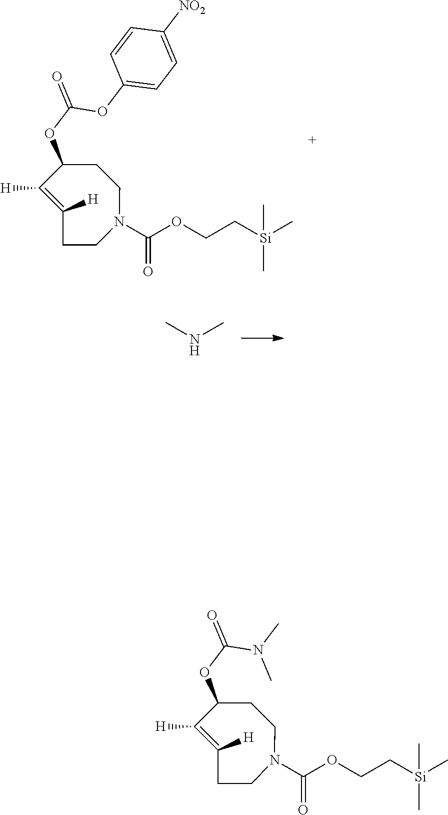

3.30 (axial isomer) (35 mg; 0.080 mmol) was dissolved in THF (3 mL). A solution of dimethylamine in THF (0.10 mL 2 M; 0.20 mmol) was added and the mixture was stirred at 20° C. for 30 min. The mixture was evaporated to dryness, dissolved in CHCl₃ (10 mL) and washed with subsequently 0.5 M aq. citric acid solution (2 times 5 mL), and 1 M aq. NaOH (2×5 mL). The organic layer was isolated, dried over Na₂SO₄, filtered, and evaporated to dryness to yield 3.31 as a colorless oil (29 mg). ¹H-NMR (CDCl₃): δ 5.70 (m, 1H), 5.52 (dd, J=16.4, 2.0 Hz, 1H), 5.42 (m, 1H), 4.30 (m, 1H), 4.17 (m, 2H), 3.63 (m, 1H), 2.97 (m, 6H), 2.74-2.23 (br.m, 5H), 1.97 (ddd, J=15.8, 13.0, 2.7 Hz, 1H), 1.01 (m, 2H), 0.06 (s, 9H) ppm. ¹³C-NMR (CDCl₃): δ 156.73, 156.16, 155.48, 155.46, 136.40, 135.69, 127.08, 126.26, 125.00, 122.20, 73.69, 73.55, 63.39, 63.20, 56.57, 55.93, 47.87, 46.77, 39.55, 37.96, 36.40, 36.02, 35.85, 35.57, 30.27, 17.94, 17.90, −1.50, −1.52 ppm.

### (S,E)-1,2,3,4,7,8-Hexahydroazocin-4-yl dimethylcarbamate (3.32)

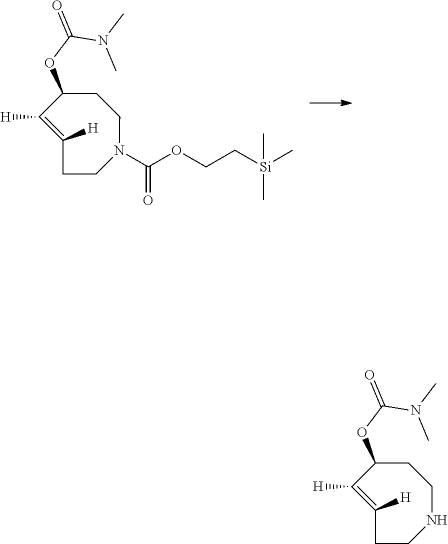

3.31 (7.2 mg; 0.021 mmol) was dissolved in MeCN (0.5 mL), and potassium fluoride (4.8 mg; 0.082 mmol) was added, followed by tetrabutylammonium fluoride (0.062 mL of a 1 M solution in THF; 0.062 mmol). The mixture was heated to 45° C. for 18 h, and then evaporated to dryness. The crude product was dissolved in CHCl₃ (1 mL), and washed with 0.1 M aq. sodium carbonate (3×1 mL). The organic layer was isolated, dried over Na₂SO₄, filtered, and evaporated to dryness to yield 3.32 as a colorless oil (4 mg). ¹H-NMR (CDCl₃): δ 5.79 (m, 1H), 5.63 (m, 1H), 5.46 (m, 1H), 3.24 (m, 1H), 3.03 (m, 1H), 2.59 (m, 1H), 2.41 (m, 1H), 2.22 (m, 1H), 2.02-1.76 (br.m, 3H) ppm. ¹³C-NMR (CDCl₃): δ 135.90, 127.09, 74.54, 58.90, 55.00, 46.45, 44.48, 38.84, 24.09, 19.76, 13.68 ppm.

### Example 4: Additional Synthesis Routes Towards Tetrazines of the Invention

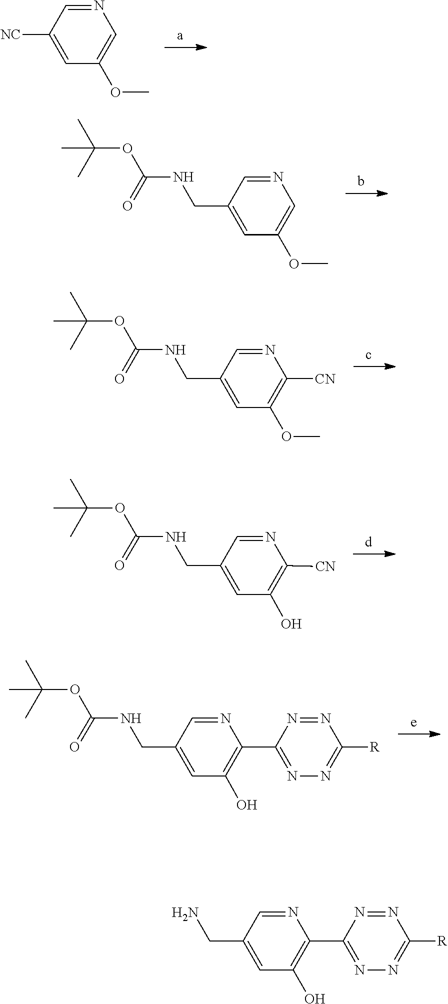

Reaction conditions: a) (i) Ra—Ni, H₂; (ii) BOC₂O, EtOH. b) (i) m-CPBA, CHCl₃; (ii) Dimethylcarbamoyl chloride, MeCN, then NaCN, H₂O. c) BBr₃, CH₂Cl₂, −78° C. d)(i) NH₂NH₂.xH₂O, 90° C.; (ii) AcOH, NaNO₂. e) TFA, CHCl₃. Step b has been reported in Philippou, WO 2019/186164 A1. When asymmetrical tetrazines are required, the appropriate nitrile is added in step d. For R═H, the asymmetrical tetrazine can be prepared by using the method described in Angew. Chem. Int. Ed. 2018, 57, 12057-12061. The methylene amine group can be conjugated to an R₈₇ group.

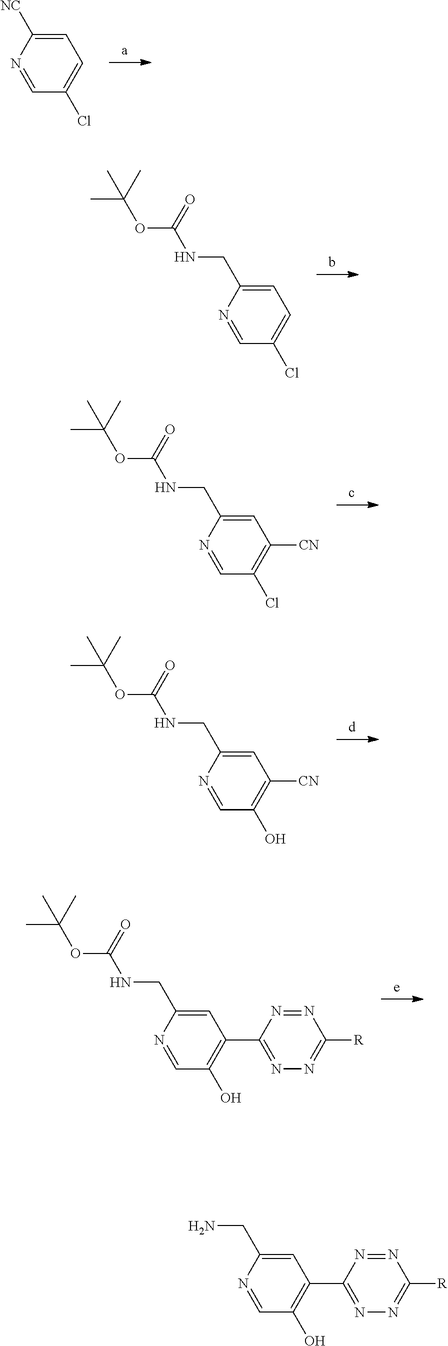

Reaction conditions: a) (i) Ra—Ni, H₂; (ii) BOC₂O, EtOH. b)(i) m-CPBA, CHCl₃; (ii) TMSCN, CHCl₃, 60° C.; (iii)N-methylmorpholine, CHCl₃, 60° C. c) (i) 2-(Trimethylsilyl)ethanol, NaH, THF, 50° C.; (ii) TBAF, THF. d) (i) NH₂NH₂.xH₂O, Zn(OTf)₂, 90° C.; (ii) AcOH, NaNO₂. e) TFA, CHCl₃. When asymmetrical tetrazines are required, the appropriate nitrile is added in step d. For R═H the asymmetrical tetrazine can be prepared using the method described in Angew. Chem. Int. Ed. 2018, 57, 12057-12061. The methylene amine group can be conjugated to an R₈₇ group.

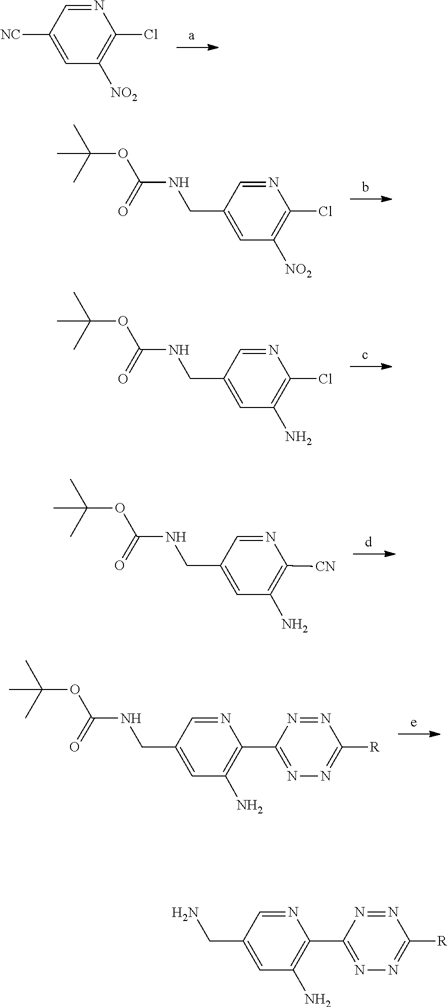

Reaction conditions: a) (i) NaBH₄, THF; (ii) BOC₂O, CH₂Cl₂. b) NH₄Cl, Fe, H₂O/EtOH, 90° C. c) Zn(CN)₂, Pd(PPh₃)₄, DMF, 100° C. d) (i) NH₂NH₂.xH₂O, 90° C.; (ii) PhI(OAc)₂, CH₂Cl₂. e) TFA, CHCl₃. When asymmetrical tetrazines are required, the appropriate nitrile is added in step d. For R═H the asymmetrical tetrazine can be prepared using the method described in Angew. Chem. Int. Ed. 2018, 57, 12057-12061. Either amine group can be conjugated to an R₈₇ group.

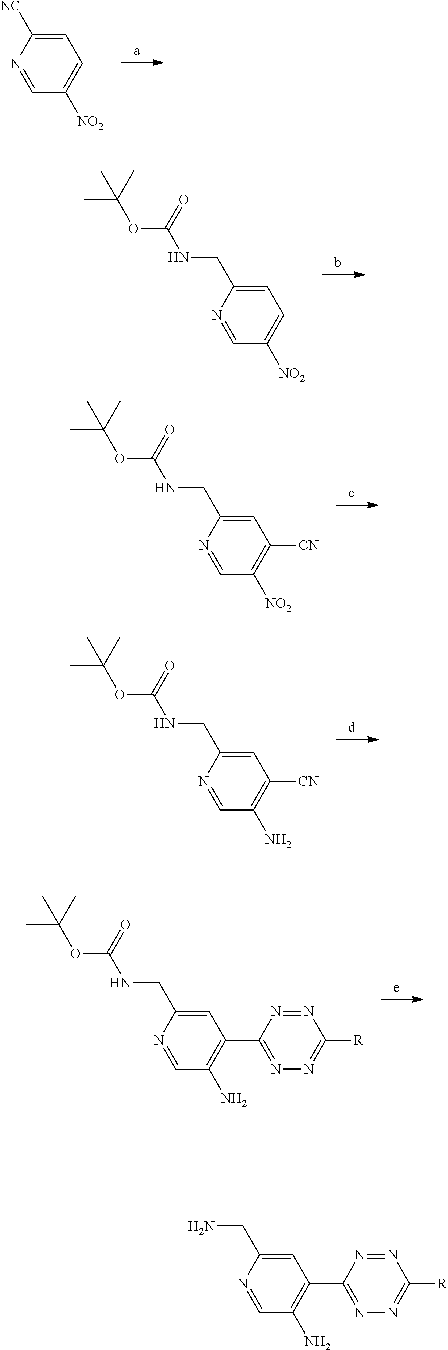

Reaction conditions: a) (i) NaBH₄, THF; (ii) BOC₂O, CH₂Cl₂. b) (i) m-CPBA, CHCl₃. (ii) TMSCN, CHCl₃, 60° C.; (iii)N-methylmorpholine, CHCl₃ 60° C. c) NH₄Cl, Fe, H₂O/EtOH, 90° C. d) (i) NH₂NH₂.xH₂O, 90° C.; (ii) PhI(OAc)₂, CH₂Cl₂. e) TFA, CHCl₃. When asymmetrical tetrazines are required, the appropriate nitrile is added in step d. For R═H the asymmetrical tetrazine can be prepared using the method described in Angew. Chem. Int. Ed. 2018, 57, 12057-12061. Either amine group can be conjugated to an R₈₇ group.

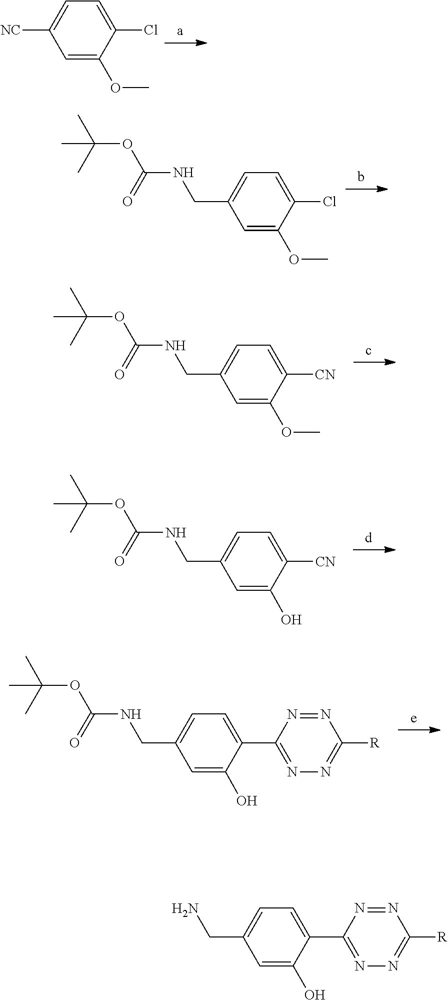

Reaction conditions: a) (i) Ra—Ni, H₂; (ii) BOC₂O, EtOH. b) Zn(CN)₂, Pd(PPh₃)₄, DMF, 100° C. c) BBr₃, CH₂Cl₂, −78° C. d) (i) NH₂NH₂.xH₂O, Zn(OTf)₂, 90° C.; (ii) AcOH, NaNO₂. e) TFA, CHCl₃. When asymmetrical tetrazines are required, the appropriate nitrile is added in step d. For R═H the asymmetrical tetrazine can be prepared using the method described in Angew. Chem. Int. Ed. 2018, 57, 12057-12061. The methylene amine group can be conjugated to an R₈₇ group.

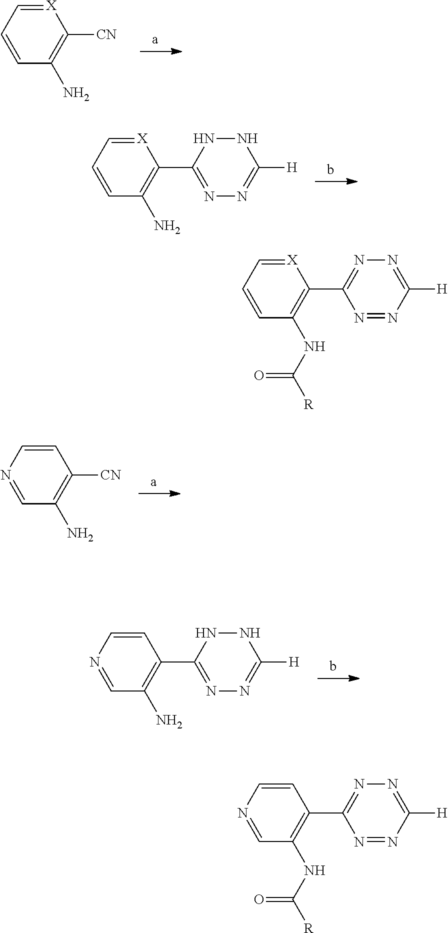

Reaction conditions: a) CH₂Cl₂, NH₂NH₂.xH₂O, S, 50° C. b) (i) RC(O)O(O)CR, dry THF, 50° C.; (ii) AcOH, NaNO₂. The reactant in b may be an anhydride or a mixed anhydride where the R-group that becomes attached optionally contains a (protected) functional group, allowing linkage to R₈₇. X═C, N.

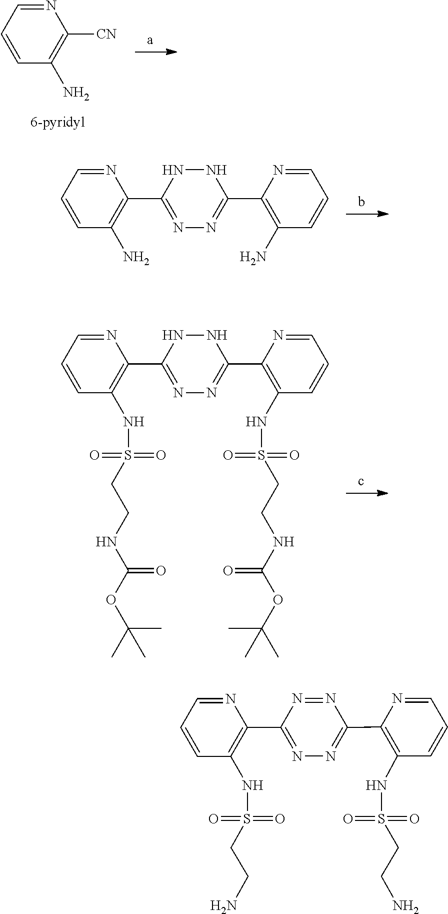

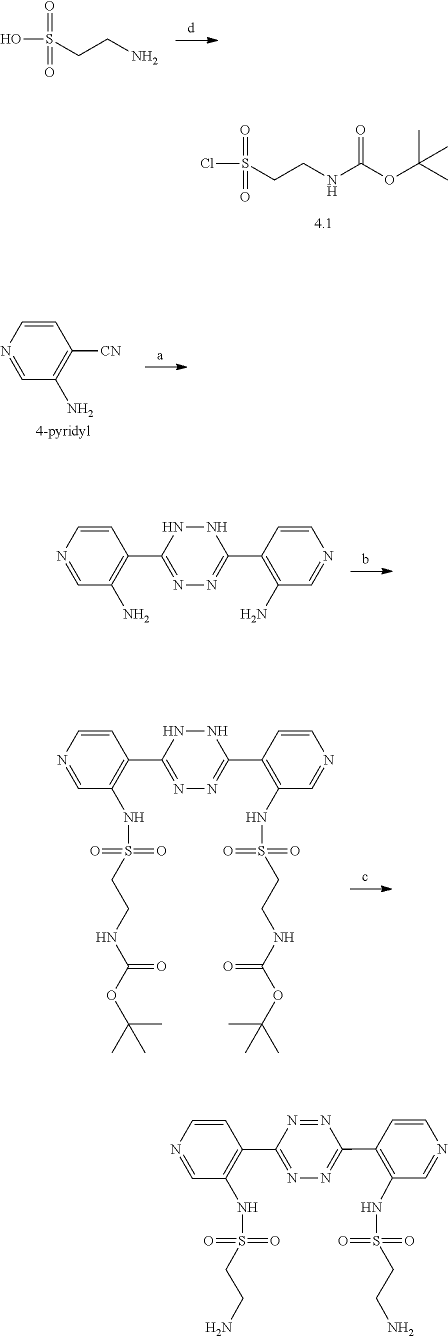 [SMILES: NCCS(=O)(=O)O ; CC(C)(C)OC(=O)NCCS(=O)(=O)Cl ; N#Cc1ccncc1N ; Nc1cnccc1C1=NN=C(c2ccncc2N)NN1 ; CC(C)(C)OC(=O)NCCS(=O)(=O)Nc1cnccc1C1=NN=C(c2ccncc2NS(=O)(=O)CCNC(=O)OC(C)(C)C)NN1 ; NCCS(=O)(=O)Nc1cnccc1-c1nnc(-c2ccncc2NS(=O)(=O)CCN)nn1]

Reaction conditions: a) NH₂NH₂.xH₂O, 90° C. (for 4-pyridyl also Zn(OTf)₂). b) (i) 1, N-methylmorpholine, DMF or THF; (ii) AcOH, NaNO₂. c) TFA, CHCl₃. d) (i) BOC₂O, Bu₄NOH, H₂O/Me₂CO; (ii) tri-phosgene, DMF (small amount), CH₂Cl₂. Compound 4.1 has been reported in, Leszczynska et al., Nucleosides, Nucleotides & Nucleic Acids 2013, 32, 599-616. Either amine group can be conjugated to an R₈₇ group.

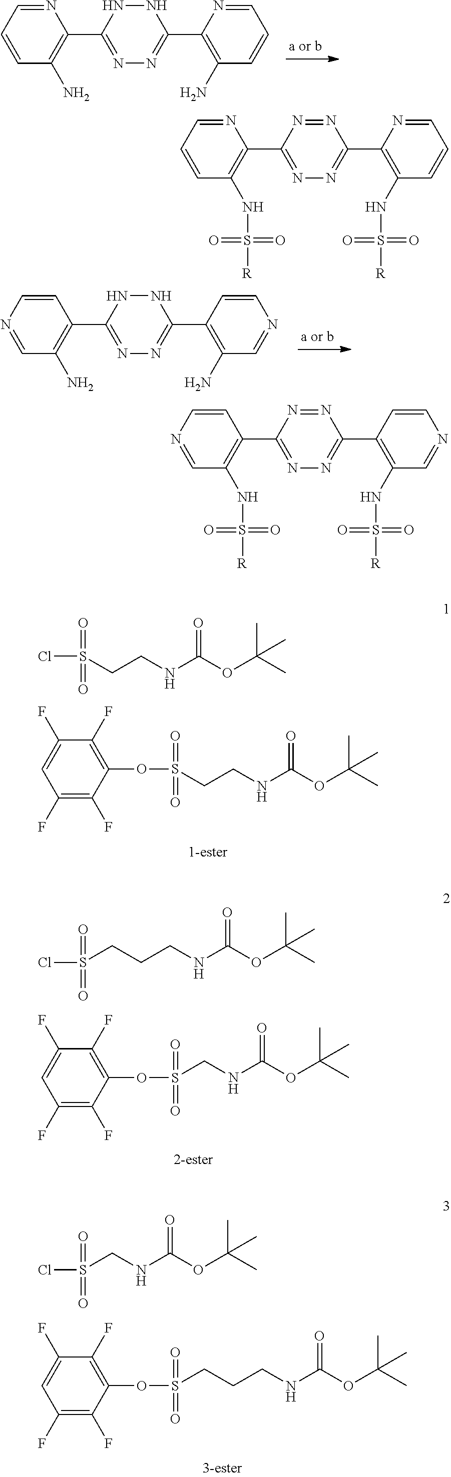

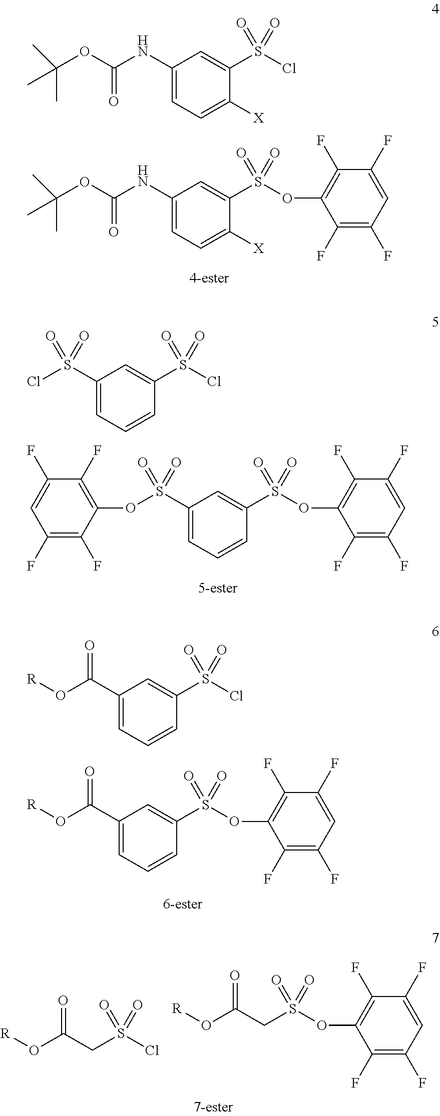

Reaction conditions: a) (i) one of 1 to 7, N-methylmorpholine (NMM), DMF or THF; (ii) AcOH, NaNO₂. b) (i) one of 1-ester to 7-ester, N-methylmorpholine (NMM), DMF, elevated temperature; (ii) AcOH, NaNO₂. Compounds 1-sulfonyl chloride to 7-sulfonyl chloride can be converted their respective TFP-esters by reaction with tetrafluoro-phenol in DMF, CHCl₃ or THF in the presence of base such as DIPEA, TEA, pyridine or NMM.

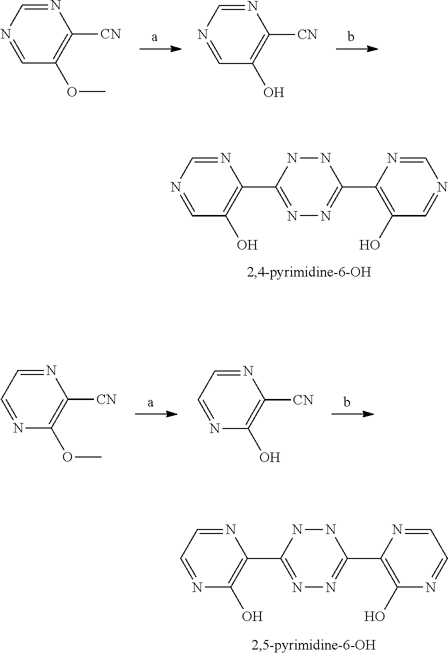

Reaction conditions: a) BBr₃, CH₂Cl₂, −78° C. b) (i) NH₂NH₂.xH₂O, 90° C.; (ii) AcOH, NaNO₂.

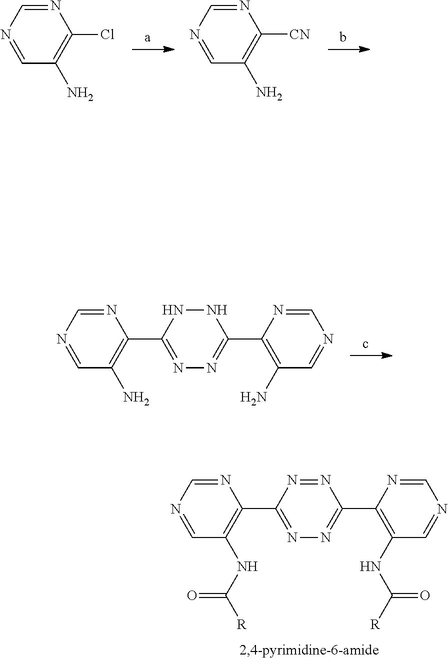

Reaction conditions: a) Zn(CN)₂, Pd(PPh₃)₄, DMF, 100° C. b) NH₂NH₂.xH₂O, 90° C. C) (i) RC(O)O(O)CR, dry THF, 50° C.; (ii) AcOH, NaNO₂. The reactant in c may be an anhydride or a mixed anhydride where the R-group that becomes attached optionally contains a (protected) functional group, allowing linkage to R₈₇.

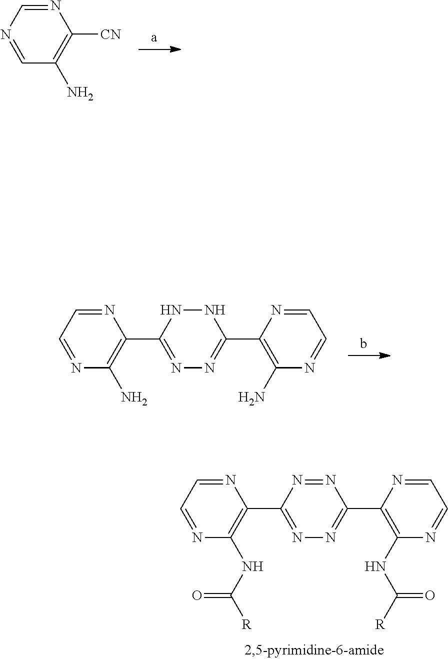

Reaction conditions: a) NH₂NH₂.xH₂O, 90° C. b) (i) RC(O)O(O)CR, dry THF, 50° C.; (ii) AcOH, NaNO₂ The reactant in b may be an anhydride or a mixed anhydride where the R-group that becomes attached optionally contains a (protected) functional group, allowing linkage to R₈₇.

### Example 5: Kinetic Measurements

The second-order rate constant of the IEDDA reaction between model TCO 3.2 and activators 2.7 or 2.8 in MeCN at 20° C. was determined (n=1) by UV-Vis spectrometry under second-order conditions. A cuvette was filled with a solution of activator (3 mL 0.083 mM in MeCN), and equilibrated at 20° C. Next, a solution of TCO 3.2 (10.0 μL, 25 mM in DMSO) was added. The absorption at 540 nm was measured every second in a time course experiment. From the decay of this absorption, the conversion x was calculated. The second-order rate constant k₂ was determined from the slope of the curve of a plot of 1/c-1/c₀ versus time. The second-order rate constant of the IEDDA reaction between model TCO 3.2 and activators 2.7, 2.8, 2.14, 2.16, or 2.17 in 25% MeCN/PBS at 20° C. was determined (n=5) by stopped-flow UV-Vis spectrometry under pseudo first-order conditions. Tetrazine solutions (0.25 mM in 25% MeCN/PBS) and TCO 3.2 (3.45 mM in in 25% MeCN/PBS) were mixed in a volume ratio of 1:1, resulting in a concentration of 0.125 mM and 1.73 mM, respectively. The absorption at 520 nm was measured every 50 ms, and from the decay of this absorption, the pseudo first-order rate constant k₁′ was determined by a nonlinear Simplex regression analysis of the data points using BioKine software. The second-order rate constant k₂ was calculated: k₂=k₁′/c,_{TCO}. As expected for a cycloaddition reaction, the reactivity in PBS is much higher than in MeCN, and importantly the tetrazines of the invention are at least 1 order of magnitude more reactive than the known and high-releasing 3,6-bismethyl-tetrazine (2.17), while giving similar release yields (see Example 7).

TABLE 2
Calculated k2 values (M⁻¹ s⁻¹) for the reaction between
tetrazine activators and TCO 3.2 in PBS (n = 5) and
MeCN (n = 1). The data in PBS are the mean ± SD (n = 5).

| activator | PBS | MeCN |
|---|---|---|
| 2.7 | 397 ± 4 | 68 |
| 2.8 | 270 ± 11 | 10 |
| 2.14 | 748 ± 20 | — |
| 2.16 | 519 ± 12 | — |
| 2.17 | 29.0 ± 0.4 | — |

### Example 6: Activator Stability

The assessment of activator stability was performed in PBS at rt. Solutions in DMSO (2.5 mM, 20 μL) were diluted with PBS (3.00 mL), filtered, and incubated at rt. The decrease of the absorption band at 520 nm was monitored using UV spectroscopy. The rate of hydrolysis and half-life time was derived by fitting a monoexponential decay through these data. The highly reactive tetrazines still show sufficient stability in aqueous solution at rt (Table 3), allowing their use in vivo.

TABLE 3
Activator half-lives in PBS at rt. Data are the mean ± SD (n = 3).

| activator | Half-life (h) |
|---|---|
| 2.7 | 37.8 ± 0.8 |
| 2.8 | 6.5 ± 0.6 |
| 2.14 | 8.7 ± 0.1 |
| 2.16 | 4.2 ± 0.1 |

### Example 7: Triggered Drug (MMAE) Release from ADC AVP0458-TCO-MMAE

In a 1.5 mL vial, the following was combined: 650 μL mouse plasma, 600 μL PBS, 20 μL ADC (1.62 μg/μL in PBS) and 7.5 μL d8-MMAE solution (0.167 μg/μL, internal standard). Next, aliquots of 20 μL were prepared to which 5.2 μL of a tetrazine solution (0.25 mM in 1:1 DMSO/H₂O) was added and the solution was incubated at 37° C. At various times, 100 μL of cold MeCN was added to the aliquot and the mixture was vortexed, stored at 4° C. for 30 min. and centrifuged. 20 μL of supernatant was taken and combined with 100 μL of 1% formic acid (aq) solution. This sample was analysed with HPLC-SIM-MS to quantify the ratio of free MMAE and d8-MMAE, which is a measure of the release yield. The positive control tetrazine, the slow reacting/high releasing 3,6-bismethyl-tetrazine (2.17) afforded high MMAE release after 24h incubation, while the negative control tetrazines, 3,6-bis(2-pyridinyl)-tetrazine and 3,6-bis(4-pyridinyl)-tetrazine (2.18 and 2.18; lacking an R₁ group) only gave 11-16% release (Table 4). The release achieved with the 2.7, 2.8, 2.14 and 2.16 is as high as that obtained with the less reactive 2.17, and in the case of 2.7 and 2.8 the release is also faster.

TABLE 4
MMAE release from ADC AVP0458-TCO-MMAE at various times after
reaction with activators in 50% mouse plasma at 37° C.

Release (%)

| activator | 2 min | 10 min | 1 h | 5 h | 24 h^{a} |
|---|---|---|---|---|---|
| 2.1 | — | — | — | — | 32.1 ± 0.7^{b} |
| 2.2 | — | — | — | — | 79.3 ± 0.4 |
| 2.3 | — | — | — | — | 88.5 ± 0.1 |
| 2.4 | — | — | — | — | 96.0 ± 0.3 |
| 2.7 | 53.8 | 80.1 | 80.9 | 83.8 | 98.4 ± 0.3 |
| 2.8 | 48.7 | 83.4 | 92.7 | 93.6 | 94.3 ± 1.3 |
| 2.14 | 0.6 | 2.1 | 16.6 | 49.9 | 80.5 ± 0.3 |
| 2.16 | 1.9 | 19.4 | 70.7 | 82.8 | 95.6 ± 0.6 |
| 2.17 | 2.5 | 44.8 | 79.4 | 87.7 | 96.4 ± 0.7 |
| 2.18 | — | — | — | — | 11.4 ± 1.8 |
| 2.19 | — | — | — | — | 16.0 ± 0.5 |

^{a}mean ± SD (n = 3);
^{b}low release due to low activator solubility in this medium.

### Example 8: Triggered Release of Construct-A from Range of TCO Triggers and Construct-A Types

A solution of the derivatives 3.2, 3.7, 3.8, 3.9, 3.10, and 3.11 (10 μL, 25 mM in DMSO) is diluted with MeCN (250 μL) and PBS (750 μL). Next, a solution of the activator 2.2 or 2.7 (20 μL 25 mM in DMSO) was added, and the pink solution was homogenized. At the specified time point, the sample was analyzed by HPLC-MS/PDA, and the release yield was determined from the observed amounts of IEDDA adducts, elimination product, and/or released Construct. In case the release was not complete, the sample was incubated at 37° C. for additional time and analyzed once again. Table 5 demonstrates that the triggered release works over a range of TCO designs, and Construct-A types.

TABLE 5
Construct release from various TCO derivatives
upon incubation with an activator

| | Released | | Time point and |
|---|---|---|---|
| TCO | construct | Activator | release % |
| 3.2 | dimethylamine | 2.7 | 5 min: 50% |
| | | | 2 h: 80% |
| | | | 20 h: 95% |
| 3.7 | 3-phenylpropan-1-ol | 2.7 | 5 min: 74% |
| | | | 24 h: 88% |
| | | | 48 h: 95% |
| 3.8 | benzoic acid | 2.7 | 5 min: 45% |
| | | | 24 h: 90% |
| 3.9 | phenol | 2.7 | 5 min: 50% |
| | | | 24 h: 93% |
| 3.10 | doxorubicin | 2.7 | 5 min: 52% |
| | | | 18 h: 100% |
| 3.11 | doxorubicin | 2.2 | 5 min: 4% |
| | | | 18 h: 74% |
| | | | 42 h: 81% |
| | | 2.7 | 5 min: 100% |
| 3.23 | dimethylamine | 2.2 | 5 min: 100% |
| | | 2.7 | 5 min: 100% |
| 3.32 | dimethylamine | 2.2 | 5 min: 98% |
| | | 2.7 | 5 min: 100% |

### Example 9: Protein-Protein Cleavage for In Vitro and In Vivo Applications

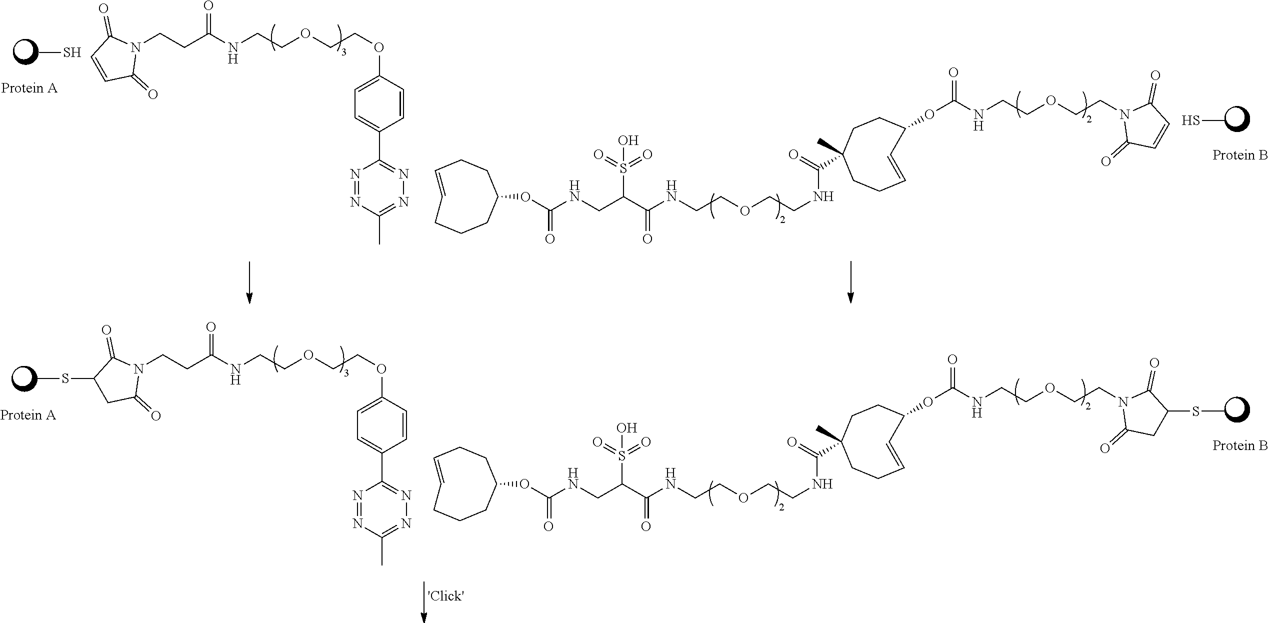

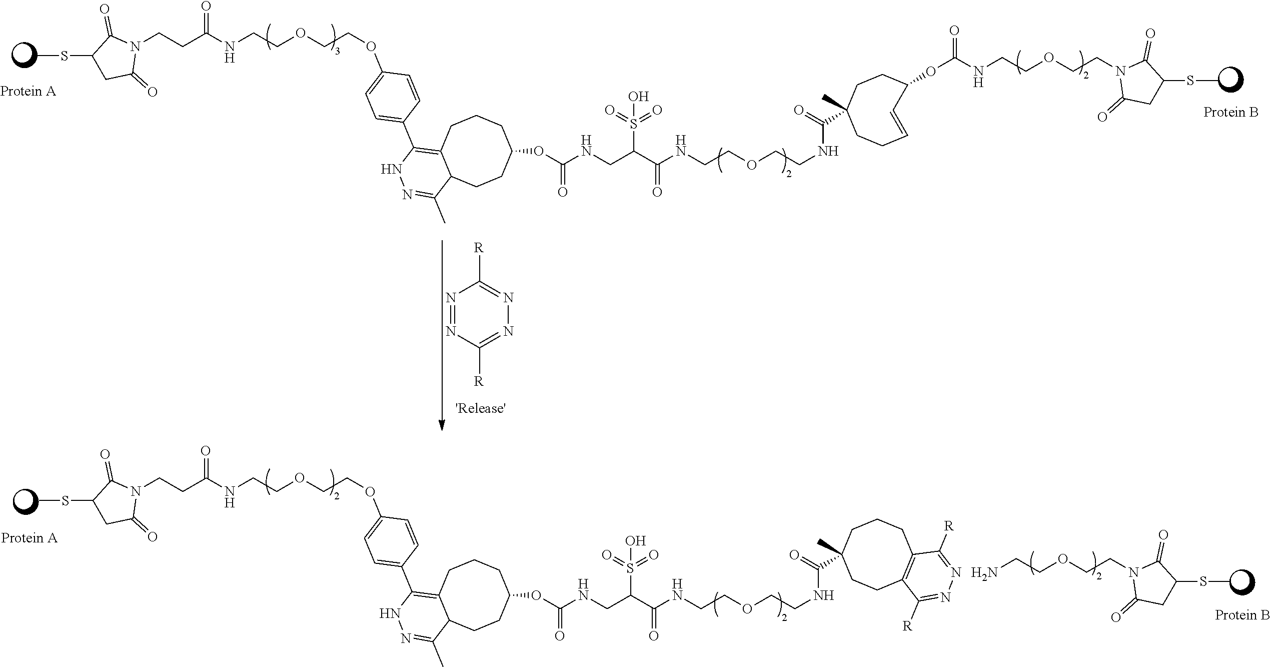

This example features the use of the invention for the cleavage of a protein-protein conjugate in vitro or in vivo. Controlled cleavage of the protein-protein bond may be used for the activation of a protein drug, or deactivation of a drug conjugate. The protein-protein conjugate is prepared via functionalization of one protein with a conjugatable tetrazine (e.g. 3-(2,5-dioxo-2,5-dihydro-1H-pyrrol-1-yl)-N-{2-[2-(2-{2-[4-(6-methyl-1,2,4,5-tetrazin-3-yl)phenoxy]ethoxy}ethoxy)ethoxy]ethyl} propanamide). The other protein is conjugated through a cysteine with cleavable linker 3.6 that enables subsequent click conjugation to the tetrazine functionalized protein. Maleimide-TCO-TCO linker 3.6 comprises two TCO moieties that can be selectively manipulated, as the TCO tag on the outside is ca 20-fold more reactive than the TCO linker, and will preferably react with the protein-bound tetrazine. Then the protein conjugate can be cleaved by addition of a slight excess of tetrazine activator, e.g. 2.16, and analyzed by SEC.

### Example 10: Protein-Protein Conjugation and Subsequent Release In Vitro

An anti-TAG72 antibody scFv fragment (MW ca. 25 kDa) was reacted with 5 molar equiv. of TCO Trigger (I) in PBS at pH ca. 9. After overnight incubation at +4° C., SDS-PAGE confirmed the presence in solution of two new protein species with a MW of ca. 50 and 75 kDa, signifying the formation of a dimeric and a trimeric species conjugated via the TCO. The mixtures were then added with an excess of activators 2.2, 2.3, 2.7, 2.14, 2.16, 2.18 or 2.19 and incubated overnight at 37° C. followed by SDS-PAGE analysis and Coomassie blue staining. Digital quantification of the band intensities showed a decrease of the species with 50 and 75 kDa MW and an increase in the 25 kDa band of the original mAb fragment, signifying TCO cleavage, as summarized in Table 6.

TABLE 6
Monomeric scFv fragment band intensity after
overnight incubation with various activators

| activator | monomer |
|---|---|
| 2.7 | +++ |
| 2.14 | +++ |
| 2.16 | +++ |
| 2.18 | ++ |
| 2.19 | ++ |

### Example 11: Protein PEGylation and Subsequent De-PEGylation In Vitro

An anti-TAG72 antibody scFv fragment (MW ca. 25 kDa) and lysozyme (MW ca. 14 kDa) were reacted with 20 molar equiv. of Masking Moiety 3.1 in PBS at pH ca. 9. After overnight incubation at +4° C., SDS-PAGE confirmed the presence in solution of two new species resulting from the conjugation of one or two TCO-PEG masking moieties to the proteins. The mixtures were then added with an excess of activators 2.2, 2.3, 2.7, 2.14, 2.16, 2.18 or 2.19 and incubated overnight at 37° C. followed by SDS-PAGE analysis and Coomassie blue staining. Digital quantification of the band intensities showed a decrease of the mono- and bis-PEGylated species and an increase of the native bands, signifying TCO cleavage as summarized in Table 7.

TABLE 7
Increase in native band intensities after overnight incubation
of the PEGylated derivatives with various activators

| | mAb | |
|---|---|---|
| activator | fragment | lysozyme |
| 2.2 | ++ | + |
| 2.7 | +++ | +++ |
| 2.14 | ++ | ++ |
| 2.16 | +++ | +++ |
| 2.18 | − | − |
| 2.19 | − | − |

### Example 12: Antibody Conjugation with Linker 3.6, Radiolabeling and Subsequent Triggered Label Release In Vitro

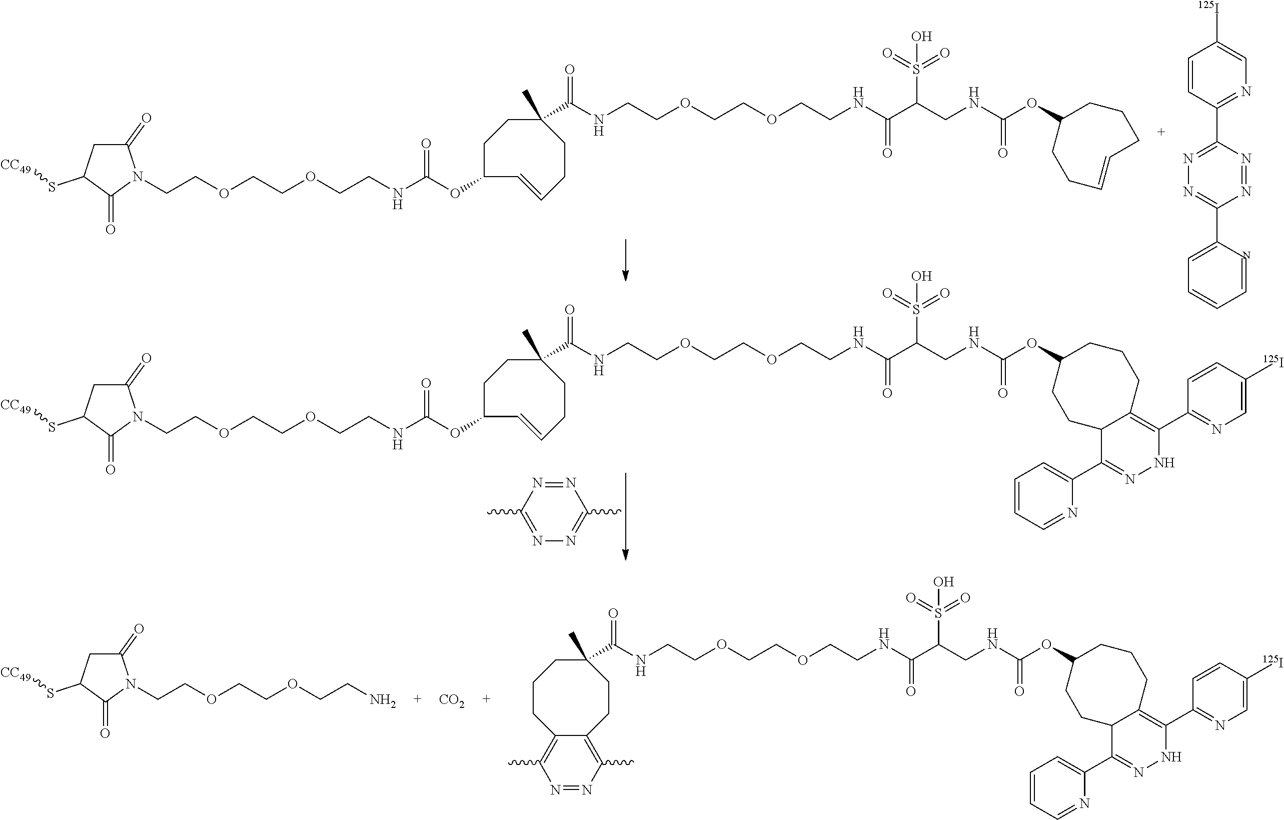

The anti-TAG72 mAb CC49 was conjugated with 3.6 in borate buffer pH 8.0 (containing 1 mM DTPA) using 3 equiv. TCEP. After conjugation, the product was dialyzed extensively (20 kDa MW cut-off membrane) in PBS. The CC49-3.6 the conjugate was radiolabeled using a previously described ¹²⁵I-labeled tetrazine [Albu et al., Bioconjug Chem 2016, 27, 207-216] in PBS at 37° C. for 1 h. Due to the higher reactivity of the TCO tag on the outside vs the cleavable TCO trigger on the inside the radiolabeling occurred selectively via the TCO tag. The crude labeling mixture was purified using a desalting cartridge (Zeba spin column, 40 kDa MW cut-off) then the isolated ¹²⁵I-labeled CC49 conjugate was incubated with activator 2.7 for 2h in PBS at 37° C., resulting in reaction with the TCO trigger, yielding 88.6% radiolabel release, as confirmed by SEC analysis.

### Example 13: Cell Proliferation Assay

The human colon carcinoma LS174T cell line was obtained from the American Type Culture Collection and cultured in RPMI-1640 medium supplemented with 2 mM glutamine and 10% heat inactivated fetal calf serum. Twenty-four hours prior to the experiment, the cells were plated in 96-well plates at a 5000 cells/well density. The cells were then incubated with serial dilutions (10 nM-1 μM; n=6) of activators 2.7, 2.8, 2.14, 2.16, 2.17, 2.18 and 2.19 in culture medium for 72h at 37° C. The % cell proliferation was assessed by an MTT assay, in comparison to cells incubated in culture medium without activator. No toxicity was observed when the LS174T cells were incubated with the tested activators (and possible metabolites formed in the presence of serum) for 3-days at a 0.01-1 μM activator concentration.

TABLE 8
LS174T cell viability (%) after 3 days incubation in the
presence of various concentrations of activators. Data
are the mean ± SEM (n = 6).

| | Concentration (μM) | | |
|---|---|---|---|
| activator | 0.01 | 0.1 | 1 |
| 2.7 | 93.6 ± 4.1 | 91.5 ± 4.4 | 98.2 ± 4.6 |
| 2.8 | 96.9 ± 3.6 | 98.4 ± 3.6 | 93.9 ± 5.2 |
| 2.14 | 107.0 ± 3.0 | 104.0 ± 2.7 | 97.0 ± 2.5 |
| 2.16 | 104.9 ± 7.4 | 96.9 ± 3.0 | 93.9 ± 2.8 |
| 2.17 | 90.2 ± 4.5 | 87.9 ± 6.4 | 85.7 ± 7.4 |
| 2.18 | 100.6 ± 3.0 | 100.8 ± 2.8 | 101.9 ± 3.6 |
| 2.19 | 87.0 ± 2.6 | 88.7 ± 2.5 | 91.3 ± 3.0 |

### Example 14: In Vivo Reactivity Between ADC AVP0458-TCO-MMAE and Tetrazine Activators

Female nude Balb/C mice were subcutaneously inoculated ca. 1×10⁶ LS174T cells in the hind limb. Biodistribution studies started when the tumors reached 0.1-0.2 cm³ size. Experiments were carried out to evaluate the reactivity between the TCO Trigger on tumor-bound ADC and activators 2.7, 2.8, 2.14 and 2.16 in LS174T xenografts, as previously performed for other activators [Rossin et al Nature Communications 2018, 9, 1484]. The activators were administered at a 20- to 100-lower dose than that used in previous activation and therapy studies (ca. 0.335 mmol/kg). The highly reactive probe ¹¹¹In-2.20 was used as a reporter to show the presence of residual (unreacted) TCO moieties in the tumors of mice treated with ADC followed by the activators, in comparison to mice that did not receive the activator. Groups of tumor bearing mice (n=4) were injected with ¹²⁵I-labeled ADC (2 mg/kg) followed 48 h later by the activators (dose 1×: ca. 3.3 μmol/kg; dose 5×: ca. 16.7 μmol/kg) and, after 1 h, by [¹¹¹In]In-2.20 (ca. 0.33 μmol/kg). One group was injected with the same amount of ¹²⁵I-labeled ADC followed only by ¹¹¹In-2.20 49 h later. One group was injected only with ¹¹¹In-2.20 (non-specific probe binding). All mice were euthanized 23 h post-probe injection and the ¹²⁵I and ¹¹¹In uptakes in tumors were measured by γ-counting.

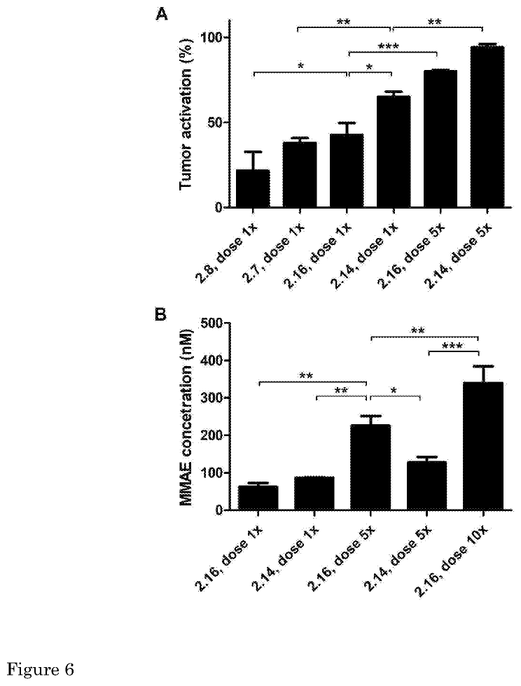

Biodistribution data revealed a relatively low in vivo reaction yield (20-40%) between the TCO and the smaller activators 2.7 and 2.8 while significantly higher yields (43-65%) were obtained with 2.14 and 2.16 where the tetrazine derivatives were functionalized with short PEG chains (i.e. an R⁸⁷ moiety) (FIG. 6A). This higher TCO binding in tumors is most likely due to a combination of higher reactivity and longer circulation in blood of the PEGylated tetrazines with respect to the OH containing counterparts. At higher dose both activators produced higher on-tumor reaction yields with the ADC, with 2.14 achieving almost complete tumor activation (94.2±1.8%).

### Example 15: MMAE Measurement in Tumors

The concentration of MMAE released in tumors was measured as previously reported [Rossin et al Nature Communications 2018, 9, 1484]. No difference in free MMAE was detected in the tumors harvested from mice 24 h after administration of 2.14 and 2.16 at dose 1× while a significantly higher release was achieved with a 5× dose of activator 2.16, most likely due to the higher release achievable with 2.16 with respect to 2.14 (Table 4, FIG. 6B). For this reason, activator 2.16 was selected for a therapy study in combination with the click-cleavable ADC. At the doses of tc-ADC (4 mg/kg) and 2.16 (dose 10×; 33.5 μmol/kg) used for therapy study, a high level of 339±45 nM free MMAE was detected in tumors.

### Example 16: Therapy Studies in Mice with LS174T Xenografts

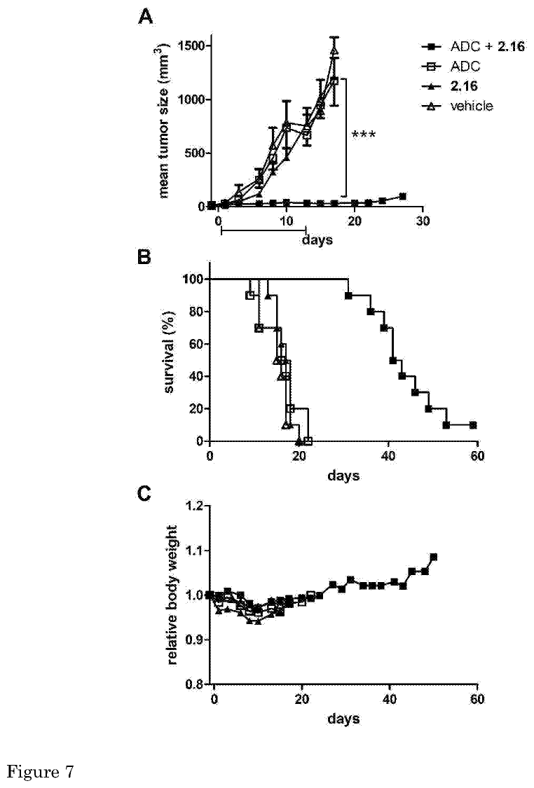

Groups of 10 female nude Balb/C mice were s.c. inoculated ca. 1×10⁶ LS174T cells in the hindlimb and the therapy study started when the tumors became palpable (5-11 days after tumor cell inoculation). One group was administered four cycles (one cycle every 4 days) of tc-ADC (ca. 4 mg/kg) followed by activator 2.16 (ca. 33.5 μmol/kg) 48 later. Control groups received four cycles of ADC alone, activator alone, or vehicle. Tumor sizes and body weights were measured 2-3 times per week. In the group that received the ADC and activator 2.16 the growth of very aggressive LS174T tumors was completely inhibited for over 3 weeks with a 42 days median overall survival (FIGS. 7A and B). On the contrary, all control mice were euthanized within 3 weeks from the beginning of the treatment due to large tumor size (>1.5 gr) or poor health conditions with a 15.5-17.5 days median survival. Overall, the repeated treatments were tolerated well by the mice, with no overt signs of hematological toxicity that could be attributed to the use of ADC and activator, either in combination or alone. During treatment the mice exhibited minor weight losses (5% max) but the animals that survived over 15 days eventually recovered (FIG. 7C).

1. A compound according to Formula (1): 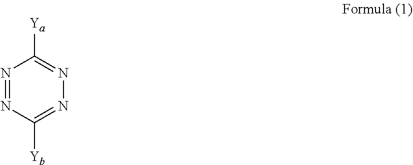
and salts thereof,
wherein, Yₐ is selected from the group consisting of Y₁, Y₂, Y₃, Y₄, Y₅ and Y₆: 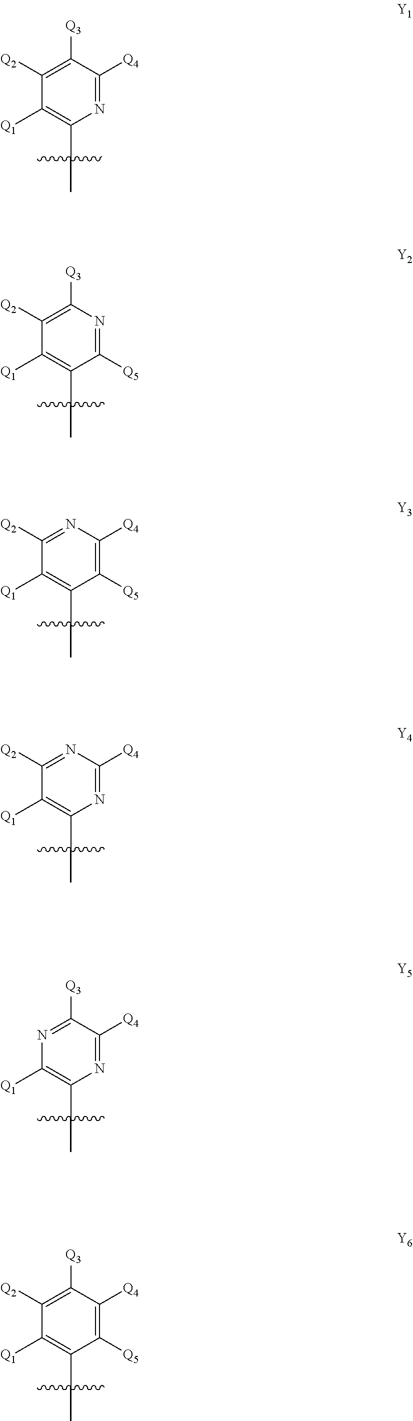
wherein,
Y_{b} is selected from the group consisting of Y₁, Y₂, Y₃, Y₄, Y₅, Y₆, hydrogen, R₃, and —(S^{P})_{D}—R₈₇;
wherein S^{P} is a spacer and D is 0 or 1;
wherein when Yₐ is Y₆, then Y_{b} is hydrogen,
wherein each Q₁ and Q₅, are individually selected from the group consisting of R₁, hydrogen, R₃ and —(S^{P})_{D}—R₈₇;
wherein each Q₂ and Q₄, are individually selected from the group consisting of R₂, hydrogen, R₃, and —(S^{P})_{D}—R₈₇;
wherein each Q₃ is individually selected from the group consisting of hydrogen, R₃, and —(S^{P})_{D}—R₈₇;
wherein, the compound of Formula (1) comprises at least one R₁ group, and at least one R₈₇;
wherein each R₈₇ is individually selected from the group consisting of biomolecule, polymer, peptide, peptoid, dendrimer, protein, carbohydrate, oligonucleotide, oligosaccharide, lipid, micelle, liposomes, polymersome, nanoparticle, microparticle, bead, gel, resin, metal complex, organometallic moiety, organic compound, albumin-binding moiety, dye moiety, fluorescent moiety, radionuclide-containing moiety and imaging probe;
wherein each R₁ individually is selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅i)₂, NX₅₀SO₂X₅₁, NX₅₀SO₃X₅₁, NX₅₀OX₅₁, SO₃H, and PO₃H₂;
wherein each R₂ individually is selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅i)₂, NX₅₀SO₂X₅₁, NX₅₀SO₃X₅₁, NX₅₀OX₅₁, SO₃H, and PO₃H₂;
wherein each X₅₀ and X₅₁ individually is selected from the group consisting of hydrogen, R₆, and —(S^{P})_{D}—R₈₇;
wherein each R₆ is independently selected from the group consisting of hydrogen, C₁-C₄ alkyl groups, C₂-C₄ alkenyl groups, and C₄₋₆ (hetero)aryl groups;
wherein for R₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂, and —NO₂; and optionally contain at most two heteroatoms selected from the group consisting of —O—, —S—, —NH—, —P—, and —Si—, wherein the N, S, and P atoms are optionally oxidized,
wherein each R₃ is individually selected from the group consisting of —F, —Cl, —Br, —I, —OR₇, —N(R₇)₂, —SO₃, —PO₃⁻, —NO₂, —CF₃, —SR₇, —S(═O)₂N(R₇)₂, OC(═O)R₇, SC(═O)R₇, OC(═S)R₇, SC(═S)R₇, NR₇C(═O)—R₇, NR₇C(═S)—R₇, NR₇C(═O)O—R₇, NR₇C(═S)O—R₇, NR₇C(═O)S—R₇, NR₇C(═S)S—R₇, OC(═O)N(R₇)₂, SC(═O)N(R₇)₂, OC(═S)N(R₇)₂, SC(═S)N(R₇)₂, NR₇C(═O)N(R₇)₂, NR₇C(═S)N(R₇)₂, C(═O)R₇, C(═S)R₇, C(═O)N(R₇)₂, C(═S)N(R₇)₂, C(═O)O—R₇, C(═O)S—R₇, C(═S)O—R₇, C(═S)S—R₇, —S(O)R₇, —S(O)₂R₇, NR₇S(O)₂R₇, —ON(R₇)₂, —NR₇OR₇, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₇, —N(R₇)₂, —SO₃R₇, —PO₃(R₇)₂, —PO₄(R₇)₂, —NO₂, —CF₃, ═O, ═NR₇, and —SR₇, and optionally contain one or more heteroatoms selected from the group consisting of O, S, NR₇, P, and Si, wherein the N, S, and P atoms are optionally oxidized, wherein the N atoms are optionally quaternized;
wherein each R₇ is individually selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

2. A compound according to claim 1, wherein for each individual Yₐ and Y_{b} at most three of Q₁, Q₂, Q₃, Q₄, and Q₅ are not hydrogen.

3. A compound according to claim 1, satisfying any one of Formulae (2), (4), or (6): 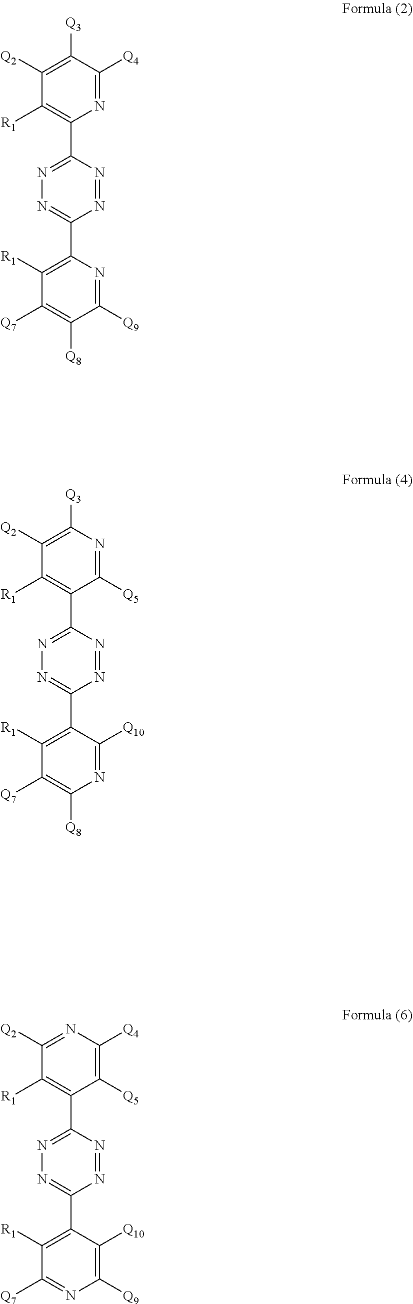
wherein Q₇ is as defined for Q₂, Q₈ is as defined for Q₃, Q₉ is as defined for Q₄, and Q₁₀ is as defined for Q₅.

4. The compound according to claim 1, wherein R₈₇ has a molecular weight of at least 100 Da

5. The compound according to claim 1, wherein R₈₇ is a polymer.

6. The compound according to claim 1, wherein R₁ is NHC(O)X₅₁.

7. The compound according to claim 1, comprising at most two R₈₇ groups.

8. The compound according to claim 3, wherein both R₁ groups in each of Formulae (2), (4), and (6) are identical.

9. A combination comprising the compound according to claim 1 and a dienophile.

10. The combination according to claim 9, wherein the dienophile is an eight-membered non-aromatic cyclic alkene.

11. The combination according to claim 10, wherein the eight-membered non-aromatic cyclic alkene carries a releasable group on the allylic position.

12. (canceled)

13. (canceled)

14. (canceled)

15. An in vitro method for releasing a moiety from a dienophile, said in vitro method comprising the step of contacting a compound as defined in claim 1 with an eight-membered non-aromatic cyclic alkene that carries a releasable moiety on an allylic position.

16. A compound according to claim 1, wherein the compound is a pharmaceutically acceptable salt.

17. A compound according to claim 1, wherein for each individual Yₐ and Y_{b} at most two of Q₁, Q₂, Q₃, Q₄, and Q₅ are not hydrogen.

18. A compound according to claim 3, wherein the compound is a pharmaceutically acceptable salt.

19. A compound according to claim 3, wherein for each individual Yₐ and Y_{b} at most two of Q₁, Q₂, Q₃, Q₄, and Q₅ are not hydrogen.

20. The compound according to claim 3, wherein R₈₇ has a molecular weight of at least 100 Da.

21. The compound according to claim 3, wherein R₈₇ has a molecular weight in a range of from 100 Da to 3000 Da.

22. The compound according to claim 3, wherein R₈₇ is a polymer.

23. The compound according to claim 22, wherein the polymer is polyethylene glycol.

24. The compound according to claim 3, wherein R₁ is NHC(O)X₅i.

25. The compound according to claim 24, wherein X₅₁ is R₈₇.

26. A compound according to claim 3, wherein R₁ is OH.

27. The compound according to claim 4, wherein R₈₇ has a molecular weight in a range of from 100 Da to 3000 Da.

28. The compound according to claim 5, wherein the polymer is polyethylene glycol.

29. The compound according to claim 6, wherein X₅₁ is R₈₇.

30. The combination according to claim 10, wherein the cyclooctene is a trans-cyclooctene.

31. A method for the targeted delivery of drugs to a patient in need thereof, the method comprising administering to the patient a compound according to claim 1 or a pharmaceutically acceptable salt thereof and an eight-membered non-aromatic cyclic alkene that carries a releasable drug D^{D} on an allylic position.

32. A method of treating a patient suffering from a disease that can be modulated by a drug, the method comprising administering to the patient a prodrug comprising an eight-membered non-aromatic cyclic alkene that carries a releasable Drug D^{D} on an allylic position and a compound according to claim 1 or a pharmaceutically acceptable salt thereof.

33. A method according to claim 32, wherein the patient suffers from a cancer, an inflammation, an infection, a cardiovascular disease or disorder, or a brain disorder.


SEQUENCE LISTING (1 sequence)

SEQ ID NO: 1 - protein, 234 aa, Artificial Sequence
  TAG72-binding diabody derived from the CC49 antibody
SVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSDIVMTQSCSSCPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKR